(19) United States
(12) Patent Application Publication
(10) Pub. No.: US 2019/0085081 A1
(43) Pub. Date: Mar. 21, 2019

(54) CHIMERIC ANTIGEN RECEPTOR

(71) Applicants: Cancer Research Technology Limited, London (GB); The University of Birmingham, Birmingham, West Midlands (GB); Cell Therapy Catapult Limited, London (GB)

(72) Inventors: Roy BICKNELL, Birmingham (GB); Steven LEE, Birmingham (GB); Natalie MOUNT, London (GB); Michaela SHARPE, London (GB)

(21) Appl. No.: 16/085,189

(22) PCT Filed: Mar. 14, 2017
(86) PCT No.: PCT/GB2017/050686

§ 371 Date: Sep. 14, 2018

(30) Foreign Application Priority Data
Mar. 15, 2016 (GB) 1604387.9
Jul. 19, 2016 (GB) 1612533.8
Jul. 25, 2016 (GB) 1612844.9

(51) Int. Cl. C07K 16/28; C12N 15/62; A61K 35/17; C12N 15/85; C07K 14/15; C07K 14/52; C07K 14/495; C07K 14/705; A61P 35/00

(52) U.S. Cl. CPC C07K 16/2851; C12N 15/62; A61K 35/17; C12N 15/85; A61P 35/00; C07K 14/52; C07K 14/495; C07K 14/70596; C07K 14/15

(57) ABSTRACT

The present invention is directed to a nucleic acid molecule comprising a polynucleotide sequence encoding a chimeric antigen receptor comprising (a) an anti-CLEC14A binding domain, (b) a transmembrane domain and (c) an intracellular signalling domain; wherein said anti-CLEC14A binding domain is capable of binding to the C-type lectin domain of CLEC14A. The invention further provides a chimeric antigen receptor encoded by the nucleic acid molecule, a vector comprising the nucleic acid molecule, a cell comprising the nucleic acid molecule, vector or CAR and the therapeutic use of the nucleic acid, vector or cell, particularly in treating CLEC14A expressing conditions.

The present invention is concerned with chimeric antigen receptors, particularly chimeric antigen receptors which specifically bind to CLEC14A. The invention further relates to polynucleotides encoding the chimeric antigen receptors and cells comprising the receptors and/or their encoding polynucleotides. Use of the chimeric antigen receptors, polynucleotides and/or cells of the invention to inhibit tumour angiogenesis and/or cancer are also encompassed.

Blood vessels are lined with a single layer of endothelial cells which form an interface between the blood stream and the surrounding tissues. New blood vessels develop from the walls of existing small vessels by the outgrowth of endothelial cells in a process called angiogenesis. Endothelial cells usually remain quiescent after the development of the vascular system, with no new vessel formation, with the exception of vessel formation in wound healing. However, during solid tumour growth, vessel formation may occur in response to the secretion of factors by tumours promoting the stimulation of endothelial cells to construct new capillary sprouts. Tumour angiogenesis is widely recognised as a rate limiting process in the growth of solid tumours and thus plays an important role in tumour progression. Tumours which do not attract a blood supply are restricted in size and thus the prevention or limitation of tumour angiogenesis may therefore represent a treatment option for solid tumours.

Endothelial cells that line the vasculature within a tumour may be exposed to a different extracellular environment compared to endothelial cells in normal tissue. For example, endothelial cells in a tumour may be subjected to hypoxic conditions, nutrient deprivation, and/or more acidic conditions. Further, tumour endothelial cells may experience different mechanical forces, such as a reduced blood flow rate and increased mechanical compression. The exposure of tumour endothelial cells to different conditions results in the display of a different transcriptome compared to cells in normal tissues, with the expression of tumour endothelial markers that may be present at a higher level in tumour endothelial cells compared to endothelial cells within normal vasculature. Thus, tumour endothelial cells may be targeted therapeutically by targeting tumour endothelial markers.

CLEC14A, a member of the type 14 family of calcium dependent C-type lectins (which additionally includes endosialin/TEM1/CD248, thrombomodulin and CD93 as members), is a single pass type I transmembrane protein of 490 amino acids in length, which comprises a signal peptide (at amino acid residues 1-21), an extracellular region (at amino acid residues 22-398), a transmembrane domain (at amino acid residues 399-421) and a cytoplasmic domain (at amino acid residues 422-490). The extracellular region of CLEC14A has one C-type lectin-like domain (at amino acid residues 22-173) and an epidermal growth factor-like region (at amino acid residues 245-287). Human and mouse CLEC14A proteins show 67% amino acid sequence identity, with a greater sequence conservation within the C-type lectin and epidermal growth factor-like domains.

The inventors have previously shown that CLEC14A is highly expressed on the surface of endothelial cells lining the vasculature of many common human cancers (including breast, liver, prostate, pancreatic, bladder and ovarian carcinomas), but in the vasculature of healthy tissue, expression is low or undetectable. It is believed that the conditions of low shear stress experienced in tumour vasculature, due to ill formation of the vessels, may be responsible for the upregulation of CLEC14A. Further, CLEC14A has been disclosed as playing a role in sprouting angiogenesis and as promoting tumour growth in mice. Thus, CLEC14A has previously been proposed as a tumour endothelial marker, which could be targeted to inhibit tumour angiogenesis.

Antibody or immunotherapies are proving to be effective for targeting some tumour types. One such immunotherapy treatment is based on the modification of immune cells, particularly T cells, with a chimeric antigen receptor (CAR) (a receptor which can specifically bind to a tumour target and which can activate/stimulate the immune cells after binding). In principal, any cell surface molecule can be targeted by using a CAR immunotherapy, thus overriding tolerance to self-antigens and providing a treatment which is not reliant on the MHC status of a patient. Using T cells engineered to express a chimeric antigen receptor targeting CD19, recent trials have demonstrated remarkable clinical responses in leukaemia and lymphoma patients. CARs are usually comprised of a monoclonal antibody-derived single chain variable fragment (scFv) consisting of a heavy and light chain joined by a flexible linker and then fused through a transmembrane domain to a cytoplasmic signalling domain (usually a CD3 zeta chain). More recently these constructs have incorporated additional cytoplasmic domains from co-stimulatory molecules such as CD28 or 4-1BB to enhance T cell survival in vivo. CARs comprising one cytoplasmic domain from a costimulatory molecule are known as second generation CARs, CARS comprising two cytoplasmic domains each from a co-stimulatory molecule are known as third generation CARS and CARS comprising two (or more) cytoplasmic domains each from a co-stimulatory molecule, together with the presence of an additional genetic modification in the nucleic acid encoding the CAR (e.g. the presence of a cytokine gene) are known as fourth generation CARs. Other genetic modifications have also been made to CARs, e.g. the addition of cytokine genes or genes to avoid immunosuppressive mechanisms at the tumour site. The present inventors have now developed a CAR immunotherapy that selectively targets CLEC14A. In this respect, the immunotherapy specifically utilises a binding domain/activity from an antibody which selectively binds to the C-type lectin domain of CLEC14A and particularly from an antibody that disrupts the interaction between CLEC14A and MMRN2. Thus, the inventors have identified that the interaction between CLEC14A and MMRN2 plays an important role in angiogenesis (MMRN2 is an endothelial specific marker of the emilin family and a component of the extracellular matrix). The inventors have further identified that the interaction between CLEC14A and MMRN2 may be disrupted by anti-CLEC14A antibodies that bind to the C-type lectin domain of CLEC14A, particularly to amino acid residues 97-108 of CLEC14A. In this respect, the inventors have primarily used the binding activity of antibodies which target these domains of CLEC14A in CAR immunotherapies, and have demonstrated efficacious results using such immunotherapies e.g. in the reduction of tumour size.

In a first aspect, the present invention thus provides a nucleic acid molecule comprising a polynucleotide sequence encoding a chimeric antigen receptor comprising

(i) an anti-CLEC14A binding domain,

(ii) a transmembrane domain and

(iii) an intracellular signalling domain;

wherein said anti-CLEC14A binding domain is capable of binding to the C-type lectin domain of CLEC14A.

Particularly, the anti-CLEC14A binding domain may be capable of disrupting the interaction between CLEC14A and MMRN2.

Thus, according to the present invention and as discussed above, CAR immunotherapies have been developed based on the discovery and isolation of antibodies which bind to the C-type lectin domain of CLEC14A and the finding that antibodies which bind to a particular epitope within the C-type lectin domain of CLEC14A are able to disrupt the interaction between CLEC14A and MMRN2, which the inventors have shown to be involved in angiogenesis. As shown in the Examples, the inventors have produced and tested several CAR immunotherapies incorporating a binding domain which binds to the C-type lectin domain of CLEC14A, and have shown such CAR immunotherapies to be effective in reducing tumour size and volume. Four specific novel antibodies (described herein as CRT1, 3, 4 and 5, respectively, and whose CDR, heavy and light chain variable sequences are shown in Table 1) have been identified by the inventors, which antibodies are capable of binding to the C-type lectin domain of CLEC14A, and the CLEC14A binding domains of these antibodies have been used in the development of the CAR immunotherapies described herein to provide specific examples of therapies of the invention. The surprisingly efficacious results produced upon using the CAR therapies of the invention indicates that the targeting of the C-type lectin domain of CLEC14A using a CAR may provide an effective treatment for tumour angiogenesis and thus for cancer. Further, the association of the upregulation of CLEC14A under conditions of low shear stress may make the CAR therapies of the invention especially effective against tumours where blood flow is particularly restricted, e.g. in tumours of the pancreas or ovary, for which few effective treatments are currently available.

In a further aspect of the present invention, the inventors have identified an additional antibody which specifically binds to the external region of CLEC14A, and which can be used in a CAR in accordance with the present invention. This specific antibody (named herein as CRT2, the light chain CDR and light chain variable sequences for which can be found in Table 1) can bind effectively to CLEC14A, and thus a CAR immunotherapy comprising the antigen binding capability of this antibody is encompassed by the present invention.

In this respect, in a second embodiment, the invention further provides a nucleic acid molecule comprising a polynucleotide sequence encoding a chimeric antigen receptor comprising

(i) an anti-CLEC14A binding domain

(ii) a transmembrane domain and

(iii) an intracellular signalling domain,

wherein said anti-CLEC14A binding domain comprises at least one of:
(a) a heavy chain CDR having the amino acid sequence of SEQ ID NO. 167 or a variant thereof having one, two or three amino acid substitutions,
(b) a heavy chain CDR having the amino acid sequence of SEQ ID NO. 168 or a variant thereof having one, two or three amino acid substitutions,
(c) a heavy chain CDR having the amino acid sequence of SEQ ID NO. 169 or a variant thereof having one, two or three amino acid substitutions,
(d) a light chain CDR having the amino acid sequence of SEQ ID NO. 129 or a variant thereof having one, two or three amino acid substitutions,
(e) a light chain CDR having the amino acid sequence of SEQ ID NO. 68 or a variant thereof having one, two or three amino acid substitutions, and/or
(f) a light chain CDR having the amino acid sequence of SEQ ID NO. 130 or a variant thereof having one, two or three amino acid substitutions.

A nucleic acid of the invention as described above, therefore comprises a polynucleotide sequence which encodes a chimeric antigen receptor (CAR). A “CAR” or “chimeric antigen receptor”, used interchangeably herein, refers to a molecule, which comprises at least three domains, namely an extracellular domain comprising an antigen binding domain (in the present invention the anti-CLEC14A binding domain), a transmembrane domain and an intracellular domain comprising an intracellular signalling domain.

Thus, when a CAR is expressed on a host cell (particularly an effector cell, as discussed further below), the antigen binding domain will be present within or as the extracellular domain. Typically, most or all of the antigen binding domain will be present extracellularly, to allow the binding of the CAR to the target antigen (e.g. at least 90, 95, 97, 99 or 100% of the antigen binding domain will be present extracellularly when the CAR is expressed in a host cell, transported to the cell membrane and presented).

The transmembrane domain links the extracellular domain comprising the antigen binding domain (i.e. the anti-CLEC14A binding domain in the present invention) to the intracellular signalling domain and typically spans the cell membrane of a host cell after CAR expression and membrane targeting. Thus, the transmembrane domain passes through the cell membrane after CAR expression and membrane targeting. The transmembrane domain may be derived from or based upon a protein having at least one transmembrane domain and/or extracellular and/or intracellular portions and thus the transmembrane domain of a CAR may be attached at the N and/or C termini to extracellular and/or intracellular sequence/polypeptide/protein from the protein from which it is derived or based upon. Thus, when the transmembrane domain is obtained or derived from a known transmembrane protein, additional sequence may be present extracellularly and/or intracellularly, together with the transmembrane domain which passes through or spans the membrane, to attach the CAR thereto. As discussed further below, the transmembrane domain may be derived from a protein or a portion of a protein which has both transmembrane and intracellular regions, e.g. CD28, and both of these domains or portions thereof maybe comprised within a CAR of the invention.

The intracellular signalling domain of the CAR, is present within the host cell (i.e. is comprised within the intracellular domain of the CAR) after expression of the CAR, typically within the cytoplasm of the cell. This domain is capable of activating one or more normal functions of the host cell in which the CAR is expressed. For example, if the host cell is a T cell, then the intracellular signalling domain may be capable of activating the cytolytic or helper activity of the T cell. The CARs of the invention may additionally comprise further domains as discussed in greater detail below.

The “Anti-CLEC14A binding domain” as used herein refers to a domain which is capable of binding to CLEC14A and particularly to a domain which is capable of binding to CLEC14A when expressed within a CAR and presented on a cell surface. Particularly, the anti-CLEC14A binding domain is capable of binding to CLEC14A expressed on the surface of a cell (e.g. as assessed by flow cytometry or immunohistochemistry), binding to a conformationally dependent (e.g. non-linear) CLEC14A epitope (e.g. as assessed by Western blotting), binding to free CLEC14A (e.g. recombinantly expressed CLEC14A on a solid support) (e.g. as assessed by ELISA) and/or binding to human CLEC14A. Most particularly, the anti-CLEC14A binding domain is capable of binding to CLEC14A expressed on the surface of a cell.

Particularly, the anti-CLEC14A binding domain selectively binds to CLEC14A and thus has a greater binding affinity for CLEC14A as compared to its binding affinity for other proteins/molecules. Preferably, the anti-CLEC14A binding domain does not bind to other proteins or binds with a greatly reduced affinity compared to the binding to CLEC14A (e.g. with an affinity of at least 10, 50, 100, 500, 1000 or 10000 times less than its affinity for CLEC14A). Thus, the anti-CLEC14A binding domain as referred to herein may bind to CLEC14A with at least 10, 50, 100, 500, 1000 or 10000 times the affinity of its binding to other proteins. The binding affinity of the anti-CLEC14A binding domain can be determined using methods well known in the art such as with the Biacore system.

It is particularly preferred that the anti-CLEC14A binding domain has a reduced binding affinity for proteins which are similar to or have regions of identity to CLEC14A, or for proteins which are known homologs to CLEC14A, compared to its binding affinity for CLEC14A. Thus, the anti-CLEC14A binding domain particularly should have a reduced binding affinity (e.g. a binding affinity reduced by at least 10, 50, 100, 1000 or 10000 times) for proteins which have at least 60, 70, 80, 90 or 95% identity to CLEC14A and particularly to CD248/TEM1/Endosialin, Thrombomodulin and/or CD93, as compared to its binding affinity to CLEC14A. Alternatively viewed, the anti-CLEC14A binding domain particularly binds to CLEC14A with an affinity of at least 10, 50, 100, 1000 or 10000 times that of its affinity to bind CD248/TEM1/Endosialin, Thrombomodulin and/or CD93.

The anti-CLEC14A binding domain may have a high binding affinity for CLEC14A i.e. may have a Kd in the range of 10-⁵M, 10⁻⁶M, 10-⁷M or 10⁻⁹M or less. The anti-CLEC14A binding domain may have a binding affinity for CLEC14A that corresponds to a K_{d} of less than 30 nM, 20 nM, 15 nM or 10 nM, more preferably of less than 10, 9.5, 9, 8.5, 8, 7.5, 7, 6.5, 6, 5.5, 5, 4.5, 4, 3.5, 3, 2.5, 2, 1.5 or 1 nM, most preferably less than 0.9, 0.8, 0.7, 0.6, 0.5, 0.4, 0.3, 0.2 or 0.1 nM.

Any appropriate method of determining K_{d} may be used. However, preferably the K_{d} is determined by testing various concentrations of the test antibody against various concentrations of antigen (CLEC14A) in vitro to establish a saturation curve, for example using the Lineweaver-Burk method, or by using commercially available binding model software, such as the 1:1 binding model in the BIAcore 1000 Evaluation software.

With regard to determinations of K_{d} values, the skilled person will appreciate that apparent K_{d} values derived from binding experiments using cells expressing a target (e.g. CLEC14A) cannot be considered to be an absolute indication of affinity, because the experimental conditions will affect the apparent binding affinity. For example, the levels of expression of CLEC14A may vary depending on the conditions under which the cells are cultured, as well as differing between different cell types. It is consequently best to compare apparent K_{d} values obtained within one set of experiments and it may not always be appropriate to compare K_{d} values obtained in one set of experiments with K_{d} values obtained in a different set of experiments, particularly if the experimental conditions varied significantly.

Reference herein to “CLEC14A” refers to both human CLEC14A and to orthologs of CLEC14A from other species e.g. from horse, dog, pig, cow, sheep, rat, mouse, guinea pig or primate e.g. monkey. Thus, the anti-CLEC14A binding domain may be capable of binding to human CLEC14A and/or to an ortholog of CLEC14A from any species e.g. from mouse. Further, the anti-CLEC14A binding domain is preferably capable of binding to a naturally occurring variant of CLEC14A e.g. to a naturally occurring variant of human CLEC14A. Although the anti-CLEC14A binding domain may bind to CLEC14A orthologs with a different affinity than its binding to human CLEC14A, it may bind to human and murine CLEC14A with a similar affinity.

By “similar affinity” is meant that the binding affinity of the anti-CLEC14A domain e.g. antibody or ligand for human CLEC14A and for one or more of the other species of interest (e.g. mouse) is comparable, e.g. is not more than a factor of 20 different. More preferably the difference between the binding affinities is less than a factor of 15, more preferably less than a factor of 10, most preferably less than a factor of 5, 4, 3 or 2.

However, in a particular embodiment, the anti-CLEC14A binding domain binds to human CLEC14A, particularly to the extracellular portion of human CLEC14A. Human CLEC14A generally has 490 amino acids, a predicted molecular weight of 51 kDa and is encoded by the clec14A gene located at 14q21.2. Human CLEC14A includes the amino acid sequence found in Genbank Accession number NP_778230 and naturally occurring variants thereof. The amino acid sequence for human CLEC14A is as set out in SEQ ID NO. 1 (as shown in Table 1). Thus, particularly, the anti-CLEC14A binding domain as used in the present invention is capable of binding to an amino acid sequence of SEQ ID NO. 1. Human CLEC14A is encoded by the cDNA sequence of SEQ ID NO. 2, which corresponds to the mRNA sequence for CLEC14A and the coding region for human CLEC4A is shown in SEQ ID NO. 3.

Particularly, the anti-CLEC14A binding domain binds to the mature CLEC14A polypeptide, after removal of the signal peptide (which occurs at residues 1-21 of the 490 amino acid human polypeptide sequence). (It will be appreciated that different domain prediction programs available in the art may result in slight differences in the locations of particular domains within a protein, e.g. by 1, 2, 3, or 4 amino acid residues e.g. with respect to domain locations in CLEC14A. The signalling domain could therefore be predicted as being at residues 1-22 for example). Thus, typically, the anti-CLEC14A binding domain binds to the 469 (or 468 if a signal peptide of 1-22 is predicted) amino acid mature CLEC14A sequence in the case of human CLEC14A (residues 22 (or 23)-490 of the full polypeptide sequence). As discussed above, the anti-CLEC14A binding domain binds to the extracellular region of CLEC14A. In the case of human CLEC14A, the anti-CLEC14A binding domain thus particularly binds to a region within residues 22 (or 23)-396 of the 490 amino acid sequence of SEQ ID NO. 1 (i.e. a region within the 375 (or 374) amino acid extracellular domain of human CLEC14A), e.g. to the C-type lectin domain at residues 22-173, (or alternatively viewed at residues 23-173, 22-175, 23-175, 32-173 or 32-175) or to the EGF-like region at residues 245-287 of the extracellular domain. Human CLEC14A further comprises a transmembrane domain and a cytoplasmic region (at residues 397-425 and 426-490, respectively) and it is preferred that the anti-CLEC14A binding domain used in the invention does not bind to these regions, or at least only binds in addition to binding to the extracellular domain of CLEC14A with an affinity as discussed above. Particularly, it is preferred that the anti-CLEC14A binding domain binds to the extracellular domain with a greater affinity than to any other domain of CLEC14A e.g. to the transmembrane and/or cytoplasmic regions (e.g. binds to the extracellular domain with an affinity at least, 10, 50, 100, 1000 or 10000 times greater than to other CLEC14A regions).

If an anti-CLEC14A binding domain used in the present invention is capable of binding to an ortholog or a naturally occurring variant to human CLEC14A, it is preferred that the binding domain bind to a region within the ortholog or variant that corresponds to the extracellular domain of human CLEC14A as defined above. Corresponding regions within orthologous proteins can be easily determined using sequence alignment programs which are well known in the art.

As discussed above, in the first aspect of the invention, the anti-CLEC14A binding domain is capable of binding the C-type lectin domain of CLEC14 A. This domain can be found at residues 22-173 or a position within 1-4 residues of 22-173, e.g. at residues 22-175, 23-173, 23-175, 32-173, or 32-175 of the human 490 amino acid CLEC14A protein sequence (as shown in SEQ ID NO. 1). Thus, in this aspect of the invention, the anti-CLEC14A binding domain will be capable of binding to or within this region of the C-type lectin domain (found within the extracellular domain of CLEC14A). As indicated above, when the anti-CLEC14A binding domain is capable of binding to a CLEC14A ortholog or variant, in this aspect, it is preferred that the anti-CLEC14A binding domain binds to a region corresponding to the C-type lectin domain found within the extracellular domain of human CLEC14A. Such corresponding regions can be identified using sequence alignment. As discussed above, the anti-CLEC14A binding domain particularly may bind to the C-type lectin domain with a greater affinity (e.g. at least 10, 50, 100, 1000 or 10000 times greater) than to any other region in CLEC14A (e.g. than to the EGF-like region at residues 245-287 of human CLEC14A).

Particularly, according to the first aspect of the invention, the anti-CLEC14A binding domain may be capable of binding to an epitope within the C-type lectin domain of CLEC14A. In some instances, the anti-CLEC14A binding domain may be capable of binding to an epitope which is found at residues 97-108 of human CLEC14A and which has an amino acid sequence of ERRRSCHTLENE (SEQ ID NO.24), or to a corresponding epitope in a CLEC14A ortholog or naturally occurring variant. However, in other instances, the anti-CLEC14A binding domain may bind to a different epitope or region within the C-type lectin domain of CLEC14A, (i.e. not to residues 97-108) e.g. to 33-44, 45-56, 57-68, 69-80, 81-92, 109-120, 121-132, 133-144, 145-156 or 157-168 or to a region which overlaps with this region (overlaps with 97-108). Thus, it is possible for the anti-CLEC14A binding domain of the first aspect to bind to any epitope or residues within the C-type lectin domain of CLEC14A.

According to the second aspect of the invention, although the anti-CLEC14A binding domain binds to the extracellular domain of CLEC14A, it may not bind to the C-type lectin domain at residues 22-173 of SEQ ID NO. 1 and/or to a position within 1-4 residues thereof e.g. to 22-175, 23-173, 23-175, 32-173 or 32-175 of SEQ ID NO. 1. Thus, in the second aspect, the anti-CLEC14A binding domain particularly binds to residues 174-396 of human CLEC14A of SEQ ID NO. 1, or to residues 175 or 176-396 of human CLEC14A of SEQ ID NO. 1). Particularly, in this aspect, the anti-CLEC14A binding domain may bind to the Sushi domain of CLEC14A (alternatively known as the complement control protein (CCP) domain) which is found at residues 174-244 (or within 1-4 amino acid residues of this position e.g. at 175-244 or 176-244), of SEQ ID NO. 1, or to an equivalent portion in a CLEC14A orthologous sequence or naturally occurring variant. Particularly, the anti-CLEC14A binding domain may bind to a portion of the Sushi domain which is proximal to the C-type lectin domain e.g. at residues 174-210, 174-200 or 174-190.

An anti-CLEC14A binding domain as defined herein may bind to CLEC14A, as discussed above, when present or comprised within a CAR molecule, and expressed upon an appropriate host cell. Thus, particularly, the CAR of the invention or encoded by a nucleic acid molecule of the invention will be capable of binding to CLEC14A via the anti-CLEC14A binding domain of the CAR. The anti-CLEC14A binding domain may further be capable of CLEC14A binding as discussed above, when expressed in isolation (e.g. as a ligand or part of a ligand molecule) or when expressed as part of an antibody molecule or fragment thereof or scFv.

Additionally, as discussed further above, the anti-CLEC14A binding domain used in the first aspect of the invention, may be capable of disrupting or inhibiting the interaction between CLEC14A and MMRN2. Particularly, the anti-CLEC14A binding domain may be capable of disrupting the interaction between human CLEC14A and human MMRN2, where the amino acid sequence for MMRN2 is as set out in SEQ ID NO.28 of Table 1 (encoded by SEQ ID NO. 29). Thus, in this aspect, the anti-CLEC14A binding domain, when utilised in isolated form (or as part of an antibody or scFv) is capable of disrupting the interaction. The disruption of the interaction means a reduction in the amount of CLEC14A and MMRN2 molecules which form an interaction, i.e. an inhibition of the level of binding between CLEC14A and MMRN2. Particularly, the anti-CLEC14A binding domain used in the first aspect of the invention may inhibit the level of binding between CLEC14A and MMRN2 by at least 10, 20, 30, 40, 50, 60, 70, 80 or 90%. In one aspect, the anti-CLEC14A binding domain may be capable of preventing any interaction between CLEC14A and MMRN2 and thus may eliminate the interaction altogether (i.e. 100% of CLEC14A and MMRN2 interactions may be inhibited). Particularly however, the level of interaction may be reduced to an undetectable level.

Alternatively viewed, the anti-CLEC14A binding domain may be capable of competing with MMRN2 for binding to CLEC14A.

In this regard, the anti-CLEC14A binding domain may bind to the MMRN2 binding region of the CLEC14A polypeptide within the C-type lectin domain of CLEC14A. Whether or not a given ligand or antibody selectively binds to the MMRN2 binding region or competes with MMRN2 for specific binding to the CLEC14A polypeptide can be determined using routine methods of the art such as epitope mapping, competition binding studies etc.

Methods for determining the level of interaction between CLEC14A and MMRN2 are known in the art and include pull-down assays, enzyme-linked immunosorbent assays (ELISA), surface plasmon resonance assays, chip-based assays, immunocytofluorescence, yeast-two-hybrid technology, and phage display. Other methods of detecting interaction between CLEC14A and MMRN2 include ultrafiltration with ion spray mass spectroscopy/HPLC methods or other physical and analytical methods. Fluorescence Energy Resonance Transfer methods (FRET), may be used in which binding of two fluorescent labelled entities (i.e. CLEC14A and MMRN2 or portions, orthologs or variants thereof) may be measured by measuring the interaction of the fluorescent labels when in close proximity to each other.

As discussed in detail below, the anti-CLEC14A binding domain may be derived from an antibody which binds to CLEC14A, or from any other ligand (e.g. peptide or polypeptide) which binds to CLEC14A. Particularly, the anti-CLEC14A binding domain may be derived from MMRN2 (e.g. human MMRN2 of SEQ ID NO. 28), which as indicated above, interacts with CLEC14A and thus is a ligand of CLEC14A. The anti-CLEC14A binding domain may comprise the full length MMRN2 amino acid sequence (e.g. of SEQ ID NO. 28 for human MMRN2) or at least a portion thereof, wherein said portion is capable of binding to CLEC14A. Thus, particularly, a portion of MMRN2 may have at least 50, 60, 70, 80, 90, 100% or more of the affinity for CLEC14A as full length MMRN2. Alternatively viewed, a portion of MMRN2 may have the affinity for CLEC14A as discussed above in relation to the anti-CLEC14A binding domain.

Particularly, the anti-CLEC14A binding domain may comprise a portion of MMRN2 which is capable of binding to CLEC14A and disrupting the interaction of MMRN2 with CLEC14A, as discussed above. Alternatively or additionally, the at least a portion of MMRN2 may be capable of binding to the C-type lectin domain of CLEC14A, and more particularly to an epitope which is found at residues 97-108 of human CLEC14A and which has an amino acid sequence of ERRRSCHTLENE (SEQ ID NO.24), or to a corresponding epitope in a CLEC14A ortholog or naturally occurring variant. A portion of MMRN2 comprised within the anti-CLEC14A binding domain may comprise at least 3 contiguous amino acids from MMRN2, and particularly at least 4, 5, 6, 7, 8, 9, 10, 15, 20 or 25 amino acids.

The anti-CLEC14A binding domain may alternatively comprise a variant of full length MMRN2 or a portion thereof, wherein said variant is capable of binding to CLEC14A (e.g. with the affinity as discussed above in relation to the anti-CLEC14A binding domain). The variant may have at least 60, 70, 80, 90, 95, 96, 97, 98 or 99% sequence identity to the full length MMRN2 or to a portion of MMRN2, and as indicated above retains the ability to bind to CLEC14A. Alternatively viewed, an MMRN2 variant may comprise one or more, e.g. two, three, four, five, ten, or fifteen amino acid substitutions, deletions and/or additions as compared to the wildtype MMRN2 sequence, e.g. conservative substitutions. It will be appreciated that the number of amino acid substitutions, deletions and/or additions made to a variant of an MMRN2 portion may be in proportion to the length of the portion, where shorter portions may comprise fewer amino acid substitutions, additions and/or deletions than longer portions.

The MMRN2 variant sequence comprised within the anti-CLEC14A binding domain may have a different binding affinity for CLEC14A than the wildtype MMRN2 sequence, e.g. a higher or lower binding affinity, or the variant may have the same or substantially the same binding affinity to CLEC14A (e.g. a comparable affinity, e.g. not more than a factor of 20 different). It will be appreciated however, that the variant MMRN2 or an anti-CLEC14A binding domain comprising a variant of MMRN2 would preferably have a binding affinity for CLEC14A as discussed previously.

The anti-CLEC14A binding domain as used in the first aspect of the invention (e.g. in a CAR of the invention), can comprise any amino acid sequence, as long as the sequence has the binding activity discussed above, i.e. as long as the binding domain can bind to the C-type lectin domain of CLEC14A. Particularly, however, the anti-CLEC14A binding domain may comprise at least one heavy or light chain complementarity determining region (CDR) which is capable of binding to the C-type lectin domain of CLEC14A. The one or more CDRs may be predicted from the heavy and/or light chain sequences of an antibody which is capable of binding to CLEC14A (i.e. to the C-type lectin domain of CLEC14A), as discussed above (i.e. with the affinities and specificities as discussed above). Particularly, the anti-CLEC14A binding domain may comprise one or more CDRs from any one of antibodies CRT1, 3, 4 or 5 as set out in Table 1.

In connection with this, in the second aspect of the invention, the anti-CLEC14A binding domain comprises at least one CDR selected from the heavy chain and/or light chain CDRs of SEQ ID NO. 167, SEQ ID NO. 168, SEQ ID NO.169, SEQ ID NO. 129, SEQ ID NO. 68 or SEQ ID NO. 130 or a variant of any one of these sequences with one, two or three amino acid substitutions, where the selected CDRs can be predicted from the light chain sequence (SEQ ID NO. 133) and the heavy chain sequence (SEQ ID NO. 173) of antibody CRT2 (which binds to CLEC14A as shown in the Examples and as set out in Table 1).

Thus, the anti-CLEC14A binding domain may comprise at least one CDR, which can be predicted from an antibody which binds to CLEC14A (or a variant of such a predicted CDR (e.g. a variant with one, two or three amino acid substitutions)) where the anti-CLEC14A binding domain and thus the CAR comprising the anti-CLEC14A binding domain are capable of binding to CLEC14A.

It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Laune et al, 1997, JBC, 272, 30937-44; Nicaise et al, 2004, Protein Science, 13: 1882-91).

In this respect, the anti-CLEC14A binding domain used in the invention may comprise one or more variable heavy chain CDRs, e.g. one, two or three variable heavy chain CDRs. Alternatively or additionally, the anti-CLEC14A binding domain may comprise one or more variable light chain CDRs, e.g. one, two or three variable light chain CDRs. Particularly, however, the anti-CLEC14A binding domain may comprise three heavy chain CDRs and three light chain CDRs (and more particularly a heavy chain variable region comprising three CDRs and a light chain variable region comprising three CDRs) wherein at least one CDR may be predicted from an antibody which binds to CLEC14A, or may be selected from one of the CDR sequences provided below.

The anti-CLEC14A binding domain of the invention may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs.

The one or more CDRs present within the anti-CLEC14A binding domain may not all be predicted from the same antibody, as long as the domain has the binding activity described above. Thus, one CDR may be predicted from the heavy or light chains of an antibody which binds to CLEC14A whilst another CDR present may be predicted from a different antibody. In this instance, it may be preferred that CDR3 be predicted from an antibody that binds to CLEC14A. Particularly however, if more than one CDR is present in the anti-CLEC14A binding domain, it is preferred that the CDRs are predicted from antibodies which bind to CLEC14A. The CDRs do not need to be from the same CLEC14A binding antibody and a combination of CDRs may be used from different CLEC14A antibodies, particularly from CLEC14A antibodies that bind to the same desired region or epitope.

In a particular embodiment, the anti-CLEC14A binding domain comprises three CDRs predicted from the variable heavy chain sequence of an antibody which binds to CLEC14A and three CDRs predicted from the variable light chain sequence of an antibody which binds to CLEC14A (preferably the same antibody).

The anti-CLEC14A binding domain may further comprise the variable heavy and light chains from an antibody which binds to CLEC14A, particularly may comprise a scFv comprising the variable heavy and light chains from an antibody which binds to CLEC14A.

Reference to a “complementarity determining region” or “CDR” as used herein refers to the regions of hypervariability within antibodies which bind to the specific antigen e.g. to CLEC14A. The CDRs of an antibody thus usually provide an antibody with its binding specificity. Three CDRs may be present in the variable region of each heavy chain of an intact antibody molecule (i.e. comprising two full length heavy and two full length light chains) and three CDRs may be present in the variable region of each light chain (heavy chain CDRs 1, 2 and 3 and light chain CDRs 1, 2 and 3, numbered from the amino to the carboxy terminus). The CDRs of the variable regions of a heavy and light chain of an antibody can be predicted from the heavy and light chain variable region sequences of the antibody, using prediction software available in the art, e.g. using the Abysis algorithm (www.bioinf.org.uk/abysis/sequence_input/key_annotation/key_annotation.cgi), or using the IMGT/V-QUEST software, e.g. the IMGT algorithm (ImMunoGeneTics) which can be found at www.IMGT.org, see for example Lefranc et al, 2009 NAR 37:D1006-D1012 and Lefranc 2003, Leukemia 17: 260-266. CDR regions identified by either algorithm are considered to be equally suitable for use in the invention. CDRs may vary in length, depending on the antibody from which they are predicted and between the heavy and light chains. Thus the three heavy chain CDRs of an intact antibody be of different lengths (or may be of the same length) and the three light chain CDRs of an intact antibody may be of different lengths (or may be of the same length). A CDR for example, may range from 2 or 3 amino acids in length to 5, 6, 7, 8, 9, 10, 11, 12, 13, 14, 15, 16, 17, 18, 19 or 20 amino acids in length. Particularly, a CDR may be from 3-14 amino acids in length, e.g. at least 3 amino acids and less than 20 amino acids.

The CDRs of the heavy and light chain variable regions (i.e. the heavy chain variable CDR 1, 2 and 3 and light chain variable CDR 1, 2 and 3) are usually separated from each other by framework regions. The heavy chain and light chain variable regions both comprise four framework regions (FR1, 2, 3 and 4, numbered from the amino to the carboxy terminus).

The term “heavy chain variable region” (VH domain) as used herein refers to the variable region of a heavy chain of an antibody molecule.

The term “light chain variable region” (VL domain) as used herein refers to the variable region of a light chain of an antibody molecule. The light chains of mammalian antibodies are assigned to one of two clearly distinct types: kappa and lambda, based on the amino acid sequences of their constant domains and some amino acids in the framework regions of their variable domains.

It should be note that the Kabat nomenclature is followed herein where necessary, in order to define the positioning of the CDRs (Kabat et al, 1991, 5^{th} Ed. Public Health Service, National Institutes of Health, Bethesda, Md., 647-669, incorporated herein by reference).

Reference to an “antibody” includes but is not limited to polyclonal, monoclonal, chimeric, single chain, Fab fragments and fragments produced by a Fab expression library. Such fragments include fragments of whole antibodies which retain their binding activity for a target, Fv, F(ab′) and F(ab′)2 fragments, as well as single chain antibodies (scFv), fusion proteins and other proteins which comprise the antigen binding site of the antibody. The term also includes antibody-like molecules which may be produced using phage-display techniques or other random selection techniques for molecules which bind to the specified polypeptide or to particular regions of it. Thus, the term antibody includes all molecules which contain a structure which is part of the recognition site (i.e. the part of the antibody that binds or combines with the epitope or antigen) of a natural antibody. Furthermore, the antibodies and fragments thereof may be humanised antibodies, where the framework region e.g. of VH and/or VL may be modified to correspond to at least one human framework region, using methods known in the art.

Reference to “scFv” or “single-chain variable fragment” as used herein includes molecules wherein the variable heavy (VH) and variable light chain (VL) of an antibody are linked via a flexible oligopeptide. A scFv is thus a fusion between at least one variable heavy and at least one variable light chain. The flexible oligopeptide which usually links the variable heavy and light chains may be from 5 amino acids in length, particularly from 8, 9, 10 or 11 amino acids to 20, 21, 22, 23, 24, 25, 26, 27, 28, 29 or 30 amino acids in length, e.g. from 10-20, from 12-18 or from 14-17 or 14-16 amino acids in length. The flexible oligopeptide may comprise glycine, serine and/or threonine residues and particularly may be comprise at least 50, 60, 70, or 80% glycine residues. The flexible linker may generally connect the C-terminus of the variable heavy chain to the N terminus of the variable light chain, or the C-terminus of the variable light chain to the N-terminus of the variable heavy chain. Engineered antibodies, such as scFv antibodies, can be made using the techniques and approaches known in the art.

Fab, Fv, scFv and dAb antibodies can all be expressed in and secreted from E. coli, which allows the easy production of large amounts of antibody fragments. Whole or intact antibodies are bivalent, i.e. have two antigen combining sites. Fab, Fv, scFv and dAb fragments are usually monovalent and thus usually have only one antigen combining site. Thus monovalent scFvs may comprise one variable heavy chain and one variable light chain. It is possible however that scFv may be divalent, trivalent or tetravalent (in addition to monovalent) and that the scFv may comprise more than one variable heavy chain and more than one variable light chain e.g. two, three or four variable heavy or variable light chains. The more than one variable heavy and/or variable light chains may be the same or may be from different antibodies. The scFv may be a diabody, tribody or a tetrabody. In this aspect, the flexible linker used may be shorter than as used above in a monovalent scFv.

Reference to “an antibody which binds to CLEC14A” refers to an antibody with the same binding affinity for CLEC14A as discussed above with respect to the anti-CLEC14A binding domain. Particularly, in accordance with the first aspect of the invention, an antibody which binds to CLEC14A binds to the C-type lectin domain of CLEC14A as previously defined.

In a further embodiment of the first aspect, the anti-CLEC14A binding domain may comprise at least one of:
(a) a heavy chain CDR1 having the amino acid sequence of SEQ ID NO. 211 or a variant thereof having one, two or three amino acid substitutions, (e.g. particularly of (S/T) SYW (UM) (E/H) (SEQ ID NO. 150), GYTF (S/T) SYW (SEQ ID NO. 151) or a variant thereof having one, two or three amino acid substitutions),
(b) a heavy chain CDR2 having the amino acid sequence of SEQ ID NO. 212 or a variant thereof having one, two or three amino acid substitutions (e.g. particularly of WIG (E/A) I (L/Y) PG (S/N) (G/S) (S/D) T (N/S) (SEQ ID NO. 152), I (L/Y) PG (S/N) (G/S) (S/D) T (SEQ ID NO. 153) or a variant thereof having one, two or three amino acid substitutions) and/or
(c) a heavy chain CDR3 having the amino acid sequence of SEQ ID NO. 213 or a variant thereof having one, two or three amino acid substitutions (e.g. particularly of (A/T) (R/H) (G/X) (G/X) (D/X) Y (D/Y) (E/G) (E/S) (Y/D) Y (V/A/L) MD (SEQ ID NO. 154), (A/T) (R/H) (G/X) (G/X) (D/X) Y (D/Y) (E/G) (E/S) (Y/D) Y (V/A/L) MDY (SEQ ID NO. 155) or a variant thereof having one, two or three amino acid substitutions)
   wherein X is no amino acid residue.

Thus, the anti-CLEC14A binding domain of the first aspect may comprise any one or more of SEQ ID Nos, 150, 151, 152, 153, 154, or 155, or one or more variants of any one or more of SEQ ID Nos 150-155 having one, two or three amino acid substitutions. The use of the “I” in the context of an amino acid sequence described herein refers to a choice of amino acid residues which may be present at a particular position. For example reference to “SIT” indicates that either an S or a T residue may be present at that position, and reference to GYTF/X indicates that either GYTF or no amino acid may be present at that position. The anti-CLEC14A binding domain may therefore comprise one or more amino acid sequences selected from SEQ ID NO. 150, 152 and/or 154 or selected from SEQ ID NO. 151, 153 and/or 155, or a variant of any one or more of these sequences as defined previously.

Particularly, in this aspect, the anti-CLEC14A binding domain may comprise two or three of the CDRs described above. Most particularly, the anti-CLEC14A binding domain may comprise a CDR having an amino acid sequence of SEQ ID 150 or 151, a CDR having an amino acid sequence of SEQ ID NO 152 or 153 and a CDR having an amino acid sequence of 154 or 155, or one or more variants of any of these sequences as defined previously e.g. the anti-CLEC14A binding domain may comprise SEQ ID Nos 150, 152 and 154 or SEQ ID Nos 151, 153 and 155, or one or more variants of any of these CDRs having one, two or three amino acid substitutions.

Further, the first aspect of the invention, the anti-CLEC14A binding domain may comprise at least one of:
(a) a light chain CDR1 having an amino acid sequence of S/X S/X S Y M/L Y/H W Y (SEQ ID NO. 156), SSVS Y/S S/X Y/X (SEQ ID NO. 157) or a variant thereof having one, two or three amino acid substitutions,
(b) a light chain CDR2 having an amino acid sequence of SEQ ID NO. 214 or a variant thereof having one, two or three amino acid substitutions (e.g. particularly of L L/W IY D/S TSNLA (SEQ ID NO. 158), D/S TS or a variant thereof having one, two or three amino acid substitutions) and/or
(c) a light chain CDR3 having an amino acid sequence of SEQ ID NO. 215 or a variant thereof having one, two or three amino acid substitutions (e.g. particularly of Q/H Q W/Y S/H S/R Y/S P L/R (SEQ ID NO. 160), Q/H Q W/Y S/H S/R Y/S P L/R T F/X (SEQ ID NO. 161) or a variant thereof having one, two or three amino acid substitutions),
   wherein X is no amino acid.

Thus, the anti-CLEC14A binding domain of the first aspect may comprise any one or more of SEQ ID Nos. 156, 157, 158, 160, 161 or D/S TS, or a variant thereof having one, two or three amino acid substitutions. The anti-CLEC14A binding domain may therefore comprise one or more amino acid sequences selected from SEQ ID NO. 156, 158 and/or 160 or selected from SEQ ID NO. 157, 161 and/or D/S TS, or a variant of one or more of these sequences as set out previously.

Particularly, in this aspect, the anti-CLEC14A binding domain may comprise two or three of the CDRs described above, e.g. one CDR selected from (a), one CDR selected from (b) and/or one CDR selected from (c). Most particularly, the anti-CLEC14A binding domain may comprise a CDR of SEQ ID 156 or 157, a CDR of SEQ ID NO 158 or D/S TS and a CDR of 160 or 161, e.g. the anti-CLEC14A binding domain may comprise SEQ ID Nos 156, 158 and 160 or SEQ ID Nos 157, 161 and D/S TS, or a variant of any of these CDRs having one, two or three amino acid substitutions.

The anti-CLEC14A binding domain of the first aspect may comprise at least one CDR selected from any one or more of SEQ ID Nos 150-155 and (/or) at least one CDR selected from any one or more of SEQ ID Nos 156-161, e.g. two CDRs from SEQ ID Nos 150-155 and two CDRs from SEQ ID Nos 156-161. Particularly, the anti-CLEC14A binding domain of the first aspect may comprise a CDR having the amino acid sequence of SEQ ID NO 150 or 151, a CDR having the amino acid sequence of SEQ ID NO. 152 or 153, a CDR having the amino acid sequence of SEQ ID NO. 154 or 155, a CDR having the amino acid sequence of SEQ ID NO. 156 or 157, a CDR having the amino acid sequence of SEQ NO. 158 or D/S TS and a CDR having the amino acid sequence of SEQ ID NO. 160 or 161, or one or more variants of any one of these sequences having one, two or three amino acid substitutions. In a most preferred aspect of the invention, the anti-CLEC14A binding domain according to the first aspect may comprise CDRs having the amino acid sequences of SEQ ID NO. 150, 152, 154, 156, 158 and/or 160 or having the amino acid sequences of SEQ ID Nos 151, 153, 155, 157, 161 and/or D/S TS, or one or more variants of any one of these sequences having one, two or three amino acid substitutions.

According to the first aspect, the invention more particularly provides a nucleic acid molecule comprising a polynucleotide sequence encoding a chimeric antigen receptor comprising

(i) an anti-CLEC14A binding domain,

(ii) a transmembrane domain and

(iii) an intracellular signalling domain;

wherein said anti-CLEC14A binding domain is capable of binding to the C-type lectin domain of CLEC14A and wherein said anti-CLEC14A binding domain comprises at least one of:
(a) a heavy chain CDR1 having an amino acid sequence of SEQ ID NO. 32, SEQ ID NO. 44, SEQ ID NO. 64, SEQ ID NO. 76 or a variant thereof having one, two or three amino acid substitutions,
(b) a heavy chain CDR2 having an amino acid sequence of SEQ ID NO. 33, SEQ ID NO. 45, SEQ ID NO. 65, SEQ ID NO. 77 or a variant thereof having one, two or three amino acid substitutions and/or
(c) a heavy chain CDR3 having an amino acid sequence of SEQ ID NO. 116, SEQ ID NO. 118, SEQ ID NO. 66, SEQ ID NO. 78, SEQ ID NO. 34, SEQ ID NO. 46, SEQ ID NO. 100, SEQ ID NO. 102 or a variant thereof having one, two or three amino acid substitutions.

Thus, the anti-CLEC14A binding domain may comprise one, two or three CDRs selected from the heavy chain variable CDR sequences set out above. Particularly, the anti-CLEC14A binding domain may comprise one CDR selected from the sequences provided in (a), one CDR selected from the sequences provided in (b) and/or one CDR from the sequences provided in (c), or one or more variants of those sequences having one, two or three amino acid substitutions. For example, the anti-CLEC14A binding domain may comprise a CDR having an amino acid sequence of SEQ ID NO. 32, a CDR having an amino acid sequence of SEQ ID No. 33 and a CDR having an amino acid sequence of SEQ ID NO. 66 or 116.

According to the first aspect, the invention further provides a nucleic acid molecule comprising a polynucleotide sequence encoding a chimeric antigen receptor comprising

(i) an anti-CLEC14A binding domain,

(ii) a transmembrane domain and

(iii) an intracellular signalling domain;

wherein said anti-CLEC14A binding domain is capable of binding to the C-type lectin domain of CLEC14A and wherein said anti-CLEC14A binding domain comprises at least one of:
(a) a light chain CDR1 having an amino acid sequence of SEQ ID NO. 35, SEQ ID NO. 47, SEQ ID NO. 67, SEQ ID NO. 79 or a variant thereof having one, two or three amino acid substitutions,
(b) a light chain CDR2 having an amino acid sequence of SEQ ID NO. 36, DTS, SEQ ID NO. 68, STS or a variant thereof having one, two or three amino acid substitutions, and/or
(c) a light chain CDR3 having an amino acid sequence of SEQ ID NO. 37, SEQ ID NO. 49, SEQ ID NO. 69, SEQ ID NO. 81 or a variant thereof having one, two or three amino acid substitutions.

Thus, the anti-CLEC14A binding domain may comprise one, two or three CDRs selected from the light chain CDR sequences set out above. Particularly, the anti-CLEC14A binding domain may comprise one CDR selected from the sequences provided in (a), one CDR selected from the sequences provided in (b) and/or one CDR from the sequences provided in (c), or one or more variants of those sequences having one, two or three amino acid substitutions. For example, the anti-CLEC14A binding domain may comprise a CDR having an amino acid sequence of SEQ ID NO. 35, a CDR having an amino acid sequence of SEQ ID No. 68 and a CDR having an amino acid sequence of SEQ ID NO. 49.

The anti-CLEC14A binding domain of the first aspect may comprise at least one CDR selected from any one or more of SEQ ID Nos 32, 33, 34, 44, 45, 46, 100, 102, 116, 118, 64, 65, 66, 76, 77, or 78 and (/or) at least one CDR selected from any one or more of SEQ ID Nos 35, 36, 37, 47, 49, 67, 68, 69, 79, 81, STS or DTS, e.g. two CDRs from SEQ ID Nos 32, 33, 34, 44, 45, 46, 100, 102, 116, 118, 64, 65, 66, 76, 77, or 78 and two CDRs from SEQ ID Nos. 35, 36, 37, 47, 49, 67, 68, 69, 7981, STS or DTS. Particularly, the anti-CLEC14A binding domain of the first aspect may comprise a CDR having the amino acid sequence of SEQ ID NO 32, 44, 64 or 76, a CDR having the amino acid sequence of SEQ ID NO. 33, 45, 65 or 77, a CDR having the amino acid sequence of SEQ ID NO. 34, 46, 100, 102, 116, 118, 66 or 78, a CDR having the amino acid sequence of SEQ ID NO. 35, 47, 67 or 79, a CDR having the amino acid sequence of SEQ NO. 36, 68, STS or DTS and a CDR having the amino acid sequence of SEQ ID NO. 37, 49, 69 or 81, or one or more variants of any one of these sequences having one, two or three amino acid substitutions.

More particularly, the anti-CLEC14A binding domain may comprise at least one of
(a) a heavy chain CDR1 having an amino acid sequence of SEQ ID NO. 209 or a variant thereof having one, two or three amino acid substitutions (e.g. particularly of SEQ ID NO. 32, SEQ ID NO. 44, or a variant thereof having one, two or three amino acid substitutions),
(b) a heavy chain CDR2 having an amino acid sequence of SEQ ID NO. 210 or a variant thereof having one, two or three amino acid substitutions (e.g. particularly of SEQ ID NO. 33, SEQ ID NO. 45, or a variant thereof having one, two or three amino acid substitutions), and/or
(c) a heavy chain CDR3 having an amino acid sequence of SEQ ID NO. 207 or a variant thereof having one, two or three amino acid substitutions (e.g. particularly of SEQ ID NO. 116, SEQ ID NO. 118, SEQ ID NO. 34, SEQ ID NO. 46, SEQ ID NO. 100, SEQ ID NO. 102 or a variant thereof having one, two or three amino acid substitutions).

Thus, as indicated above, the anti-CLEC14A binding domain may comprise two or three of the above described CDRs and may particularly comprise one CDR selected from (a), one CDR selected from (b) and/or one CDR selected from (c). Thus, preferably, the anti-CLEC14A binding domain may comprise at least one heavy chain CDR selected from SEQ ID NO. 32, 33 and/or 34; SEQ ID NO. 44, 45 and/or 46; SEQ ID NO. 32, 33 and/or 100; SEQ ID NO. 44, 45 and/or 102; SEQ ID NO. 32, 33 and/or 116 or SEQ ID NO. 44, 45 and/or 118 or a variant of any one or more of these sequences having one, two or three amino acid substitutions.

In a particularly preferred embodiment, the anti-CLEC14A binding domain of the first aspect may comprise CDRs having amino acid sequences of

SEQ ID NO. 32, SEQ ID NO. 33 and SEQ ID NO. 34,

SEQ ID NO. 44, SEQ ID NO. 45 and SEQ ID NO. 46,

SEQ ID NO. 32, SEQ ID NO. 33 and SEQ ID NO. 100,

SEQ ID NO. 44, SEQ ID NO. 45 and SEQ ID NO. 102,

SEQ ID NO. 32, SEQ ID NO. 33 and SEQ ID NO. 116, or

SEQ ID NO. 44, SEQ ID NO. 45 and SEQ ID NO. 102,

wherein any of the above listed sequences may comprise one, two or three amino acid substitutions.

Further, the anti-CLEC14A binding domain may comprise at least one of
(a) a light chain CDR1 having an amino acid sequence of SEQ ID NO. 35, SEQ ID NO. 47 or a variant thereof having one, two or three amino acid substitutions,
(b) a light chain CDR2 having an amino acid sequence of SEQ ID NO. 36, DTS or a variant thereof having one, two or three amino acid substitutions, and/or
(c) a light chain CDR3 having an amino acid sequence of SEQ ID NO. 208 or a variant thereof having one, two or three amino acid substitutions (e.g. particularly of SEQ ID NO. 37, SEQ ID NO 49, or a variant thereof having one, two or three amino acid substitutions).

Thus, as indicated above, the anti-CLEC14A binding domain may comprise two or three of the above described CDRs and may particularly comprise one CDR selected from (a), one CDR selected from (b) and/or one CDR selected from (c). Thus, preferably, the anti-CLEC14A binding domain may comprise at least one light chain CDR selected from SEQ ID NO. 35, 36 and/or 37; SEQ ID NO. 47, 49 and/or DTS; or SEQ ID NO. 47, and/or DTS; or a variant of any one or more of these sequences having one, two or three amino acid substitutions.

In a particularly preferred embodiment, the anti-CLEC14A binding domain of the first aspect may comprise CDRs having amino acid sequences of SEQ ID NO. 35, SEQ ID NO. 36, and SEQ ID NO. 37; or SEQ ID NO. 47, DTS and SEQ ID NO 0.49, wherein any one or more of the above sequences may have one, two or three amino acid substitutions.

The anti-CLEC14A binding domain of the first aspect may comprise at least one CDR selected from any one or more of SEQ ID Nos 32, 33, 34, 44, 45, 46, 100, 102, 116 or 118 and at least one CDR selected from any one or more of SEQ ID Nos 35, 36, 37, 47, or 49 or DTS e.g. two CDRs from SEQ ID Nos 32, 33, 34, 44, 45, 46, 100, 102, 116 or 118 and two CDRs from SEQ ID Nos. 35, 36, 37, 47, or 49 or DTS. Particularly, the anti-CLEC14A binding domain of the first aspect may comprise a CDR having the amino acid sequence of SEQ ID NO 32, or 44, a CDR having the amino acid sequence of SEQ ID NO. 33, or 45, a CDR having the amino acid sequence of SEQ ID NO. 34, 46, 100, 102, 116, or 118, a CDR having the amino acid sequence of SEQ ID NO. 35 or 47, a CDR having the amino acid sequence of SEQ NO. 36 or DTS and a CDR having the amino acid sequence of SEQ ID NO. 37, or 49, or one or more variants of any one of these sequences having one, two or three amino acid substitutions.

Particularly, the anti-CLEC14A binding domain may comprise CDRs having amino acid sequences of

SEQ ID NO. 32, SEQ ID NO. 33, SEQ ID NO. 34, SEQ ID NO. 35, SEQ ID NO. 36 and/or SEQ ID NO. 37;

SEQ ID NO. 44, SEQ ID NO. 45, SEQ ID NO. 46, SEQ ID NO. 47, DTS, and/or SEQ ID NO. 49;

SEQ ID NO. 32, SEQ ID NO. 33, SEQ ID NO. 100, SEQ ID NO. 35, SEQ ID NO. 36, and/or SEQ ID NO. 37;

SEQ ID NO. 44, SEQ ID NO. 45, SEQ ID NO. 102, SEQ ID NO. 47, DTS, and/or SEQ ID NO. 49;

SEQ ID NO. 32, SEQ ID NO. 33, SEQ ID NO. 116, SEQ ID NO. 35, SEQ ID NO. 36, and/or SEQ ID NO. 37; or

SEQ ID NO. 44, SEQ ID NO. 45, SEQ ID NO. 118, SEQ ID NO. 47, DTS and/or SEQ ID NO. 49,

wherein any one or more of the above SEQ ID Nos may comprise one, two or three amino acid substitutions.

Alternatively viewed, the anti-CLEC14A binding domain may comprise
(a) a heavy chain CDR1 having an amino acid sequence of SEQ ID NO. 209;
(b) a heavy chain CDR2 having an amino acid sequence of SEQ ID NO. 210;
(c) a heavy chain CDR3 having an amino acid sequence of SEQ ID NO. 207;
(d) a light chain CDR1 having an amino acid sequence of SEQ ID NO. 35 or 47;
(e) a light chain CDR2 having an amino acid sequence of SEQ ID NO. 36 or DTS and/or
(f) a light chain CDR3 having an amino acid sequence of SEQ ID NO. 208.

Alternatively, the anti-CLEC14A binding domain of the first aspect may comprise at least one of
(a) a heavy chain CDR1 having an amino acid sequence of SEQ ID NO. 216 or a variant thereof having one, two or three amino acid substitutions (e.g. particularly of SEQ ID NO. 64, SEQ ID NO. 76 or a variant thereof having one, two or three amino acid substitutions),
(b) a heavy chain CDR2 having an amino acid sequence of SEQ ID NO. 217 or a variant thereof having one, two or three amino acid substitutions (e.g. particularly of SEQ ID NO. 65, SEQ ID NO. 77 or a variant thereof having one, two or three amino acid substitutions), and/or
(c) a heavy chain CDR3 having an amino acid sequence of SEQ ID NO. 218 or a variant thereof having one, two or three amino acid substitutions (e.g. particularly of SEQ ID NO. 66, SEQ ID NO. 78 or a variant thereof having one, two or three amino acid substitutions).

In this embodiment, the anti-CLEC14A binding domain may particularly comprise one CDR from (a), one CDR from (b) and/or one CDR from (c), e.g. at least one CDR selected from SEQ ID NO. 64, SEQ ID NO. 65 and SEQ ID NO. 66 or at least one CDR selected from SEQ ID NO. 76, SEQ ID NO. 77 and SEQ ID NO. 78, or a variant of any of these sequences having one, two or three amino acid substitutions.

Thus, in a preferred embodiment, the anti-CLEC14A binding domain of the first aspect may comprise CDRs having the sequences of

SEQ ID NO. 64, SEQ ID NO. 65 and/or SEQ ID NO. 66, or

SEQ ID NO. 76, SEQ ID NO. 77 and/or SEQ ID NO. 78,

wherein any of the above listed sequences may have one, two, or three amino acid substitutions.

Further, the anti-CLEC14A binding domain of the first aspect may comprise at least one of
(a) a light chain CDR1 having an amino acid sequence of SEQ ID NO. 219 or a variant thereof having one, two or three amino acid substitutions (e.g. particularly of SEQ ID NO. 67, SEQ ID NO. 79 or a variant thereof having one, two or three amino acid substitutions),
(b) a light chain CDR2 having an amino acid sequence of SEQ ID NO. 220 or a variant thereof having one, two or three amino acid substitutions (e.g. particularly of SEQ ID NO. 68, STS or a variant thereof having one, two or three amino acid substitutions), and/or
(c) a light chain CDR3 having an amino acid sequence of SEQ ID NO. 221 or a variant thereof having one, two or three amino acid substitutions (e.g. particularly of SEQ ID NO. 69, SEQ ID NO. 81 or a variant thereof having one, two or three amino acid substitutions).

In this embodiment, the anti-CLEC14A binding domain may particularly comprise one CDR from (a), one CDR from (b) and/or one CDR from (c), e.g. at least one CDR selected from SEQ ID NO. 67, 68 or 69, or at least one CDR selected from SEQ ID NO. 79, 81 or STS, or a variant of any of these sequences having one, two or three amino acid substitutions.

Thus, particularly, in this aspect, the anti-CLEC14A binding domain may comprise CDRs having the sequences of
SEQ ID NO. 67, SEQ ID NO. 68 and/or SEQ ID NO. 69, or
SEQ ID NO. 79, STS and/or SEQ ID NO. 81,
wherein any one or more of the above sequences may comprise one, two or three amino acid substitutions.

The anti-CLEC14A binding domain of the first aspect may comprise at least one CDR selected from any one or more of SEQ ID Nos 64, 65, 66, 76, 77 and 78 and at least one CDR selected from any one or more of SEQ ID Nos 67, 68, 69, 79, 81 or STS e.g. two CDRs from SEQ ID Nos 64, 65, 66, 76, 77 and 78 and two CDRs from SEQ ID Nos. 67, 68, 69, 79, 81 or STS. Particularly, the anti-CLEC14A binding domain of the first aspect may comprise a CDR having the amino acid sequence of SEQ ID NO 64 or 76, a CDR having the amino acid sequence of SEQ ID NO.65 or 77, a CDR having the amino acid sequence of SEQ ID NO.66 or 78, a CDR having the amino acid sequence of SEQ ID NO.67 or 79, a CDR having the amino acid sequence of SEQ NO. 68 or STS and a CDR having the amino acid sequence of SEQ ID NO. 69 or 81, or one or more variants of any one of these sequences having one, two or three amino acid substitutions.

Particularly, the anti-CLEC14A binding domain may comprise CDRs having amino acid sequences of

SEQ ID NO 64, SEQ ID NO. 65, SEQ ID No. 66, SEQ ID NO. 67, SEQ ID NO 0.68 and/or SEQ ID NO. 69 or

SEQ ID NO. 76, SEQ ID NO. 77, SEQ ID NO. 78, SEQ ID NO. 79, STS and/or SEQ ID NO. 81

wherein any one or more of the above sequences may comprise one, two or three amino acid substitutions.

As discussed previously, according to the second aspect of the invention, the anti-CLEC14A binding domain comprises at least one of
(a) a heavy chain CDR having the amino acid sequence of SEQ ID NO. 167 or a variant thereof having one, two or three amino acid substitutions,
(b) a heavy chain CDR having the amino acid sequence of SEQ ID NO. 168 or a variant thereof having one, two or three amino acid substitutions,
(c) a heavy chain CDR having the amino acid sequence of SEQ ID NO. 169 or a variant thereof having one, two, or three amino acid substitutions,
(d) a light chain CDR having the amino acid sequence of SEQ ID NO. 129 or a variant thereof having one, two or three amino acid substitutions,
(e) a light chain CDR having the amino acid sequence of SEQ ID NO. 68 or a variant thereof having one, two or three amino acid substitutions, and/or
(f) a light chain CDR having the amino acid sequence of SEQ ID NO. 130 or a variant thereof having one, two or three amino acid substitutions.

Thus, in the second aspect, the anti-CLEC14A binding domain may comprise one, two or three CDRs from (a), (b) (c), (d), (e) and/or (f) and particularly one CDR from (a), one CDR from (b) and one CDR from (c), and/or one CDR from (d), one CDR from (e) and one CDR from (f), or a variant of any one or more of these sequences having one, two or three amino acid substitutions.

Particularly therefore, according to the second aspect, the anti-CLEC14A binding domain comprises CDRs having the amino acid sequences of SEQ ID NO. 167, SEQ ID NO. 168 and SEQ ID NO. 169 and/or SEQ ID NO. 129, SEQ ID NO. 68 and SEQ ID NO. 130.

As discussed above, the anti-CLEC14A binding domain may comprise a variant sequence of any of the CDR sequences discussed above. In this regard, one or more of any CDR sequences present in the anti-CLEC14A binding domain may be a variant sequence. For example, when the anti-CLEC14A binding domain comprises 1-3 CDRs from a variable heavy chain antibody sequence, any one, all or none of those CDRs may be a variant. Alternatively, when the anti-CLEC14A binding domain comprises 1-3 CDRs from a light chain antibody sequence, any one, all or none of those CDRs may be a variant. Thus, when the anti-CLEC14A binding domain comprises 6 CDRs (e.g. 3 from a heavy chain and 3 from a light chain), one, two, three, four, five or six of the CDR sequences present may be variant sequences and each individual CDR within the anti-CLEC14A binding domain may have one, two or three amino acid substitutions. The anti-CLEC14A binding domain may comprise both non-variant CDR sequences and variant CDR sequences. Alternatively, the anti-CLEC14A binding domain may comprise all variant CDR sequences or no variant CDR sequences.

As discussed previously, a variant CDR may comprise one, two or three amino acid substitutions. However, it will be appreciated that for shorter CDR sequences, it may be preferable to have fewer amino acid substitutions. For example, where the CDR sequence is only three amino acids in length, although three amino acid substitutions may be present (e.g. conservative substitutions), it may be preferably for the CDR to have, less than three substitutions, e.g. two, one or no amino acid substitutions.

As discussed further below the variants may have any amino acid substitution but preferably may have a conservative amino acid substitution. Particularly, an anti-CLEC14A binding domain (and the CAR within which it is comprised) comprising a variant CDR should remain capable of binding to CLEC14A as defined previously. It will be appreciated that the use of a variant CDR may change the binding activity of the anti-CLEC14A binding domain (e.g. the binding affinity for CLEC14A may increase or decrease, or the binding domain may recognise a different epitope of CLEC14A). However, as stated above, the use of one or more variant CDR sequences should still allow CLEC14A binding as previously defined, even if that binding is not identical to the binding of the anti-CLEC14A binding domain comprising one or more non-variant CDRs. Alternatively, the binding affinity may be similar, as previously described. It may be preferred to use one or more variant CDRs in an anti-CLEC14A binding domain which results in the anti-CLEC14A binding domain (and CAR of the invention), having a reduced binding affinity for CLEC14A. In this way off target CLEC14A binding may be reduced and particularly may be minimised (e.g. binding to non-tumour tissue), whilst on-target binding is maintained (i.e. binding to the tumour vasculature). Thus, in a further embodiment of the invention, a method for identifying an anti-CLEC14A binding domain having reduced binding affinity to CLEC14A, is encompassed, wherein said method comprises introducing one or more amino acid substitutions (particularly one, two or three amino acid substitutions) into a CDR sequence as defined herein comprised within an anti-CLEC14A binding domain and testing the variant domain for its binding affinity to CLEC14A.

The invention further provides for an anti-CLEC14A binding domain comprising a heavy chain variable region and/or a light chain variable region comprising any one or more of the above defined CDR sequences or a variant thereof. Thus, the above defined CDRs may be present within a heavy and/or light chain variable region within the anti-CLEC14A binding domain. As discussed previously, the anti-CLEC14A binding domain may comprise a VH having three CDRs and/or a VL having three CDRs wherein at least one of the CDRs is selected from one of the CDR sequences set out above, or a variant thereof having one, two or three amino acid substitutions.

In accordance with the first aspect of the invention, the anti-CLEC14A binding domain may comprise an amino acid sequence of

(a) SEQ ID NO. 56,

(b) SEQ ID NO. 88,

(c) SEQ ID NO. 90,

(d) SEQ ID NO. 104

(e) SEQ ID NO. 106 or

(f) SEQ ID NO. 121,

or a variant of any one of (a), (b), (c), (d), (e) or (f) having at least 80% identity thereto e.g. from one to twenty e.g. from one to ten amino acid substitutions.

In accordance with the second aspect of the invention, the anti-CLEC14A binding domain may comprise an amino acid sequence of SEQ ID NO. 173, or a variant thereof having at least 80% identity thereto e.g. having from one to twenty, e.g. from one to ten amino acid substitutions.

Thus, according to this aspect, the anti-CLEC14A binding domain may comprise a variable heavy chain sequence as set out above or a variant thereof having at least 80% identity thereto as defined further below e.g. having one, two, three, four, five, six, seven, eight, nine, ten, fifteen or twenty amino acid substitutions. It will be appreciated that the heavy chain variable sequences (e.g. those of SEQ ID NO. 56, 88, 90, 104, 106, 121 and 173) may comprise one or more CDRs (e.g. 3 CDRs). In this respect, although the above heavy chain variable region sequences may be altered e.g. by up to 20% (e.g. by up to twenty amino acid substitutions), it is preferred that any CDRs which occur within the heavy chain variable region are only subjected to a maximum of three amino acid substitutions each (e.g. one, two or three amino acid substitutions per CDR). Thus, with respect to SEQ ID NO. 56, although variants having at least 80% identity thereto are encompassed, it is preferred that the sequences of SEQ ID NO. 32, SEQ ID NO. 33 and SEQ ID NO. 34 comprised therein only have up to three amino acid substitutions each. With respect to SEQ ID NO. 88, it is preferred that the sequences SEQ ID NO. 64, 65 and 66 comprised therein only have up to three amino acid substitutions each. This also applies to SEQ ID Nos 76, 77 and 78 comprised within SEQ ID NO. 90, SEQ ID Nos 32, 33 and 100 comprised within SEQ ID NO. 104. SEQ ID Nos 44, 45 and 102 comprised within SEQ ID NO. 106, SEQ ID Nos 32, 33 and 116 comprised within SEQ ID NO. 121 and SEQ ID Nos 167, 168 and 169 comprised within SEQ ID No 173.

In accordance with the first aspect of the invention, the anti-CLEC14A binding domain may comprise an amino acid sequence of
(a) SEQ ID NO. 57
(b) SEQ ID NO. 89
(c) SEQ ID NO. 91
(d) SEQ ID NO. 105
(e) SEQ ID NO. 107 or
(f) SEQ ID NO. 122,
or a variant of any one of (a), (b), (c), (d), (e) or (f) having at least 80% identity thereto, e.g. having from one to twenty e.g. from one to ten amino acid substitutions.

In accordance with the second aspect of the invention, the anti-CLEC14A binding domain may comprise an amino acid sequence of SEQ ID NO. 133, or a variant thereof having at least 80% identity thereto e.g. having from one to twenty, e.g. from one to ten amino acid substitutions.

Thus, according to these aspects, the anti-CLEC14A binding domain may comprise a variable light chain sequence as set out above or a variant thereof having at least 80% identity thereto, e.g. having at least one, two, three, four, five, six, seven, eight, nine, ten, fifteen or twenty amino acid substitutions. It will be appreciated that the light chain variable sequences (e.g. those of SEQ ID NO.57, 89, 91, 105, 107 or 122 or 133) may comprise one or more CDRs (e.g. 3 CDRs). In this respect, although the above light chain variable region sequences may be altered e.g. by up to 20%, it is preferred that any CDRs which occur within the light chain variable region are only subjected to a maximum of three amino acid substitutions each (e.g. one, two or three amino acid substitutions per CDR). Thus, with respect to SEQ ID NO. 57, although variants having at least 80% identity thereto are encompassed, it is preferred that the sequences of SEQ ID NO. 35, SEQ ID NO. 36 and SEQ ID NO. 37 comprised herein only have up to three amino acid substitutions each. With respect to SEQ ID NO. 89, it is preferred that the sequences SEQ ID NO. 67, 68 and 69 comprised herein only have up to three amino acid substitutions each. This also applies to SEQ ID Nos 79, 81 and STS comprised within SEQ ID NO. 91, SEQ ID Nos 35, 36 and 37 comprised within SEQ ID NO. 105, SEQ ID Nos 47 and 49 and DTS comprised within SEQ ID NO. 107, SEQ ID Nos 35, 36 and 37 comprised within SEQ ID NO. 122 and SEQ ID Nos 129, 68 and 130 comprised within SEQ ID NO. 133.

In particular, according to the first aspect of the invention, the anti-CLEC14A binding domain may comprise heavy and light chain variable sequences which bind to CLEC14A (i.e. to the C-type lectin domain on CLEC14A). Hence, the anti-CLEC14A binding domain may comprise any one of SEQ ID Nos 56, 88, 90, 104, 106 or 121, or a variant thereof having at least 80% identity thereto, and any one of SEQ ID Nos 57, 89, 91, 105, 107 or 122, or a variant thereof having at least 80% identity thereto. Thus, in this aspect, the anti-CLEC14A binding domain may comprise

(a) SEQ ID NO. 56 or a variant thereof having at least 80% identity thereto and SEQ ID NO. 57 or a variant thereof having at least 80% identity thereto (b) SEQ ID NO. 88 or a variant thereof having at least 80% identity thereto and SEQ ID NO. 89 or a variant thereof having at least 80% identity thereto
(c) SEQ ID NO. 90 or a variant thereof having at least 80% identity thereto and SEQ ID NO. 91 or a variant thereof having at least 80% identity thereto
(d) SEQ ID NO 104 or a variant thereof having at least 80% identity thereto and SEQ ID NO. 105 or a variant thereof having at least 80% identity thereto
(e) SEQ ID NO 106 or a variant thereof having at least 80% identity thereto and SEQ ID NO. 107 or a variant thereof having at least 80% identity thereto, or
(f) SEQ ID NO. 121 or a variant thereof having at least 80% identity thereto and SEQ ID NO. 122 or a variant thereof having at least 80% identity thereto.

As discussed above, it is preferred that any CDRs present within the heavy and light chain variable regions of SEQ ID Nos 56, 57, 88, 89, 90, 91, 104, 105, 106, 107, 121 and 122 only have up to one, two or three amino acid substitutions per CDR.

In the second aspect of the invention, the anti-CLEC14A binding domain may comprise heavy and light chain variable sequences which bind to CLEC14A (i.e. to the C-type lectin domain on CLEC14A). Hence, the anti-CLEC14A binding domain may comprise a heavy chain variable sequence of SEQ ID NO. 173 and a light chain variable sequence of SEQ ID NO. 133, or a variant of either or both sequences having at least 80% identity thereto.

As discussed previously, a “variant” sequence according to the invention refers to a sequence which has a number of amino acid substitutions as compared to the defined or reference sequence (i.e. that provided by the SEQ ID NO.). Thus a variant sequence may have different amino acid residues as compared to the original sequence. Although any amino acid substitution may be made to obtain a variant sequence, as discussed previously, any such variant sequence should retain the functional activity, e.g. binding affinity, of the original sequence to some degree. Thus, although the variant may have increased or decreased functional activity (e.g. binding affinity) compared to the original sequence, some function should remain. In the case of the anti-CLEC14A binding domain of the invention comprising a variant CDR or a variant heavy or light chain, it is preferred that the anti-CLEC14A binding domain comprising the variant sequence can still bind to CLEC14A. Although the actual binding affinity of the variant may be different to an anti-CLEC14A binding domain (increased or decreased), binding to CLEC14A should still selectively occur.

An anti-CLEC14A binding domain comprising a variant sequence (e.g. CDR or heavy/light chain) should therefore preferably have substantially the same binding affinity as an anti-CLEC14A binding domain comprising non-variant sequence. For example, the variants may have at least 20, 30, 40, 50, 60, 70, 80, 85, 90, 95, 100, 105, 110, 115, 120 or 125% or more of the binding affinity of an anti-CLEC14A binding domain having non-variant sequence. Methods for detecting and measuring the binding affinity to CLEC14A are known in the art. For example, pull-down assays, enzyme linked immunosorbent assays (ELISA), surface plasmon resonance assays, chip-based assays, immunocytofluorescence, yeast-two-hybrid technology and phage display may be used.

The amino acid substitutions described herein may be conservative amino acid substitutions, for example where an amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., glycine, cysteine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus conservative amino acid substitutions include (original residue↔Substitution) Ala (A)↔Val, Gly or Pro; Arg (R)↔Lys or His; Asn (N)↔Gln; Asp (D)↔Glu; Cys (C)↔Ser; Gln (Q)↔Asn; Glu (G)↔Asp; Gly (G)↔Ala; His (H)↔Arg; Ile (I)↔Leu; Leu (L)↔Ile, Val or Met; Lys (K)↔Arg; Met (M)↔Leu; Phe (F)↔Tyr; Pro (P)↔Ala; Ser (S)↔Thr or Cys; Thr (T)↔Ser; Trp (W)↔Tyr; Tyr (Y)↔Phe or Trp; and Val (V)↔Leu or Ala.

In a further embodiment of the first aspect, the anti-CLEC14A binding domain may comprise a scFv comprising the heavy and light chains defined above, wherein said heavy and light chains are joined by a linker sequence as previously defined. Particularly in this aspect, the anti-CLEC14A binding domain may comprise an amino acid sequence of

(a) SEQ ID NO. 58

(b) SEQ ID NO. 96

(c) SEQ ID NO. 112, or

(d) SEQ ID NO. 125,

or a sequence which has at least 80% identity thereto.

In a further embodiment of the second aspect, the anti-CLEC14A binding domain may comprise a scFv comprising the heavy and light chains defined above (SEQ ID Nos 133 and 173), wherein said heavy and light chains are joined by a linker sequence as previously defined. Particularly in this aspect, the anti-CLEC14A binding domain may comprise an amino acid sequence of SEQ ID NO. 175 or a sequence which has at least 80% identity thereto.

Thus a skilled person will appreciate that it may be possible to use a variant sequence to the scFv sequences of SEQ ID NO. 58, 96, 112, 125 or 175 in the anti-CLEC14A binding domain of the invention. Such a variant, as discussed above, will preferably retain the binding affinity of the unmodified scFv sequence or will substantially retain the binding affinity of the unmodified scFv sequence, e.g. may have at least 20, 30, 40, 50, 60, 70, 80, 90, 100, 110 or 120% of the binding affinity of the unmodified scFv sequence.

An amino acid or nucleotide sequence of the invention having at least 80% identity to an unmodified amino acid or nucleotide sequence includes sequences having at least 85, 90, 95, 96, 97, 98 or 99% identity. For example, with respect to the above scFv sequences of SEQ ID No. 58, 96, 112, 125, or 175 sequences having at least 85, 90, 95, 96, 97, 98 or 99% identity thereto are encompassed. Sequence identity may be assessed by any convenient method. However, for determining the degree of identity between sequences, computer programs that make multiple alignments of sequences are useful, for instance Clustal W. If desired, the Clustal W algorithm can be used together with BLOSUM 62 scoring matrix and a gap opening penalty of 10 and gap extension penalty of 0.1, so that the highest order match is obtained between two sequences wherein at least 50% of the total length of one of the sequences is involved in the alignment. Other methods to calculate the percentage identity between two amino acid sequences are generally art recognized and include, for example, those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN, FASTA, gapped BLAST, BLASTP, BLASTN, or GCG are also useful for this purpose. Furthermore, the Dali server at the European Bioinformatics institute offers structure-based alignments of protein sequences.

By way of providing a reference point, sequences according to the present invention having 80%, 85%, 90%, 95%, 96%, 97%, 98% or 99% sequence identity etc. may be determined using the ALIGN program with default parameters (for instance available on Internet at the GENESTREAM network server, IGH, Montpellier, France).

Variant sequences having at least 80% sequence identity to the defined sequences of the invention, as discussed above, will preferably only have up to 3 amino acid substitutions within a particular CDR comprised therein. Therefore, although variants may show at least 80% identity to a defined sequence across its whole length, preferably any CDR comprised therein will only have a maximum of 3 amino acid substitutions, e.g. none, one, or two amino acid substitutions. Thus, it is preferable in this instance for the variation to occur in regions of the heavy or light variable chain sequences or scFv sequence outside of any CDRs e.g. within the framework region. For variation outside of any CDRs, e.g. in the framework regions, the variation may include amino acid substitutions, deletions and/or additions.

It will be appreciated that with respect to VH, VL and scFv, e.g. as within the anti-CLEC14A binding domain, variation may include humanisation of the framework regions. VH, VL or scFvs may be humanised in known ways, for example by inserting the CDR regions of murine sequences into the framework of human antibodies. Humanised antibodies can be made using the techniques and approaches described in Verhoeyen et al (1988) Science, 239, 1534-1536, and in Kettleborough et al, (1991) Protein Engineering, 14(7), 773-783. In some instances, Fv framework residues of the human immunoglobulin are replaced by corresponding non-human residues. In general, the humanised antibody will contain variable domains in which all or most of the CDR regions correspond to those of a non-human immunoglobulin, and framework regions which are substantially or completely those of a human immunoglobulin consensus sequence.

According to the first aspect, the polynucleotide sequence may comprise at least one of:
(a) SEQ ID NO. 38, SEQ ID NO. 50, SEQ ID NO. 82, a degenerate variant thereof, or a variant thereof having one, two or three nucleotide substitutions,
(b) SEQ ID NO. 39, SEQ ID NO. 51 or SEQ ID NO. 83, a degenerate variant thereof, or a variant thereof having one, two or three nucleotide substitutions, and/or
(c) SEQ ID NO. 40, SEQ ID NO. 52, SEQ ID NO. 84, SEQ ID NO. 101, SEQ ID NO. 103, SEQ ID NO. 117, SEQ ID NO. 120, a degenerate variant thereof, or a variant thereof having one, two or three nucleotide substitutions.

The nucleotide sequences defined above each encode a heavy chain CDR which may be present within the anti-CLEC14A binding domain described herein. The nucleotide sequences set out in (a) encode the heavy chain CDR1 sequences of antibodies CRT1, 3, 4 and 5 (thus SEQ ID NO 38 encodes SEQ ID NO. 32, SEQ ID NO. 50 encodes SEQ ID NO. 44 and SEQ ID NO. 82 encodes SEQ ID NO. 76), the nucleotide sequences set out in (b) encode the heavy chain CDR2 sequences of antibodies CRT1, 3, 4 and 5 (thus SEQ ID NO. 39 encodes SEQ ID NO. 33, SEQ ID NO. 51 encodes SEQ ID NO. 45 and SEQ ID NO. 83 encodes SEQ ID NO. 77) and the nucleotide sequences set out in (c) encode the heavy chain CDR3 sequences of antibodies CRT1, 3, 4 and 5 (thus SEQ ID NO. 40 encodes SEQ ID NO. 34, SEQ ID NO. 52 encodes SEQ ID NO. 46, SEQ ID NO. 84 encodes SEQ ID NO. 78, SEQ ID NO. 101 encodes SEQ ID NO. 100, SEQ ID NO. 117 encodes SEQ ID NO. 116 and SEQ ID NO. 120 encodes SEQ ID NO. 118). Thus, the polynucleotide may comprise any one of the defined nucleotide sequences, for example 2 or 3 of these sequences. Particularly, the polynucleotide sequence may comprise one nucleotide sequence from (a), one from (b) and/or one from (c).

Further, degenerate variants are encompassed. It will be appreciated, that due to the degeneracy in the genetic code that several different nucleotide sequences may encode the same amino acid sequence. Thus, for particular amino acids, more than one nucleotide codon can encode that amino acid. Degenerate variants thus encompass nucleotide variants that encode the same amino acid sequence as the nucleotide sequences defined by a SEQ ID NO. Particularly, degenerate variants may encompass codon optimised variants, where codon optimisation may be performed to enhance expression of the encoded sequence in a particular organism. This is standard practice in the art and a skilled person would be well aware of how to codon optimise a nucleotide sequence according to host.

Additionally or alternatively, the polynucleotide as defined in the first aspect may comprise at least one of the following nucleotide sequences:
(a) SEQ ID NO. 41, SEQ ID NO. 53, SEQ ID NO. 85, a degenerate variant thereof or a variant thereof having one, two or three nucleotide substitutions,
(b) SEQ ID NO. 42, GACACATCC, AGCACATCC, a degenerate variant thereof or a variant thereof having one, two or three nucleotide substitutions and/or
(c) SEQ ID NO. 43, SEQ ID NO. 55, SEQ ID NO. 87, a degenerate variant thereof or a variant thereof having one, two or three nucleotide substitutions.

The nucleotide sequences defined above each encode a light chain CDR which may be present within the anti-CLEC14A binding domain described herein. The nucleotide sequences set out in (a) encode the light chain CDR1 sequences of antibodies CRT1, 3, 4 and 5 (thus SEQ ID NO. 41 encodes SEQ ID NO. 35, SEQ ID NO. 53 encodes SEQ ID NO. 47, and SEQ ID NO. 85 encodes SEQ ID NO. 79), the nucleotide sequences set out in (b) encode the light chain CDR2 sequences of antibodies CRT1, 3, 4 and 5 (thus, SEQ ID NO. 42 encodes SEQ ID NO. 36, GACACATCC encodes DTS and AGCACATCC encodes STS) and the nucleotide sequences set out in (c) encode the light chain CDR3 sequences of antibodies CRT1, 3, 4 and 5 (thus SEQ ID NO. 43 encodes SEQ ID NO. 37, SEQ ID NO. 55 encodes SEQ ID NO. 49 and SEQ ID NO. 87 encodes SEQ ID NO. 81). Thus, the polynucleotide may comprise any one of the defined nucleotide sequences, for example 2 or 3 of these sequences. Particularly, the polynucleotide sequence may comprise one nucleotide sequence from (a), one from (b) and/or one from (c).

The polynucleotide sequence may comprise at least one nucleotide sequence selected from any one or more of SEQ ID Nos 38, 39, 40, 50, 51, 52, 82, 83, 84, 101, 103, 117 or 120 encoding a heavy chain CDR and at least one nucleotide sequence selected from any one or more of SEQ ID Nos 41, 42, 43, 53, 55, 85, 87, AGCACATCC or GACACATCC, encoding a light chain CDR e.g. at least two nucleotide sequences selected from SEQ ID Nos 38, 39, 40, 50, 51, 52, 82, 83, 84, 101, 103, 117 or 120 and at least two nucleotide sequences selected from SEQ ID Nos 41, 42, 43, 53, 55, 85, 87, AGCACATCC or GACACATCC. Particularly, the polynucleotide sequence may comprise one nucleotide sequence of SEQ ID Nos 38, 50 or 82; one nucleotide sequence of SEQ ID Nos 39, 51 or 83; one nucleotide sequence of SEQ ID Nos 40, 52, 84, 101, 103, 117, or 120; one nucleotide sequence of 41, 53, or 85; one nucleotide sequence of 42, or AGCACATCC or GACACATCC and one nucleotide sequence of 43, 55 or 87, or one or more variants of these sequences having one, two or three nucleotide substitutions.

More particularly, the polynucleotide may comprise at least one of the following nucleotide sequences
(a) SEQ ID NO. 38, SEQ ID NO. 50, a degenerate variant thereof or a variant thereof having one, two or three nucleotide substitutions,
(b) SEQ ID NO. 39, SEQ ID NO. 51, a degenerate variant thereof or a variant thereof having one, two or three nucleotide substitutions, and/or
(c) SEQ ID NO. 40, SEQ ID NO. 52, SEQ ID NO. 101, SEQ ID NO. 103, SEQ ID NO. 117, SEQ ID NO. 120, a degenerate variant thereof or a variant thereof having one, two or three nucleotide substitutions.

Thus, as indicated above, the polynucleotide may comprise two or three of the above described nucleotide sequences, and may particularly comprise one nucleotide sequence from (a), one from (b) and/or one from (c). More particularly, the polynucleotide sequence may comprise SEQ ID Nos 38, 39 and/or 40; SEQ ID Nos 50, 51 and/or 52; SEQ ID NOS 38, 39 and/or 101, SEQ ID Nos 50, 51 and/or 103; SEQ ID Nos 38, 39 and/or 117 or SEQ ID Nos 50, 51 and/or 120, or a variant of any of these sequences having one, two or three nucleotide substitutions.

In a particularly preferred embodiment, the polynucleotide of the first aspect may comprise nucleotide sequences of

### SEQ ID NO. 38, SEQ ID NO. 39 and SEQ ID NO. 40

### SEQ ID NO. 50, SEQ ID NO. 51 and SEQ ID NO. 52

### SEQ ID NO. 38, SEQ ID NO. 39 and SEQ ID NO. 101

### SEQ ID NO. 50, SEQ ID NO. 51 and SEQ ID NO. 103

### SEQ ID NO. 38, SEQ ID NO. 39 and SEQ ID NO. 117 or

### SEQ ID NO. 50, SEQ ID NO. 51 and SEQ ID NO. 120,

wherein any of the above sequences may comprise one, two or three nucleotide substitutions.

Further, the polynucleotide may comprise at least one of the following nucleotide sequences:
(a) SEQ ID NO. 41, SEQ ID NO. 53, a degenerate variant thereof or a variant thereof having one, two or three nucleotide substitutions,
(b) SEQ ID NO. 42 or GACACATCC, a degenerate variant thereof or a variant thereof having one, two or three nucleotide substitutions and/or
(c) SEQ ID NO. 43, SEQ ID NO. 55, a degenerate variant thereof or a variant thereof having one, two or three nucleotide substitutions.

Thus, as indicated above, the polynucleotide may comprise two or three of the above described nucleotide sequences, and may particularly comprise one nucleotide from (a), one from (b) and/or one from (c). Thus, preferably, the polynucleotide may comprise at least one nucleotide sequence selected from SEQ ID NO. 41, 42 and/or 43; or at least one nucleotide sequence selected from SEQ ID NO. 53, 55 and/or GACACATCC, or a variant of any one or more of these sequences having one, two or three nucleotide substitutions.

In a particular embodiment, the polynucleotide sequence may comprise SEQ ID NO. 41, SEQ ID NO. 42 and SEQ ID NO. 43 or SEQ ID NO. 53, GACACATCC and SEQ ID NO. 55, wherein any one of more of the nucleotide sequences may have one, two or three amino acid substitutions or be a degenerate sequence.

The polynucleotide of the first aspect may comprise at least one nucleotide sequence selected from any one or more of SEQ ID Nos 38, 39, 40, 50, 51, 52, 101, 103, 117 or 120 and at least one nucleotide sequence selected from any one or more of SEQ ID Nos 41, 42, 43, 53, or 55 or GACACATCC, e.g. at least two nucleotide sequences selected from any one or more of SEQ ID Nos 38, 39, 40, 50, 51, 52, 101, 103, 117 or 120 and at least two nucleotide sequences selected from any one or more of SEQ ID Nos 41, 42, 43, 53, or 55 or GACACATCC. Particularly, the polynucleotide sequence may comprise a nucleotide sequence of SEQ ID NO. 38 or 50; a nucleotide sequence of SEQ ID NO. 39 or 51; a nucleotide sequence of SEQ ID NO. 40, 52, 101, 103, 117 or 120, a nucleotide sequence of SEQ ID NO. 41 or 53; a nucleotide sequence of SEQ ID NO. 42 or GACACATCC and a nucleotide sequence of SEQ ID NO. 43 or 55.

Particularly, the polynucleotide sequence may comprise nucleotide sequences of:

### SEQ ID NO. 38, 39, 40, 41, 42 and 43;

### SEQ ID NO. 50, 51, 52, 53, 55 and GACACATCC;

### SEQ ID NO. 38, 39, 101, 41, 42 and 43;

### SEQ ID NO. 50, 51, 103, 53, 55 and GACACATCC;

### SEQ ID NO. 38, 39, 117, 41, 42 and 43; or

### SEQ ID NO. 50, 51, 120, 53, 55 and GACACATCC,

wherein any one or more of the above SEQ ID Nos may comprise one, two or three amino acid substitutions or be a degenerate sequence thereof.

Alternatively, the polynucleotide sequence may comprise at least one of
(a) SEQ ID NO. 82, a degenerate variant thereof or a variant thereof having one, two or three nucleotide substitutions,
(b) SEQ ID NO. 83, a degenerate variant thereof or a variant thereof having one, two or three nucleotide substitutions, and/or
(c) SEQ ID NO. 84, a degenerate variant thereof or a variant thereof having one, two or three nucleotide substitutions

Particularly, the polynucleotide sequence may comprise two of the sequences of SEQ ID NO. 82, 83 or 84 or may comprise all of SEQ ID Nos 82, 83 and 84.

Further, the polynucleotide may comprise at least one of
(a) SEQ ID NO. 85, a degenerate variant thereof or a variant thereof having one, two or three nucleotide substitutions,
(b) AGCACATCC, a degenerate variant thereof or a variant thereof having one, two or three nucleotide substitutions, and/or
(c) SEQ ID NO. 87, a degenerate variant thereof or a variant thereof having one, two or three nucleotide substitutions.

Particularly, the polynucleotide sequence may comprise two of the sequences of SEQ ID NO. 85, 87 or AGCACATCC or may comprise all of SEQ ID NOs 85, 87 and AGCACATCC. The polynucleotide sequence of the first aspect may comprise at least one nucleotide sequence selected from any one or more of SEQ ID NO. 82, 83 and 84 and at least one nucleotide sequence selected from SEQ ID NO. 85, 87 and AGCACATCC, e.g. two nucleotide sequences from SEQ ID Nos 82, 83 and 84 and two nucleotide sequences from SEQ ID Nos 85, 87 and AGCACATCC. Particularly, the polynucleotide sequence may comprise all of SEQ ID Nos 82, 83, 84, 85, 87 and AGCACATCC, wherein any one or more of the sequences may comprise one, two or three nucleotide substitutions.

According to the second aspect of the invention, the polynucleotide sequence may comprise at least one of
(a) SEQ ID NO. 170, a degenerate variant thereof or a variant having one, two or three nucleotide substitutions,
(b) SEQ ID NO. 171, a degenerate variant thereof or a variant having one, two or three nucleotide substitutions, and/or
(c) SEQ ID NO. 172, a degenerate variant thereof or a variant having one, two or three nucleotide substitutions.

Thus, the polynucleotide sequence may comprise two nucleotide sequences selected from SEQ ID NO. 170, SEQ ID NO. 171 and SEQ ID NO. 172. Particularly, according to the second aspect, the polynucleotide sequence may comprise SEQ ID NO. 170, SEQ ID NO. 171 and SEQ ID NO. 172.

As discussed previously, according to the second aspect, of the invention, the polynucleotide sequence may comprise at least one of
(a) SEQ ID NO. 131, a degenerate variant thereof or a variant having one, two or three nucleotide substitutions,
(b) SEQ ID NO. 74, a degenerate variant thereof or a variant having one, two or three nucleotide substitutions, and/or
(c) SEQ ID NO. 132, a degenerate variant thereof or a variant having one, two or three nucleotide substitutions.

Thus in the second aspect, the polynucleotide sequence may comprise two nucleotide sequences selected from SEQ ID NO. 131, SEQ ID NO. 74 and SEQ ID NO. 132. Particularly, according to the second aspect, the polynucleotide sequence may comprise SEQ ID NO 131, 74 and 132.

Further, according to the second aspect, the polynucleotide sequence may comprise any one or more of SEQ ID Nos 170, 171, 172, 131, 74 and/or 132. Particularly, the polynucleotide sequence may comprise all of SEQ ID Nos 170, 171, 172, 131, 74 and 132, or a degenerate variant of any one or more of those sequence, or a variant of any one of more of the sequences having one, two or three nucleotide substitutions.

As discussed above, the polynucleotide sequence may comprise a variant of any of the nucleotide sequences set out above. In this regard, one or more of the nucleotide sequences may be a variant sequence. For example, when the polynucleotide comprises 1-3 of the defined nucleotide sequences which encode heavy chain CDRs, any one, all or none of those sequences may be a variant. Alternatively, when the polynucleotide sequence comprises 1-3 of the defined nucleotide sequences which encode light chain CDRs, any one, all of none of those sequences may be a variant. Thus, when the polynucleotide comprises 6 nucleotide sequences encoding light and heavy chain CDRs, (e.g. 3 encoding a light chain and 3 encoding a heavy chain), one, two, three, four, five or six of the CDR encoding nucleotide sequences may be variant sequences and each individual nucleotide sequence may comprise one, two or three nucleotide substitutions. The polynucleotide sequence may comprise both variant and non-variant nucleotide sequences, or alternatively, may comprise all variant or no variant nucleotide sequences.

It will be appreciated by a skilled person that nucleotide substitutions within a sequence may or may not result in an amino acid change in the encoded protein or polypeptide sequence, due to the degeneracy of the nucleic acid code. Thus, multiple codons may encode the same amino acid. In this respect, a nucleotide variant of the invention may encode the same amino acid sequence as a non-variant sequence. If the nucleotide substitution does result in an amino acid substitution, it is preferred as discussed above, that the substitution is conservative (although the invention is not limited to conservative amino acid substitutions) and that the encoded anti-CLEC14A binding domain has the function discussed previously above.

In accordance with the first aspect of the invention, the polynucleotide sequence may comprise any one of

(a) SEQ ID NO. 59,

(b) SEQ ID NO. 92,

(c) SEQ ID NO. 94,

(d) SEQ ID NO. 108,

(e) SEQ ID NO. 110, or

(f) SEQ ID NO. 123,
or a variant of any one of (a), (b), (c), (d), (e) or (f) having from one to ten nucleotide substitutions.

Thus, according to the first aspect of the present invention, the polynucleotide sequence may comprise a nucleotide sequence as set out above which encodes a heavy chain variable region, or a variant thereof having 1, 2, 3, 4, 5, 6, 7, 8, 9 or 10 nucleotide substitutions. It will be appreciated that the encoded heavy chain variable region will comprise at least one CDR (particularly 3 CDRs). In this respect, although the above nucleotide sequence may be varied by up to 10 nucleotides, it is preferred that the portions of the nucleotide sequences of SEQ ID Nos 59, 92, 94, 108, 110 and 123 which encode CDRs are only varied by up to three nucleotide substitutions per CDR. Particularly, it is preferred that any variation occurs to the nucleotide sequence of SEQ ID Nos 59, 92, 94, 108, 110 and 123 outside of any regions which encode a CDR, e.g. to regions or portions of the sequence which encode the framework regions. Thus, for SEQ ID NO. 59, although up to 10 nucleotide substitutions may be made to this sequence, a maximum of three substitutions may be made to each of SEQ ID Nos 38, 39 and 40, or SEQ ID Nos 50, 51 and 52 comprised within SEQ ID NO. 59. For SEQ ID NO. 92, a maximum of three nucleotide substitutions may be made to each of SEQ ID Nos 70, 71 or 72; for SEQ ID NO. 94, a maximum of three nucleotide substitutions may be made to each of SEQ ID Nos 82, 83 and 84; for SEQ ID NO. 108, a maximum of three nucleotide substitutions may be made to each of SEQ ID Nos 38, 39 and 101; for SEQ ID NO. 110, a maximum of three nucleotide substitutions may be made to each of SEQ ID Nos 50, 51 and 52 and for SEQ ID NO. 123, a maximum of three nucleotide substitutions may be made to each of SEQ ID Nos 38, 39 and 117.

In accordance with the first aspect, the polynucleotide sequence may comprise any one of:

(a) SEQ ID NO. 60,

(b) SEQ ID NO. 93,

(c) SEQ ID NO. 95,

(d) SEQ ID NO. 109,

(e) SEQ ID NO. 111 or

(f) SEQ ID NO. 124, or
a variant of any one of (a), (b), (c), (d), (e) or (f) having from one to ten nucleotide substitutions, or a degenerate variant thereof.

In accordance with the second aspect of the invention, the polynucleotide sequence may comprise SEQ ID NO. 174 and/or SEQ ID NO. 134, or a variant thereof having from one to ten nucleotide substitutions.

Thus, the polynucleotide sequence may comprise a nucleotide sequence as set out above (SEQ ID Nos 60, 93, 95, 109, 111, 124 or 134) which encodes a light chain variable region, or a variant thereof having 1, 2, 3, 4, 5, 6, 7, 8, 9 or 10 nucleotide substitutions. It will be appreciated that the encoded light chain variable region will comprise at least one CDR (particularly 3 CDRs). In this respect, although the above nucleotide sequence may be varied by up to 10 nucleotides, it is preferred that the portion of the nucleotide sequences of SEQ ID Nos 60, 93, 95, 109, 111, 124 or 134 which encode a CDR is only varied by up to three nucleotide substitutions. Particularly, it is preferred that any variation occurs to the nucleotide sequence of SEQ ID Nos 60, 93, 95, 109, 111, 124 or 134 outside of any regions which encode a CDR, e.g. to regions or portions of the sequence which encode the framework regions. Thus, for SEQ ID NO. 60, although up to 10 nucleotide substitutions may be made to this sequence, a maximum of three substitutions may be made to each of SEQ ID Nos 41, 42 and 43, or SEQ ID Nos 53, 55 and GACACATCC comprised within SEQ ID NO. 60. For SEQ ID NO. 93, a maximum of three nucleotide substitutions may be made to each of SEQ ID Nos 73, 74 and 75; for SEQ ID NO. 95, a maximum of three nucleotide substitutions may be made to each of SEQ ID Nos 85, 87 and AGCACATCC; for SEQ ID NO. 109, a maximum of three nucleotide substitutions may be made to each of SEQ ID Nos 41, 42 and 43; for SEQ ID NO. 111, a maximum of three nucleotide substitutions may be made to each of SEQ ID Nos 53, 55 and GACACATCC; for SEQ ID NO. 124, a maximum of three nucleotide substitutions may be made to each of SEQ ID Nos 53, 55 and GACACATCC and for SEQ ID NO. 134, a maximum of three nucleotide substitutions may be made to each of SEQ ID Nos 131, 74 and 132.

In a particular embodiment of the invention, the polynucleotide may comprise a sequence which encodes both heavy and light chain variable regions of an antibody, which bind to CLEC14A (to the C-type lectin domain of CLEC14A according to the first aspect of the invention). Thus, the polynucleotide may comprise any one of SEQ ID Nos 59, 92, 94, 108, 110, 123, and 174 or a variant thereof having from 1-10 nucleotide substitutions, and any one of SEQ ID Nos 60, 93, 95, 109, 111, 124 and 134, or a variant thereof having from 1-10 nucleotide substitutions. Thus, in this aspect, the polynucleotide may comprise
(a) SEQ ID NO. 59, a degenerate variant thereof or a variant thereof having from 1-10 nucleotide substitutions and SEQ ID NO. 60, a degenerate variant thereof or a variant thereof having from 1-10 nucleotide substitutions;
(b) SEQ ID NO. 92, a degenerate variant thereof or a variant thereof having from 1-10 nucleotide substitutions and SEQ ID NO. 93, a degenerate variant thereof or a variant thereof having from 1-10 nucleotide substitutions;
(c) SEQ ID NO. 94, a degenerate variant thereof or a variant thereof having from 1-10 nucleotide substitutions and SEQ ID NO. 95, a degenerate variant thereof or a variant thereof having from 1-10 nucleotide substitutions;
(d) SEQ ID NO. 108, a degenerate variant thereof or a variant thereof having from 1-10 nucleotide substitutions and SEQ ID NO. 109, a degenerate variant thereof or a variant thereof having from 1-10 nucleotide substitutions;
(e) SEQ ID NO. 110, a degenerate variant thereof or a variant thereof having from 1-10 nucleotide substitutions and SEQ ID NO. 111, a degenerate variant thereof or a variant thereof having from 1-10 nucleotide substitutions;

(f) SEQ ID NO. 123, a degenerate variant thereof or a variant thereof having from 1-10 nucleotide substitutions and SEQ ID NO. 124, a degenerate variant thereof or a variant thereof having from 1-10 nucleotide substitutions; or
(g) SEQ ID NO. 174, a degenerate variant thereof or a variant thereof having from 1-10 nucleotide substitutions and SEQ ID NO. 134, a degenerate variant thereof or a variant thereof having from 1-10 nucleotide substitutions.

In connection with this, the polynucleotide may comprise a nucleotide sequence which encodes a scFv comprising light and heavy variable chains of an antibody which bind to CLEC14A, wherein said light and heavy chains may be joined by a linker sequence as previously defined. Particularly, the polynucleotide sequence may comprise one of the following sequences which encode a scFv:

(a) SEQ ID NO. 61

(b) SEQ ID NO. 97

(c) SEQ ID NO. 113

(d) SEQ ID NO. 126, or

(e) SEQ ID NO. 176
or a degenerate variant thereof or a sequence with at least 80% identity to any one of (a), (b), (c) or (d).

Thus, a skilled person will appreciate that it may be possible to use a variant sequence to the above scFv encoding nucleotide sequences (SEQ ID Nos 61, 97, 113, 126 or 176), wherein such a variant sequence, as discussed above, will preferably encode a scFv which retains the binding affinity of the unmodified scFv or substantially retains the binding affinity of the unmodified scFv sequence, e.g. may have at least 50, 60, 70, 80, 90, 100, 110 or 120% of the binding affinity of the scFv sequence. Particularly, the scFv may have the binding affinity of the anti-CLEC14A binding domain, as discussed previously above.

As discussed herein, degenerate variants of the defined nucleotide sequences are also encompassed. Particularly, codon optimised nucleotide sequences are encompassed which are optimised for expression within cells of a particular organism. For example, polynucleotide sequences which are codon optimised for expression in human or murine cells may be developed and are encompassed by the present invention.

More particularly, a polynucleotide sequence encoding a scFv comprised in the anti-CLEC14A binding domain may be codon optimised. In this respect, the anti-CLEC14A binding domain as defined herein may be encoded by or may comprise an amino acid sequence encoded by a polynucleotide comprising any one of SEQ ID Nos 177, 178, 179, 180, 181, 182, 183, 184, 185 or 186, or a variant having at least 80% identity thereto, wherein said variant encodes a scFv which is capable of binding to CLEC14A, as defined previously. In this aspect, SEQ ID Nos 177 and 178 relate to human and murine codon optimised sequences of SEQ ID NO. 61, respectively; SEQ ID Nos 179 and 180 relate to human and murine codon optimised sequences of SEQ ID NO. 176, respectively; SEQ ID Nos 181 and 182 relate to human and murine codon optimised sequences of SEQ ID NO. 97, respectively; SEQ ID Nos 183 and 184 relate to human and murine codon optimised sequences of SEQ ID NO. 113, respectively and SEQ ID Nos 185 and 186 relate to human and murine codon optimised sequences of SEQ ID NO. 126, respectively.

Sequence identity can be determined as previously discussed. Further, as already discussed, it is preferred that the variation occurs in regions of the nucleotide sequences that do not encode the CDR regions. These regions are as discussed above.

Although the above defined nucleotide sequences are DNA, in an alternative embodiment of the invention, the nucleotide sequences may be RNA. Thus corresponding RNA sequences to the DNA sequences described herein are encompassed. A skilled person will appreciate how to derive a RNA sequence encoding the same protein/polypeptide product to those DNA sequences set out above e.g. “T” should be substituted with “U”.

The term “nucleic acid sequence” or “nucleic acid molecule” or “polynucleotide” or “nucleotide sequence” as used herein refers to a sequence of nucleoside or nucleotide monomers composed of naturally occurring bases, sugars and intersugar (backbone) linkages. The term also includes modified or substituted sequences comprising non-naturally occurring monomers or portions thereof. The nucleic acid, polynucleotide or nucleotide sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid, polynucleotide or nucleotide sequences may be double stranded or single stranded. The nucleic acid, polynucleotide or nucleotide sequences may be wholly or partially synthetic or recombinant.

As discussed above, the polynucleotide described herein encodes a CAR which comprises an anti-CLEC14A binding domain, a transmembrane domain and an intracellular signalling domain.

“A transmembrane domain” as used herein may be based on or derived from the transmembrane domain of any transmembrane protein. Typically it may be, or may be derived from, a transmembrane domain from CD8α, CD28, CD4, CD3ζ CD45, CD9, CD16, CD22, CD33, CD64, CD80, CD86, CD134 (OX40), CD137 (4-1BB), and CD154, preferably human CD8α, CD28, CD4, CD3ζ CD45, CD9, CD16, CD22, CD33, CD64, CD80, CD86, CD134, CD137, and CD154. In one embodiment, the transmembrane domain may be, or may be derived from, a transmembrane domain from CD8a, CD28, CD4, or CD3ζ, preferably from human CD28, CD4, or CD3ζ. In another embodiment the transmembrane domain may be synthetic in which case it would comprise predominantly hydrophobic residues such as leucine and valine. Thus, the transmembrane domain is capable of spanning or being present within the cell membrane of a cell. As discussed above, the transmembrane domain may be derived from a protein comprising an extracellular and/or intracellular portions and thus the transmembrane domain as used herein may be attached to extracelluar and/or intracellular residues derived from the protein of origin, in addition to the portion within or spanning the cell membrane. For example, the transmembrane domain may be attached to a hinge or spacer region derived from the protein of origin e.g. a transmembrane domain derived from CD8α may be attached to a spacer or hinge domain derived from CD8α. The presence of a transmembrane domain within a cell membrane can be assessed using any suitable method known in the art, including fluorescence labelling with fluorescence microscopy.

The transmembrane domain may in one embodiment link the anti-CLEC14A binding domain of the CAR to the intracellular signalling domain, where the intracellular signalling domain may be derived from a different protein from the transmembrane domain or may be derived from the same protein as the transmembrane domain (e.g. the transmembrane domain and the intracellular domain may have the same sequence as transmembrane domains and intracellular domains which are naturally found within the same protein). Thus in one embodiment, the transmembrane domain and the intracellular signalling domain may be from the same protein or derived from the same protein. In another embodiment, the transmembrane domain may be derived from a protein which also comprises a co-stimulatory portion and thus the CAR may comprise both the transmembrane domain from that protein and also the portion which is capable of providing a co-stimulatory signal.

The transmembrane domain as used herein may have a sequence which differs from the sequence of a naturally occurring transmembrane domain, as long as the domain is still capable of being present within the membrane. For example, a transmembrane domain may have at least 70, 80, 90, 95, 96, 97, 98 or 99% sequence identity to a transmembrane domain of a naturally occurring protein, as long as the modified domain is capable of spanning a cell membrane. Sequence identity may be measured as discussed previously.

In a preferred embodiment the transmembrane domain is the CD28 transmembrane domain having the amino acid sequence of SEQ ID NO. 146 or an amino acid sequence having at least 95% sequence identity thereto. Alternatively viewed, the CD28 transmembrane domain may be encoded by a nucleotide sequence of SEQ ID NO. 147, or a nucleotide sequence having at least 95% sequence identity thereto.

In a further embodiment, the transmembrane domain is the CD8a transmembrane domain encoded by the nucleotide sequence as set out in SEQ ID NO. 119, or a nucleotide sequence having at least 95% sequence identity thereto. This transmembrane sequence may further be attached to a hinge domain from CD8a as shown in SEQ ID NO. 165, or a sequence having at least 95% sequence identity thereto.

The “intracellular signalling domain” as used herein refers to the part of the CAR protein that participates in transducing the message of effective CAR binding to a target antigen (CLEC14A) into the interior of a cell (host cell e.g. an immune effector cell) to elicit cell function (e.g. effector cell function) e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of the cell. Effector function of the T cell, for example, may be cytolytic activity or help or activity including the secretion of a cytokine. An “effector cell” is thus a cell having such an effector function. Thus, the term “intracellular signalling domain” refers to the portion of a protein which transduces the effector function signal and that directs the cell to perform a specialized function. While the entire intracellular signalling domain of a naturally occurring protein can be employed in the present invention, in many cases it is not necessary to use the entire domain. To the extent that a variant e.g. truncated portion of a naturally occurring intracellular signalling domain is used, such variant (e.g. truncated portion) may be used in place of the entire domain as long as it transduces the effector function signal, e.g. has at least 50, 60, 70, 80, 90 or 95% of the ability to transduce the effector function as the full length domain. A variant (e.g. truncated) intracellular signalling domain may further have an increased ability to transduce the effector function signal e.g. at least 105, 110, 120, 130 or 140% ability to transduce the effector function compared to the full length intracellular signalling domain. The ability to transduce the effector function may be measured by measuring the effector function of a cell after interaction with target e.g. by measuring cytokine release, cell proliferation etc. Thus, the term intracellular signalling domain is meant to include any truncated portion of an intracellular signalling domain sufficient to transduce effector function signal.

A variant intracellular signalling domain may have at least 70, 80, 90 or 95% sequence identity to a naturally occurring intracellular signalling domain. It will be appreciated that if a truncated domain is being used, the % sequence identity may be less than 70% as compared to the full length sequence. The intracellular signalling domain is also known as the, “signal transduction domain,” and is typically derived from portions of the human CD3ζ or FcRy chains.

Other examples of intracellular signalling domains for use in the CAR encoded by the polynucleotide described herein include the cytoplasmic sequences of the T cell receptor (TCR) and co-receptors that act in concert to initiate signal transduction following antigen receptor engagement, as well as any variants of these sequences as discussed above. It is known that signals generated through the TCR alone are generally insufficient for full activation of a T cell and that a secondary and/or costimulatory signal may also be required. Thus, T cell activation can be said to be mediated by two distinct classes of signalling sequence: those that initiate antigen-dependent primary activation through the TCR (intracellular signalling domains) and those that act in an antigen-independent manner to provide a secondary or costimulatory signal (such as a costimulatory domain). Costimulatory domains promote activation of effector functions and may also promote persistence of the effector function and/or survival of the cell.

Intracellular signalling domains that act in a stimulatory manner may contain signalling motifs which are known as immunoreceptor tyrosine-based activation motifs (ITAMs) (e.g. 2, 3, 4, 5, or more ITAMs). For example, CD3 zeta, Fc receptor gamma, Fc receptor beta, CD3 gamma, CD3 delta, CD3 epsilon, CD5, CD22, CD79a, CD79b and CD66d comprise one or more ITAMs. Thus, in one embodiment, the intracellular signalling domain used herein may comprise one or more ITAMs, e.g. from anyone or more of CD3 zeta, Fc receptor gamma, Fc receptor beta, CD3 gamma, CD3 delta, CD3 epsilon, CD5, CD22, CD79a, CD79b and CD66d. It will be appreciated as discussed above, that a variant of an ITAM may be used within the intracellular signalling domain, as long as the intracellular signalling domain is capable of inducing effector function as previously discussed.

Particularly, a CAR as defined herein may comprise an intracellular signalling domain derived from CD3 zeta, and more particularly, an intracellular signalling domain comprising the sequence of SEQ ID NO. 148 or an amino acid sequence with at least 95% identity thereto, or encoded by a nucleotide sequence of SEQ ID NO. 149, or a nucleotide sequence with at least 95% identity thereto.

As indicated previously, the polynucleotide may encode a CAR comprising additional portions or domains i.e. in addition to the anti-CLEC14A binding domain, the transmembrane domain and the intracellular signalling domain. Thus, particularly, the CAR may additionally comprise at least one costimulatory domain. As discussed above, the presence of at least one costimulatory domain is often preferable to provide optimal effector function from a cell within which the CAR is expressed. Thus, although the CARs may comprise only an intracellular signalling domain, in a particular embodiment, a costimulatory domain will also be present.

The “costimulatory domain” refers to a portion or region of an intracellular domain of a costimulatory molecule. A costimulatory molecule may be a cell surface molecule other than an antigen receptor or its ligands, that is required for an efficient response of cells to an antigen (e.g. immune cells to an antigen). Examples of costimulatory molecules include CD28, 4-1BB (CD137), OX40, ICOS, DAP10, CD27, CD30, CD40, ICOS, lymphocyte function-associated antigen-1 (LFA-1), CD2, CD7, LIGHT, NKG2C, B7-H3, and a ligand that specifically binds with CD83 and the like.

As discussed above, if the costimulatory molecule additionally comprises a transmembrane portion, it is possible that the transmembrane domain and the costimulatory domain of a CAR described herein, may be derived from the same protein. In a particular embodiment, the CAR may comprise a transmembrane domain and a costimulatory domain from CD28.

The intracellular signalling domain and costimulatory domain(s) present within a CAR of the invention, may be linked to each other in any order (e.g. random or a specified order). Optionally, a short oligo- or polypeptide linker for example between 2 and 10 amino acids (e.g. 2, 3, 4, 5, 6, 7, 8, 9 or 10 amino acids) in length may form the linkage between intracellular signalling sequences of the intracellular signalling domain and the one or more costimulatory domains. In one embodiment, a glycine-serine doublet can be used as a suitable linker. In another embodiment, a single amino acid, such as an alanine or a glycine can be used as a suitable linker.

The CAR encoded by the nucleic acid of the invention may comprise two or more, for example 3, 4, 5 or more costimulatory signalling domains. In an embodiment, the costimulatory signalling domains may be separated by a linker as described above, e.g. by a glycine or alanine residue.

Particularly, the CAR of the invention may comprise an intracellular signalling domain from CD3 zeta, e.g. one comprising amino acid sequence SEQ ID NO. 148 or an amino acid sequence having at least 95% identity thereto, and a costimulatory domain of CD28, 4-1BB, OX40, ICOS or DAP-10. The costimulatory domain of OX40 particularly has an amino acid sequence as set out in SEQ ID NO. 168 or has at least 95% identity thereto. Alternatively viewed, the costimulatory domain of OX40 may be encoded by a nucleotide sequence as set forth in SEQ ID NO. 48 or a sequence which has at least 95% identity thereto. The costimulatory domain of 4-1BB may be encoded by a nucleotide sequence as set out in SEQ ID NO. 80 or a sequence which has at least 95% identity thereto and the costimulatory domain of CD28 may be encoded by a nucleotide sequence as set out in SEQ ID NO. 54 or a sequence which has at least 95% identity thereto. More particularly, the CAR of the invention may comprise an intracellular signalling domain from CD3 zeta, e.g. one comprising amino acid sequence SEQ ID NO. 148 or an amino acid sequence having at least 95% identity thereto and the costimulatory domains of CD28 and OX40, the costimulatory domains of CD28 and 4-1BB or the costimulatory domains of 4-1BB and OX40.

Further, the polynucleotide of the invention may encode a CAR comprising 1) transmembrane and costimulatory domains from CD28 and an intracellular signalling domain from CD3 zeta; 2) a transmembrane domain from CD8a, a costimulatory domain from 4-1BB and an intracellular signalling domain from CD3 zeta; 3) a transmembrane domain from CD8a, a costimulatory domain from OX40 and an intracellular signalling domain from CD3 zeta; 4) a transmembrane domain from CD28, costimulatory domains from CD28 and 4-1BB and an intracellular signalling domain from CD3 zeta; 5) a transmembrane domain from CD28, costimulatory domains from CD28 and OX40 and an intracellular signalling domain from CD3 zeta; 6) a transmembrane domain from CD8a, costimulatory domains from 4-1BB and OX40 and an intracellular signalling domain from CD3 zeta; 7) a transmembrane domain from CD8a, a costimulatory domain from CD28 and an intracellular signalling domain from CD3 zeta; 8) a transmembrane domain from CD8a, costimulatory domains from CD28 and 4-1BB and an intracellular signalling domain from CD3 zeta or 9) a transmembrane domain from CD8a, costimulatory domain from CD28 and OX40 and an intracellular signalling domain from CD3 zeta. Particularly, any one of the constructs comprising a transmembrane domain from CD8a may be further comprise a hinge or spacer domain which is also derived from CD8a, e.g. one as defined in SEQ ID NO. 165 or a sequence which has at least 95% identity thereto.

The polynucleotide may further encode a CAR comprising a leader sequence. The term “leader sequence” refers to a peptide sequence which targets the CAR to the cell membrane. The leader sequence may be present to the N-terminus of the anti-CLEC14A binding domain, and/or it is possible for the leader sequence to be cleaved from the CAR during cellular processing and localisation of the CAR to the cell membrane. A typical leader sequence that may be used in a CAR as described herein is the oncostatin M leader sequence of SEQ ID NO. 135, the CD8a leader sequence encoded by SEQ ID NO. 162, or a variant thereof having at least 70, 80, 85, 90, 95, 96, 97, 98 or 99% identity thereto, which is capable of targeting the CAR to the cell membrane. The essential portion of a leader sequence typically comprises a stretch of hydrophobic amino acids that have a tendency to form a single alpha-helix.

The CAR may further comprise a hinge domain or spacer region (used interchangeably herein) between the anti-CLEC14A binding domain and the transmembrane domain. The hinge domain and/or spacer may have flexibility to allow it to orientate in different directions, which may aid antigen binding to the anti-CLEC14A binding domain. In certain embodiments, a hinge region and/or spacer may be an immunoglobulin hinge region and may be a wild type immunoglobulin hinge region or an altered wild type immunoglobulin hinge region, for example a truncated hinge region. Other exemplary hinge regions and/or spacers which may be used include the hinge region and/or spacer derived from the extracellular regions of type 1 membrane proteins such as CD8a, CD4, CD28 and CD7, which may be wild-type hinge regions/spacers from these molecules or may be altered. Preferably the hinge region/spacer is, or is derived from, the hinge region/spacer of human CD8α, CD4, CD28 or CD7. IgD, CH3 and Fc spacers or hinges may also be used in a CAR of the invention.

An “altered wild type hinge or spacer region” or “altered hinge or spacer region” refers to (a) a wild type hinge/spacer region with up to 30% amino acid changes (e.g. up to 25%, 20%, 15%, 10%, or 5% amino acid changes e.g. substitutions or deletions), (b) a portion of a wild type hinge/spacer region that is at least 10 amino acids (e.g., at least 12, 13, 14 or 15 amino acids) in length with up to 30% amino acid changes (e.g., up to 25%, 20%, 15%, 10%, or 5% amino acid changes, e.g. substitutions or deletions), or (c) a portion of a wild type hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length). When an altered wild type hinge region is interposed between and connecting the CLEC14A-specific binding domain and another region (e.g., a transmembrane domain) in the chimeric antigen receptors described herein, it allows the chimeric fusion protein to maintain specific binding to CLEC14A.

In certain embodiments, one or more cysteine residues in a wild type immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Hinge regions comprising the CH₂ and CH₃ constant region domains are described in the art for use in CARs (for example the CH₂CH₃ hinge, referred to as an “Fc hinge” or “IgG hinge”, as shown in SEQ ID NO.163. Alternatively viewed, the CH2CH3 hinge may be encoded by SEQ ID NO. 164. However, it is preferred that when the hinge domain is based on or derived from an immunoglobulin it does not comprise a CH₃ domain, e.g. it may comprise or consist of the CH₂ domain or a fragment or part thereof, without including CH₃.

In one embodiment the hinge domain has or comprises the amino acid sequence of SEQ ID NO. 165 (which represents the hinge domain of CD8a) or an amino acid sequence having at least 95% sequence identity thereto.

In another preferred embodiment the hinge domain has or comprises the amino acid sequence of SEQ ID NO. 166 (which represents a shortened IgG hinge) or an amino acid sequence having at least 95% sequence identity thereto.

The hinge domain may be attached to the transmembrane domain by a linker sequence, which may be a linker sequence as defined above. An exemplary linker sequence is KDPK (SEQ ID NO. 159). Such a sequence, or a sequence having at least 95% sequence identity thereto, may be included in a CAR encoded by a polynucleotide described above. More particularly such a sequence may be included between the extracellular domain (e.g. the scFv part) and the transmembrane domain. A hinge domain for use in a particular CAR may be determined empirically.

A hinge domain or spacer region as used herein may be at least 10 amino acids in length, for example, at least 20, 30, 40, 50, 60, 70, 80, 90, 100, 150 or 200 amino acids in length.

In a particular embodiment of the invention, a hinge domain may not be employed and the anti-CLEC14A binding domain may be directly attached to the transmembrane domain.

Thus, according to the first aspect of the present invention, the polynucleotide may encode a CAR comprising the sequence of any one of

(a) SEQ ID NO. 62,

(b) SEQ ID NO. 98,

(c) SEQ ID NO. 114, or

(d) SEQ ID NO. 127, or
a variant thereof having at least 80% identity to any of (a), (b), (c) or (d).

Thus, the CAR may have at least 85, 90, 95, 96, 97, 98 or 99% identity to any one of SEQ ID Nos 62, 98, 114 or 127. Particularly a variant CAR should retain the activity of the CAR having a sequence of SEQ ID NO. 62, 98, 114 or 127 e.g. should have at least 50, 60, 70, 80, 90, or 95% of the activity of a non-variant CAR. This may be measured as the binding affinity of the CAR, which can be determined as previously discussed with respect to the anti-CLEC14A binding domain, or may be measured as the ability to stimulate effector function within a cell, which may be determined as discussed above in relation to the intracellular signaling domain.

Alternatively viewed, the polynucleotide encoding a CAR may comprise a nucleotide sequence of any one of

(a) SEQ ID NO. 63,

(b) SEQ ID NO. 99,

(c) SEQ ID NO. 115, or

(d) SEQ ID NO. 128,
or a variant thereof having at least 80% identity to any one of (a), (b), (c) or (d).

As discussed above, the variant may have at least 85, 90, 95, 96, 97, 98 or 99% identity to any one of SEQ ID Nos 63, 99, 115 or 128, and the encoded CAR should retain the activity of a CAR encoded by a non-variant sequence, as discussed above.

The present invention provides a CAR (polypeptide) encoded by a nucleic acid molecule of the invention. The term “polypeptide” or “protein” are used interchangeably herein and mean a polymer of amino acids, not limited to any particular length. The term does not exclude modifications such as myristylation, sulfation, glycosylation, phosphorylation and addition or deletion of signal sequences. The terms “peptide”, “polypeptide” or “protein” thus mean one or more chains of amino acids, wherein each chain comprises amino acids covalently linked by peptide bonds.

The present invention further encompasses a vector comprising a nucleic acid of the invention. The vector may for example be an expression vector (e.g. a mRNA expression vector or an expression vector for transfer into an immune cell (e.g. a viral vector)) or a cloning vector. Possible expression vectors include but are not limited to transposons, cosmids, plasmids, or modified viruses (e.g., replication defective retroviruses, adenoviruses and adeno-associated viruses and lentiviruses), so long as the vector is compatible with the host cell used. Particularly, the expression vector may be a gamma retrovirus, such as that described in Engels et al, Human Gene Therapy, 14:1155-1168, 2003, or Schambach et al, Mol. Ther. 2:435-445, 2000, which are incorporated herein by reference The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates a recombinant expression vector containing a nucleic acid molecule of the invention, and the necessary regulatory sequences for the transcription and translation of the protein sequence encoded by the nucleic acid molecule of the invention.

Suitable regulatory sequences may be derived from a variety of sources, including bacterial, fungal, viral, mammalian, or insect genes. Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

An example of a promoter that is capable of expressing a CAR molecule in a cell (a mammalian cell) is the EF1a promoter, or the CMV promoter. Further examples of promoters include the SV40 early promoter, mouse mammary tumour virus (MMTV), HIV long terminal repeat promoter, MoMuLV promoter, an avian leukaemia virus promoter, an Epstein-Barr virus immediate early promoter, a Rous sarcoma virus promoter, or a MPSV LTR (as described in Engel et al, supra).

As indicated above, transcription of the CAR may be controlled using an inducible system. Particularly, an inducible promoter may be used to control expression of the CAR, where for example, expression may be induced by a small molecule or drug (e.g. which binds to a promoter, regulatory sequence or to a transcriptional repressor or activator molecule) or by using an environmental trigger.

Particularly, CAR expression may be controlled using tetracycline or a derivative such as doxycycline, e.g. using a Tet-on system, where one or more tet operator sequences (e.g. at least 2, 3, 4 or 5) may be incorporated into or near to a promoter. Gene expression from the promoter may then be controlled by the addition of tetracycline or one of its derivatives (e.g. doxycycline), which may bind to a tetracycline transactivator protein, allowing its association with the tet operator sequence. The tetracycline transactivator protein may be expressed from the same or an additional vector to the CAR of the invention. Variations of the Tet-on system are well known in the art and may be utilised in the present invention.

Further, CAR expression may be controlled by the addition of tamoxifen e.g. using a system where an activator is fused to a mutated ERT domain. In this respect, a Cre/loxP system may be utilised, and particularly a modified version of this system, where Cre is fused to a mutated form of the ligand binding domain of the estrogen receptor (ERT), which only binds to tamoxifen. This fusion is inactive until addition of tamoxifen which activates Cre and allows recombination between the lox P sites, which allows transcription of the CAR. Such a system allows the inducible expression of the CAR by addition of tamoxifen.

Other drug inducible systems are well known in the art, e.g. systems activated on the addition of ponasterone A (e.g. using a gene for the ecdysone receptor and a promoter with a binding site for the receptor), systems activated on the addition of coumermycin, and any such systems can be used in accordance with the present invention for CAR expression.

As discussed above, expression systems may also be employed in the invention, where CAR expression is controlled by an environmental trigger, e.g. hypoxia, radiation, increased temperature etc. Particularly, a hypoxia inducible promoter may be used for CAR expression in the present invention, e.g. a chimeric promoter comprising hypoxia responsive elements.

Novel inducible promoters may further be developed for use in the present invention e.g. inducible promoters which are activated by a small molecule or drug.

The recombinant expression vectors of the invention may also contain a selectable marker gene that facilitates the selection of host cells transformed or transfected with a recombinant molecule of the invention. Examples of selectable marker genes are genes encoding a protein such as neomycin and hygromycin that confer resistance to certain drugs, β-galactosidase, chloramphenicol acetyltransferase, firefly luciferase, or an immunoglobulin or portion thereof such as the Fc portion of an immunoglobulin, preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors may also contain genes that encode a fusion moiety that provides increased expression of the recombinant protein; increased solubility of the recombinant protein; and aid in the purification of the target recombinant protein by acting as a ligand in affinity purification (for example appropriate “tags” to enable purification and/or identification may be present, e.g., His tags or myc tags). For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The terms “transformed with”, “transfected with”, “transformation”, “transduction” and “transfection” are intended to encompass introduction of nucleic acid (e.g., a vector) into a cell by one of many possible techniques known in the art. The term “transformed host cell” or “transduced host cell” as used herein is intended to also include cells capable of glycosylation that have been transformed with a recombinant expression vector of the invention. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofection, electroporation or microinjection. Suitable methods for transforming and transfecting host cells can be found in Sambrook et al., 1989 (supra), and other laboratory textbooks.

The vectors comprising the nucleic acids of the invention may be transduced or transfected into any cell type, e.g. in order to carry out in vitro investigations of the encoded CAR molecule, or to produce additional vector or RNA/viral vector for transduction into a cell for administration to a patient. For example, the vectors may be transduced into a wide variety of eukaryotic host cells and prokaryotic cells, e.g. yeast cells or mammalian cells or Escherichia coli. The present invention thus further provides a cell comprising a nucleic acid or a vector of the invention. The invention additionally provides a cell comprising a CAR of the invention.

Mammalian cells may include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), NS-1 cells, NS0 (ATCC CRL-11177), and Per.C6® (Crucell, Leiden, Netherlands). Suitable expression vectors for directing expression in mammalian cells generally include a promoter (e.g., derived from viral material such as polyoma, Adenovirus 2, cytomegalovirus and Simian Virus 40 or derived from any viral LTR), as well as other transcriptional and translational control sequences. Examples of mammalian expression vectors include pCDM8, pMT2PC and pMP71.

For therapeutic uses, a vector of the invention may be transduced into a mammalian cell, particularly an immune cell, such as a T cell (e.g. a human T cell). A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art, e.g. a lentiviral vector such as HIV, SIV or FIV. Particularly, as indicated above, a retroviral vector such as a gamma retrovirus may be used, e.g. MP71.

The vectors of the invention comprising the nucleic acid of the invention may further comprise additional nucleotide sequences which may encode a further protein or polypeptide, in addition to the CAR molecule. Such additional proteins or polypeptides may be encoded by a nucleotide sequence under the control of the same promoter as the nucleotide sequence encoding the CAR molecule or under the control of a different promoter to the nucleotide sequence encoding the CAR molecule. If the additional protein/polypeptide is encoded by a nucleotide sequence under the control of the same promoter as the nucleotide sequence encoding the CAR molecule (e.g. where both sequences are downstream of the promoter), a further nucleotide sequence may be present between the nucleotide sequence encoding the CAR and the nucleotide sequence encoding the further protein/polypeptide, enabling their separation after expression e.g. their cleavage. Such further nucleotide sequences are well known in the art and include those encoding an intein. Alternatively, if expression from the same promoter is desired, an IRES or 2A peptide sequence can be employed in the vector of the invention.

In this respect, it may be desirable to additionally express a polypeptide from the CAR expressing vector of the invention, to allow detection of the expression of the CAR in the cell. Thus, it may be possible to identify the successful transduction of a cell with the vector and the successful expression of a CAR molecule by detecting the expression of a further polypeptide under the control of the same (or a different promoter) to the nucleotide sequence encoding the CAR. Particularly, the CAR molecules of the invention may additionally comprise a CD34 molecule or a modified CD34 molecule, e.g. a truncated CD34 molecule, where such a molecule comprises an extracellular portion which allows its detection by well-known techniques, e.g. immunofluorescence using a suitable antibody and label. In a particular embodiment, the vector of the invention may additionally comprise the nucleotide sequence of SEQ ID NO.145, or a nucleotide sequence having at least 80% sequence identity thereto. Alternatively viewed, the vector may additionally encode an amino acid sequence of SEQ ID NO. 144 or a sequence having at least 80% sequence identity thereto. Other molecules which may be co-expressed with the CAR molecule of the invention to allow the identification of CAR-transduced cells include luciferase.

In this respect, the invention particularly encompasses a nucleic acid molecule comprising a polynucleotide encoding a CAR comprising the sequence of any one of SEQ ID Nos 136, 138, 140 or 142, or a sequence having at least 80% identity thereto (and retaining the functional activity of the non-modified CAR as described previously). More particularly the nucleic acid molecules of the invention may comprise a polynucleotide sequence of SEQ ID NO. 137, 139, 141 or 143, or a sequence having at least 80% identity thereto. Particularly such modified sequences will encode a CAR which retains or substantially retains the functional activity of the non-modified CAR as previously described. Vectors comprising these sequences are also encompassed. CARS comprising the sequence of any one of SEQ ID Nos 136, 138, 140, or 142 comprise the oncostatin M leader sequence. It will be appreciated, as discussed above, that this could be substituted with another leader sequence, and particularly may be substituted with a leader sequence of CD8α. Thus, the sequences of SEQ ID Nos 136, 138, 140 or 142 may be modified to substitute the oncostatin M leader sequence of SEQ ID NO. 135 with the CD8α leader encoded by SEQ ID NO. 162.

As discussed previously, the present invention provides nucleic acid molecules which when transduced into an appropriate cell type are capable of expressing CAR molecules which can bind to CLEC14A. The nucleic acid molecules of the invention can thus be used to treat conditions associated with an increased expression of CLEC14A, and particularly can be used to inhibit angiogenesis (e.g. tumour angiogenesis). The treatment of such conditions relies upon the binding of the expressed CAR molecules to target antigen, i.e. to CLEC14A, which is expressed within tumour vasculature. It will be appreciated that binding of the CAR molecules to non-target CLEC14A may not be desirable and that any such binding whether at the time of administration of transduced cells to a patient, or at a subsequent time should be avoided. In order to achieve this, it may be desirable to ensure that transduced cells either do not survive long term in the patient (i.e. after treatment of the condition) or that transduced cells only transiently express the CAR molecules which target CLEC14A.

In order to achieve transient expression of CAR molecules, it is possible to transduce a cell with RNA (e.g. mRNA), encoding a CAR as described herein and to use those cells for administration to a patient. mRNA expression vectors for production of mRNA may be prepared according to methods known in the art (e.g. using Gateway Technology) and are known in the art (e.g. pCIpA102, Sæbøse-Larssen et al, 2002, J. Immunol. Methods 259, p 191-203 and pCIpA120-G, Wälchli et al, 2011, PLoS ONE 6 (11) e27930). In this respect, the invention particularly provides cells comprising a RNA molecule encoding a CAR as described herein.

Further, the mRNA can be produced in vitro by e.g. in vitro transcription. The mRNA may then be introduced into the immune effector cells, e.g. as naked mRNA, e.g. by electroporation (as described for example in Almasbak et al., Cytotherapy 2011, 13, 629-640, Rabinovich et al., Hum. Gene Ther., 2009, 20, 51-60 and Beatty et al., Cancer Immunol. Res. 2014, 2, 112-120).

Alternatively or additionally, it may be desirable to transduce cells with a nucleotide sequence which results in cell death once activated (a so called “suicide gene”). In this way, once cells have been used to treat the condition, the suicide gene can be activated, resulting in the death and removal of the CAR expressing cells from the patient. The suicide gene may be expressed from the same vector as the CAR molecule of the invention, e.g. using an element as previously discussed (a separate promoter, or the same promoter together with an intein, IRES or 2A peptide), or may be expressed from a different vector which may be transduced into the cells at the same time, prior to or subsequently to the vector or nucleic acid molecule encoding the CAR. Examples of suicide genes which may used in the present invention include Caspase 9, RQR8 and/or TK. One or more of these genes may be transduced into a cell within the vector encoding the CAR, or at the same time, prior to or subsequently to the vector or nucleic acid molecule encoding the CAR. It will be appreciated, that it is desirable for expression of any suicide gene to be controlled inducibly

It may also be desirable to make further modifications to cells transduced with or to be transduced with a vector or nucleic acid of the invention. Particularly modifications to immune cells which prolong or enhance their response to CLEC14A may be desirable. For example, it is known that TGFβ is secreted by tumours and that this may suppress the induction of T cells. In this respect, it may be desirable for the modified immune cells e.g. T cells of the invention (i.e. those transduced with a nucleic acid or a vector of the invention), to be capable of neutralising the effect of TGFβ, e.g. by expressing a dominant-negative TGFβ receptor II. Additionally, or alternatively, a cell of the invention may be transduced with a nucleic acid encoding a cytokine, e.g. IL-15, or IL2, IL7, IL12 etc, which may enhance the effector function of the cell. Again, as discussed above, any additional nucleic acid sequences may be expressed from the same or a different vector to the CAR molecule.

It will further be appreciated that a cell of the invention may comprise more than one nucleic acid or vector of the invention. Particularly, a cell of the invention may comprise 2, 3, 4 or 5 or more nucleic acids or vectors of the invention which each express a different CAR molecule. Thus, a cell of the invention may comprise different CAR molecules which are capable of binding to CLEC14A, e.g. at the same or different positions on CLEC14A. In this aspect, a cell of the invention may comprise a CAR molecule comprising a scFv which binds to CLEC14A and a CAR molecule comprising a ligand (e.g. MMRN2 or a portion or variant thereof) which binds to CLEC14A.

Further, a cell of the invention may comprise at least one other receptor (particularly exogenous) (e.g. multiple receptors) in addition to the expressed CAR of the invention, which may be used together with the CAR in a combinatorial approach to bind to the target cells (e.g. CLEC14A expressing tumour vasculature). Thus, in such an approach, binding of both the CAR and the at least one other receptor to the target cell may be required to stimulate an immune response against the target cell (e.g. each CAR/receptor may only provide a partial signal for immune cell stimulation, which alone may not be sufficient for immune cell stimulation but together allows for immune cell stimulation). In the case where the cell of the invention is a T cell, both CAR binding to CLEC14A and the at least one other receptor binding to its ligand on the CLEC14A expressing cell may be necessary to stimulate the T cell. The at least one other receptor may be a further CAR molecule.

In a variation of this embodiment, a CAR within a cell of the invention may be inducibly expressed. Particularly, in this embodiment, the binding of the at least one other receptor expressed on the cell to its target may allow or control the expression of the CAR molecule. Thus, in this instance, the binding of the at least one other receptor to its ligand is required before CAR expression occurs, and immune cell stimulation thus requires the binding of the at least one other receptor to its ligand and the subsequent binding of the CAR to the target cell. Such a particular system may comprise the additional expression of a SynNotch receptor, which is engineered with an extracellular ligand binding domain directed to an antigen of interest e.g. CD19, and an orthogonal transcription factor (e.g. TetR or Gal4). Upon binding to the antigen of interest, the orthogonal transcription factor is cleaved from the tail of the SynNotch receptor and activates the expression of the CAR. Thus, a cell of the invention may further comprise a nucleic acid or vector encoding a receptor which binds to an antigen other than CLEC14A, particularly to a tumour associated antigen other than CLEC14A.

Alternatively, a combinatorial approach may also be used where a further receptor in addition to the CAR of the invention is expressed on a cell of the invention, wherein said further receptor is capable of binding to off target cells or tissue (e.g. to non-tumour cells). In this case, if the further receptor binds to its ligand, a negative signal is produced, preventing immune cell stimulation (e.g. T cell stimulation).

A further combination approach may use a further receptor in combination with a CAR of the invention where both receptors bind to different targets and induce different effects to treat a tumour. Thus, both anti-tumour effects may be completely independent of each other but together may present an effective therapy against a tumour. In this regard, a CAR of the invention may be used in combination with a TCR therapy, where immune cells may be transduced with one or more nucleic acid molecules encoding a CAR of the invention and a TCR which is capable of binding to a particular MHC/peptide combination which may be found on a tumour cell (e.g. on a particular type of tumour cell or on any tumour cell). Alternatively, immune cells transduced with a nucleic acid encoding a CAR and a separate population of immune cells transduced with a nucleic acid encoding a TCR may be provided separately, sequentially or simultaneously. Gene therapy treatments using one or more nucleic acids encoding a CAR of the invention and a TCR which recognises a tumour MHC/peptide combination are also envisaged.

Regardless of the method used to introduce exogenous nucleic acids into a host cell, the presence of the nucleic acid within the cell can be determined using a variety of assays which are well known in the art, such as Southern and Northern blotting, RT-PCR and PCR. Further, as discussed previously, the expression of the CAR or other polypeptide may be detected using immunofluorescence techniques, ELISAs or by Western blotting.

In this respect, the invention further provides a method of producing a cell expressing a CAR molecule comprising the step of transducing a cell with a nucleic acid or a vector of the invention.

As discussed previously, the cell of the invention comprising a nucleic acid, vector and/or CAR of the invention, may be an immune cell, particularly a mammalian immune cell, such as a human immune cell. Immune cells are capable of having an effector function as previously described and include T cells and NK cells. The T-cell may be any type of T-cell, including an alpha-beta T cell, a gamma-delta T cell, a memory T cell (e.g. a memory T cell with stem cell-like properties). The NK cell may be an invariant NK cell.

The term “mammalian” as used herein refers to any mammal, but particularly refers to a human, a domestic animal (e.g. a cat, dog etc), a horse, a mouse, a rat, a primate, such as a monkey, a cow, a pig etc.

The T cells may be obtained from a number of sources, including from peripheral blood mononuclear cells, bone marrow, lymph node tissue, cord blood, thymus tissue, tissue from a site of infection, ascites, pleural effusion, spleen tissue and tumours. Particularly in the present invention, immune or T cells may be obtained from a subject having a condition which may be treated with a nucleic acid, vector or cell of the invention, e.g. a subject with a condition associated with an increased level of expression of CLEC14A, or more particularly with a tumour expressing CLEC14A in the tumour vasculature. T cells (or immune cells) may be obtained by any method known in the art.

T cells may also be obtained “off the shelf” and thus may not necessarily be obtained from a subject having a condition which may be treated with a nucleic acid, vector or cell of the invention. Thus T cells for use in the present invention may have previously been stored and/or modified prior to transduction with a nucleic acid or vector of the invention.

Particularly, T cells may be obtained from a unit of blood (particularly anticoagulated blood) collected from a subject using any suitable techniques in the art such as Ficoll separation. Alternatively, immune cells (e.g. T cells) may be obtained from a subject (typically a mammalian subject) by apheresis, where the apheresis product typically comprises lymphocytes (including T cells, monocytes, granulocytes, B cells, other nucleated white blood cells, red blood cells and platelets). It will be appreciated that cells collected by apheresis may be washed to remove the plasma fraction and to place the cells in an appropriate buffer or media for subsequent processing, e.g. the cells may be washed using PBS, or using a wash solution lacking divalent ions e.g. lacking calcium and/or magnesium. Washing steps may be achieved by methods known in the art e.g. by using a semi-automated “flow-through” centrifuge (e.g. the Cobe 2991 cell processor, the Baxter CytoMate, or the Haemonetics Cell Saver 5). After washing, the cells may be resuspended in a variety of biocompatible buffers, such as for example, Ca-free, Mg-free PBS, PlasmaLyte A, RPMI 1640 (Sigma) or PBS or other saline solution with or without buffer Typically, in the UK, cells for transfusion are treated in accordance with the guidelines found at http://www.transfusionguidelines.org.uk/red-book. Further procedures acceptable in Europe may be found in the Guide on the preparation, Use and Quality assurance of Blood Components, Current edition, EDQM. In the US, typically the AABB Blood and Blood components guidelines are followed. The WHO requirements also exist for the collection, processing QC of blood, blood components and plasma derivatives.

T cells may be isolated from peripheral blood lymphocytes by lysing red blood cells and depleting the monocytes, e.g. by centrifugation through a PERCOLL™ gradient or by counter-flow centrifugal elutriation. It is possible to select specific populations of T cells for use in the present invention. However, selection is not compulsory and a mixed population of cells (e.g. comprising different types of T-cells) may be transduced with the nucleic acid or vector of the invention (e.g. for use in inhibiting tumour angiogenesis in a subject). Subpopulations of cells which may be selected include CD3+, CD28+, CD4+, CD8+, CD45RA+ and CD45RO+ T cells. Selection of particular populations may be achieved using beads coupled with antibodies which selectively bind to antigens expressed on T cells populations. A combination of antibodies directed to surface markers uniquely expressed in particularly T cell populations may be used for selection.

It may be desirable to store the immune cells prior to their use, particularly prior to use in a therapeutic method of the invention. In this respect, it is possible to freeze or to incubate the cells of the invention (e.g. on a rotator at 2-10° C.). The cells may be stored in such a way either before and/or after transduction with a nucleic acid or vector of the invention.

As previously discussed, the transduced immune cells of the invention have therapeutic utility and particularly may be used to inhibit tumour angiogenesis. Prior to using the cells therapeutically, it may be desirable to subject the cells to a step of activation or expansion, using methods which are well known in the art. Any such steps may be carried out before or after transduction with a nucleic acid or vector of the invention. T cells may be expanded using an agent that stimulates a CD3/TCR complex associated signal and a ligand that stimulates a costimulatory molecule on the surface of the T cells. For example T cells may contacted with an anti-CD3 antibody and an anti-CD28 antibody under conditions appropriate for stimulating proliferation of the T cells.

The invention additionally specifically provides a population of cells, wherein at least one cell of said population comprises a nucleic acid or vector of the invention. The population of cells may comprise cells comprising different nucleic acids or vectors of the invention. Thus, one cell of the population may comprise a nucleic acid of the invention encoding a first CAR and a second cell of the population may comprise a nucleic acid of the invention encoding a second CAR.

Further, the invention provides an “off the shelf” cell product, where a cell (particularly a T cell) of the invention (i.e. comprising a nucleic acid or vector of the invention) may be stored and provided for later use (e.g. in therapy). Typically such cells may be allogeneic immune cells (e.g. T cells).

The invention further provides a composition or pharmaceutical composition comprising a nucleic acid, vector, cell or population of cells of the invention, and additionally a pharmaceutical composition comprising a nucleic acid, vector, cell or population of cells of the invention for use in therapy (or combating disease). The composition or pharmaceutical composition of invention may comprise an additional or further active (e.g. therapeutic) agent. The nucleic acid, vector, CAR, cell, population of cells or composition of the invention may be used to treat conditions associated with an increased level of expression of CLEC14A, and particularly, the nucleic acid, vector, CAR, cell, population of cells or composition of the invention may be used to inhibit angiogenesis within a tumour (e.g. wherein the tumour vasculature expresses CLEC14A). Thus, in this aspect, the present invention provides a nucleic acid, vector, CAR, cell, population of cells or composition of the invention for use in therapy. Further, the invention provides use of a nucleic acid, vector, CAR, cell, population of cells or composition of the invention in the manufacture of a medicament for use in combating disease. Alternatively viewed, the present invention provides a method of combating disease comprising the step of administering a nucleic acid, a vector, CAR, a cell, population of cells or composition of the invention to a subject in need thereof. More particularly, the invention provides a nucleic acid, vector, CAR, cell, population of cells or composition of the invention for use in treating a condition associated with expression of CLEC14. Further, the invention provides use of a nucleic acid, vector, CAR, cell, population of cells or composition of the invention in the manufacture of a medicament for treating a condition associated with expression of CLEC14A. Alternatively viewed, the present invention provides a method of treating a condition associated with expression of CLEC14A comprising a step of administering a nucleic acid, a vector, CAR, a cell, population of cells or composition of the invention to a subject in need thereof, e.g. a subject having the condition.

Although it is possible to use the nucleic acid and/or vector of the invention to directly treat a patient as described above, e.g. in a gene therapy method, in a particular embodiment, a cell comprising a nucleic acid or vector of the invention is used in therapy. It is preferred that such a cell is an immune cell, particularly a T-cell. Thus, the invention particularly provides a T-cell comprising a vector or nucleic acid of the invention for use in therapy, e.g. to treat a condition associated with expression of CLEC14A.

By “combating” we include the meaning that the method can be used to alleviate symptoms of a disorder (i.e. the method is used palliatively), or to treat the disorder, or to prevent the disorder (i.e. the method is used prophylactically).

A “condition associated with expression of CLEC14A” refers to any disease condition, which it is desired to treat, prevent or ameliorate, where CLEC14A is expressed. As discussed previously, CLEC14A expression in normal healthy tissues and normal vasculature is very low or undetectable. Thus, generally, the expression of CLEC14A (i.e. any detectable expression), in a tissue, particularly vasculature, may be associated with a disease condition. In this regard, any detection of CLEC14A expression (e.g. mRNA or protein) may be indicative of disease. CLEC14A can be measured and detected as previously described, e.g. using immunofluorescence. The detection of expression of CLEC14A therefore refers to the detection of an increased amount of CLEC14A in a tissue of a subject as compared to the amount of CLEC14A present in a healthy subject, in a corresponding tissue. Alternatively, the increase in expression of CLEC14A may be an increase compared to the expression of CLEC14A in the same subject prior to disease, or in a non-diseased part of the tissue within the same subject. The level of CLEC14A may be increased by at least 50, 60, 70, 80, 90, 100, 200, 300, 400 or 500%, or alternatively viewed may be increased by at least 100, 200, 300, 400, 500, 600, 700, 800, 900 or 1000 fold.

As previously discussed, CLEC14A is expressed within tumour vasculature and is associated with angiogenesis and thus, the condition associated with expression of CLEC14A may include any condition comprising unwanted angiogenesis. Particularly, the condition includes the treatment of solid tumours, (i.e. CLEC14A expressing solid tumours), menorrhagia, endometriosis, arthritis (both inflammatory and rheumatoid), macular degeneration, Paget's disease, retinopathy and its vascular complications (e.g. proliferative and of prematurity and diabetic retinopathy), benign vascular proliferations, fibroses, obesity and inflammation.

The treatment of a condition associated with expression of CLEC14A includes the treatment of an existing condition associated with expression of CLEC14A or the prevention of a condition associated with expression of CLEC14A. “Treatment” as used herein refers to the improvement or the prevention of a worsening of a disease state within a subject. For example, treatment includes the reduction in size of a tumour, a reduction in growth rate of a tumour, a reduction in the rate of metastasis of a tumour, or a maintenance of the size, growth rate or rate of metastasis of a tumour. By “reduction” is meant a reduction of at least 5, 10, 20, 30, 40, 50, 60, 70, 80 or 90%. By “maintenance” is meant no substantial increase, e.g. an increase of no more than 10, 5, 3, 2 or 1%. Tumour size can be determined by any method known in the art, e.g. tumour imaging with an appropriate antibody, MRI etc, tumour growth rate can be determined by measuring an increase in tumour size over time and determining how much a tumour grows over a particular time period. The rate of metastasis can be determined by measuring the time period over which tumour growth begins at new sites within a subject.

“Tumour” as used herein refers to all forms of neoplastic cell growth, and particularly includes solid tumours. A solid tumour for treatment in the present invention includes tumours of the breast, ovary, liver, bladder, prostate, kidney, pancreas, stomach, oesophagus, rectum, lung (e.g. mesothelioma), brain, cervix, colon, skin (e.g. melanoma), uterus, nervous system (e.g. neuroblastoma), thyroid and sarcomas, such as osteosarcomas. Particularly, a pancreatic or ovarian tumour may be treated in the present invention, e.g. using a T-cell of the invention.

Treatment of a condition associated with expression of CLEC14A includes the inhibition of angiogenesis. The term “inhibiting angiogenesis” is intended to mean reducing the rate or level of angiogenesis. The reduction can be a low level reduction of about 10%, or about 20%, or about 30%, or about 40% of the rate or level of angiogenesis. Preferably, the reduction is a medium level reduction of about 50%, or about 60%, or about 70%, or about 80% reduction of the rate or level of angiogenesis. More preferably, the reduction is a high level reduction of about 90%, or about 95%, or about 99%, or about 99.9% of the rate or level of angiogenesis. Most preferably, inhibition can also include the elimination of angiogenesis or its reduction to an undetectable level. Methods and assays for determining the rate or level of angiogenesis, and hence for determining whether and to what extent an antibody inhibits angiogenesis, are known in the art and are described in further detail herein in the Examples.

Typically, the angiogenesis that is inhibited is tumour angiogenesis. Thus, the individual may have a solid tumour, which can be treated by inhibiting tumour angiogenesis, i.e. the solid tumour is associated with new blood vessel production.

As discussed previously, it is preferred that an immune cell comprising a nucleic acid or vector of the invention (e.g. a T-cell) be used in the therapeutic methods of the invention. The immune cells (e.g. T cells) may be autologous or allogeneic.

By “autologous” it is meant that the cells to be used in the treatment method or use (i.e. to be transduced with nucleic acid or vector) originate or are obtained from a subject upon whom the method of treatment is to be carried out. Thus, autologous cells are obtained from a subject, transduced with nucleic acid or vector and returned to the same subject.

By “allogeneic” it is meant that the cells to be used in the treatment method or use (i.e. to be transduced with nucleic acid or vector) originate or are obtained from a different subject to the subject upon whom the method of treatment is to be carried out. Thus, allogeneic cells are obtained from a first subject, transduced with nucleic acid or vector and administered to a second subject.

Methods, formulations and amounts of cells, particularly T cells, for administration to a subject are well known in the art are discussed further below. Particularly, T cells may be for administration intratumorally or by infused iv (intravenously). Typical doses may be in the region of 10⁶-10⁸ cells per kg. The invention also provides a pharmaceutical composition comprising a nucleic acid, vector or cell of the invention.

The term “subject” as used herein refers to a human or non-human subject, e.g. a mammal as previously defined.

Although the nucleic acid, vector or cells of the invention may be efficacious in combating disease when used in isolation, it is possible to use a further therapeutic agent in combination with the nucleic acid, vector or cells of the invention to combat disease. Particularly, it may be desirable to inhibit tumour angiogenesis and consequently reduce the size of a tumour in a subject, by the administration of the nucleic acid, vector or cells of the invention and then to subsequent treat the tumour with a cytotoxic agent.

Accordingly, in a further embodiment of the invention, at least one further or additional therapeutic agent (e.g. an anti-cancer and/or anti-angiogenesis compound/agent) may be administered to a subject. Thus, the nucleic acid, vector, CAR, cell or population of cells of the invention and the further therapeutic agent (e.g. anti-cancer and/or anti-angiogenesis compound/agent) may be administered to the subject. A composition or pharmaceutical composition of the invention may therefore comprise a further active or therapeutic agent, together with a nucleic acid, vector, CAR, cell and/or population of cells of the invention. However, it is appreciated that the nucleic acid, vector, CAR, cell or population of cells of the invention and further therapeutic agent (e.g. anti-cancer and/or anti-angiogenesis compound/agent) may be administered separately, for instance by separate routes of administration. Additionally, the nucleic acid, vector, CAR, cell or population of cells of the invention and the at least one further therapeutic agent (e.g. anti-cancer and/or anti-angiogenesis compound/agent) can be administered sequentially or (substantially) simultaneously. They may be administered within the same pharmaceutical formulation or medicament or they may be formulated and administered separately. For sequential administration, the further therapeutic agent may be administered at least 1 minute, 10 minutes, 1 hour, 6 hours, 12 hours, 1 day, 5 days, 10 days, 2 weeks, 4 weeks or 6 weeks before or after the administration of nucleic acid/vector/cell.

In a particular embodiment, the invention provides a method of combating a disease or condition associated with expression of CLEC14A, e.g. for inhibiting angiogenesis, particularly tumour angiogenesis, e.g. a method of treating cancer, said method comprising administering a nucleic acid/vector/CAR/cell/cell population of the invention as defined herein, particularly an effective amount of said nucleic acid/vector/CAR/cell/cell population and separately, simultaneously or sequentially administering of one or more additional active (e.g. therapeutic) agents (e.g. anti-cancer and/or anti-angiogenesis compound/agent) to a subject in need thereof.

Alternatively viewed, there is provided a nucleic acid, vector, CAR, cell or cell population of the invention as defined herein for use in combating a disease or a condition associated with expression of CLEC14A (e.g. for use in inhibiting angiogenesis) wherein said nucleic acid, vector, CAR, cell or cell population is for administration separately, simultaneously or sequentially in combination with one or more additional active (e.g. therapeutic) agents (e.g. anti-cancer and/or anti-angiogenesis compound/agent).

Thus, there is provided the use of a nucleic acid, vector, CAR, cell or cell population of the invention as defined herein in the manufacture of a medicament for use in combating a disease or condition associated with expression of CLEC14A, e.g. for inhibiting angiogenesis, particularly tumour angiogenesis, e.g. for treating cancer, wherein said nucleic acid, vector, CAR, cell or cell population is for administration in combination with one or more additional active agents (e.g. therapeutic) agents (e.g. anti-cancer and/or anti-angiogenesis compound/agent).

Thus, in one embodiment the medicament may further comprise one or more additional active (e.g. therapeutic) agents (e.g. anti-cancer and/or anti-angiogenesis compound/agent). The additional active agent may further be an immune checkpoint inhibitor.

The medicament may be in the form of a single composition comprising both the nucleic acid, vector, antibody or ligand based CAR or immune effector cell of the invention as defined herein and the one or more additional active (e.g. therapeutic) agents (e.g. anti-cancer and/or anti-angiogenesis compound/agent), or it may be in the form of a kit or product containing them for separate (e.g. simultaneous or sequential) administration.

In some embodiments, the further therapeutic agent is an anti-cancer agent. The further anti-cancer agent may be selected from alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; cell cycle inhibitors; proteosome inhibitors such as Bortezomib (Velcade®); signal transductase (e.g. tyrosine kinase) inhibitors such as Imatinib (Glivec®), COX-2 inhibitors, and hormone agonists/antagonists such as flutamide and tamoxifen.

The clinically used anti-cancer agents are typically grouped by mechanism of action: Alkylating agents, Topoisomerase I inhibitors, Topoisomerase II inhibitors, RNA/DNA antimetabolites, DNA antimetabolites and Antimitotic agents. The US NIH/National Cancer Institute website lists 122 compounds (http://dtp.nci.nih.gov/docs/cancer/searches/standard_mechanism.html), all of which may be used in conjunction with the antibody, composition or immune effector cell of the invention. They include Alkylating agents including Asaley, AZQ, BCNU, Busulfan, carboxyphthalatoplatinum, CBDCA, CCNU, CHIP, chlorambucil, chlorozotocin, cis-platinum, clomesone, cyanomorpholino-doxorubicin, cyclodisone, dianhydrogalactitol, fluorodopan, hepsulfam, hycanthone, melphalan, methyl CCNU, mitomycin C, mitozolamide, nitrogen mustard, PCNU, piperazine, piperazinedione, pipobroman, porfiromycin, spirohydantoin mustard, teroxirone, tetraplatin, picoplatin (SP-4-3) (cis-aminedichloro(2-methylpyridine)Pt(II)), thio-tepa, triethylenemelamine, uracil nitrogen mustard, Yoshi-864; anitmitotic agents including allocolchicine, Halichondrin B, colchicine, colchicine derivative, dolastatin 10, maytansine, rhizoxin, taxol, taxol derivative, thiocolchicine, trityl cysteine, vinblastine sulphate, vincristine sulphate; Topoisomerase I Inhibitors including camptothecin, camptothecin, Na salt, aminocamptothecin, 20 camptothecin derivatives, morpholinodoxorubicin; Topoisomerase II Inhibitors including doxorubicin, amonafide, m-AMSA, anthrapyrazole derivative, pyrazoloacridine, bisantrene HCL, daunorubicin, deoxydoxorubicin, mitoxantrone, menogaril, N,N-dibenzyl daunomycin, oxanthrazole, rubidazone, VM-26, VP-16; RNA/DNA antimetabolites including L-alanosine, 5-azacytidine, 5-fluorouracil, acivicin, 3 aminopterin derivatives, an antifol, Baker's soluble antifol, dichlorallyl lawsone, brequinar, ftorafur (pro-drug), 5,6-dihydro-5-azacytidine, methotrexate, methotrexate derivative, N-(phosphonoacetyl)-L-aspartate (PALA), pyrazofurin, trimetrexate; DNA antimetabolites including, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine and thiopurine.

In some preferred embodiments, the at least one further anti-cancer agent is selected from cisplatin; carboplatin; picoplatin; 5-flurouracil; paclitaxel; mitomycin C; doxorubicin; gemcitabine; tomudex; pemetrexed; methotrexate; irinotecan, fluorouracil and leucovorin; oxaliplatin, 5-fluorouracil and leucovorin; and paclitaxel and carboplatin.

When the further anti-cancer agent has been shown to be particularly effective for a specific tumour type, it may be preferred that the nucleic acid, vector or cell of the invention is used in combination with that further anti-cancer agent to treat that specific tumour type.

In some embodiments, the anti-angiogenesis compound may be selected from any one of the following: bevacizumab (Avastin®); itraconazole; carboxyamidotriazole; TNP-470 (an analog of fumagillin); CM101; IFN-α; IL-12; platelet factor-4; suramin; SU5416; thrombospondin; VEGFR antagonists; angiostatic steroids+heparin; Cartilage-Derived Angiogenesis Inhibitory Factor; matrix metalloproteinase inhibitors; angiostatin; endostatin; 2-methoxyestradiol; tecogalan; tetrathiomolybdate; thalidomide; prolactin; αVβ3 inhibitors; linomide; tasquinimod; ranibizumab; sorafenib; (Nexavar®); sunitinib (Sutent®); pazopanib (Votrient®); and everolimus (Afinitor®).

The further therapeutic agent may be a hypoxia-activated cytotoxic agent, such as tirapazamine, or a cytokine which may enhance the efficacy/persistence/expansion of CAR-expressing cells (e.g. T cells). Alternatively, or additionally, the further therapeutic agent may be one which ameliorates one or more side effects associated with the administration of CAR-expressing cells.

As mentioned previously, the further therapeutic agent may be an immune checkpoint inhibitor. Such inhibitors generally function by blocking the interaction between an immune cell and a target cell (e.g. tumour cell) which prevents or downregulates the stimulation of the immune cell. Particularly, checkpoint inhibitors prevent or reduce the interaction between a protein expressed on a T cell and a protein expressed on a tumour cell, which interaction would prevent or reduce stimulation of the T cell. A checkpoint inhibitor may for example prevent the interaction between PD1 and PDL1 and particularly may constitute an agent which binds to PD1. Alternatively, a checkpoint inhibitor may bind to CTLA-4. Such checkpoint inhibitors are well known in the art and include monoclonal antibodies such as Penbrolizumab, Nivolumab or Ipilimumab.

The further active agent may also be a sphingosine-1-phosphase agonist, e.g. FTY720, which is capable of sequestering lymphocytes in the lymphoid organs by blocking signals from the sphingosine-1 phosphate receptor. In this way, such compounds may limit the competition for cytokines such as IL-7 and IL-15 and may thus allow an increased proliferation of the administered CAR expressing cell therapy. Particularly, such compounds may be administered before the nucleic acid, expression vector, CAR or cell of the invention, e.g. at least 12 hours, 24 hours, 36 hours or 48 hours before.

Additionally, the further or additional active (e.g. therapeutic) agent may be a TCR molecule (e.g. expressed on an immune cell such as a T cell or in soluble form), a nucleic acid molecule comprising a polynucleotide encoding a TCR molecule or a vector comprising said nucleic acid molecule. In a particularly preferred embodiment, the additional therapeutic agent is a cell (e.g. a T cell), comprising a nucleic acid (e.g. a RNA or a vector) encoding a TCR molecule. Thus, as discussed previously, in a particular embodiment of the invention, a nucleic acid encoding a CAR as described herein, a vector comprising the nucleic acid, a cell comprising the nucleic acid or expressing a CAR of the invention or the CAR itself, may be useful for treatment together with a T-cell receptor (TCR) molecule therapy (e.g. as a gene or cell therapy or as a soluble TCR). This embodiment of the invention encompasses a combination product which may have an enhanced ability to target a solid tumour. Thus, particularly, the TCR molecule therapy may target a different tumour associated antigen to a CAR of the invention which targets CLEC14A and such a combination product or therapy may thus present a particularly effective medicament for the treatment of solid tumours.

“TCR” or “T-cell receptor” molecule as used herein refers to a molecule which is capable of being expressed on the surface of T cells and which is capable of binding to a particular MHC or HLA/antigen peptide complex (e.g. presented on the surface of an antigen presenting cell or a tumour cell). Thus TCRs usually recognise antigens or fragments of antigens when found in a complex with a particular MHC or HLA. A TCR molecule as used herein may comprise two protein or polypeptide chains (e.g. may comprise an alpha TCR chain and a beta TCR chain, or a gamma TCR chain and a delta TCR chain), or the TCR may be a single chain molecule, where the alpha and beta chains or the gamma and delta chains are expressed and comprised within a single protein or polypeptide chain. Single chain TCR molecules are described in Chung et al (1994), Proc. Natl. Acad. Sci. USA, 91, 12654-12658, which is incorporated herein by reference.

Each TCR alpha, beta, gamma or delta chain generally comprises a variable region, wherein each variable region typically comprises at least one complementarity determining region (e.g. two and particularly three complementarity determining regions), which is capable of recognising and binding to the tumour associated antigen peptide/MHC complex. Complementarity determining regions (CDRs) may be separated from each other by one or more framework regions (FRs), and typically a TCR alpha, beta, gamma or delta chain variable region as defined herein may comprise three CDRs and three FRs. Particularly, a TCR molecule as described herein may comprise an alpha chain variable region comprising in N to C terminal order FR1α-CDR1α-FR2α-CDR2α-FR3α-CDR3α and a beta chain variable region comprising in N to C terminal order FR1β-CDR1β-FR2β-CDR2β-FR3β-CDR3β.

Further, the alpha, beta, gamma or delta chain may comprise a constant region (e.g. having extracellular and transmembrane domains) or a portion of the constant region (e.g. having only an extracellular domain). Particularly, a TCR comprising two separate alpha/beta or gamma/delta polypeptide chains may comprise chains wherein one or both of said chains have a constant domain (e.g. having extracellular and transmembrane domains). In a particular embodiment, both alpha/beta or gamma/delta TCR chains comprise a variable and a constant domain.

Single chain TCRs as used herein may comprise a single constant region, e.g. may comprise an alpha chain variable region, a beta chain variable region and a beta chain constant region, or an alpha chain variable region, a beta chain variable region and an alpha chain constant region, comprised within a single polypeptide chain.

The TCR molecule as defined herein may be a soluble TCR molecule or a membrane bound TCR. Soluble TCR molecules generally comprise a truncated constant region or have no constant region, wherein any truncation is sufficient to remove the transmembrane portion of the constant region. Soluble TCRs lacking a transmembrane portion may be of utility in targeting other molecules to the cells displaying the tumour associated antigen peptide/MHC complex. Particularly, however, a membrane bound TCR may be used in the combination therapy of the present invention. Thus, particularly, a TCR molecule as defined herein may comprise a constant region transmembrane domain (e.g. one transmembrane domain or two transmembrane domains, one comprised in each chain).

It will be appreciated that the constant regions of the alpha/beta and gamma/delta chains of TCRs are relatively conserved between TCRs. There are thus only two variant beta constant regions (which are different in only 4 amino acids), a single alpha and delta chain constant region, and three variant gamma constant regions in native TCR molecules. Although a TCR molecule as used herein may comprise any of the native constant regions, particularly, the TCR may comprise one or more modifications to any constant region or a portion thereof which is comprised within the TCR. Modifications which improve the pairing of the TCR chains or which improve the production of a soluble or single chain TCR molecule are particularly preferred.

The TCR molecule as used herein may comprise an additional di-S bond which is not present within a naturally occurring molecule, by the substitution of one or more residues in the alpha/beta and gamma/delta chains with a cysteine residue. In this respect, the substitution of an amino acid with a cysteine residue in the beta chain constant region and the substitution of an amino acid with a cysteine residue in the alpha chain constant region may allow the formation of a non-naturally occurring di-S bond between the substituted cysteine residues which may prevent or reduce mispairing of the alpha and beta chains with endogenous alpha and beta chains in T cells. Particularly, the modification may be made to the extracellular portion of the constant region of both chains comprised within the TCR.

More particularly, a TCR molecule as used herein may comprise a substitution at Thr 48 in the constant region of the alpha chain for cysteine and a substitution at Ser 57 in the constant region of the beta chain for cysteine; a substitution at Thr 45 in the constant region of the alpha chain for cysteine and a substitution at Ser 77 in the constant region of the beta chain for cysteine; a substitution at Tyr 10 in the constant region of the alpha chain for cysteine and a substitution at Ser 17 in the constant region of the beta chain for cysteine; a substitution at Thr 45 in the constant region of the alpha chain for cysteine and a substitution at Asp 59 in the constant region of the beta chain for cysteine; and/or a substitution at Ser 15 in the constant region of the alpha chain for cysteine and a substitution at Glu 15 in the constant region of the beta chain for cysteine.

Thus, the TCR molecule as used herein may have a Thr 48 to cysteine substitution in the alpha chain constant region and a Ser 57 to cysteine substitution in the beta chain constant region. Naturally occurring amino acid sequences for the alpha and beta chain constant regions are set out in SEQ ID Nos 187 and 188, respectively, and thus particularly, the modifications discussed above may be made to the stated positions within these sequences.

Other modifications may be made to the TCR to improve the pairing between the chains. For example, a leucine zipper may be utilised, the chains may be murinized or partially murinized e.g. at least one or two amino acids may be murinized, a TCR-like molecule may be constructed (e.g. by fusing the TCR to CD3 zeta) or an amino acid pair at the interface of the alpha and beta constant regions may be inversely exchanged. Particularly, the amino acid pair which are inversely exchanged interact with each other at their surfaces in the native TCR constant regions of the alpha and beta chains. This interacting amino acid pair may be subjected to mutagenesis such that the amino acid of the alpha chain constant domain is replaced by an amino acid which has a sterically projecting group as compared to the naturally occurring amino acid and the amino acid of the beta chain constant domain is replaced by an amino acid which has a sterically recessed group as compared to the naturally occurring amino acid (or vice versa, i.e. the alpha chain amino acid may be substituted with an amino acid having a sterically recessed group and the beta chain amino acid may be substituted with an amino acid having a sterically projecting group). Amino acids which may have a sterically recessed group as compared to a naturally occurring amino acid may be selected from glycine, serine, threonine, valine and alanine. Amino acids which may have a sterically projecting group as compared to a naturally occurring amino acid may be selected from glutamine, glutamic acid, alpha-methylvaline, histidine, hydroxylysine. tryptophan, lysine, arginine, phenylalanine and tyrosine. Particularly, a glycine residue in the alpha constant region may be substituted with an arginine and an arginine residue in the beta constant region may be substituted with a glycine residue, e.g. a glycine to arginine substitution may be made at position 85.1 in the alpha chain constant region and an arginine to glycine substitution may be made at position 88 in the beta chain constant region (using the ImMunoGeneTics information system (IMGT) nomenclature for the numbering of the TCR constant domains). Thus, the arginine to glycine substitution may occur at position 73 of the beta constant region of SEQ ID NO. 188.

Further, it may be desirable to remove a naturally occurring di-S which occurs between the TCR chains (e.g. between the alpha and beta chains). Thus, an interchain native di-S bond in a TCR may be removed by substituting the cysteine residues involved in the bond e.g. to serine or alanine residues, or by deleting the residues. An additional or alternative modification which may be desirable is the removal or substitution of an unpaired cysteine residue which occurs in the native beta TCR chain. Such a modification may be preferred wherein the TCR is a single chain TCR.

A “tumour associated antigen” as used herein refers to any antigen whose expression is associated with a tumour (e.g. with any tumour type or with more than one tumour type). Thus, particularly, the expression of a tumour associated antigen may be upregulated or enhanced in a tumour or tumour cell, as compared to healthy tissue or cells of the same type. Expression of a tumour associated antigen may be upregulated in a tumour or tumour cell by at least 2, 3, 4, 5, 10, 20, 50 or 100 fold or alternatively viewed by at least 20, 30, 40, 50, 60, 70, 80, 90 or 100% as compared to expression of that antigen in healthy tissue or cells of the same type (e.g. from cells or tissue obtained from the same organ which are of the same type). Thus, detection of a tumour associated antigen in a tissue or cell may be indicative of a tumour. Particularly, a tumour associated antigen may be expressed at only very low levels, or may be undetectable in healthy tissue or in healthy tissue related to a particular organ.

Expression levels of a tumour associated antigen, typically refer to the amount of protein expressed with a particular cell/tissue. Methods of measuring protein expression levels or detecting overexpressed proteins are well known in the art and include for example, Western blotting, immunostaining etc.

Many tumour associated antigens are known in the art which are upregulated or over expressed in tumour cells as compared to healthy tissue, and any of these antigens may be targeted by a TCR as the additional therapeutic agent in a therapy of the invention. Particularly, in accordance with the present invention, it is desirable to target a solid tumour and thus preferably, the TCR molecule as discussed herein may recognise and bind a tumour associated antigen which is overexpressed or upregulated on a solid tumour. For example, NY-ESO (a tumour associated antigen related to melanoma and testis cancer), the MAGEA family (and particularly MAGEA 10) (a tumour associated antigen related to testis cancer), AFP (a tumour associated antigen related to hepatocellular carcinoma) and WT1 (a tumour associated antigen expressed on several tumour types) may be targeted in a combination therapy of the invention.

It will be appreciated that a TCR molecule recognises and binds to a peptide/MHC or HLA complex and thus a TCR molecule as used herein will typically recognise a portion or peptide fragment of a tumour associated antigen. Such peptide fragments may be from 5-25 amino acids long, e.g. from 5-10, 8-15, 10-18, 15-25 amino acids long, e.g. may be at least 5, 6, 7, 8, 9, 10, 11, 12, 13, 14, or 15 amino acids long and are typically contiguous peptide sequences comprised within a tumour associated antigen. Further, a TCR molecule will only recognise a peptide from a tumour associated antigen when in combination with a particular MHC or HLA (e.g. MHC class I or II or HLA-A1 or HLA-A2). Particularly, a TCR molecule as defined herein may recognise a peptide tumour associated antigen when in combination with HLA-A2.

Binding of a TCR to a peptide/MHC or HLA complex can be determined by various methods known in the art, e.g. by measuring various parameters associated with T cell activation, when the TCR is expressed on a T cell, by tetramer assay or by cytotoxicity assay.

As discussed previously, the TCR used herein preferably binds to a different tumour associated antigen than the encoded CAR, i.e. the TCR preferably does not bind to CLEC14A.

In a particular embodiment of the invention, the TCR molecule may recognise and bind to a WT1 peptide/MHC or HLA complex and more particularly to a WT1 peptide/HLA A2 complex. For example, the TCR may bind to a WT1 peptide 235-243 (CMTWNQMNL) SEQ ID NO. 202-HLA A*2402 combination. However, in a particularly preferred embodiment, the TCR recognises and binds to the RMFPNAPYL (SEQ ID NO. 189) peptide of WT1/HLA A2 complex. It will be appreciated that more than one TCR may be capable of binding to this complex and one or more of such TCRs may be used in a combination therapy of the invention. Further, in connection with this aspect, the combination therapy may utilise a double chain TCR (i.e. having separate alpha and beta or gamma and delta chains) and/or a single chain TCR to bind to the WT1 complex.

Thus, a TCR molecule as used in the combination therapy described herein (e.g. as the further therapeutic agent), may comprise an alpha chain and a beta chain,

wherein the alpha chain comprises CDR1alpha of SEQ ID NO. 190, CDR2 alpha of SEQ ID NO. 191 and CDR3 alpha of SEQ ID NO. 192 or 193, and

wherein the beta chain comprises CDR1 beta of SEQ ID NO. 194, CDR2 beta of SEQ ID NO. 195 and CDR3 beta of SEQ ID NO. 196 or 197,

or a variant thereof wherein one or more of the CDRs comprise one, two or three amino acid substitutions,

wherein said TCR molecule is capable of binding to an HLA A2/RMFPNAPYL complex.

It should be noted that in some nomenclature systems the CDR3 of the beta chains may be defined to be longer than in the nomenclature system used in the Immunogenetics (IMGT) database described below. Additionally, in some nomenclature systems, the CDR3 of the alpha chains may be defined to be shorter than in the IMGT system. Similarly, the constant region may or may not include framework residues flanking the CDR3 region in the different nomenclature systems.

Thus, using the IMGT system, CDR3 alpha may have the amino acid sequence of SEQ ID NO. 192 and the constant region includes the framework amino acid sequence FGKGTHLIIQP.

Using a different nomenclature system (Garcia) (Garcia et al, 1999, Ann. Rev. Immunol. 17, 369-397, incorporated herein by reference), CDR3 alpha has the amino acid sequence of SEQ ID NO. 193, the framework region immediately C-terminal to this has the amino acid sequence of FGKGTHLIIQP and the constant region begins with the amino acid sequence YIQ.

Using the IMGT nomenclature system, CDR3 beta may have the amino acid sequence SEQ ID NO. 196 and the constant region immediately C-terminal to this includes the framework amino acid sequence SET.

Using the Garcia nomenclature system, CDR3 beta has the amino acid sequence SEQ ID NO. 197 and the framework region immediately C-terminal to this has the amino acid sequence FGPGTRLLVL and the immediately C-terminal constant region begins with the amino acid sequence EDL.

It will be appreciated that a skilled person can readily design and synthesise TCRs for use in the present invention, using either or any nomenclature system, provided that the framework region is compatible with the CDRs. The amino acid sequences, including variable regions (and thus framework regions) of numerous TCR alpha and beta chains are well known in the art, some of which are described at IMGT (Immunogenetics) database at http://imgt.cines.fr. This information together with for example, Garcia et al (supra), may be used to design and produce TCRs comprising CDRs and FRs.

As indicated above, the variant TCRs may be used in the present invention, where one or more CDRs may comprise one, two or three amino acid substitutions. As discussed herein, in relation to the CAR sequences, particularly the substitutions may be conservative substitutions, and any variant molecules should be capable of binding to the HLA A2/RMFPNAPYL complex. The affinity of binding of the variant may be increased or decreased as compared to the TCR having the CDRs as defined above, but binding should still occur. Methods for detecting binding of the TCR to its tumour associated antigen peptide/MHC complex target are described above.

Particularly, the TCR molecule for use as a further therapeutic agent in accordance with the present invention may comprise a TCR alpha chain as set out in SEQ ID NO. 200 and a TCR beta chain as set out in SEQ ID NO. 201. Alternatively, the TCR molecule may comprise a TCR alpha chain as set out in SEQ ID NO. 198 and a TCR beta chain as set out in SEQ ID NO. 199, wherein said alpha and beta chain sequences maybe further modified to stabilise or to enhance the pairing of said alpha and beta chains.

The nucleic acid molecules encoding a TCR molecule as described herein may be in the form of DNA or RNA. Thus, a composition of the invention may comprise RNA molecules which encode a CAR of the invention and a TCR molecule as described herein.

Alternatively, a vector may comprise the nucleic acid molecule comprising the polynucleotide sequence which encodes the TCR. Suitable vectors for expression of the CAR are described herein, and such vectors may also be utilised for TCR expression. Although it is envisaged that separate vectors may be employed for CAR and TCR expression, it is possible that a vector may encode both the CAR of the invention and the TCR molecule. In this respect, the expression of each gene may be controlled by a different promoter, or a single promoter may be utilised as described elsewhere herein.

In this respect, a vector comprising a polynucleotide sequence encoding a CAR of the invention and comprising a polynucleotide sequence encoding a TCR molecule capable of binding to a tumour associated antigen peptide/MHC or HLA complex is provided by the invention.

Further, a cell comprising a nucleic acid molecule of the invention comprising a polynucleotide sequence encoding a CAR and a nucleic acid molecule comprising a polynucleotide sequence encoding a TCR molecule capable of binding to a tumour associated antigen peptide/MHC or HLA complex is provided by the invention. A cell comprising a vector comprising a polynucleotide sequence encoding a CAR of the invention and a vector comprising a polynucleotide sequence encoding a TCR molecule capable of binding to a tumour associated antigen peptide/MHC or HLA complex is also encompassed.

The cell of the invention may alternatively or additionally comprise a vector comprising a polynucleotide sequence encoding a CAR of the invention and comprising a polynucleotide sequence encoding a TCR molecule capable of binding to a tumour associated antigen peptide/MHC or HLA complex.

Thus a cell may express a CAR molecule of the invention and a TCR molecule as defined herein (e.g. from the same or different vectors).

A population of cells is also provided by the invention, wherein at least one cell comprises a nucleic acid of the invention comprising a polynucleotide sequence encoding a CAR and at least one cell comprises a nucleic acid comprising a polynucleotide sequence encoding a TCR molecule as defined previously. Thus, in this embodiment, the population of cells may comprise cells which express only the CAR molecule and cells which express only the TCR molecule. Additionally, such a cell population may also comprise cells which express both the CAR molecule and TCR molecule.

Typically, as discussed above, a cell of the invention may be an immune cell and particularly a T cell.

This aspect of the invention further encompasses kits comprising the nucleic acid molecules or vectors encoding the CAR molecule and TCR molecule (either as separate polynucleotide sequences, or from the same or different vectors).

In a particular embodiment of the invention, a composition is provided comprising
(i) a nucleic acid molecule comprising a polynucleotide sequence encoding a TCR molecule wherein said TCR molecule comprises an alpha chain and a beta chain,

wherein the alpha chain comprises CDR1alpha of SEQ ID NO. 190, CDR2 alpha of SEQ ID NO. 191 and CDR3 alpha of SEQ ID NO. 192 or 193, and

wherein the beta chain comprises CDR1 beta of SEQ ID NO. 194, CDR2 beta of SEQ ID NO. 195 and CDR3 beta of SEQ ID NO. 196 or 197,

or a variant thereof wherein one or more of the CDRs comprises one, two or three amino acid substitutions,

wherein said TCR molecule is capable of binding to an HLA A2/RMFPNAPYL complex, and

(ii) a nucleic acid molecule comprising a polynucleotide sequence encoding a chimeric antigen receptor comprising an anti-CLEC14A binding domain, a transmembrane domain and a signalling domain, wherein said anti-CLEC14A binding domain comprises an amino acid sequence of SEQ ID NO 58, 96, 112, 125 or 175, (preferably SEQ ID NO 58, preferably SEQ ID NO. 96 or preferably SEQ ID NO. 125)

or a variant thereof having at least 80% sequence identity to any one of SEQ ID Nos 58, 96, 112, 125 or 175.

A cell is further specifically provided comprising a nucleic acid molecule as defined in (i) and (ii) above, particularly a T cell. Further a population of cells may comprise a first cell comprising a nucleic acid as defined in (i) above and a second cell comprising a nucleic acid as defined in (ii) above.

It will be evident from the discussion above that the invention provides various compositions, e.g. pharmaceutical, therapeutic, comprising a nucleic acid, vector, cell or population of cells of the invention and a pharmaceutically acceptable diluent, carrier or excipient. In this respect, it is appreciated that the agents of the invention (i.e. nucleic acid, vector or cell) will typically be formulated for administration to an individual (i.e. subject) as a pharmaceutical composition, i.e. together with a pharmaceutically acceptable carrier, diluent or excipient.

By “pharmaceutically acceptable” is included that the formulation is sterile and pyrogen free. Suitable pharmaceutical carriers, diluents and excipients are well known in the art of pharmacy. The carrier(s) must be “acceptable” in the sense of being compatible with the medicament and not deleterious to the recipients thereof. Typically, the carriers will be saline or infusion media (alternatively termed a solution for infusion) which will be sterile and pyrogen free; however, other acceptable carriers may be used. The compositions of the invention may comprise a suitable cryopreservation agent, for example DMSO.

In some embodiments the pharmaceutical compositions or formulations of the invention are for parenteral administration, more particularly for intravenous administration. In a preferred embodiment, the pharmaceutical composition is suitable for intravenous administration to a patient, for example by injection.

Formulations suitable for parenteral administration include aqueous and non-aqueous sterile injection solutions which may contain anti-oxidants, buffers, bacteriostats and solutes which render the formulation isotonic with the blood of the intended recipient; and aqueous sterile suspensions

The liquid pharmaceutical compositions may typically comprise cells of the invention e.g. T cells, in infusion media, which may comprise plasmalyte A plus HSA (e.g. at 4%) The cells of the invention are generally infused using a sterile isotonic solution. The parenteral preparation can be enclosed in ampoules, disposable syringes or multiple dose vials made of glass or plastic. An injectable pharmaceutical composition is preferably sterile.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The composition of the invention may be for administration in a single or in multiple doses. Particularly, the composition may be for administration in a single, one off dose.

The agents or compositions of the invention may be administered by any parenteral route, in the form of a pharmaceutical formulation comprising the active ingredient. Depending upon the disorder and patient to be treated, as well as the route of administration, the compositions may be administered at varying doses (e.g. measured in cells/kg or m²). The physician in any event will determine the actual dosage which will be most suitable for any individual patient and it will vary with the age, weight and response of the particular patient. Typically however, for cells of the invention, doses up to 1×10⁹/kg (or equivalent in m²) may be provided for intravenous administration, e.g. doses of at least 1×10⁸ cells/kg. For intratumoral administration, doses up to 1×10¹⁰ cells/kg are envisaged.

In human therapy, the agent or composition of the invention will generally be administered in admixture with a suitable pharmaceutical excipient, diluent or carrier selected with regard to the intended route of administration and standard pharmaceutical practice. Typically cells of the invention may be administered in infusion buffer.

The agent or composition of the invention can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The formulations may be presented in unit-dose or multi-dose containers, for example sealed ampoules, bags and vials.

In some embodiments the agent or composition of the invention may be administered by the ocular route. For ophthalmic use, the agent or composition of the invention can be formulated as, e.g., micronised suspensions in isotonic, pH adjusted, sterile saline, or, preferably, as solutions in isotonic, pH adjusted, sterile saline, optionally in combination with a preservative such as a benzylalkonium chloride.

The nucleic acid molecule agents of the invention (e.g. nucleic acid molecules, vectors etc.) may be administered as a suitable genetic construct as described below and delivered to the patient where it is expressed. Typically, the nucleic acid in the genetic construct is operatively linked to a promoter which can express the compound in the cell. The genetic constructs of the invention can be prepared using methods well known in the art, for example in Sambrook et al (2001).

Genetic constructs for delivery of polynucleotides can be DNA or RNA. Gene therapy methods of treatment, involving the direct administration of a nucleic acid molecule or expression vector of the invention to a subject are thus encompassed by the invention. Further, methods of treatment involving the direct administration of the CAR are also encompassed. Such methods may be advantageous, since these avoid the ex vivo handling of cells.

Preferably, the genetic construct is adapted for delivery to a human cell. Means and methods of introducing a genetic construct into a cell are known in the art, and include the use of immunoliposomes, liposomes, viral vectors (including vaccinia, modified vaccinia, lentivirus, parvovirus, retroviruses, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art. In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Although for cancer/tumours of specific tissues it may be useful to use tissue-specific promoters in the vectors encoding a polynucleotide inhibitor, this is not essential, as the risk of expression of the nucleic acid molecule agent in the body at locations other than the cancer/tumour would be expected to be tolerable in compared to the therapeutic benefit to a patient suffering from a cancer/tumour. It may be desirable to be able to temporally regulate expression of the polynucleotide inhibitor in the cell, although this is also not essential.

The agents of the invention (e.g. nucleic acid, vector, cell or population of cells) for administration may be appropriately modified for use in a pharmaceutical composition. For example agent may be stabilized in the compositions of the invention against degradation for example by the use of appropriate additives such as salts or non-electrolytes, acetate, EDTA, citrate, Tris, phosphate or acetate buffers, mannitol, glycine, HSA (human serum albumin) or polysorbate. Numerous stabilizing agents are known in the art. Cells may particularly be cryopreserved and thawed at an appropriate time, before being infused into a subject.

The invention further includes kits comprising one or more of the nucleic acids, vectors, cells or compositions of the invention. Preferably said kits are for use in the methods and uses as described herein, e.g., the therapeutic methods as described herein, or are for use in vitro assays or methods. Preferably said kits comprise instructions for use of the kit components.

Any reference to “tumour(s)” herein also refers to “cancer(s)” or “carcinoma(s)”. Metastatic cancers can also be treated, as can the reduction of metastases from a primary tumour. So-called minimal residual disease (MRD), which is left in post-surgery patients, may be amenable for immunotherapy with an agent as defined herein.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”. The operable limits and parameters of combinations, as with the amounts of any single agent, will be known to those of ordinary skill in the art in light of the present disclosure.

The documents cited herein are hereby incorporated by reference.

The invention will now be described in more detail by reference to the following Examples and Figures:

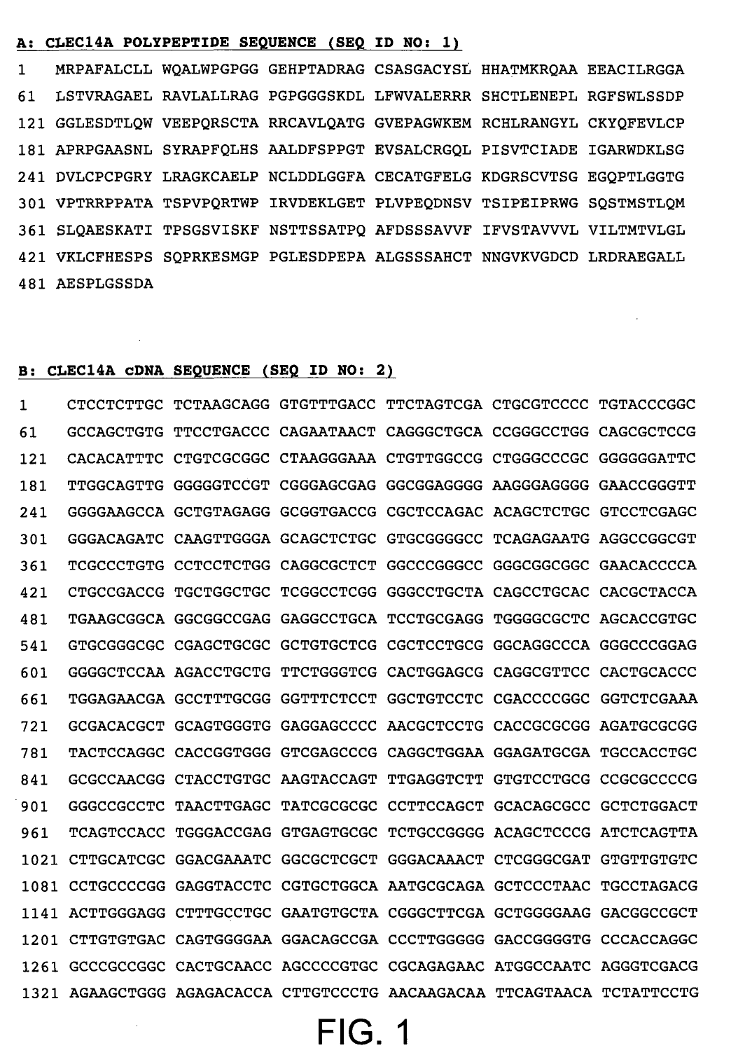

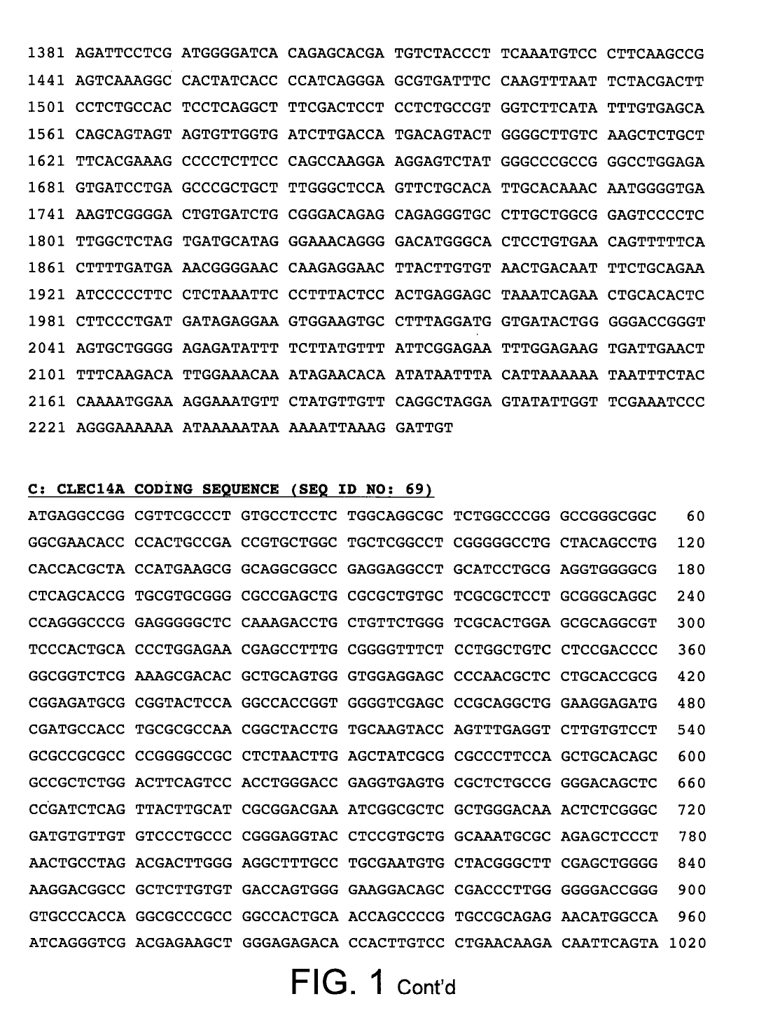

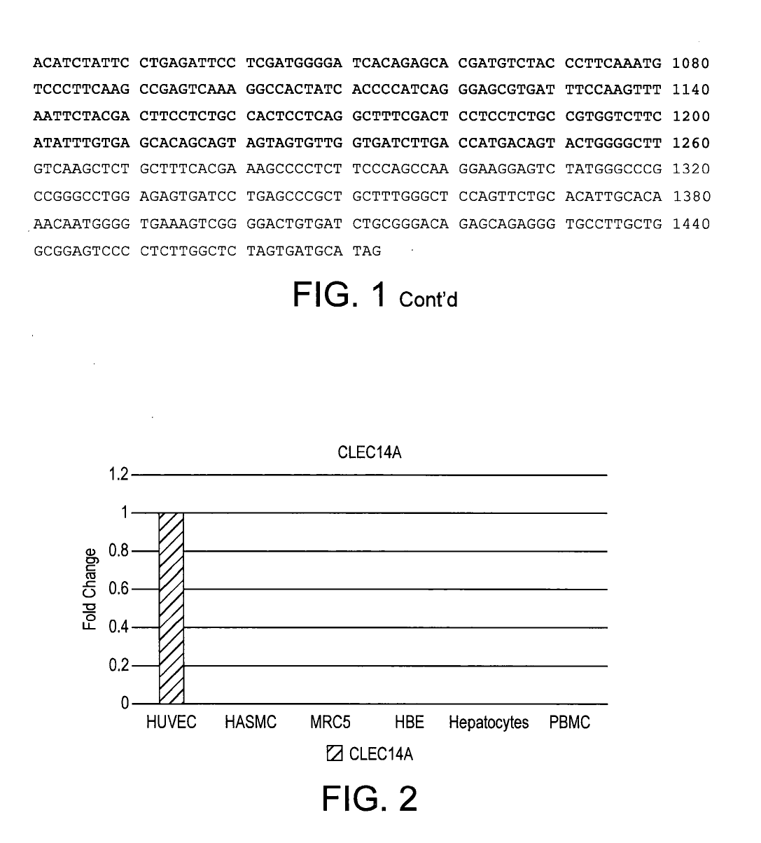

FIG. 1 shows the polypeptide sequence of human CLEC14A from Genbank Accession No. NP_778230 (SEQ ID NO. 1) (FIG. 1A); cDNA of human CLEC14A from Genbank Accession No NM_175060 (SEQ ID NO. 2) (FIG. 1B) and coding region of human CLEC14A cDNA from positions 348-1820 of NM_175060 (SEQ ID NO. 3).

FIG. 2 shows a graph of the relative expression of CLEC14A in HUVECs and other primary cells. CLEC14A was expressed specifically in endothelial cells (HUVECs) and not in human aortic smooth muscle cells (HASMC), human lung fibroblasts (MRC50, human bronchial epithelial cells (HBE), hepatocytes, or peripheral blood mononuclear cells (PBMC).

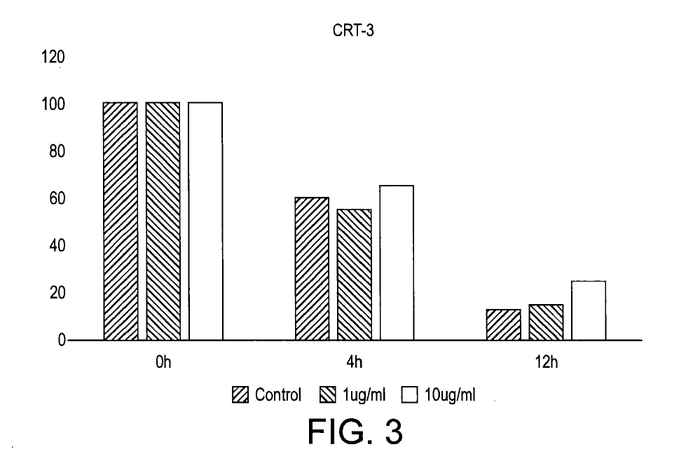

FIG. 3 shows graphical results of a HUVEC scratch wound healing assay with anti-CLEC14A monoclonal antibody CRT-3 showing a retardation of wound closure.

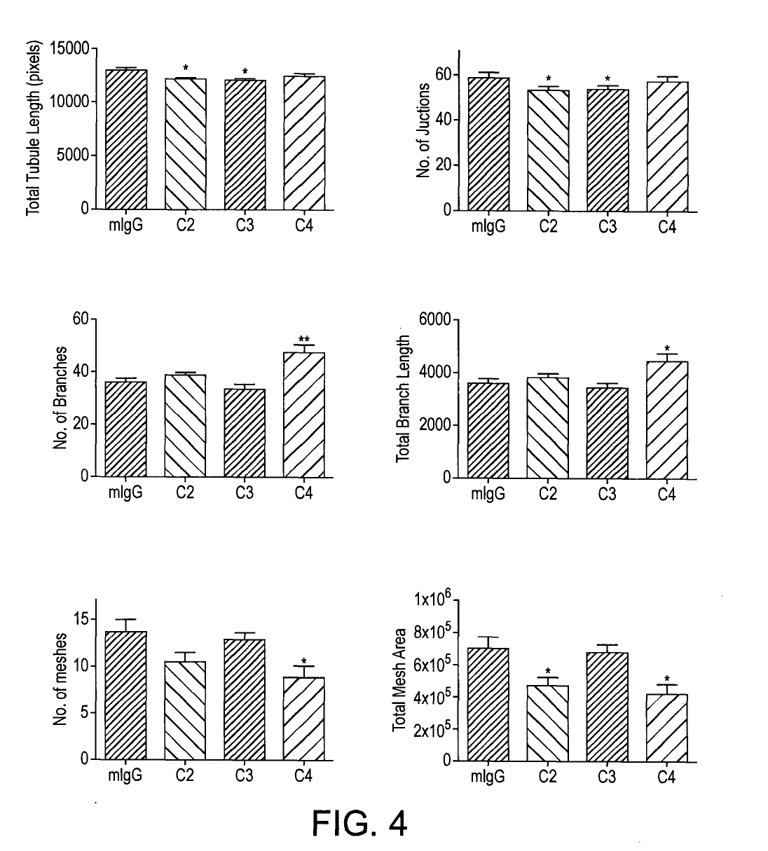

FIG. 4 shows analysis of tubule formation assays with CLEC14A antibody treated HUVECs. HUVECs were treated with 20 μg/ml CRT2, 3 or 4 or mouse IgG isotype control. Images of tubules were taken at 16 hours and analysed for total tubule length, number of junctions, number of branches, branch length, number of meshes and total mesh area. Data shown represents three experiments with five data points analysed for each. Error bars show SEM. *p,0.05. **p<0.01.

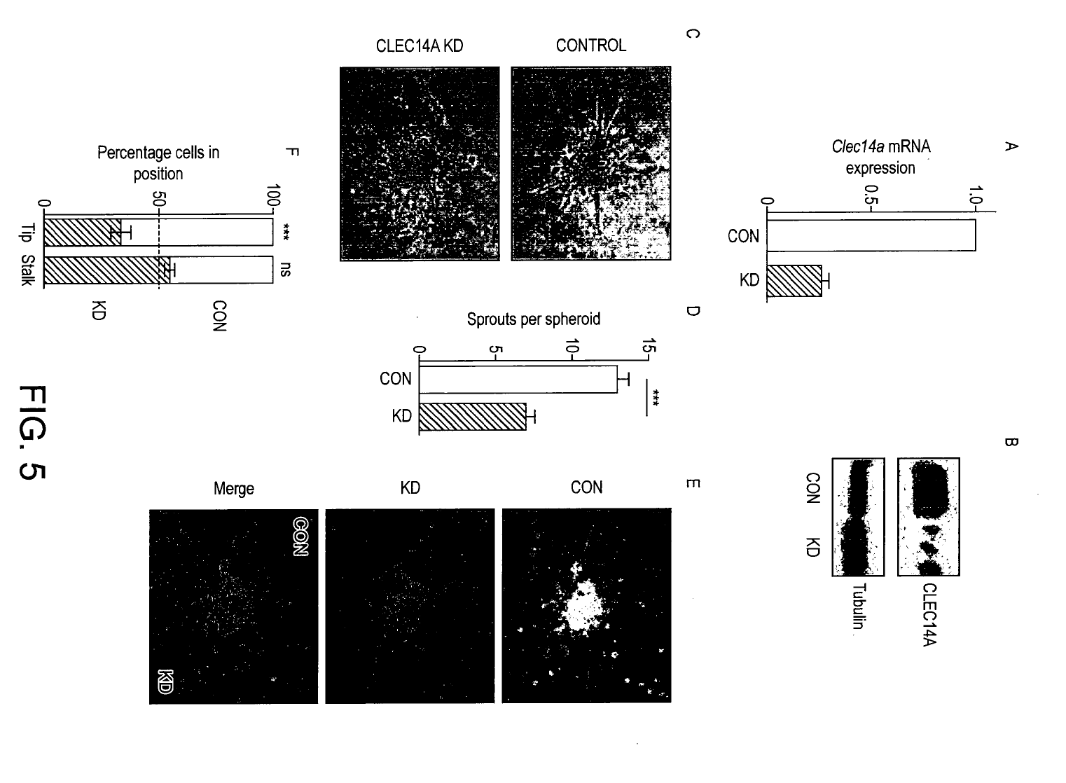

FIG. 5 shows siRNA knockdown of CLEC14A which reveals a role for CLEC14A in endothelial sprouting. FIG. 5A shows siRNA duplex targeting of CLEC14A can efficiently knockdown CLEC14A mRNA expression in HUVEC, as determined by qPCR. Relative expression was determined by normalising expression to flotilin2. FIG. 5B shows knockdown of CLEC14A at the protein level by Western blot analysis. Tubulin was used as a loading control. FIG. 5C shows representative images of sprout outgrowth after 16 hours for control or clec14a targeted siRNA treated HUVEC. FIG. 5D shows quantitation of sprouts for 27 spheroids (9 spheroids from 3 cords) for control and CLEC14A knockdown HUVEC; Mann-Whitney statistical test p<0.001. FIG. 5E shows representative images of sprout outgrowth after 24 hours for mixed control (green) and clec14a targeted siRNA treated HUVEC (red). FIG. 5F shows quantitation of the percentage of tip and stalk cells derived from control (CON) and CLEC14A knockdown (KD) HUVEC; two-way ANOVA statistical test with Bonferroni post-tests ***=p<0.001, ns=not significant.

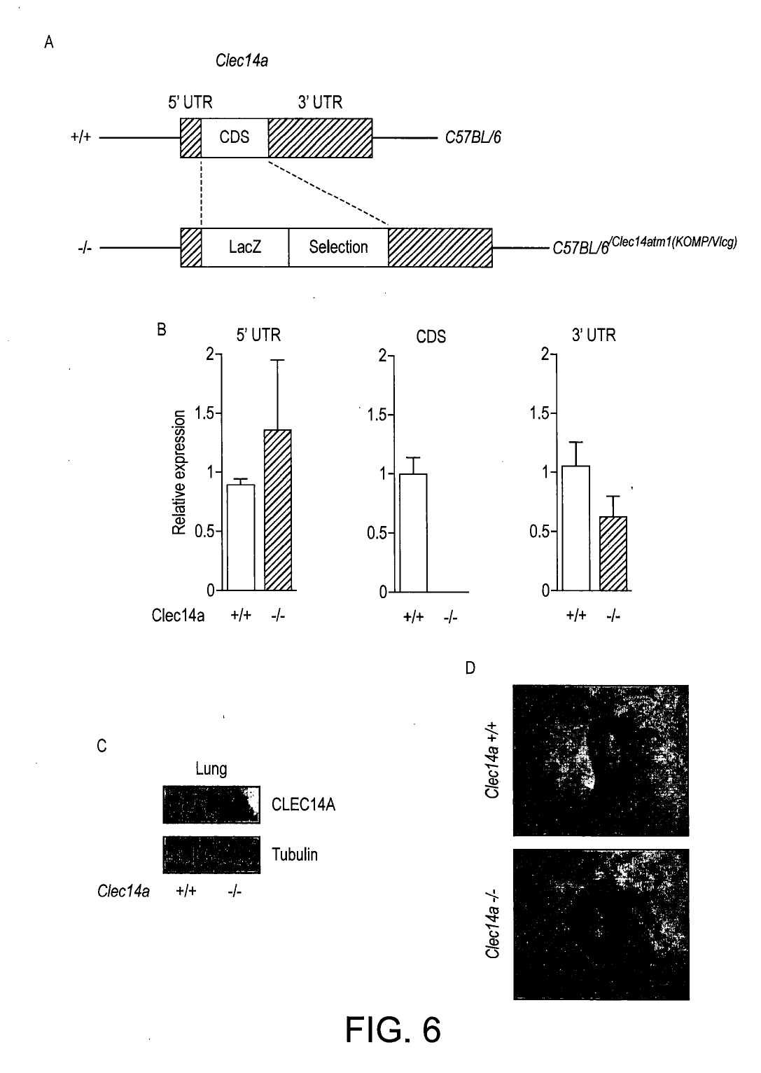

FIG. 6 shows that loss of CLEC14A inhibits sprouting in vitro and in vivo. FIG. 6A shows a schematic diagram of clec14a gene in C57BL/6 (clec14a +/+) or C57BL/6^{(Clec14atm1(KOMP)Vlcg)} (clec14a −/−) mice. FIG. 6B shows quantitative PCR analysis of cDNA generated from three clec14a +/+ mice (white bars) and three clec14a −/− mice (black bars) for the 5′ untranslated region (UTR), coding sequence (CDS) and 3′ UTR of clec14a. Relative expression was determined by normalising expression to flotilin2. FIG. 6C shows Western blot analysis of CLEC14A protein expression in lung lysates from clec14a +1+ and clec14a −/− mice using polyclonal antisera against murine CLEC14A. Tubulin was used as a loading control. FIG. 6D shows representative images of the aortic ring sprouting assay from clec14a +/+ and clec14a −/− mice. FIG. 6E shows quantitation of tubes formed per ring, and FIG. 6F shows quantitation of the maximal distance migrated by an endothelial tube from the aortic ring, data from 48 rings per genotype, 6 mice for each genotype; Mann-Whitney statistical test p<0.001. FIG. 6G shows representative images of haematoxylin and eosin stained sections of sponge implant from clec14a +1+ and clec14a −/− mice, sections at the centre of the sponge were analysed. FIG. 6H shows quantitation of cellular invasion into the sponge implants shown in FIG. 6G; Mann-Whitney statistical test p<0.05. FIG. 6I shows quantitation of vessel density; Mann-Whitney statistical test p<0.001. FIG. 6J shows sections of liver and sponge tissue stained with x-gal from clec14a −/− mice, counterstained with haematoxylin and eosin.

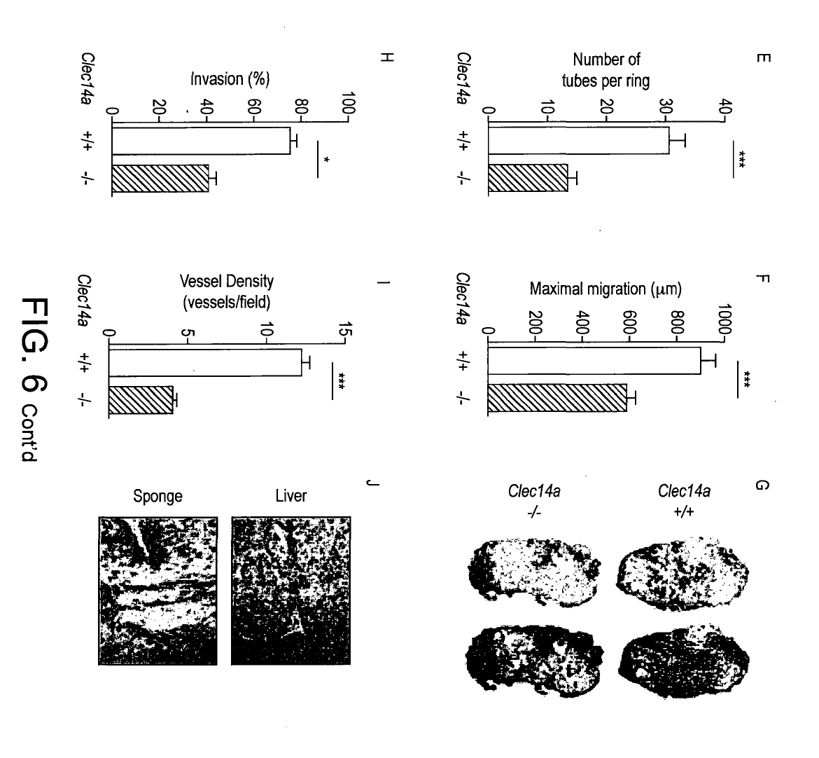

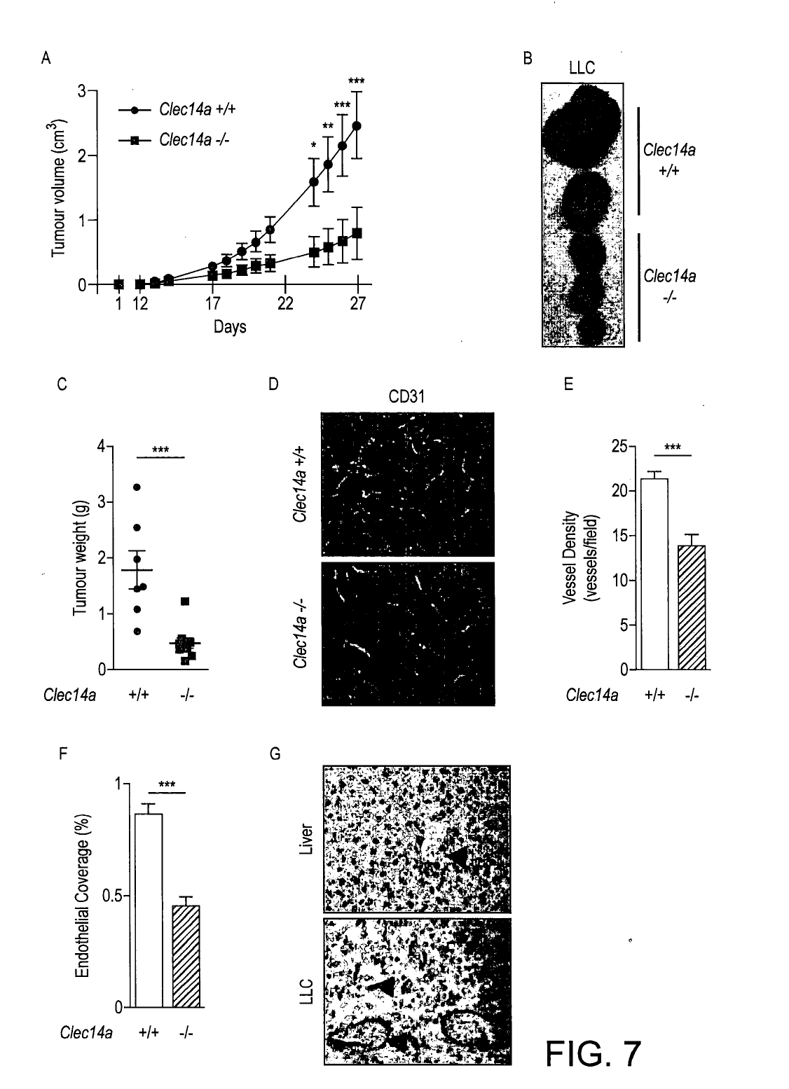

FIG. 7 shows loss of CLEC14A inhibits tumour growth. FIG. 7A shows Lewis lung carcinoma (LLC) tumour growth in clec14a +/+(black line with dots) and clec14a −/− (black line with squares) mice; two-way ANOVA statistical analysis, *=p<0.05, **=p<0.01, ***=p<0.001. FIG. 7B shows representative images of LLC tumours. FIG. 7C shows endpoint tumour weight for 7 clec14a +1+(dots) and 7 clec14a −/− (squares) mice; Mann-Whitney statistical test p<0.001. FIG. 7D shows representative images of immunofluorescent staining of LLC tumour sections stained for murine CD31. FIG. 7E shows quantitation of vessel density and FIG. 7F shows percentage endothelial coverage from clec14a +/+ and clec14a −/− mice; Mann-Whitney statistical test p<0.0001. FIG. 7G shows sections of liver and LLC tumour tissue from clec14a −/− mice stained with x-gal, counterstained with haematoxylin and eosin.

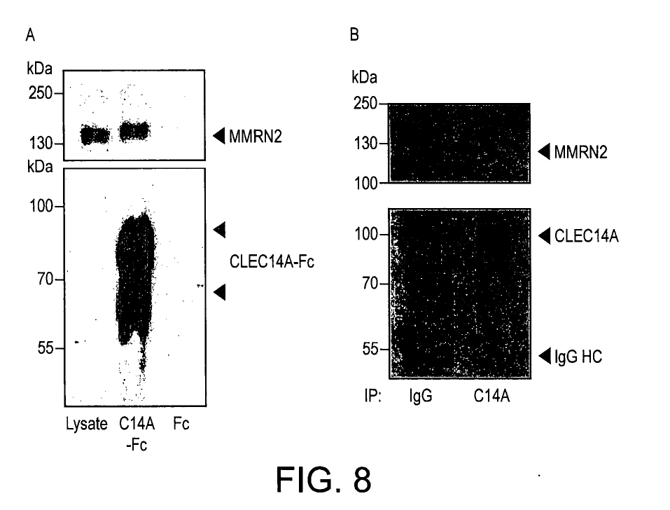

FIG. 8 shows MMRN2 binds to CLEC14A. In FIG. 8A 20 μg CLEC14A-ECD-Fc or Fc was used to precipitate interacting partners. Precipitates and HUVEC lysates were separated on an SDS-PAG and blotted for MMRN2 (top panel) or CLEC14A-ECD-Fc (bottom panel). In FIG. 8B CLEC14A was immunoprecipitated from HUVEC lysates using polyclonal antisera against CLEC14A. Immunoprecipitates were analysed by Western blot for MMRN2 (top panel) and CLEC14A (bottom panel).

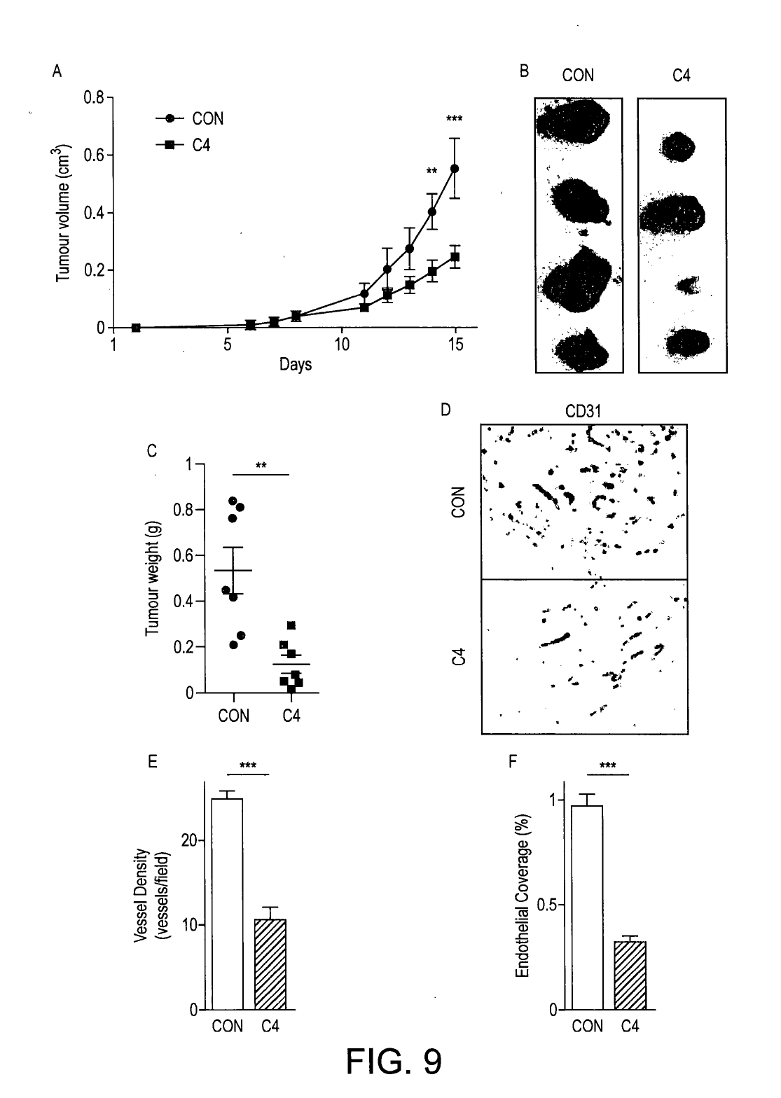

FIG. 9 shows MMRN2-CLEC14A interaction blocking antibody inhibits tumour growth. FIG. 9A shows results where mice injected with LLC were treated with 100 μg injections of mIgG1 (black line with dots; n=7) or C4 antibody (black line with squares; n=7); two-way ANOVA statistical analysis, **=p<0.01, ***=p<0.001. FIG. 9B shows representative images of LLC tumours. FIG. 9C shows endpoint tumour weight for 7 mIgG1 treated mice (dots) and 7 C4 antibody treated mice (squares); Mann-Whitney statistical test p<0.001. FIG. 9D shows representative images of immunofluorescent staining of LLC tumour sections stained for murine CD31. FIG. 9E shows quantitation of vessel density and FIG. 9F shows percentage endothelial coverage from mice treated with mIgG1 or C4 antibody; Mann-Whitney statistical test p<0.001.

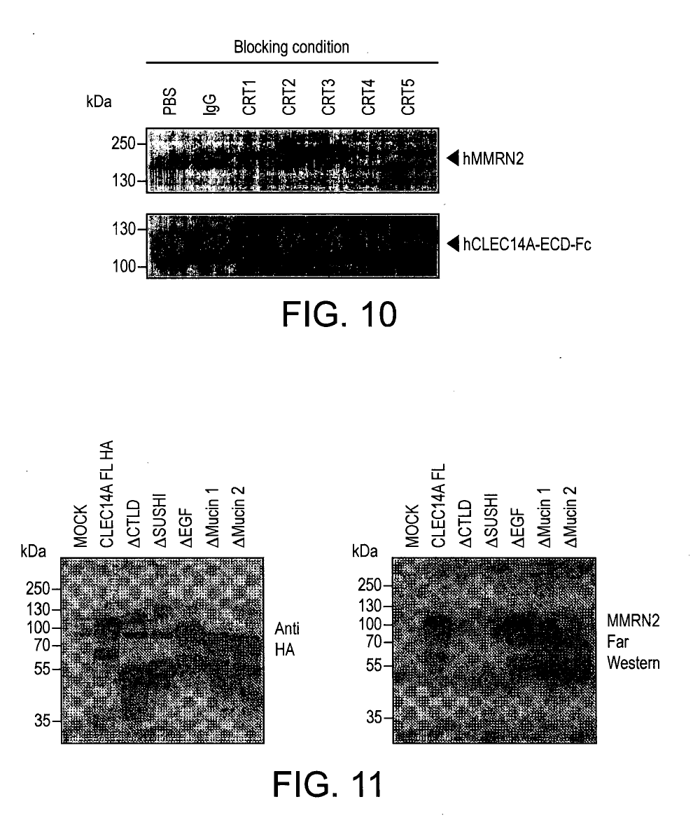

FIG. 10 shows that CLEC14A monoclonal antibodies C1, C4 and C5 block CLEC14A-MMRN2 interaction. Human CLEC14A-ECD-Fc was bound to protein A beads, blocked in 20% FCS and then incubated with each blocking condition. This was then added to lysates of HEK293T cells overexpressing full-length human MMRN2 with a His tag. Pre-incubating with CRT1, CRT4 and CRT5 decreased the levels of MMRN2 pulled down by CLEC14A-ECD-Fc.

FIG. 11 shows that MMRN2 directly binds to either the C-type lectin or sushi domain of CLEC14A under non-reduced conditions. HEK293T cells were mock transfected or transfected with pCS2 vectors containing CLEC14A wild type (WT) or constructs with each major domain deleted (Δ) with an N-terminal HA tag. Upon far western blotting with MMRN2 protein lysate, binding can be seen in all mutants except those missing the C-type lectin domain (CTLD) or the sushi domain. An anti-HA blot was included to show all mutant proteins were expressed.

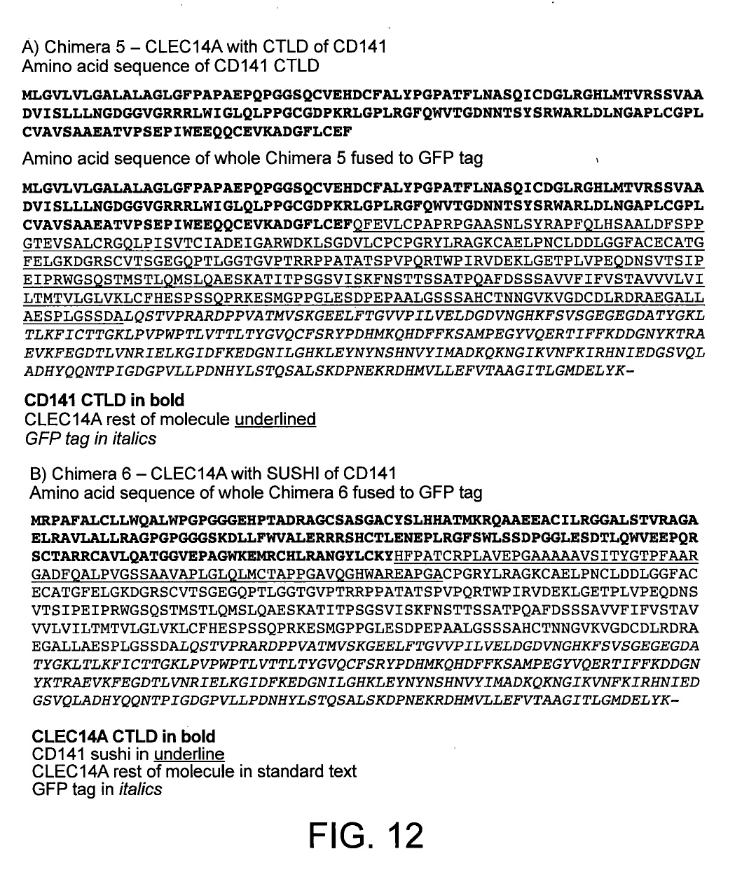

FIG. 12 shows protein sequences of the chimeric proteins Chimera 5 and Chimera 6.

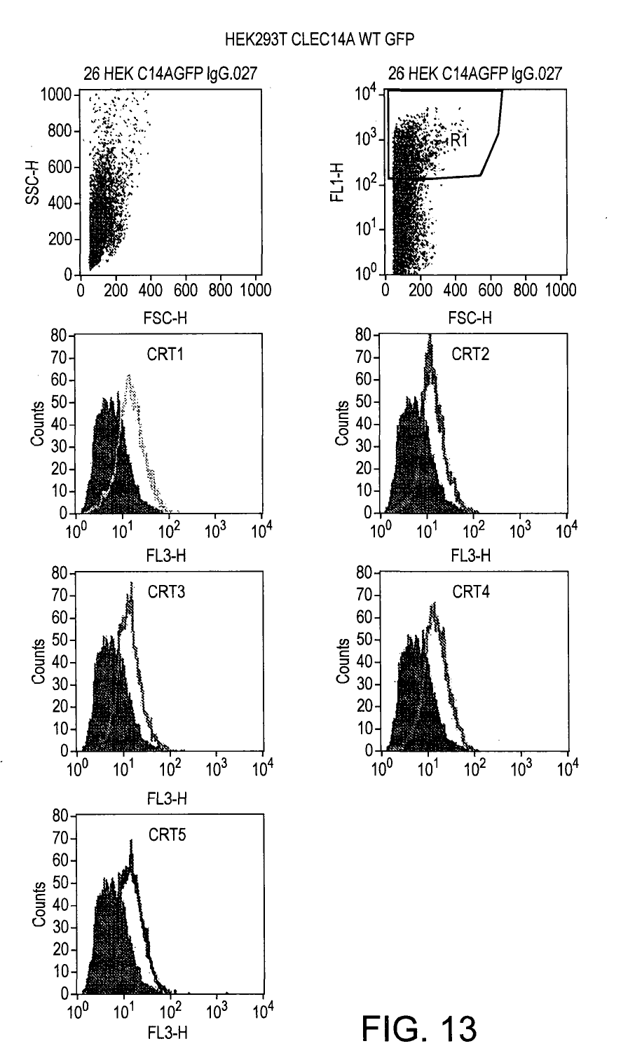

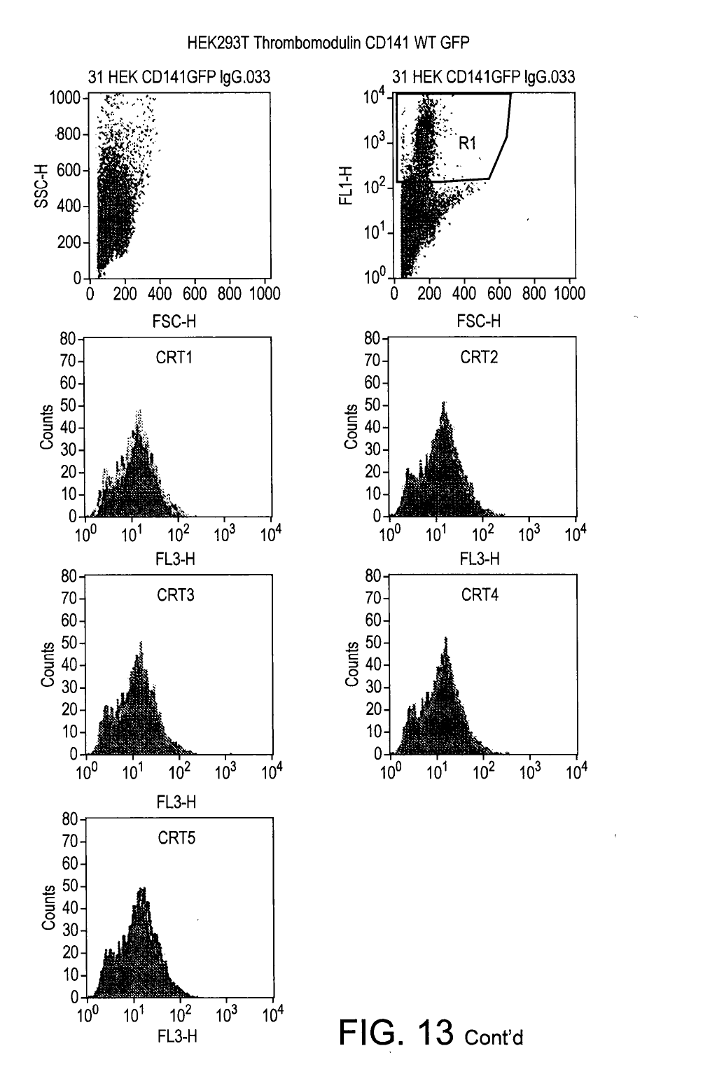

FIG. 13 shows analysis of the binding of CRT antibodies using flow cytometry. All constructs have a C-terminus GFP tag so green cells were gated and stained red. All CRT antibodies bind to CLEC14A wild type with a C terminal GFP tag expressed in HEK293T cells. None of the CRT antibodies bind to wild type thrombomodulin with GFP tag expressed in HEK293T cells.

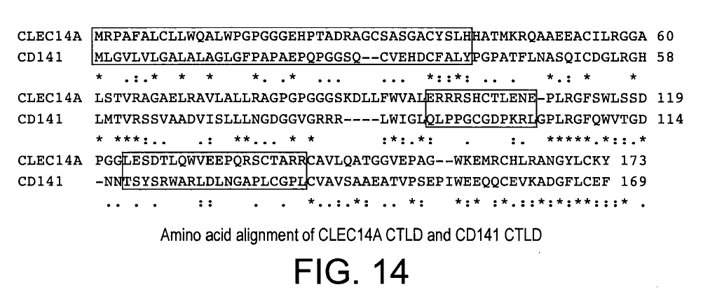

FIG. 14 shows an alignment of CLEC14A regions 1-42 of CD141; CLEC14A regions 97-108 of CD141; and CLEC14A regions 122-142 of CD141.

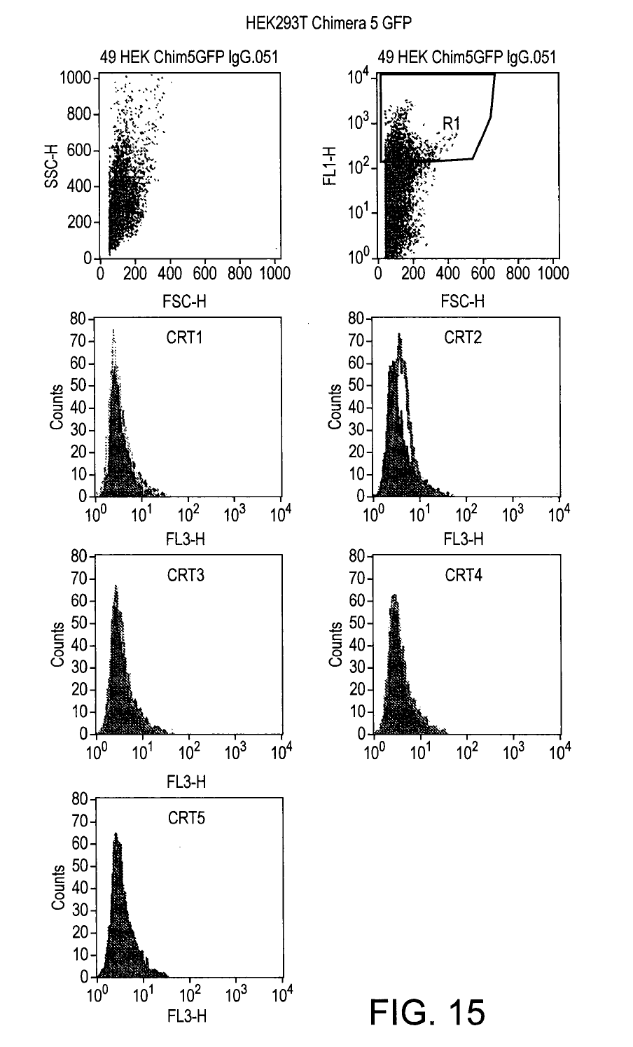

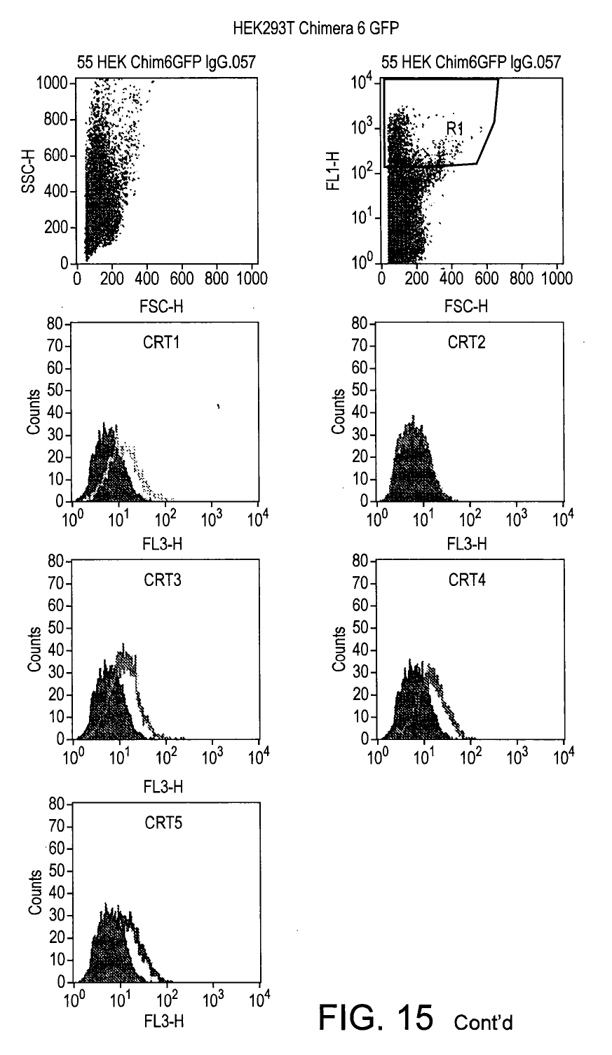

FIG. 15 shows that Chimera 5 (CTLD of thrombomodulin, rest CLEC14A) is not recognised by any of the CRT antibodies except a slight shift in fluorescence with CRT2. Chimera 6 (Sushi of thrombomodulin to ensure correct folding of CTLD of CLEC14A) results in binding of all CRT antibodies except CRT2.

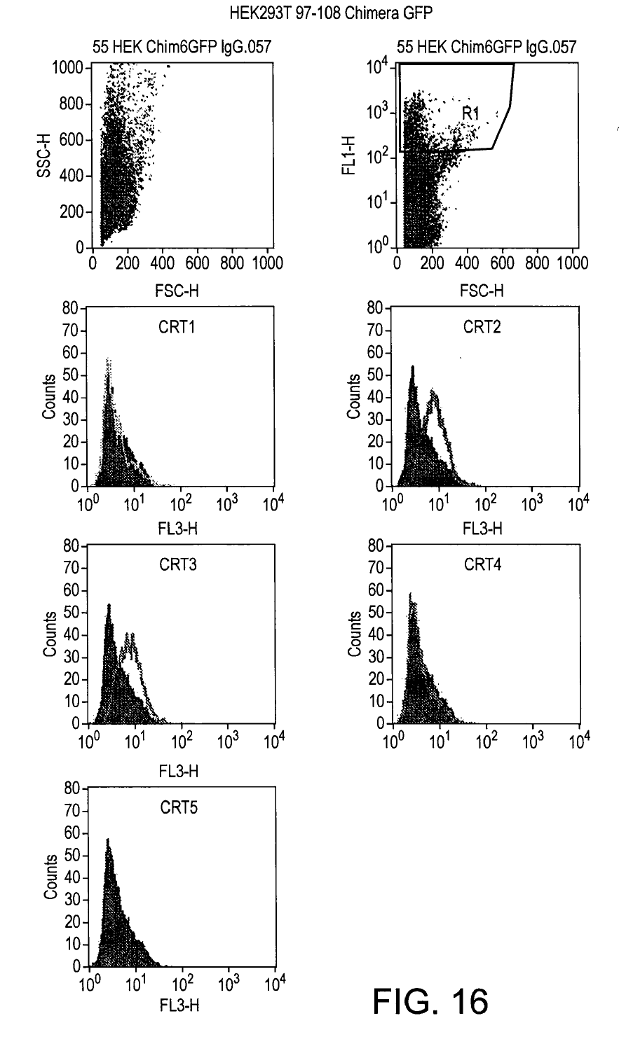

FIG. 16 shows flow cytometry analysis of binding by antibodies CRT1-5 when Residues 97-108 were swapped with corresponding regions from thrombomodulin. This resulted in correct folding as CRT2 and CRT3 can still bind. However CRT1, CRT4 and CRT5 cannot recognise this mutant suggesting this to be the binding region.

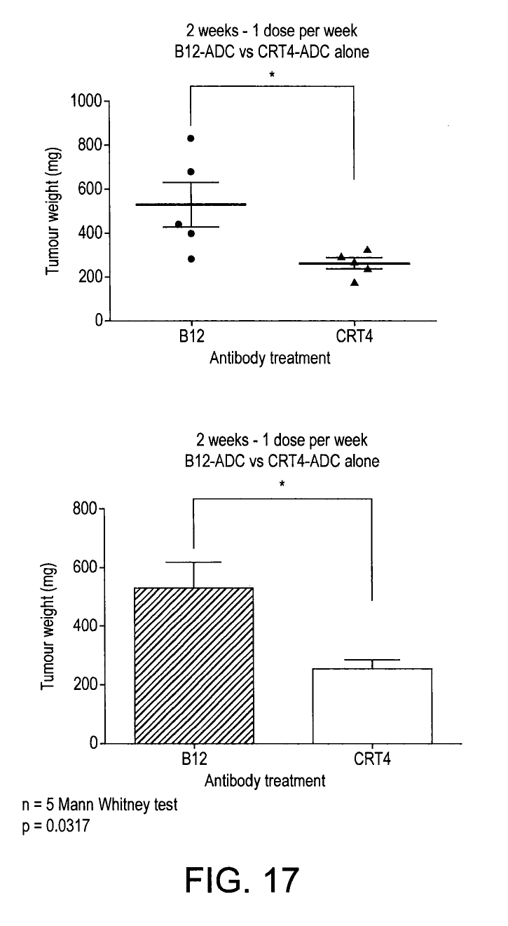

FIG. 17 shows the reduced tumour weight of lung carcinoma when a CRT-4 antibody drug conjugate treatment is employed. FIG. 17A shows the results when 1 million Lewis lung carcinoma cells were injected subcutaneously into the right flank of mice and allowed to grow to a visible size. 1 mg/kg of antibody CRT4-ADC (right image) or control B12-ADC (left image) was administered through tail vein injections. Mice were observed for an hour and culled 24 hours later. All organs were dissected and fixed. n=1. FIG. 17B shows end point tumour weights of antibody drug conjugate treatments. There was a significant difference between the wet weights of CRT4-ADC treatment group when compared with B12-ADC treatment group. Mann Whitney test p=0.0317. Error bars SEM, n=5. Data pooled from two separate experiments of the same method.

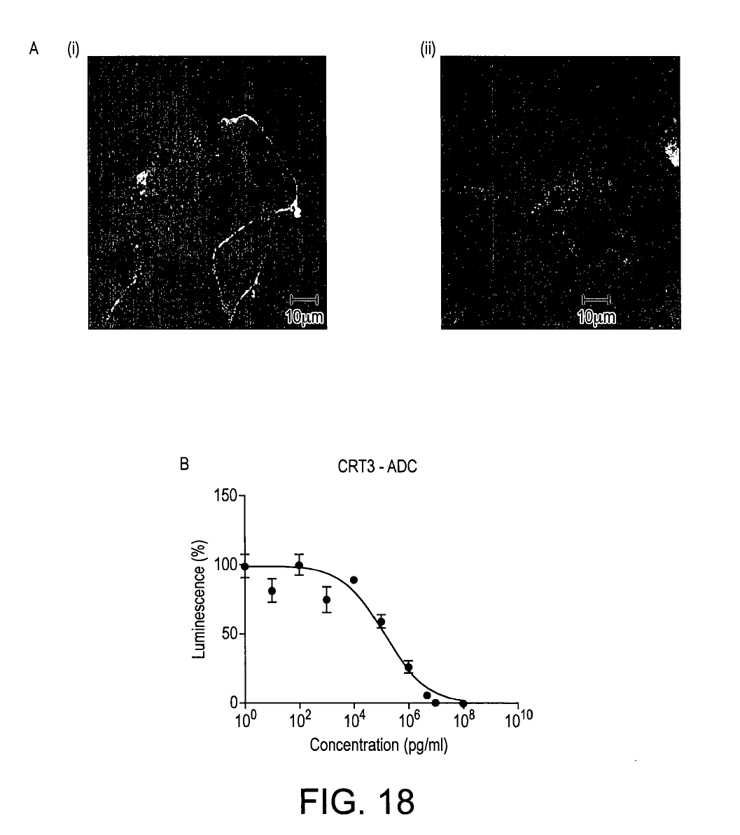

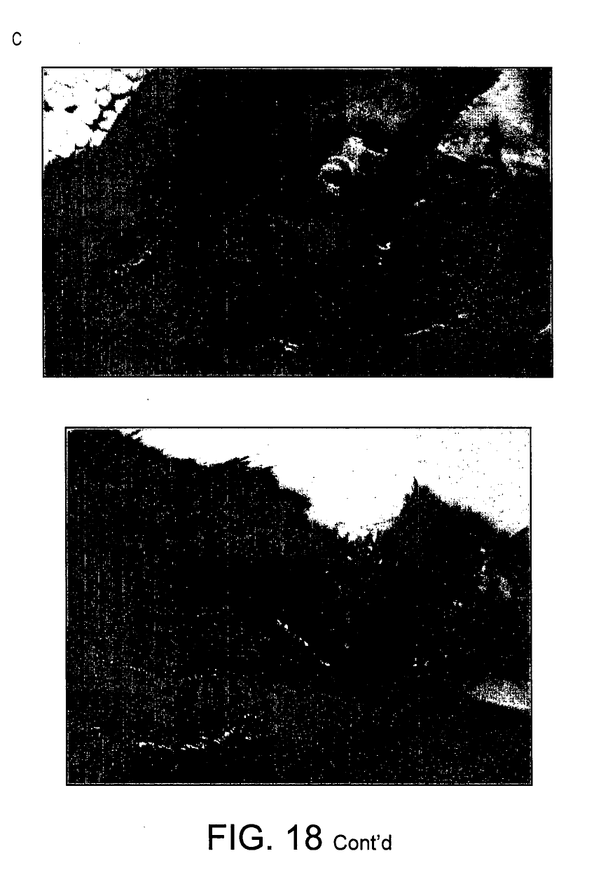

FIG. 18 shows the internalisation of a CRT-3 antibody drug conjugate and the effect of this on a tumour 24 hours after administration. FIG. 18A shows the internalisation of CRT-3 antibody drug conjugate in HUVECs where fluorescent imaging shows the localisation of the CRT-3 after 0 and 90 minutes and FIG. 18B shows the cytotoxicity measured by Cell Titre Glo luminescent cell viability assay in HUVEC treated CRT-3-ADC (IC50=137.6 ng/ml). FIG. 18C shows extensive haemorrhage at the site of the tumour in the CRT3-ADC treated mouse and not in a control, demonstrating tumour-specific disruption of angiogenesis.

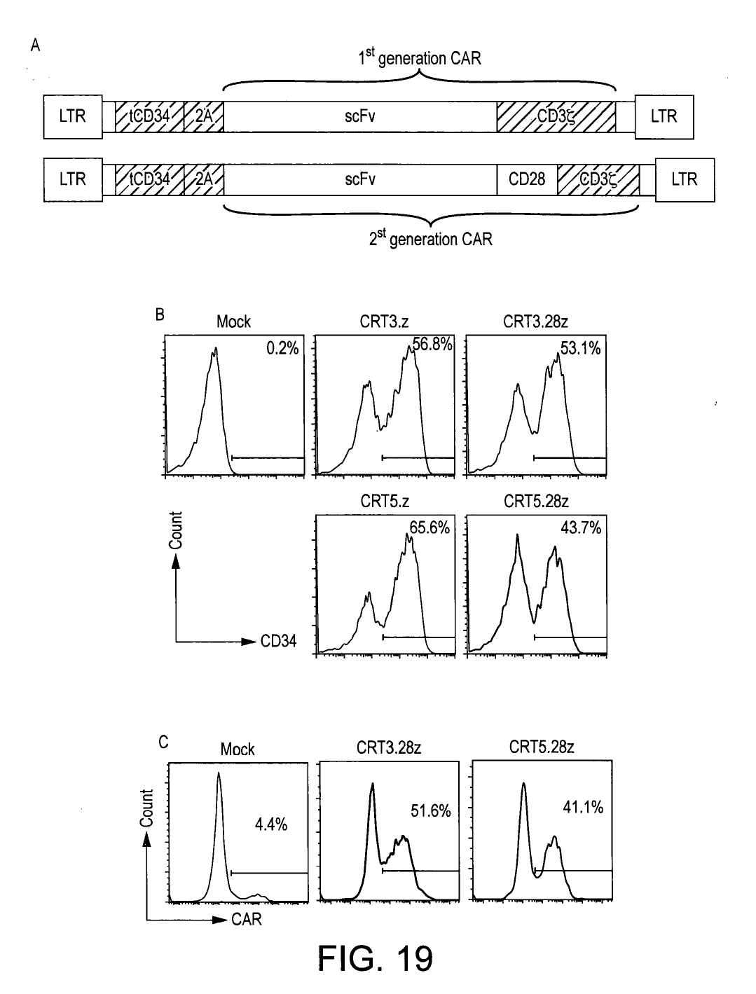

FIG. 19 shows a retroviral CAR vector (based on pMP71) that co-expresses a truncated CD34 marker gene and an scFv fragment/CD3 zeta chain chimeric receptor (FIG. 19A). Expression is driven from the LTR promoter and the 2A peptide linker ensures equimolar expression of both the CD34 and the CAR. Second generation CAR constructs included the CD28 costimulatory domain. FIG. 19B shows CD34 staining analysed by flow cytometry demonstrating successful transduction of T cells using retroviral constructs that co-express a CLEC14A-specific CAR. First generation CARs based on the antibodies CRT3 and CRT5 are referred to as CRT3.z and CRT5.z respectively. Second generation CARs based on the antibodies CRT3 and CRT5 are referred to as CRT3.28z and CRT5.28z respectively. Note equivalent expression was seen in CD4 and CD8 T cell subsets (data not shown). FIG. 19C shows cells stained directly for expression of CAR using CLEC14A-Fc (% values show specific binding of CLEC14A-Fc having subtracted background staining with Fc alone).

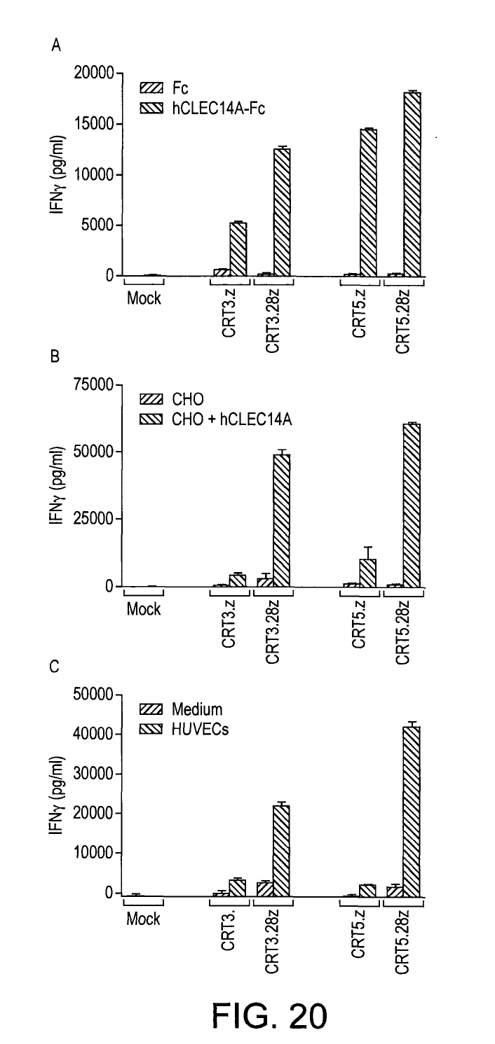

FIG. 20 shows the ability of T cells transduced to express 1^{st} or 2^{nd} generation CARs based on antibodies CRT3 or CRT5 or mock-transduced (control) T cells to respond to CLEC14A expressed either as (A) plate-bound recombinant Fc fusion protein, (B) expressed on engineered CHO cells, or (C) expressed on human umbilical vein endothelial cells (HUVECs) which naturally express CLEC14A when grown in static culture. T cell response was measured using an ELISA for interferon gamma production. Data shown are representative of that obtained from 3-7 repeat experiments. T cells were adjusted to equalise the frequency of transgene expressing cells. All histograms show mean response+SD.

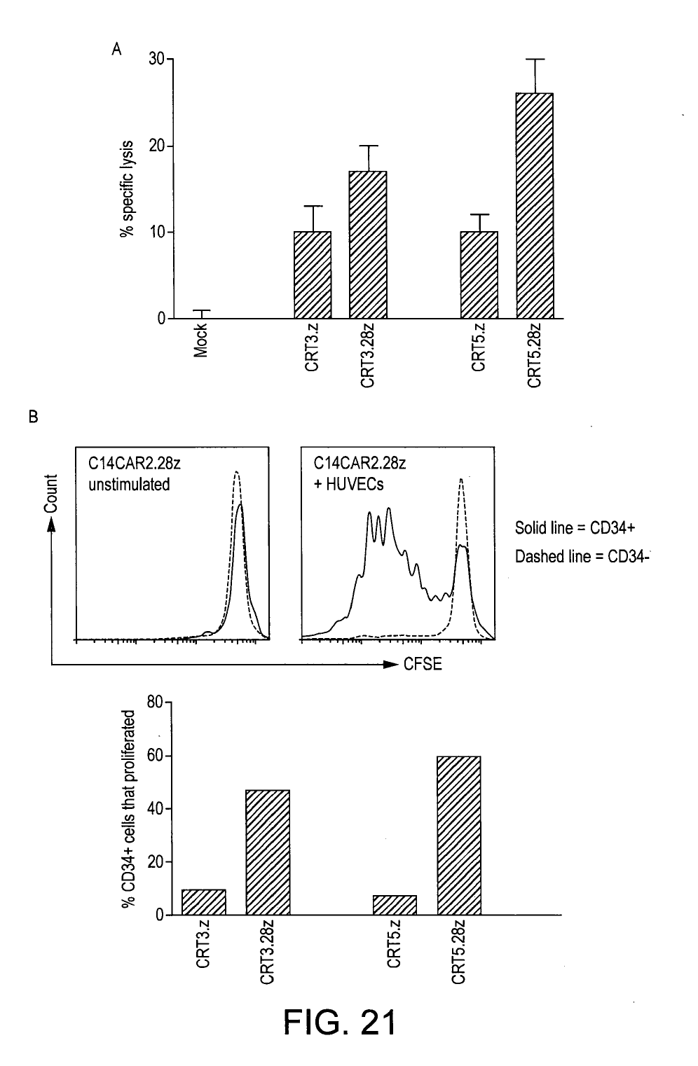

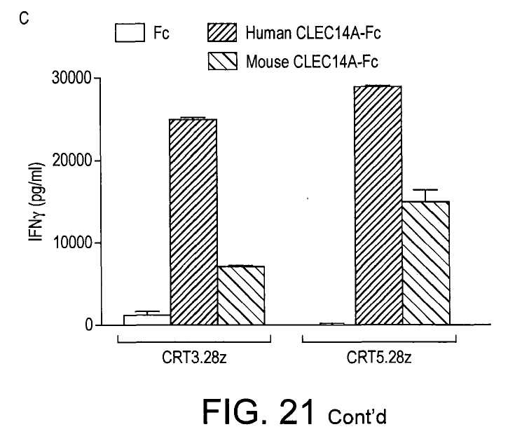

FIG. 21 shows further in vitro functional testing of CLEC14A-specific CAR-transduced T cells. T cells transduced to express 1^{st} or 2^{nd} generation CARs based on antibodies CRT3 or CRT5, or mock-transduced (control) T cells, were tested for their ability to respond to CLEC14A in the following functional assays: (A) Cytotoxicity, using CHO cells engineered to express human CLEC14A (having subtracted background levels of lysis of CHO alone (control cells)). Data shown are representative of 5 repeat experiments. (B) Proliferation, using CFSE-labelled CAR-transduced T cells we measured the proliferation of CAR+ (CD34+) and CAR− (CD34−) cell subsets when co-cultured for 4 days with HUVECs. Data shown are representative of 2 repeat experiments. (C) The response of (CLEC14A-specific CAR-transduced T cells to both human and mouse CLEC14A was assessed using interferon gamma release. T cells were adjusted to equalise the frequency of transgene expressing cells. Data shown are representative of 6 repeat experiments. All histograms show mean response+SD.

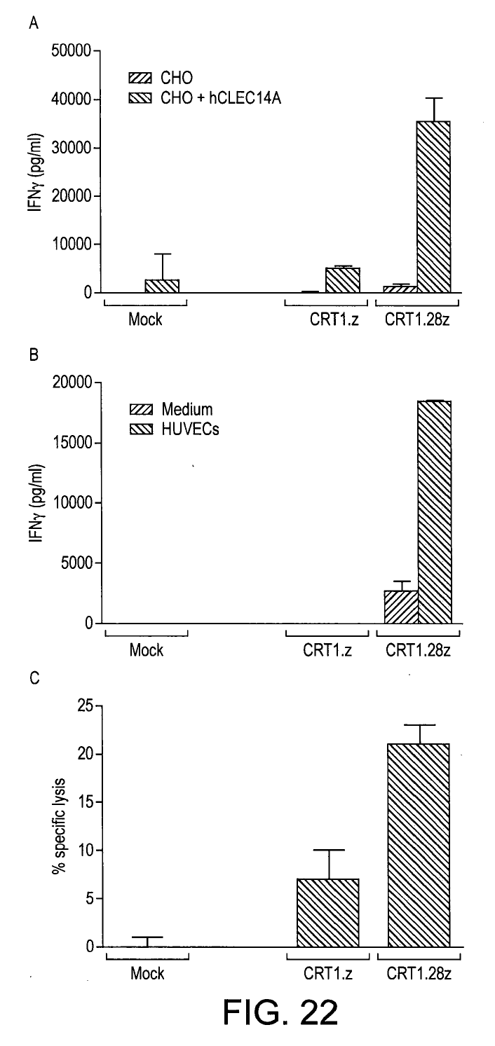

FIG. 22 shows in vitro functional testing of T cells transduced with a third CLEC14A-specific CAR based on antibody CRT1. T cells transduced to express 1^{st} or 2^{nd} generation CARs based on antibody CRT1, or mock-transduced (control) T cells, were tested for their ability to respond to CLEC14A expressed on (A) engineered CHO cells or (B) human umbilical vein endothelial cells (HUVECs) which naturally express CLEC14A when grown in static culture. T cell response was measured using an ELISA for interferon gamma production. (C) Cytotoxicity, using CHO cells engineered to express human CLEC14A (having subtracted background levels of lysis of CHO alone (control cells)). Data shown are representative of that obtained from at least 3 repeat experiments. T cells were adjusted to equalise the frequency of transgene expressing cells. All histograms show mean response+SD.

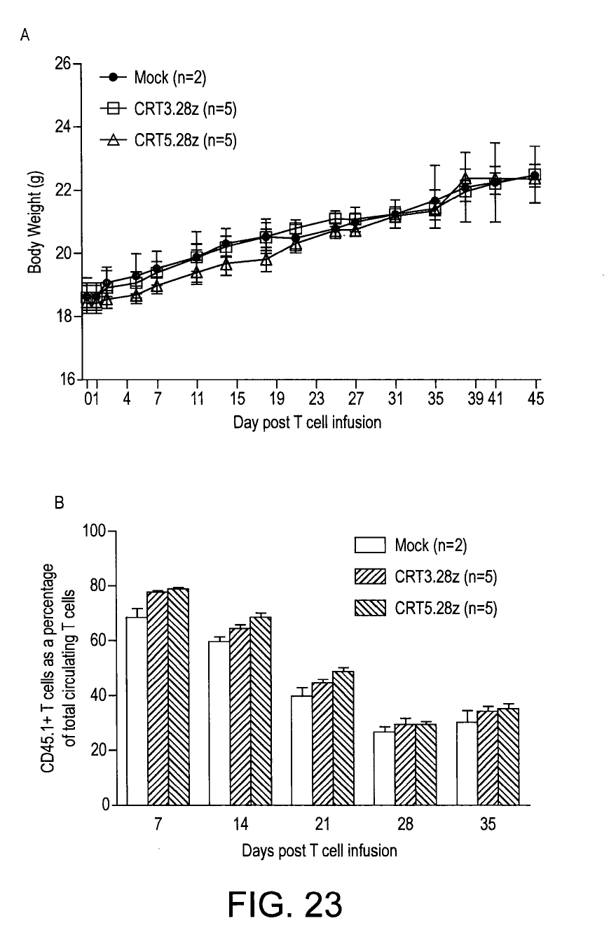

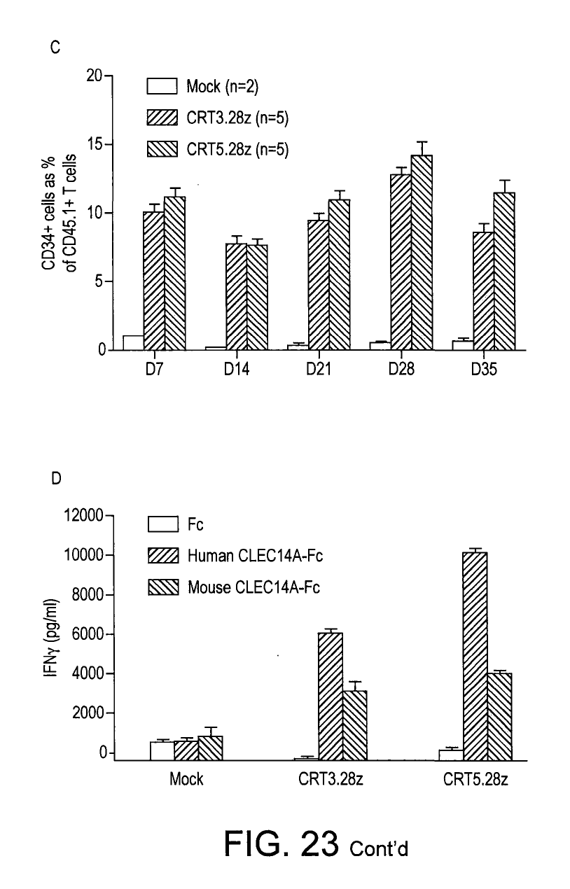

FIG. 23 shows toxicity testing in vivo using healthy C57/BL6 mice injected with CLEC14A-specific CAR-transduced mouse T cells. T cells transduced to express 1^{st} or 2^{nd} generation CARs based on antibodies CRT3 or CRT5, or mock-transduced (control) T cells, were injected into the tail vein of healthy C57BL6 mice that had previously been irradiated (4 Gy) to aid T cell engraftment. T cells were derived from a C57BL6 congenic strain (BoyJ) which carry the marker CD45.1. 20 million T cells (containing 4 million engineered T cells) were infused per mouse. Mice were monitored for the next 45 days and showed no visible signs of toxicity. (A) Body weights increased normally during this time. (B) Weekly tail bleeds demonstrated that the infused CD45.1+ T cells persisted for at least five weeks post infusion and throughout this time they comprised at least 30% of the total circulating T cell pool (note over time the host's own T cells recover). (C) Staining the same samples for CD34 as well as CD45.1 demonstrated that the proportion of engineered (CD34+) T cells relative to the total infused T cells population remained relatively constant during this time. (D) At the end of the experiment, splenocytes were harvested from a CAR-treated mouse and CD34+ cells were isolated using immunomagnetic bead selection. Testing these cells immediately ex vivo demonstrated that they were still capable of responding to both human and mouse CLEC14A as measured by ELISA for interferon gamma release. Graphs A-C show mean+SEM. Graph D shows mean+SD.

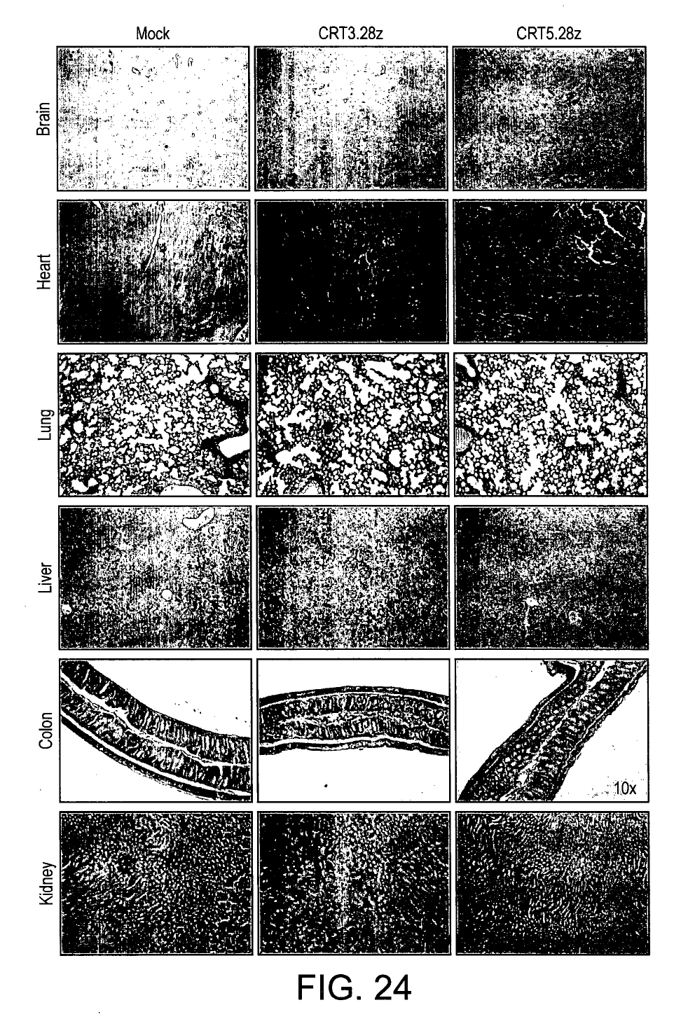

FIG. 24 shows toxicity testing in vivo using healthy C57/BL6 mice injected with CLEC14A-specific CAR-transduced mouse T cells. At the end of the experiment mice were culled and major organs harvested. Histological examination revealed no evidence of pathology.

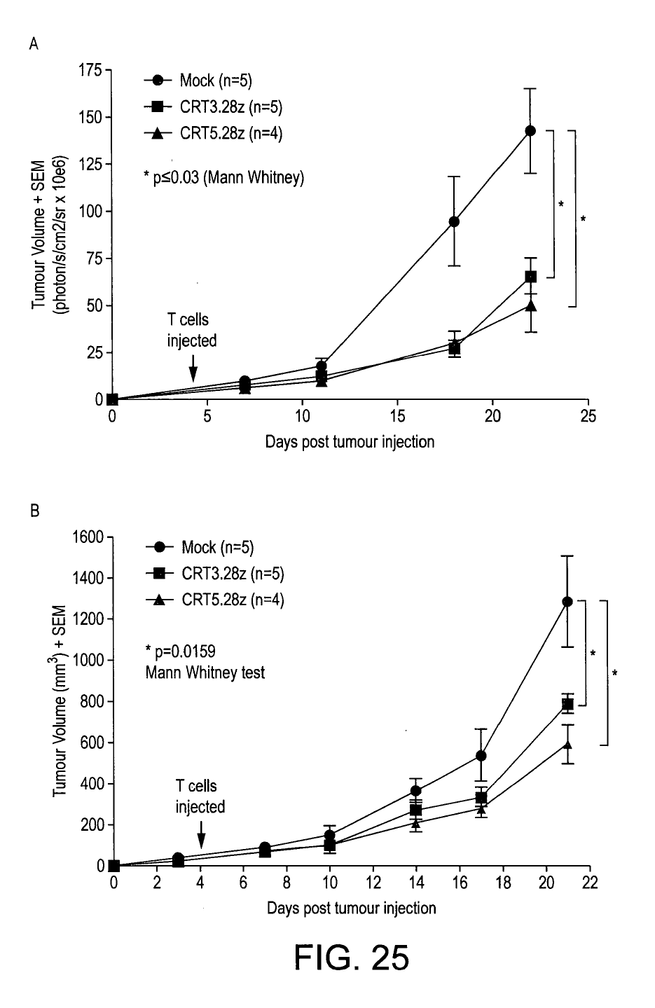

FIG. 25 shows the anti-tumour response of CLEC14A-specific CAR-transduced mouse T cells when injected into mice carrying Lewis Lung carcinoma tumours. C57BL6 mice were injected subcutaneously with Lewis Lung Carcinoma cells (1 million cells/mouse) and 4 days later mice received 4 Gy total body irradiation to aid T cell engraftment. T cells transduced to express 2^{nd} generation CARs based on antibodies CRT3 or CRT5, or mock-transduced (control) T cells, were then injected into the tail vein. Mice received a total of 20 million T cells (CD8:CD4=5:2) with CRT3.28z and CRT5.28z expressed on 2.2 and 1.4 million of these cells. Tumour growth was then monitored using (A) Bioluminescence or (B) Calipers.

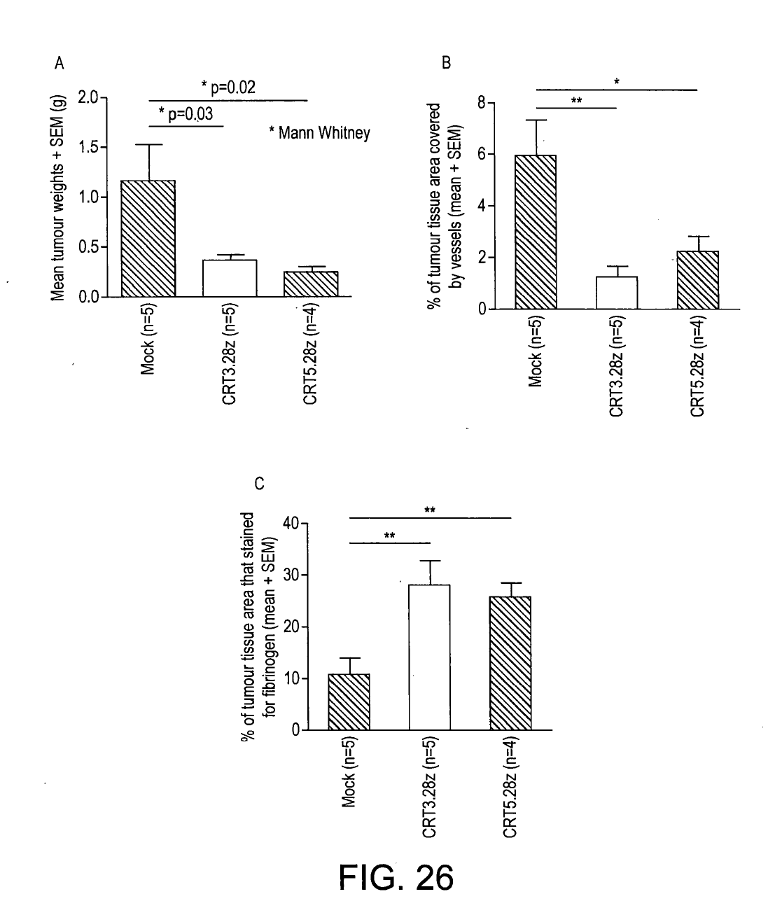

FIG. 26 shows the anti-tumour response of CLEC14A-specific CAR-transduced mouse T cells when injected into mice carrying Lewis Lung carcinoma tumours. At the end of the experiment tumours were excised and weighed (A). Histological analysis demonstrated that tumours from mice treated with the CARs showed significantly reduced vascular density (B, staining for MECA-32) and greater levels of vascular leakage (C, staining for fibrinogen).

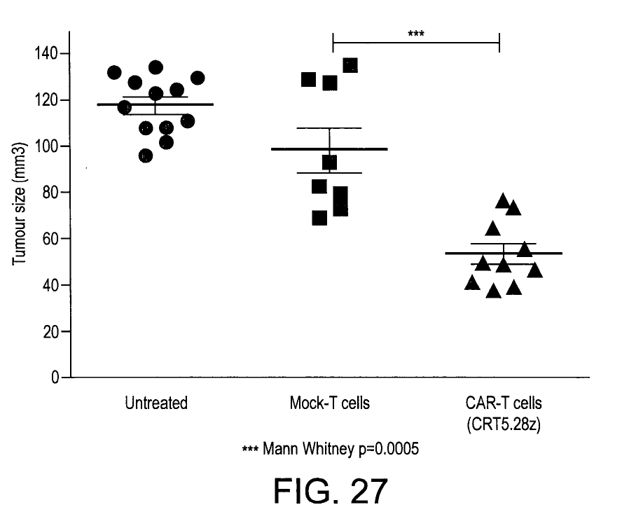

FIG. 27 shows the anti-tumour response of CLEC14A-specific CAR-transduced mouse T cells when injected into RipTag2 mice (where the rat insulin promoter (RIP) directs expression of the SV40 Large T antigen transgene (TAg) to beta cells of the pancreatic islets). Tumours arise at around 10 weeks of age and usually result in the death of the animal by approx. 14 weeks. Mouse T cells expressing a second generation CAR based on CRT5 (or mock-transduced control cells) were infused into 10 week old riptag2 mice. Mock-treated mice were culled at 14 weeks of age and tumour size measured. CAR-treated mice were culled 2 weeks later (16 weeks old) and again tumour size was measured. Results demonstrated a highly significant inhibition of tumour size in mice treated with CAR-transduced T cells (A) compared with mock-transduced T cell-treated animals. There was also some evidence of a survival benefit, since 12 mice were treated in each of the study, and of those that received mock-transduced T cells, 4 died before 14 weeks of age, whereas all 12 mice treated with CAR-transduced T cells were alive at 14 weeks of age, and all but two of them were still alive two weeks later. Data show tumour size in individual mice. Horizontal line indicates mean tumour size+SEM. Note untreated mice were not irradiated.

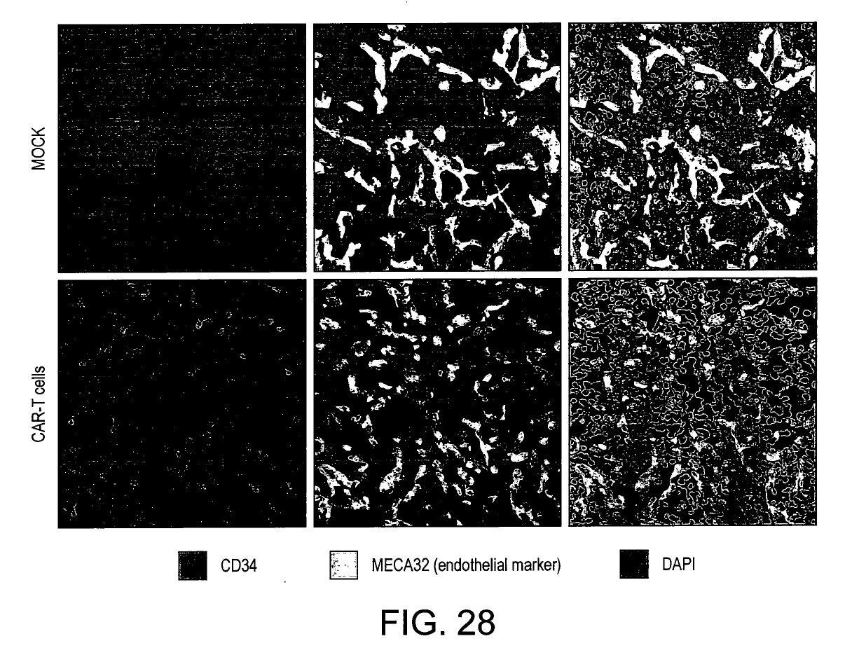

FIG. 28 shows confocal imaging of RIP-Tag2 tumours 4 weeks after intravenous injection of CAR-T cells. The imaging shows that CD34+(CAR-transduced) T cells accumulate in the tumours.

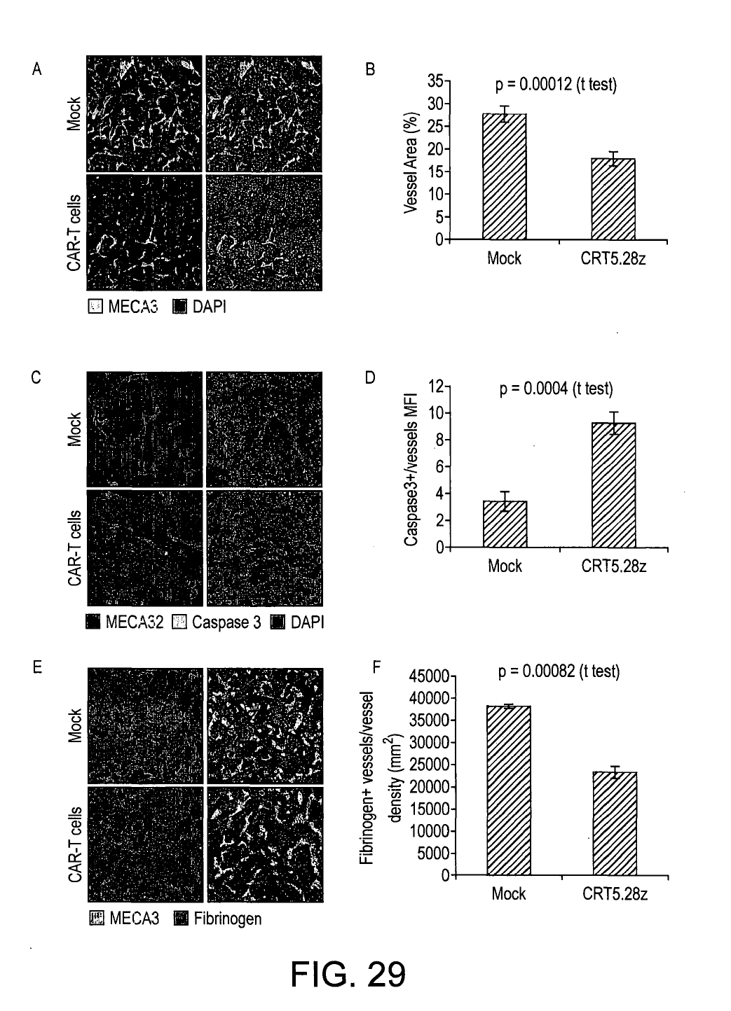

FIG. 29 shows histological analysis of RipTag2 tumours treated with CAR- (or Mock-) engineered T cells. FIG. 29A shows that vascular density is reduced in CAR-T cell treated tumours as indicated by staining for the endothelial marker MECA32. FIG. 29B shows a summary of vascular density in CAR- vs Mock T cell treated tumours. FIGS. 29C and D show that mice treated with CAR-transduced T cells display an increase in apoptotic vessels in the tumour as indicated by caspase 3 staining. FIGS. 29 E and F show that mice treated with CAR-transduced T cells display a decrease in fibrinogen staining in the tumour vasculature.

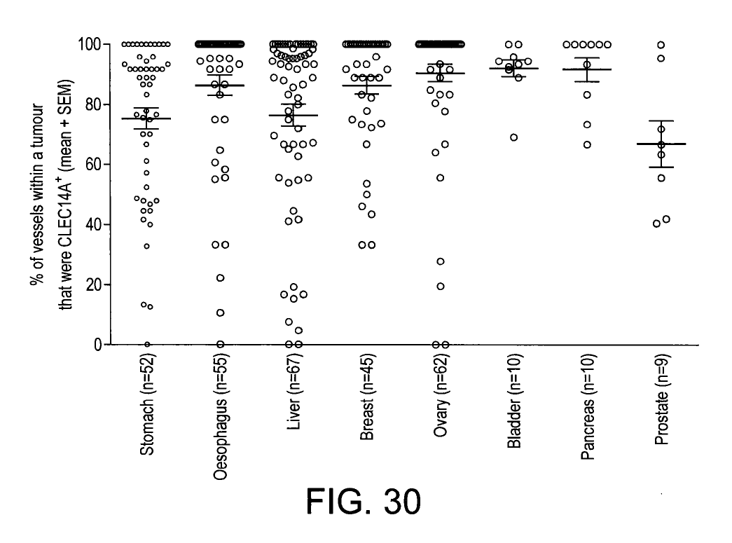

FIG. 30 shows further characterisation of CLEC14A expression in human tumour tissue arrays calculating the percentage of vessels within a tumour that expressed CLEC14A. n=number of cases studied for each cancer type. Each case is represented by a circle, with horizontal lines showing mean percentage values+SEM for each cancer type.

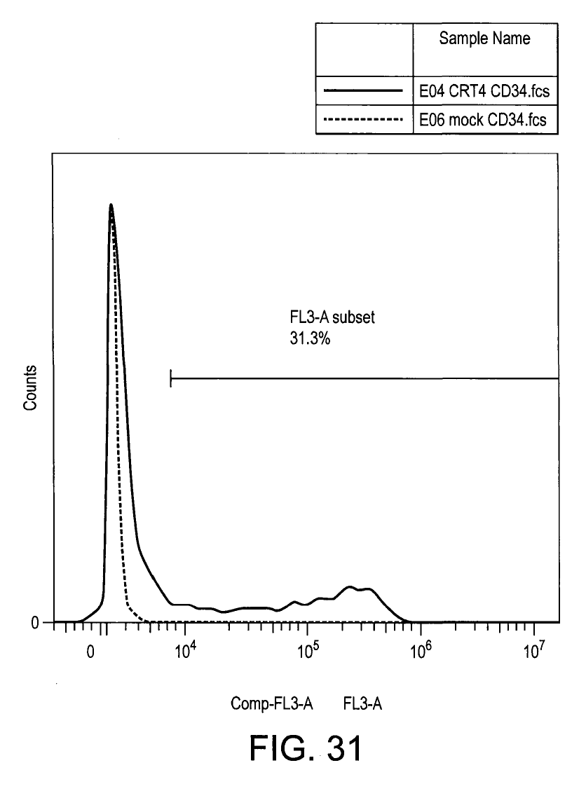

FIG. 31 shows the transduction of T-cells with CRT4 CAR.

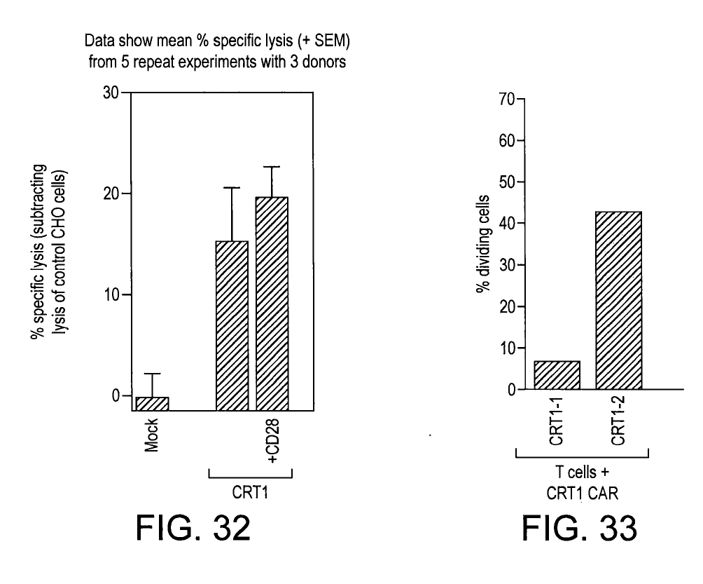

FIG. 32 shows that both first and generation CRT1 CAR T cells can mediate cell lysis of CHO cells expressing CLEC14A.

FIG. 33 shows the proliferative activity of T cells expressing first and second generation CRT-1 CAR constructs.

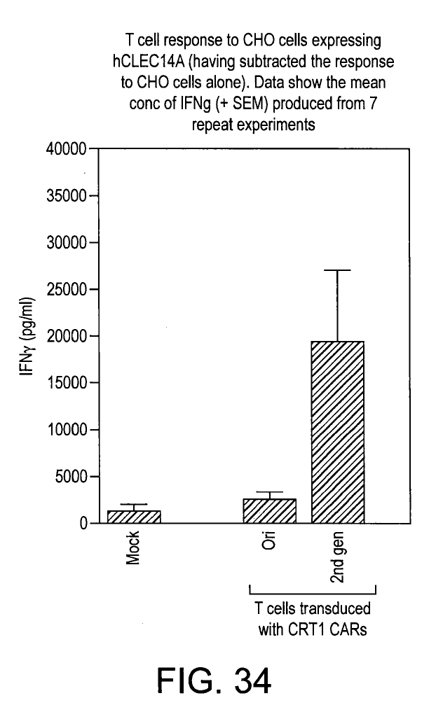

FIG. 34 shows the interferon gamma release activity of T cells expressing first and second generation CRT-1 CAR constructs in response to CHO cells expressing humanCLEC14A (having subtracted the response to CHO cells alone). Data show the mean conc. of IFNγ (+SEM) produced from 7 repeat experiments.

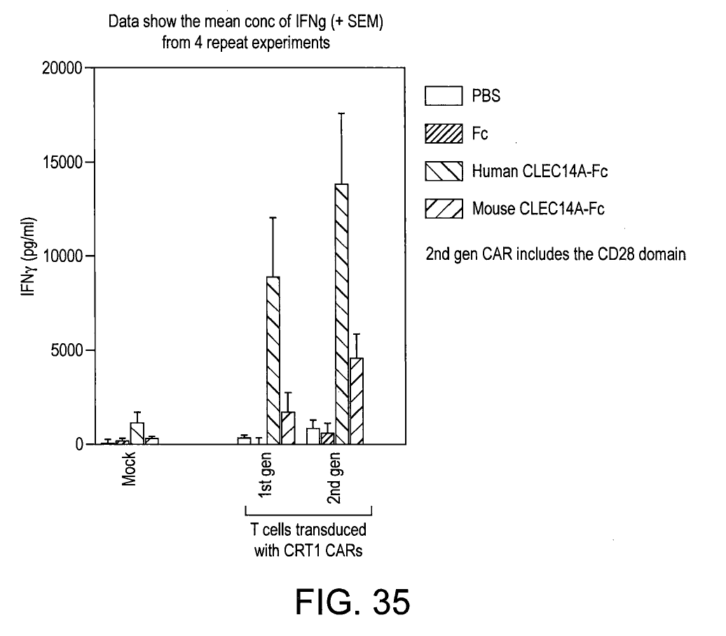

FIG. 35 shows the interferon gamma release activity of T cells expressing first and second generation CRT-1 CAR constructs in response to Human or mouse CLEC14A-Fc fusion proteins (or Fc protein alone).

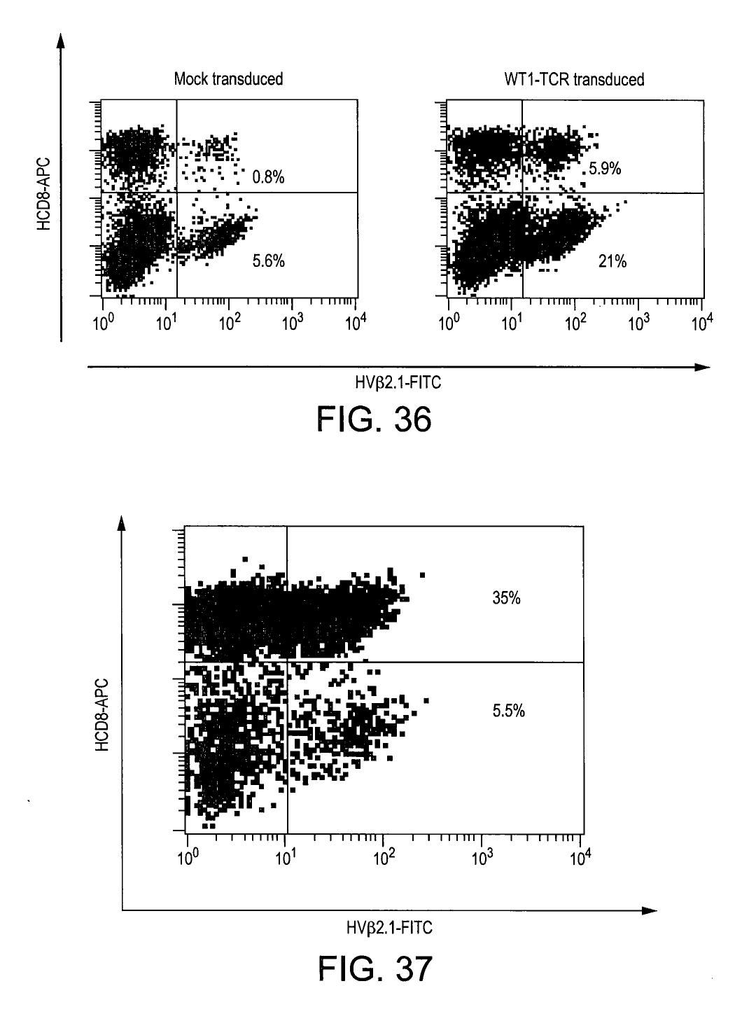

FIG. 36 shows retroviral TCR gene transfer into human T cells and TCR expression in human PBMC after transduction. Peripheral blood lymphocyte activation was carried out using anti-CD3 antibodies, IL-7 and IL-2, followed by transduction with retroviral vectors (encoding a TCR specific for WT1) three days later. At day 6, TCR expression was monitored using TCR-V-beta 2.1 antibodies. The percentage of un-manipulated human T cells expressing V-beta 2.1 was shown using mock transduced T cells. Both CD8 negative and CD8 positive T cells after transduction had an increased percentage of V-beta 2.1 cells.

FIG. 37 shows an expansion of CD8 positive T cells expressing V-beta 2.1 is achieved by repeated stimulation of TCR transduced T cells with T2 cells presenting the WT126 peptide. Thus an increase of CD8+-Vb2.1+ T cells occurs after antigen stimulation.

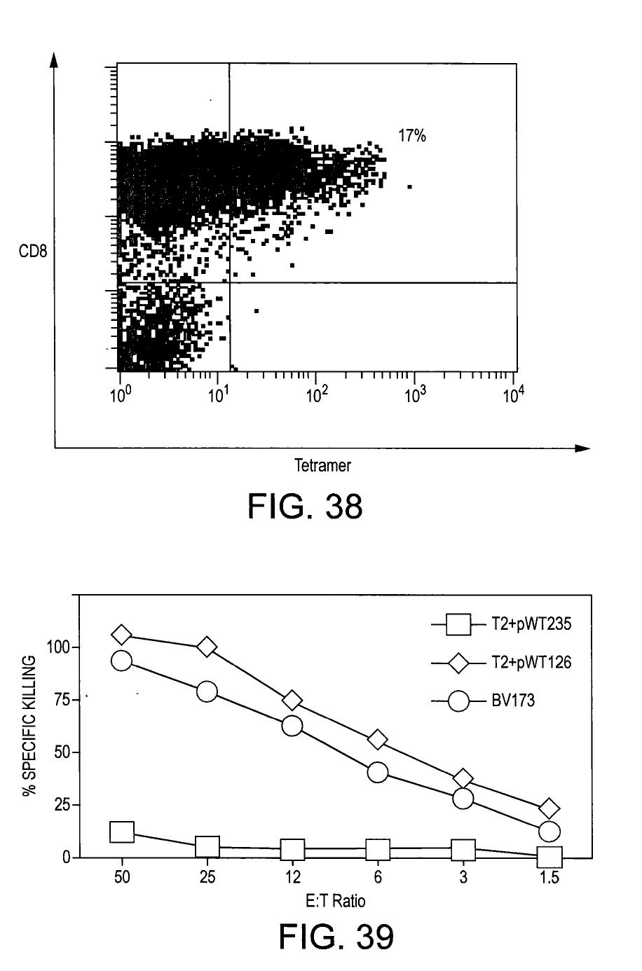

FIG. 38 shows that HLA-A2/pWT126 tetramers and TCR-transduced T cells stain together.

FIG. 39 shows that T cells transduced with the TCR are able to kill T2 cells presenting the WT126 peptide but not T2 cells presenting the pWT235 peptide. The transduced T cells also further kill HLA-A2 BV173 leukaemia cells which endogenously express WT1. Thus, TCR transduced bulk T cells show pWT126-specific killing activity.

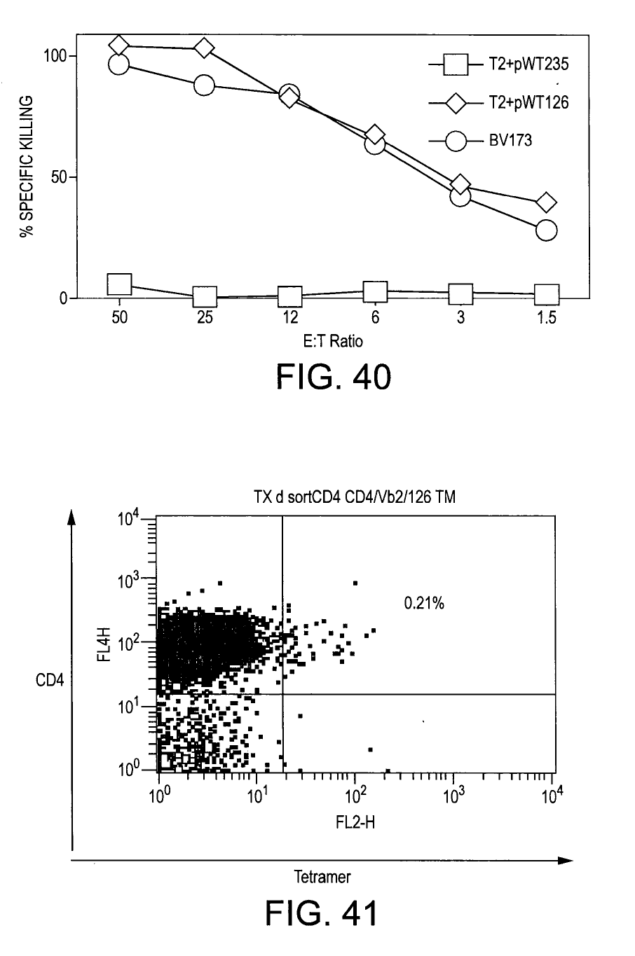

FIG. 40 shows that HLA-A2 positive T2 cells presenting the WT126 peptide are killed by purified TCR-transduced CD8 positive T cells but that T2 cells coated with the WT235 peptide are not killed. Further, CD8 positive transduced T cells also kill HLA-A2 BV173 leukaemia cells which endogenously express WT1. Thus, transduced CD8+ T cells show pWT126-specific killing activity. (Key—unfilled square=T2+pWT235, filled diamond=T2+pWT126, unfilled circle=BV173).

FIG. 41 shows that a small amount of purified CD4 positive transduced T cells stain together with HLA-A2/pWT126 tetramers.

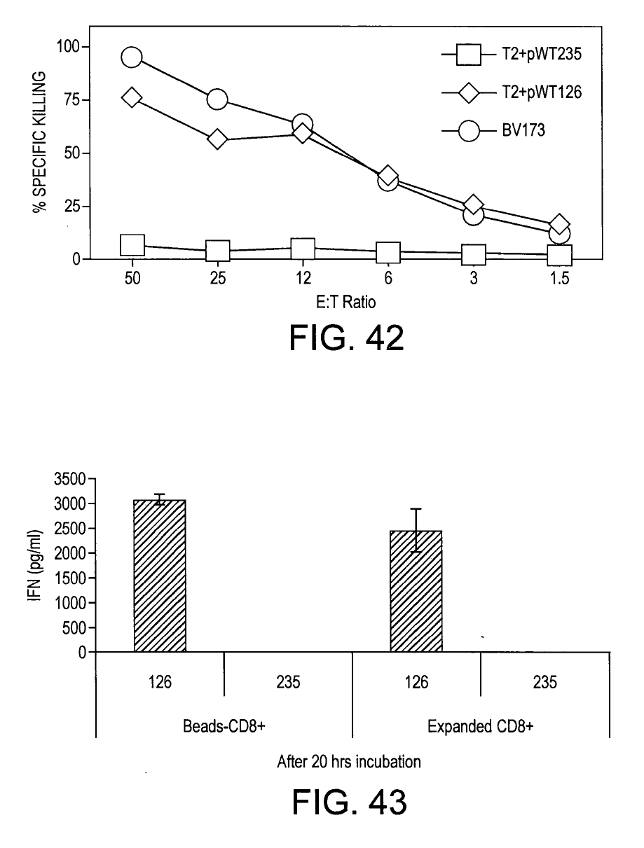

FIG. 42 shows that HLA-A2 positive T2 cells presenting the WT126 peptide are killed by purified TCR-transduced CD4 positive T cells but that T2 cells coated with the WT235 peptide are not killed. Further, CD4 positive transduced T cells also kill HLA-A2 BV173 leukaemia cells which endogenously express WT1.

FIG. 43 shows that IFN-γ is produced by the purified TCR-transduced CD8 positive cells after stimulation with the HLA-A2 positive T2 cells coated with the WT126 but not by equivalent cells coated with the pWT235 peptide. IFN-γ is also produced by the CD8 positive transduced T cells after stimulation with HLA-A2 positive BV173 leukaemia cells which express WT1 endogenously. Thus, TCR transduced CD8+ T cells show pWT126-specific IFNγ production.

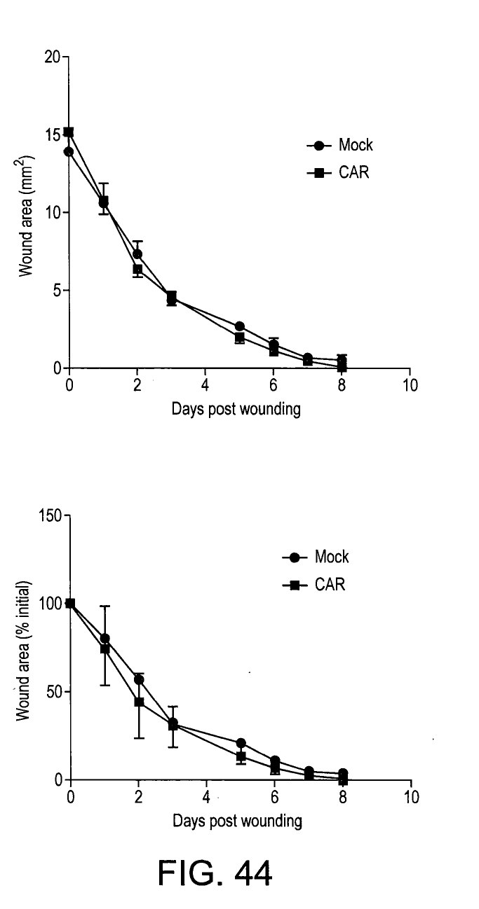

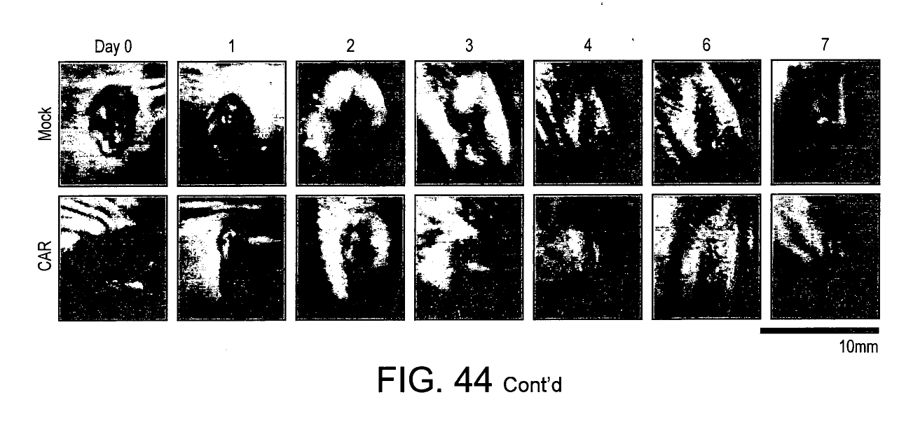

FIG. 44 shows that the CRT5 CAR does not impede healing of a skin wound in tumour bearing mice.

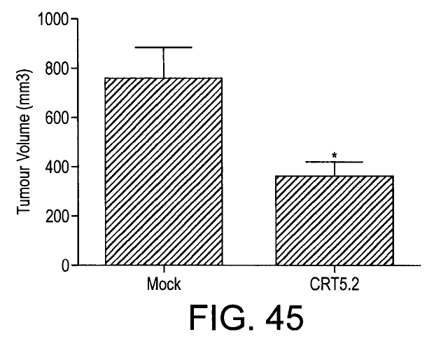

FIG. 45 shows PDAC tumour volumes 3 weeks after treatment with control (n=5) or CRT5 CAR (with CD28 costimulatory domain) expressing cells.

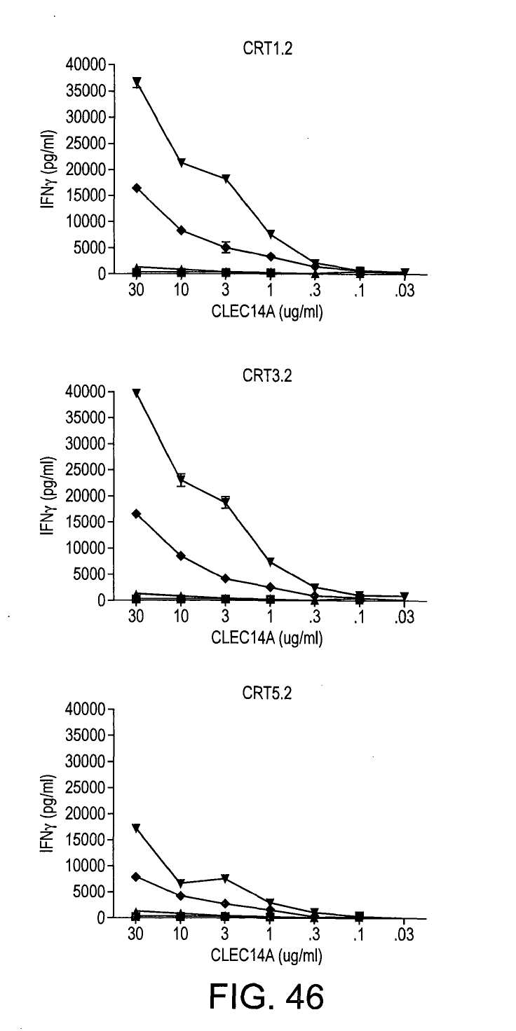

FIG. 46 shows CRT1, 3 and 5 CAR (with CD28 costimulatory domain) T cell response to titrated concentrations of human and mouse recombinant CLEC14A.

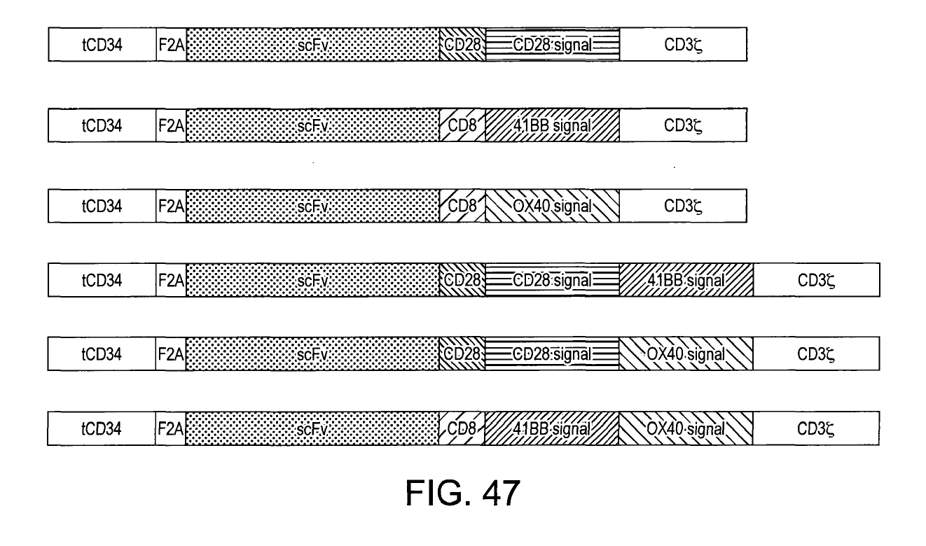

FIG. 47 shows the design of constructs which encode CARs with different costimulatory domains; 1) tCD34-F2A-scFv-CD28 TM-CD28 signal-CD3zeta, 2) tCD34-F2A-scFv-CD8 TM-4-1BB signal-CD3 zeta, 3) tCD34-F2A-scFv-CD8 TM-OX40 signal-CD3 zeta, 4) tCD34-F2A-scFv-CD28 TM-CD28 signal-4-1BB signal-CD3zeta, 5) tCD34-F2A-scFv-CD28 TM-CD28 signal-OX40 signal-CD3 zeta, 6) tCD34-F2A-scFv-CD8 TM-4-1BB signal-OX40 signal-CD3zeta. The tCD34 is included to identify successfully transduced cells and thus constructs may exclude this and F2A. A hinge or spacer region may additionally be included e.g. one from CD8a.

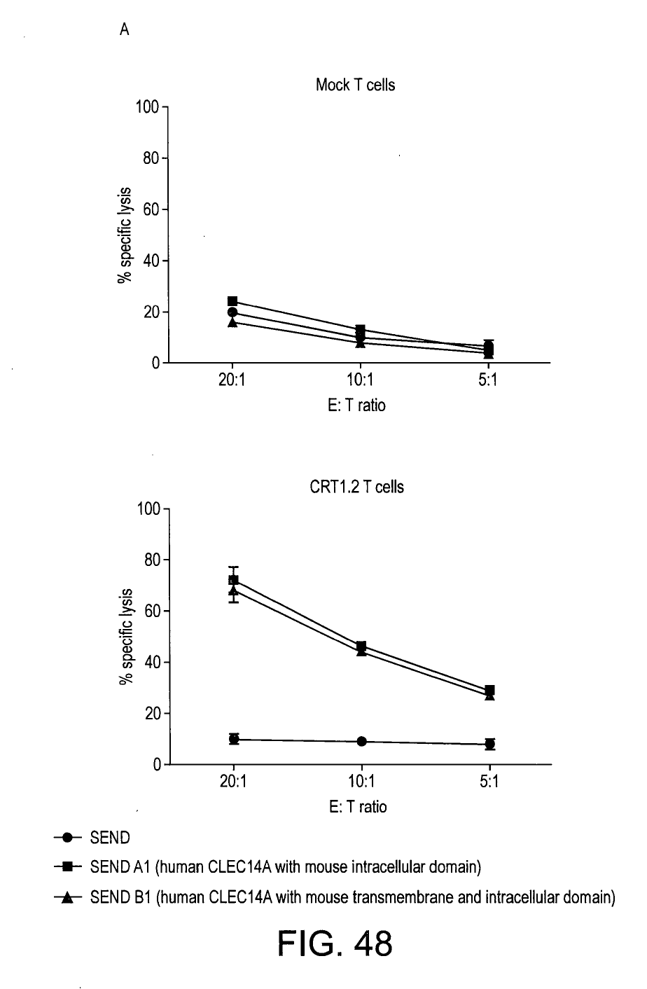

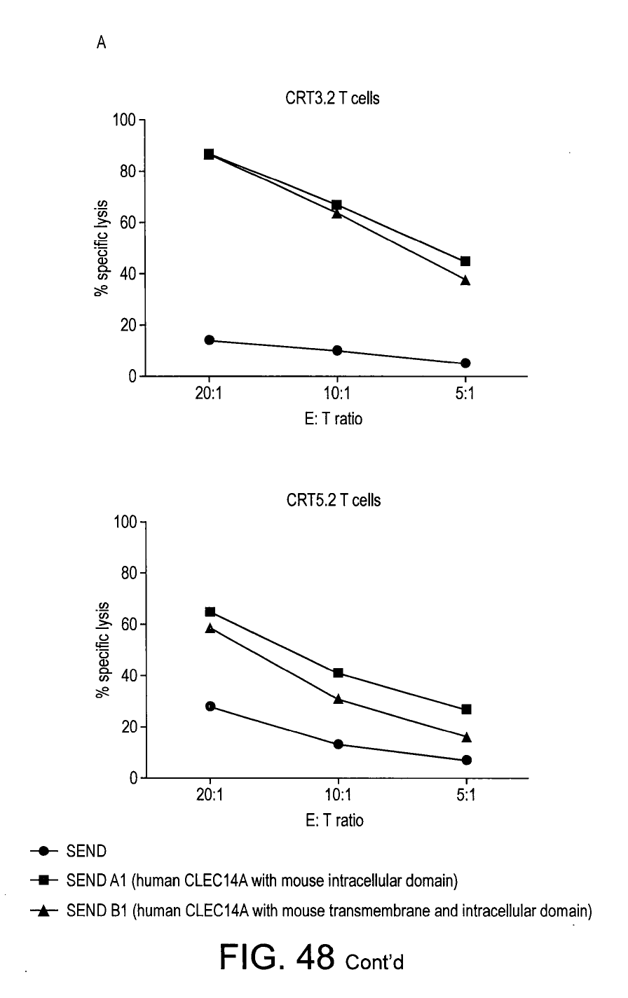

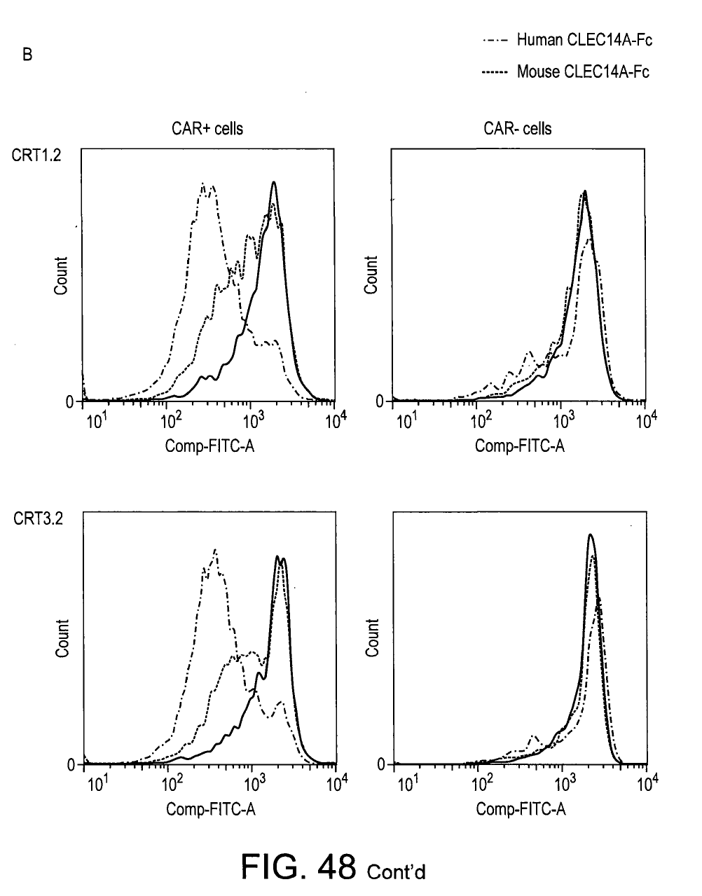

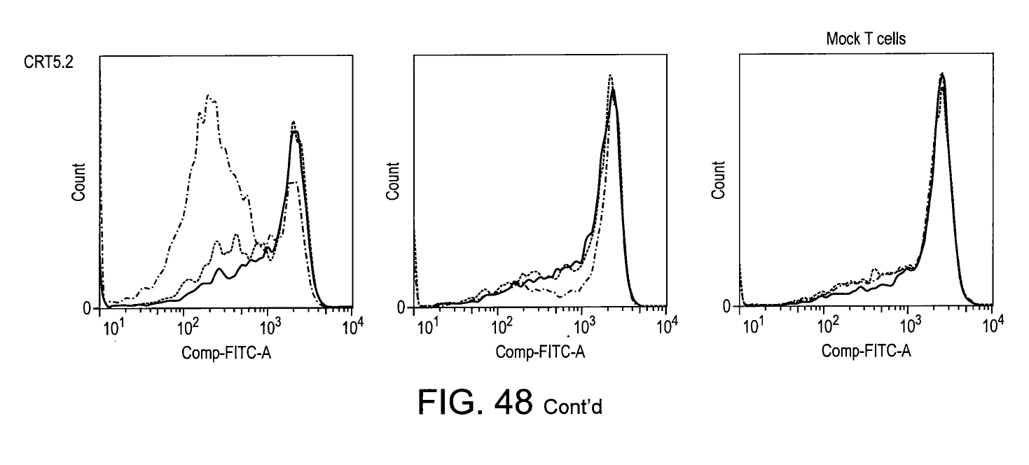

FIG. 48 shows the results of a cytotoxicity assay with CRT1, 3 and 5 CARs vs mouse endothelial cells expressing CLEC14A (FIG. 48A). The results of a proliferative assay for CRT 1, 3 and 5 CARs are shown in FIG. 48B.

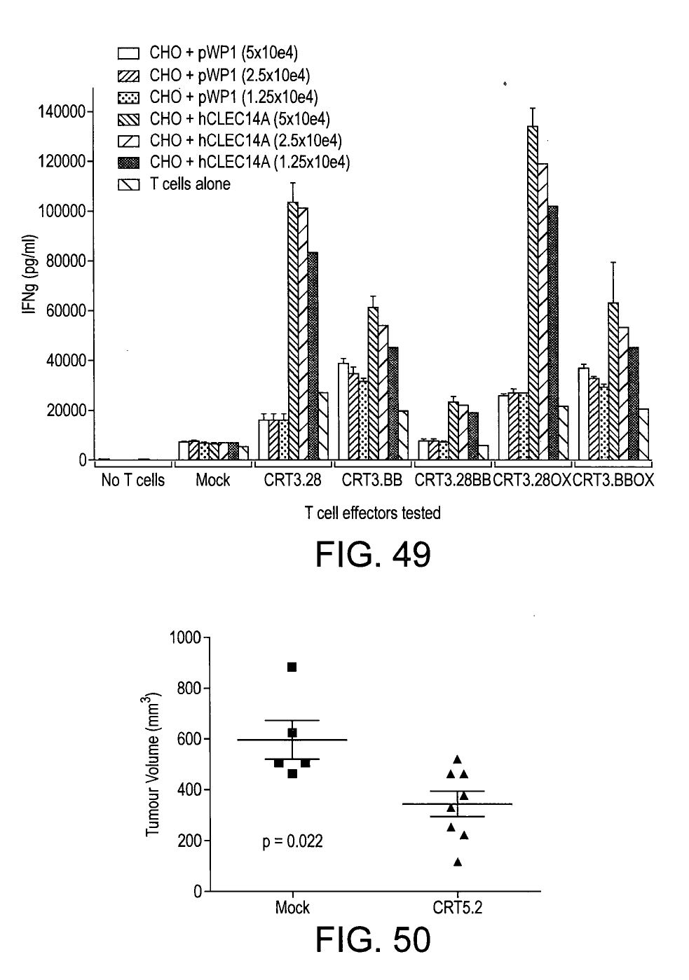

FIG. 49 shows the functional testing of CRT3 CAR T cells comprising different costimulatory domains and the IFNgamma production in response to titrated numbers of CHO cells expressing human CLEC14A.

FIG. 50 shows orthotopic PDAC tumour volumes 3 weeks after treatment with mock (n=5) or CRT5 CAR (CD28 costimulatory domain) T cells (n=8) (p=0.022; Mann Whitney).

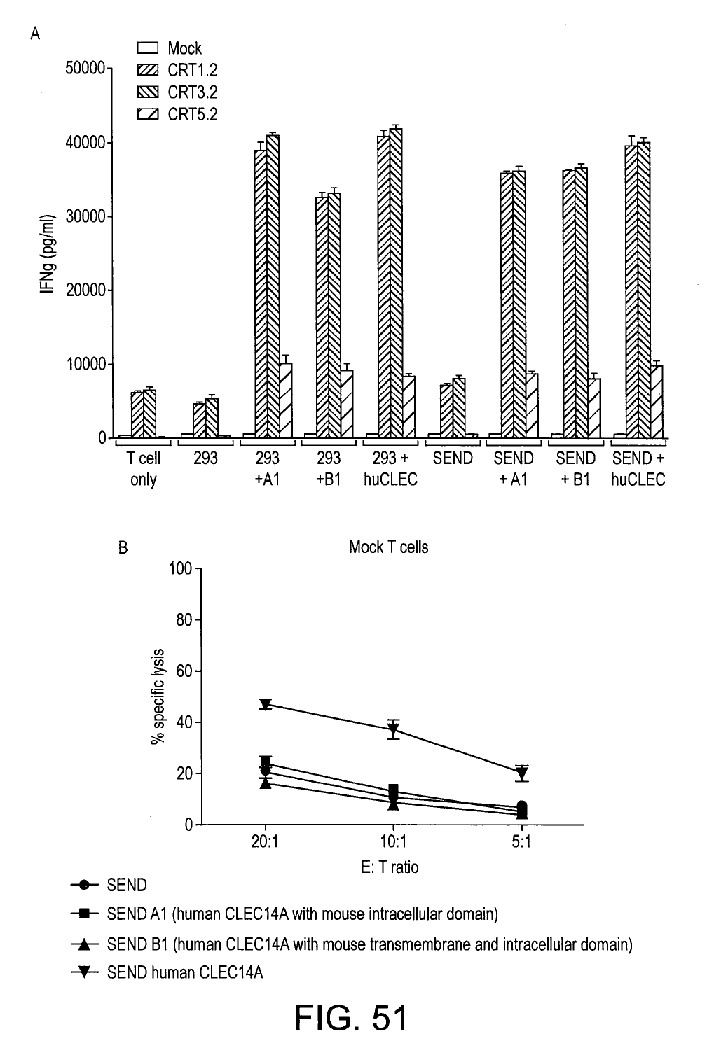

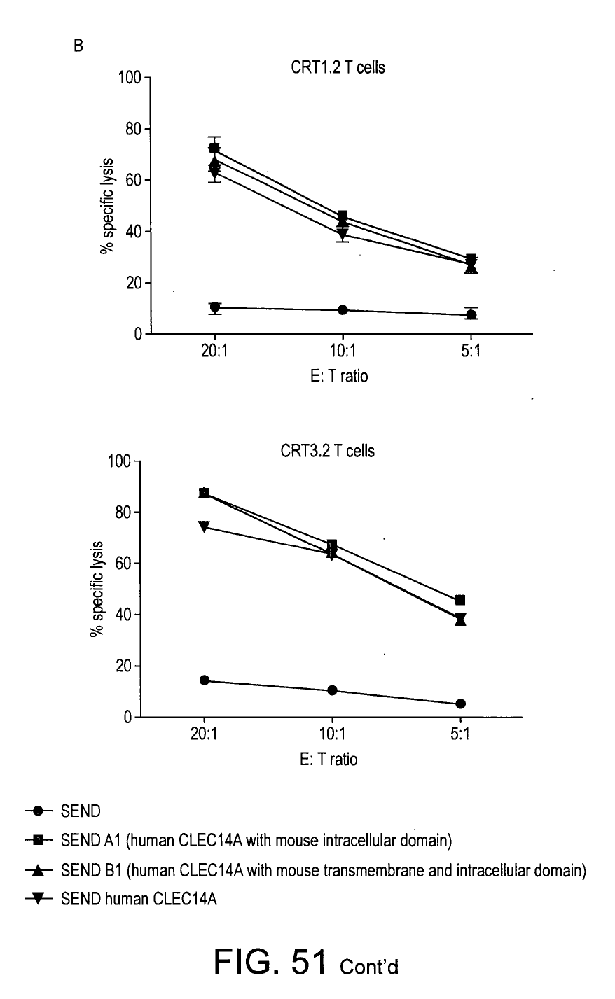

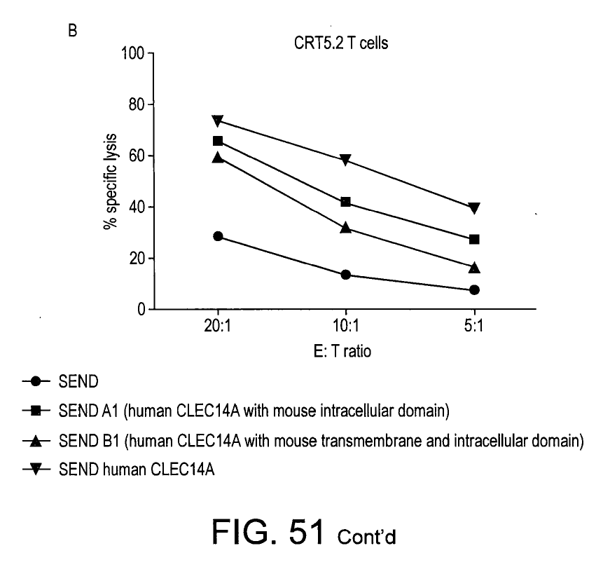

FIG. 51 shows the IFN gamma release by CRT1, 3 and 5 CAR (CD28 costimulatory domain) T cells after incubated with 293 or SEND cells engineered to express CLEC14A chimera (A1—human CLEC14A with mouse intracellular domain, B1—human CLEC14A with mouse transmembrane and intracellular domains, huCLEC—human CLEC14A). Cytotoxicity data are shown in FIG. 51B for the CAR T cells after incubation with SEND cells.

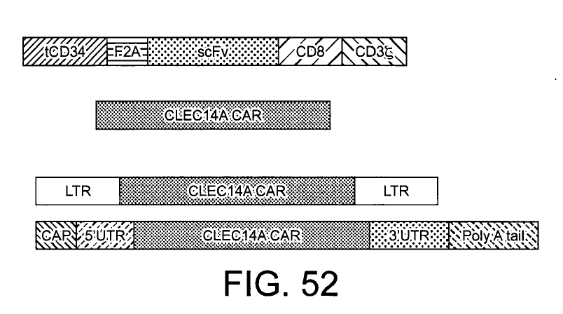

FIG. 52 shows a schematic of a suitable vector to generate RNA for electroporation by in vitro transcription.

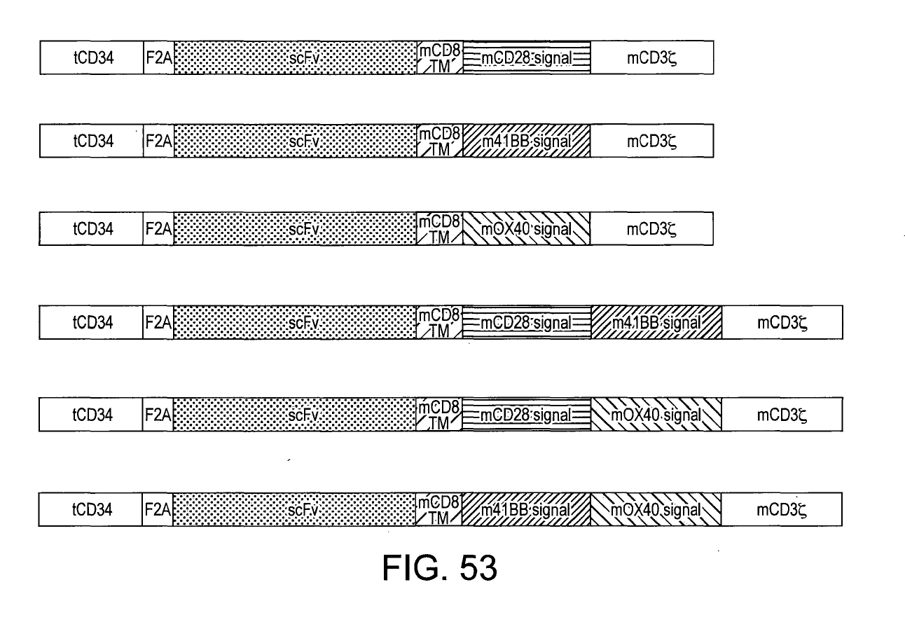

FIG. 53 shows constructs which encode CARs which can be used to transduce murine T cells. The constructs comprise transmembrane, costimulatory and intracellular signalling sequences from murine proteins (see SEQ ID NOs 227-232). The constructs may further comprise a hinge or spacer domain from murine CD8α.

TABLE 1
showing sequences

| SEQ | Description of | |
|---|---|---|
| ID NO | sequence | Sequence |
| 1 | CLEC14A | MRPAFALCLLWQALWPGPGGGEHPTADRAGCSASGACY |
| | polypeptide | SLHHATMKRQAAEEACILRGGALSTVRAGAELRAVLALLRA |
| | | GPGPGGGSKDLLFWVALERRRSHCTLENEPLRGFSWLSS |
| | | DPGGLESDTLQWVEEPQRSCTARRCAVLQATGGVEPAG |
| | | WKEMRCHLRANGYLCKYQFEVLCPAPRPGAASNLSYRAP |
| | | FQLHSAALDFSPPGTEVSALCRGQLPISVTCIADEIGARWD |
| | | KLSGDVLCPCPGRYLRAGKCAELPNCLDDLGGFACECAT |
| | | GFELGKDGRSCVTSGEGQPTLGGTGVPTRRPPATATSPV |
| | | PQRTWPIRVDEKLGETPLVPEQDNSVTSIPEIPRWGSQST |
| | | MSTLQMSLQAESKATITPSGSVISKFNSTTSSATPQAFDSS |
| | | SAVVFIFVSTAVVVLVILTMTVLGLVKLCFHESPSSQPRKES |
| | | MGPPGLESDPEPAALGSSSAHCTNNGVKVGDCDLRDRAE |
| | | GALLAESPLGSSDA |
| 2 | CLEC14A cDNA | CTCCTCTTGCTCTAAGCAGGGTGTTTGACCTTCTAGTCG |
| | | ACTGCGTCCCCTGTACCCGGCGCCAGCTGTGTTCCTGA |
| | | CCCCAGAATAACTCAGGGCTGCACCGGGCCTGGCAGC |
| | | GCTCCGCACACATTTCCTGTCGCGGCCTAAGGGAAACT |
| | | GTTGGCCGCTGGGCCCGCGGGGGGATTCTTGGCAGTT |
| | | GGGGGGTCCGTCGGGAGCGAGGGCGGAGGGGAAGGG |
| | | AGGGGGAACCGGGTTGGGGAAGCCAGCTGTAGAGGGC |
| | | GGTGACCGCGCTCCAGACACAGCTCTGCGTCCTCGAGC |
| | | GGGACAGATCCAAGTTGGGAGCAGCTCTGCGTGCGGG |
| | | GCCTCAGAGAATGAGGCCGGCGTTCGCCCTGTGCCTCC |
| | | TCTGGCAGGCGCTCTGGCCCGGGCCGGGCGGCGGCG |
| | | AACACCCCACTGCCGACCGTGCTGGCTGCTCGGCCTCG |
| | | GGGGCCTGCTACAGCCTGCACCACGCTACCATGAAGCG |
| | | GCAGGCGGCCGAGGAGGCCTGCATCCTGCGAGGTGGG |
| | | GCGCTCAGCACCGTGCGTGCGGGCGCCGAGCTGCGCG |
| | | CTGTGCTCGCGCTCCTGCGGGCAGGCCCAGGGCCCGG |
| | | AGGGGGCTCCAAAGACCTGCTGTTCTGGGTCGCACTGG |
| | | AGCGCAGGCGTTCCCACTGCACCCTGGAGAACGAGCCT |
| | | TTGCGGGGTTTCTCCTGGCTGTCCTCCGACCCCGGCGG |
| | | TCTCGAAAGCGACACGCTGCAGTGGGTGGAGGAGCCC |
| | | CAACGCTCCTGCACCGCGCGGAGATGCGCGGTACTCCA |
| | | GGCCACCGGTGGGGTCGAGCCCGCAGGCTGGAAGGAG |
| | | ATGCGATGCCACCTGCGCGCCAACGGCTACCTGTGCAA |
| | | GTACCAGTTTGAGGTCTTGTGTCCTGCGCCGCGCCCCG |
| | | GGGCCGCCTCTAACTTGAGCTATCGCGCGCCCTTCCAG |
| | | CTGCACAGCGCCGCTCTGGACTTCAGTCCACCTGGGAC |
| | | CGAGGTGAGTGCGCTCTGCCGGGGACAGCTCCCGATC |
| | | TCAGTTACTTGCATCGCGGACGAAATCGGCGCTCGCTG |
| | | GGACAAACTCTCGGGCGATGTGTTGTGTCCCTGCCCCG |
| | | GGAGGTACCTCCGTGCTGGCAAATGCGCAGAGCTCCCT |
| | | AACTGCCTAGACGACTTGGGAGGCTTTGCCTGCGAATG |
| | | TGCTACGGGCTTCGAGCTGGGGAAGGACGGCCGCTCTT |
| | | GTGTGACCAGTGGGGAAGGACAGCCGACCCTTGGGGG |
| | | GACCGGGGTGCCCACCAGGCGCCCGCCGGCCACTGCA |
| | | ACCAGCCCCGTGCCGCAGAGAACATGGCCAATCAGGGT |
| | | CGACGAGAAGCTGGGAGAGACACCACTTGTCCCTGAAC |
| | | AAGACAATTCAGTAACATCTATTCCTGAGATTCCTCGAT |
| | | GGGGATCACAGAGCACGATGTCTACCCTTCAAATGTCC |
| | | CTTCAAGCCGAGTCAAAGGCCACTATCACCCCATCAGG |
| | | GAGCGTGATTTCCAAGTTTAATTCTACGACTTCCTCTGC |
| | | CACTCCTCAGGCTTTCGACTCCTCCTCTGCCGTGGTCTT |
| | | CATATTTGTGAGCACAGCAGTAGTAGTGTTGGTGATCTT |
| | | GACCATGACAGTACTGGGGCTTGTCAAGCTCTGCTTTCA |
| | | CGAAAGCCCCTCTTCCCAGCCAAGGAAGGAGTCTATGG |
| | | GCCCGCCGGGCCTGGAGAGTGATCCTGAGCCCGCTGC |
| | | TTTGGGCTCCAGTTCTGCACATTGCACAAACAATGGGGT |
| | | GAAAGTCGGGGACTGTGATCTGCGGGACAGAGCAGAG |
| | | GGTGCCTTGCTGGCGGAGTCCCCTCTTGGCTCTAGTGA |
| | | TGCATAGGGAAACAGGGGACATGGGCACTCCTGTGAAC |
| | | AGTTTTTCACTTTTGATGAAACGGGGAACCAAGAGGAAC |
| | | TTACTTGTGTAACTGACAATTTCTGCAGAAATCCCCCTTC |
| | | CTCTAAATTCCCTTTACTCCACTGAGGAGCTAAATCAGA |
| | | ACTGCACACTCCTTCCCTGATGATAGAGGAAGTGGAAGT |
| | | GCCTTTAGGATGGTGATACTGGGGGACCGGGTAGTGCT |
| | | GGGGAGAGATATTTTCTTATGTTTATTCGGAGAATTTGG |
| | | AGAAGTGATTGAACTTTTCAAGACATTGGAAACAAATAG |
| | | AACACAATATAATTTACATTAAAAAATAATTTCTACCAAAA |
| | | TGGAAAGGAAATGTTCTATGTTGTTCAGGCTAGGAGTAT |
| | | ATTGGTTCGAAATCCCAGGGAAAAAAATAAAAATAAAAA |
| | | ATTAAAGGATTGT |
| 3 | CLEC14A coding | ATGAGGCCGGCGTTCGCCCTGTGCCTCCTCTGGCAGGC |
| | region | GCTCTGGCCCGGGCCGGGCGGCGGCGAACACCCCACT |
| | | GCCGACCGTGCTGGCTGCTCGGCCTCGGGGGCCTGCT |
| | | ACAGCCTGCACCACGCTACCATGAAGCGGCAGGCGGC |
| | | CGAGGAGGCCTGCATCCTGCGAGGTGGGGCGCTCAGC |
| | | ACCGTGCGTGCGGGCGCCGAGCTGCGCGCTGTGCTCG |
| | | CGCTCCTGCGGGCAGGCCCAGGGCCCGGAGGGGGCT |
| | | CCAAAGACCTGCTGTTCTGGGTCGCACTGGAGCGCAGG |
| | | CGTTCCCACTGCACCCTGGAGAACGAGCCTTTGCGGGG |
| | | TTTCTCCTGGCTGTCCTCCGACCCCGGCGGTCTCGAAA |
| | | GCGACACGCTGCAGTGGGTGGAGGAGCCCCAACGCTC |
| | | CTGCACCGCGCGGAGATGCGCGGTACTCCAGGCCACC |
| | | GGTGGGGTCGAGCCCGCAGGCTGGAAGGAGATGCGAT |
| | | GCCACCTGCGCGCCAACGGCTACCTGTGCAAGTACCAG |
| | | TTTGAGGTCTTGTGTCCTGCGCCGCGCCCCGGGGCCG |
| | | CCTCTAACTTGAGCTATCGCGCGCCCTTCCAGCTGCAC |
| | | AGCGCCGCTCTGGACTTCAGTCCACCTGGGACCGAGGT |
| | | GAGTGCGCTCTGCCGGGGACAGCTCCCGATCTCAGTTA |
| | | CTTGCATCGCGGACGAAATCGGCGCTCGCTGGGACAAA |
| | | CTCTCGGGCGATGTGTTGTGTCCCTGCCCCGGGAGGTA |
| | | CCTCCGTGCTGGCAAATGCGCAGAGCTCCCTAACTGCC |
| | | TAGACGACTTGGGAGGCTTTGCCTGCGAATGTGCTACG |
| | | GGCTTCGAGCTGGGGAAGGACGGCCGCTCTTGTGTGA |
| | | CCAGTGGGGAAGGACAGCCGACCCTTGGGGGGACCGG |
| | | GGTGCCCACCAGGCGCCCGCCGGCCACTGCAACCAGC |
| | | CCCGTGCCGCAGAGAACATGGCCAATCAGGGTCGACGA |
| | | GAAGCTGGGAGAGACACCACTTGTCCCTGAACAAGACA |
| | | ATTCAGTAACATCTATTCCTGAGATTCCTCGATGGGGAT |
| | | CACAGAGCACGATGTCTACCCTTCAAATGTCCCTTCAAG |
| | | CCGAGTCAAAGGCCACTATCACCCCATCAGGGAGCGTG |
| | | ATTTCCAAGTTTAATTCTACGACTTCCTCTGCCACTCCTC |
| | | AGGCTTTCGACTCCTCCTCTGCCGTGGTCTTCATATTTG |
| | | TGAGCACAGCAGTAGTAGTGTTGGTGATCTTGACCATGA |
| | | CAGTACTGGGGCTTGTCAAGCTCTGCTTTCACGAAAGC |
| | | CCCTCTTCCCAGCCAAGGAAGGAGTCTATGGGCCCGCC |
| | | GGGCCTGGAGAGTGATCCTGAGCCCGCTGCTTTGGGCT |
| | | CCAGTTCTGCACATTGCACAAACAATGGGGTGAAAGTC |
| | | GGGGACTGTGATCTGCGGGACAGAGCAGAGGGTGCCT |
| | | TGCTGGCGGAGTCCCCTCTTGGCTCTAGTGATGCATAG |
| 4 | human CLEC14A | TAGTAGGAATTCGAGAGAATGAGGCCGGCGTTCGCCCT |
| | fwd | G |
| 5 | human CLEC14A | AGAACCGCGGCCGCTGGAGGAGTCGAAAGCCTGAGGA |
| | rev | GT |
| 6 | murine CLEC14A | TAGTAGGAATTCGAGAGAATGAGGCCAGCGCTTGCCCT |
| | fwd | G |
| 7 | murine CLEC14A | CTACTAGCGGCCGCTCGTGGAAGAGGTGTCGAAAGT |
| | rev | |
| 8 | human CLEC14A | TAGTAGTTAATTAAGAGAGAATGAGGCCGGCGTTC |
| | fwd | |
| 9 | murine CLEC14A | TAGTAGTTAATTAAGAGAGAATGAGGCCAGCGCTT |
| | fwd | |
| 10 | human Fc rev | CTACTAGTTTAAACTCATTTACCCGGAGACAGGGA |
| 11 | MMRN2 fwd | CCGGACCGGTCAGGCTTCCAGTACTAGCC |
| 12 | MMRN2 rev | CGGGGTACCGGTCTTAAACATCAGGAAGC |
| 13 | 5′UTR fwd | TTCCTTTTCCAGGGTTTGTG |
| 14 | 5′UTR rev | GCCTACAAGGTGGCTTGAAT |
| 15 | CDS fwd | AAGCTGTGCTCCTGCTCTTG |
| 16 | CDS rev | TCCTGAGTGCACTGTGAGATG |
| 17 | 3′ UTR fwd | CTGTAGAGGGCGGTGACTTT |
| 18 | 3′ UTR rev | AGCTGCTCCCAAGTCCTCT |
| 19 | mACTB fwd | CTAAGGCCAACCGTGAAAAG |
| 20 | mACTB rev | ACCAGAGGCATACAGGGACA |
| 21 | CD141 residues | MLGVLVLGALALAGLGFPAPAEPQPGGSQCVEHDCFALY |
| | 1-42 | PGP |
| 22 | CD141 residues | QLPPGCGDPKRL |
| | 97-108 | |
| 23 | CD141 residues | TSYSRWARLDLNGAPLCGPL |
| | 122-142 | |
| 24 | CLEC14A | ERRRSCHTLENE |
| | residues 97-108 | |
| 25 | CD141 CTLD | MLGVLVLGALALAGLGFPAPAEPQPGGSQCVEHDCFALYP |
| | amino acid | GPATFLNASQICDGLRGHLMTVRSSVAADVISLLLNGDGG |
| | | VGRRRLWIGLQLPPGCGDPKRLGPLRGFQWVTGDNNTSY |
| | | SRWARLDLNGAPLCGPLCVAVSAAEATVPSEPIWEEQQC |
| | | EVKADGFLCEF |
| 26 | Chimera 5 GFP | MLGVLVLGALALAGLGFPAPAEPQPGGSQCVEHDCFALY |
| | fusion amino acid | PGPATFLNASQICDGLRGHLMTVRSSVAADVISLLLNGDG |
| | —C-type lectin | GVGRRRLWIGLQLPPGCGDPKRLGPLRGFQWVTGDNNT |
| | domain of CD141 | SYSRWARLDLNGAPLCGPLCVAVSAAEATVPSEPIWEEQ |
| | (bold), CLEC14A | QCEVKADGFLCEFQFEVLCPAPRPGAASNLSYRAPFQLH |
| | (non-bold, non- | SAALDFSPPGTEVSALCRGQLPISVTCIADEIGARWDKLSG |
| | italics), GFP | DVLCPCPGRYLRAGKCAELPNCLDDLGGFACECATGFEL |
| | (italics) | GKDGRSCVTSGEGQPTLGGTGVPTRRPPATATSPVPQRT |
| | | WPIRVDEKLGETPLVPEQDNSVTSIPEIPRWGSQSTMSTL |
| | | QMSLQAESKATITPSGSVISKFNSTTSSATPQAFDSSSAVV |
| | | FIFVSTAVVVLVILTMTVLGLVKLCFHESPSSQPRKESMGP |
| | | PGLESDPEPAALGSSSAHCTNNGVKVGDCDLRDRAEGAL |
| | | LAESPLGSSDALQSTVPRARDPPVATMVSKGEELFTGVVPI |
| | | LVELDGDVNGHKFSVSGEGEGDATYGKLTLKFICTTGKLP |
| | | VPWPTLVTTLTYGVQCFSRYPDHMKQHDFFKSAMPEGYV |
| | | QERTIFFKDDGNYKTRAEVKFEGDTLVNRIELKGIDFKEDG |
| | | NILGHKLEYNYNSHNVYIMADKQKNGIKVNFKIRHNIEDGSV |
| | | QLADHYQQNTPIGDGPVLLPDNHYLSTQSALSKDPNEKRD |
| | | HMVLLEFVTAAGITLGMDELYK |
| 27 | Chimera 6 GFP | MRPAFALCLLWQALWPGPGGGEHPTADRAGCSASGAC |
| | fusion amino acid | YSLHHATMKRQAAEEACILRGGALSTVRAGAELRAVLAL |
| | of CLEC14A, with | LRAGPGPGGGSKDLLFWVALERRRSHCTLENEPLRGFS |
| | substituted sushi | WLSSDPGGLESDTLQWVEEPQRSCTARRCAVLQATGGV |
| | of CD141 | EPAGWKEMRCHLRANGYLCKYHFPATCRPLAVEPGAAA |
| | (underlined) and | AAVSITYGTPFAARGADFQALPVGSSAAVAPLGLQLMCTA |
| | GFP in italics | PPGAVQGHWAREAPGACPGRYLRAGKCAELPNCLDDLG |
| | | GFACECATGFELGKDGRSCVTSGEGQPTLGGTGVPTRRP |
| | | PATATSPVPQRTWPIRVDEKLGETPLVPEQDNSVTSIPEIP |
| | | RWGSQSTMSTLQMSLQAESKATITPSGSVISKFNSTTSSA |
| | | TPQAFDSSSAVVFIFVSTAVVVLVILTMTVLGLVKLCFHESP |
| | | SSQPRKESMGPPGLESDPEPAALGSSSAHCTNNGVKVGD |
| | | CDLRDRAEGALLAESPLGSSDALQSTVPRARDPPVATMVS |
| | | KGEELFTGVVPILVELDGDVNGHKFSVSGEGEGDATYGKL |
| | | TLKFICTTGKLPVPWPTLVTTLTYGVQCFSRYPDHMKQHD |
| | | FFKSAMPEGYVQERTIFFKDDGNYKTRAEVKFEGDTLVNRI |
| | | ELKGIDFKEDGNILGHKLEYNYNSHNVYIMADKQKNGIKVN |
| | | FKIRHNIEDGSVQLADHYQQNTPIGDGPVLLPDNHYLSTQS |
| | | ALSKDPNEKRDHMVLLEFVTAAGITLGMDELYK |
| 28 | MMRN2 amino | MILSLLFSLGGPLGWGLLGAWAQASSTSLSDLQSSRTPGV |
| | acid | WKAEAEDTGKDPVGRNWCPYPMSKLVTLLALCKTEKFLIH |
| | | SQQPCPQGAPDCQKVKVMYRMAHKPVYQVKQKVLTSLA |
| | | WRCCPGYTGPNCEHHDSMAIPEPADPGDSHQEPQDGPV |
| | | SFKPGHLAAVINEVEVQQEQQEHLLGDLQNDVHRVADSLP |
| | | GLWKALPGNLTAAVMEANQTGHEFPDRSLEQVLLPHVDTF |
| | | LQVHFSPIWRSFNQSLHSLTQAIRNLSLDVEANRQAISRVQ |
| | | DSAVARADFQELGAKFEAKVQENTQRVGQLRQDVEDRLH |
| | | AQHFTLHRSISELQADVDTKLKRLHKAQEAPGTNGSLVLAT |
| | | PGAGARPEPDSLQARLGQLQRNLSELHMTTARREEELQY |
| | | TLEDMRATLTRHVDEIKELYSESDETFDQISKVERQVEELQ |
| | | VNHTALRELRVILMEKSLIMEENKEEVERQLLELNLTLQHLQ |
| | | GGHADLIKYVKDCNCQKLYLDLDVIREGQRDATRALEETQ |
| | | VSLDERRQLDGSSLQALQNAVDAVSLAVDAHKAEGERAR |
| | | AATSRLRSQVQALDDEVGALKAAAAEARHEVRQLHSAFAA |
| | | LLEDALRHEAVLAALFGEEVLEEMSEQTPGPLPLSYEQIRV |
| | | ALQDAASGLQEQALGWDELAARVTALEQASEPPRPAEHL |
| | | EPSHDAGREEAATTALAGLARELQSLSNDVKNVGRCCEAE |
| | | AGAGAASLNASLDGLHNALFATQRSLEQHQRLFHSLFGNF |
| | | QGLMEANVSLDLGKLQTMLSRKGKKQQKDLEAPRKRDKK |
| | | EAEPLVDIRVTGPVPGALGAALWEAGSPVAFYASFSEGTA |
| | | ALQTVKFNTTYINIGSSYFPEHGYFRAPERGVYLFAVSVEF |
| | | GPGPGTGQLVFGGHHRTPVCTTGQGSGSTATVFAMAELQ |
| | | KGERVWFELTQGSITKRSLSGTAFGGFLMFKT |
| 29 | MMRN2 | ATGATCCTGAGCTTGCTGTTCAGCCTTGGGGGCCCCCT |
| | nucleotide | GGGCTGGGGGCTGCTGGGGGCATGGGCCCAGGCTTCC |
| | | AGTACTAGCCTCTCTGATCTGCAGAGCTCCAGGACACCT |
| | | GGGGTCTGGAAGGCAGAGGCTGAGGACACCGGCAAGG |
| | | ACCCCGTTGGACGTAACTGGTGCCCCTACCCAATGTCC |
| | | AAGCTGGTCACCTTACTAGCTCTTTGCAAAACAGAGAAA |
| | | TTCCTCATCCACTCGCAGCAGCCGTGTCCGCAGGGAGC |
| | | TCCAGACTGCCAGAAAGTCAAAGTCATGTACCGCATGG |
| | | CCCACAAGCCAGTGTACCAGGTCAAGCAGAAGGTGCTG |
| | | ACCTCTTTGGCCTGGAGGTGCTGCCCTGGCTACACGGG |
| | | CCCCAACTGCGAGCACCACGATTCCATGGCAATCCCTG |
| | | AGCCTGCAGATCCTGGTGACAGCCACCAGGAACCTCAG |
| | | GATGGACCAGTCAGCTTCAAACCTGGCCACCTTGCTGC |
| | | AGTGATCAATGAGGTTGAGGTGCAACAGGAACAGCAGG |
| | | AACATCTGCTGGGAGATCTCCAGAATGATGTGCACCGG |
| | | GTGGCAGACAGCCTGCCAGGCCTGTGGAAAGCCCTGC |
| | | CTGGTAACCTCACAGCTGCAGTGATGGAAGCAAATCAAA |
| | | CAGGGCACGAGTTCCCTGATAGATCCTTGGAGCAGGTG |
| | | CTGCTACCCCACGTGGACACCTTCCTACAAGTGCATTTC |
| | | AGCCCCATCTGGAGGAGCTTTAACCAAAGCCTGCACAG |
| | | CCTTACCCAGGCCATAAGAAACCTGTCTCTTGACGTGGA |
| | | GGCCAACCGCCAGGCCATCTCCAGAGTCCAGGACAGTG |
| | | CCGTGGCCAGGGCTGACTTCCAGGAGCTTGGTGCCAAA |
| | | TTTGAGGCCAAGGTCCAGGAGAACACTCAGAGAGTGGG |
| | | TCAGCTGCGACAGGACGTGGAGGACCGCCTGCACGCC |
| | | CAGCACTTTACCCTGCACCGCTCGATCTCAGAGCTCCAA |
| | | GCCGATGTGGACACCAAATTGAAGAGGCTGCACAAGGC |
| | | TCAGGAGGCCCCAGGGACCAATGGCAGTCTGGTGTTGG |
| | | CAACGCCTGGGGCTGGGGCAAGGCCTGAGCCGGACAG |
| | | CCTGCAGGCCAGGCTGGGCCAGCTGCAGAGGAACCTC |
| | | TCAGAGCTGCACATGACCACGGCCCGCAGGGAGGAGG |
| | | AGTTGCAGTACACCCTGGAGGACATGAGGGCCACCCTG |
| | | ACCCGGCACGTGGATGAGATCAAGGAACTGTACTCCGA |
| | | ATCGGACGAGACTTTCGATCAGATTAGCAAGGTGGAGC |
| | | GGCAGGTGGAGGAGCTGCAGGTGAACCACACGGCGCT |
| | | CCGTGAGCTGCGCGTGATCCTGATGGAGAAGTCTCTGA |
| | | TCATGGAGGAGAACAAGGAGGAGGTGGAGCGGCAGCT |
| | | CCTGGAGCTCAACCTCACGCTGCAGCACCTGCAGGGTG |
| | | GCCATGCCGACCTCATCAAGTACGTGAAGGACTGCAAT |
| | | TGCCAGAAGCTCTATTTAGACCTGGACGTCATCCGGGA |
| | | GGGCCAGAGGGACGCCACGCGTGCCCTGGAGGAGACC |
| | | CAGGTGAGCCTGGACGAGCGGCGGCAGCTGGACGGCT |
| | | CCTCCCTGCAGGCCCTGCAGAACGCCGTGGACGCCGT |
| | | GTCGCTGGCCGTGGACGCGCACAAAGCGGAGGGCGAG |
| | | CGGGCGCGGGCGGCCACGTCGCGGCTCCGGAGCCAA |
| | | GTGCAGGCGCTGGATGACGAGGTGGGCGCGCTGAAGG |
| | | CGGCCGCGGCCGAGGCCCGCCACGAGGTGCGCCAGCT |
| | | GCACAGCGCCTTCGCCGCCCTGCTGGAGGACGCGCTG |
| | | CGGCACGAGGCGGTGCTGGCCGCGCTCTTCGGGGAGG |
| | | AGGTGCTGGAGGAGATGTCTGAGCAGACGCCGGGACC |
| | | GCTGCCCCTGAGCTACGAGCAGATCCGCGTGGCCCTG |
| | | CAGGACGCCGCTAGCGGGCTGCAGGAGCAGGCGCTCG |
| | | GCTGGGACGAGCTGGCCGCCCGAGTGACGGCCCTGGA |
| | | GCAGGCCTCGGAGCCCCCGCGGCCGGCAGAGCACCTG |
| | | GAGCCCAGCCACGACGCGGGCCGCGAGGAGGCCGCC |
| | | ACCACCGCCCTGGCCGGGCTGGCGCGGGAGCTCCAGA |
| | | GCCTGAGCAACGACGTCAAGAATGTCGGGCGGTGCTGC |
| | | GAGGCTGAGGCCGGGGCCGGGGCCGCCTCCCTCAACG |
| | | CCTCCCTTGACGGCCTCCACAACGCACTCTTCGCCACT |
| | | CAGCGCAGCTTGGAGCAGCACCAGCGGCTCTTCCACAG |
| | | CCTCTTTGGGAACTTCCAAGGGCTCATGGAAGCCAACG |
| | | TCAGCCTGGACCTGGGGAAGCTGCAGACCATGCTGAGC |
| | | AGGAAAGGGAAGAAGCAGCAGAAAGACCTGGAAGCTCC |
| | | CCGGAAGAGGGACAAGAAGGAAGCGGAGCCTTTGGTG |
| | | GACATACGGGTCACAGGGCCTGTGCCAGGTGCCTTGG |
| | | GCGCGGCGCTCTGGGAGGCAGGATCCCCTGTGGCCTT |
| | | CTATGCCAGCTTTTCAGAAGGGACGGCTGCCCTGCAGA |
| | | CAGTGAAGTTCAACACCACATACATCAACATTGGCAGCA |
| | | GCTACTTCCCTGAACATGGCTACTTCCGAGCCCCTGAG |
| | | CGTGGTGTCTACCTGTTTGCAGTGAGCGTTGAATTTGGC |
| | | CCAGGGCCAGGCACCGGGCAGCTGGTGTTTGGAGGTC |
| | | ACCATCGGACTCCAGTCTGTACCACTGGGCAGGGGAGT |
| | | GGAAGCACAGCAACGGTCTTTGCCATGGCTGAGCTGCA |
| | | GAAGGGTGAGCGAGTATGGTTTGAGTTAACCCAGGGAT |
| | | CAATAACAAAGAGAAGCCTGTCGGGCACTGCATTTGGG |
| | | GGCTTCCTGATGTTTAAGACCTGA |
| 30 | siRNA duplex D1 | GAACAAGACAATTCAGTAA |
| 31 | siRNA duplex D2 | CAATCAGGGTCGACGAGAA |

CRT1 Version (V1) - complementarity determining regions

| | | |
|---|---|---|
| 32 | CRT1 V1 HC | SSYWIE |
| | CDR1 | |
| 33 | CRT1 V1 HC | WIGEILPGSGSTN |
| | CDR2 | |
| 34 | CRT1 V1 HC | ARGGDYDEEYYVMD |
| | CDR3 | |
| 35 | CRT1 V1 LC | SYMYWY |
| | CDR1 | |
| 36 | CRT1 V1 LC | LLIYDTSNLA |
| | CDR2 | |
| 37 | CRT1 V1 LC | QQWSSYPL |
| | CDR3 | |
| 38 | CRT1 V1 HC | AGTAGCTACTGGATAGAG |
| | CDR1 | |
| | (nucleotide) | |
| 39 | CRT1 V1 HC | TGGATTGGAGAGATTTTACCTGGAAGTGGTAGTACTAAT |
| | CDR2 | |
| | (nucleotide) | |
| 40 | CRT1 V1 HC | GCAAGAGGGGGGGATTACGACGAAGAATACTATGTCAT |
| | CDR3 | GGAC |
| | (nucleotide) | |
| 41 | CRT1 V1 LC | AGTTACATGTACTGGTAC |
| | CDR1 | |
| | (nucleotide) | |
| 42 | CRT1 V1 LC | CTCCTGATTTATGACACATCCAACCTGGCT |
| | CDR2 | |
| | (nucleotide) | |
| 43 | CRT1 V1 LC | CAGCAGTGGAGTAGTTACCCGCTC |
| | CDR3 | |
| | (nucleotide) | |

CRT1 Version 2 (V2) - Complementarity determining regions

| | | |
|---|---|---|
| 44 | CRT1 V2 HC | GYTFSSYW |
| | CDR1 | |
| 45 | CRT1 V2 HC | ILPGSGST |
| | CDR2 | |
| 46 | CRT1 V2 HC | ARGGDYDEEYYVMDY |
| | CDR3 | |
| 47 | CRT1 V2 LC | SSVSY |
| | CDR1 | |
| | CRT1 V2 LC | DTS |
| | CDR2 | |
| 49 | CRT1 V2 LC | QQWSSYPLT |
| | CDR3 | |
| 50 | CRT1 V2 HC | GGCTACACATTCAGTAGCTACTGG |
| | CDR1 | |
| | (nucleotide) | |
| 51 | CRT1 V2 HC | ATTTTACCTGGAAGTGGTAGTACT |
| | CDR2 | |
| | (nucleotide) | |
| 52 | CRT1 V2 HC | GCAAGAGGGGGGGATTACGACGAAGAATACTATGTCAT |
| | CDR3 | GGACTAC |
| | (nucleotide) | |
| 53 | CRT1 V2 LC | TCAAGTGTAAGTTAC |
| | CDR1 | |
| | (nucleotide) | |
| | CRT1 V2 LC | GACACATCC |
| | CDR2 | |
| | (nucleotide) | |
| 55 | CRT1 V2 LC | CAGCAGTGGAGTAGTTACCCGCTCACG |
| | CDR3 | |
| | (nucleotide) | |

CRT1 heavy and light variable regions, scFv, CARs

| | | |
|---|---|---|
| 56 | CRT1 HC protein | MAEVQLQQSGAELMKPGASVKISCKATGYTFSSYWIEWVK |
| | | QRPGHGLEWIGEILPGSGSTNYNEKFKGKATFTADTSSNT |
| | | AYMQLSSLTSEDSAVYYCARGGDYDEEYYVMDYWGQGT |
| | | SVTV |
| 57 | CRT1 LC protein | QIVLTQSPAIMSASPGEKVTMTCSASSSV-- |
| | | SYMYWYQQKPGSSPRLLIYDTSNLASGVP |
| | | VRFSGSGSGTSYSLTISRMEAEDAATYYCQQWSSYPLTFG |
| | | AGTKLELKR |
| 58 | CRT1 ScFv | MAEVQLQQSGAELMKPGASVKISCKATGYTFSSYWIEWVK |
| | protein | QRPGHGLEWIGEILPGSGSTNYNEKFKGKATFTADTSSNT |
| | | AYMQLSSLTSEDSAVYYCARGGDYDEEYYVMDYWGQGT |
| | | SVTVSSGGGGSGGGGSGGGGSQIVLTQSPAIMSASPGEK |
| | | VTMTCSASSSVSYMYWYQQKPGSSPRLLIYDTSNLASGVP |
| | | VRFSGSGSGTSYSLTISRMEAEDAATYYCQQWSSYPLTFG |
| | | AGTKLELKRAAA |
| 59 | CRT1 HC | ATGGCCGAGGTTCAGCTTCAGCAGTCTGGAGCTGAGCT |
| | nucleotide | GATGAAGCCTGGGGCCTCAGTGAAGATATCCTGCAAGG |
| | | CTACTGGCTACACATTCAGTAGCTACTGGATAGAGTGGG |
| | | TAAAGCAGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAACACAGCCTACATGCAACTCAGCAGCCTGACATCT |
| | | GAGGACTCTGCCGTCTATTACTGTGCAAGAGGGGGGGA |
| | | TTACGACGAAGAATACTATGTCATGGACTACTGGGGTCA |
| | | AGGAACCTCAGTCACTGTC |
| 60 | CRT1 LC | CAAATTGTTCTCACCCAGTCTCCAGCAATCATGTCTGCA |
| | nucleotide | TCTCCAGGGGAGAAGGTCACCATGACCTGCAGTGCCAG |
| | | CTCAAGTGTAAGTTACATGTACTGGTACCAGCAGAAGCC |
| | | AGGATCCTCCCCCAGACTCCTGATTTATGACACATCCAA |
| | | CCTGGCTTCTGGAGTCCCTGTTCGCTTCAGTGGCAGTG |
| | | GGTCTGGGACCTCTTACTCTCTCACAATCAGCCGAATGG |
| | | AGGCTGAAGATGCTGCCACTTATTACTGCCAGCAGTGG |
| | | AGTAGTTACCCGCTCACGTTCGGTGCTGGGACCAAGCT |
| | | GGAGCTGAAACGT |
| 61 | CRT1 ScFv | ATGGCCGAGGTTCAGCTTCAGCAGTCTGGAGCTGAGCT |
| | nucleotide | GATGAAGCCTGGGGCCTCAGTGAAGATATCCTGCAAGG |
| | | CTACTGGCTACACATTCAGTAGCTACTGGATAGAGTGGG |
| | | TAAAGCAGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAACACAGCCTACATGCAACTCAGCAGCCTGACATCT |
| | | GAGGACTCTGCCGTCTATTACTGTGCAAGAGGGGGGGA |
| | | TTACGACGAAGAATACTATGTCATGGACTACTGGGGTCA |
| | | AGGAACCTCAGTCACTGTCTCCTCAGGTGGAGGCGGTT |
| | | CAGGCGGAGGTGGCTCTGGCGGTGGCGGATCGCAAAT |
| | | TGTTCTCACCCAGTCTCCAGCAATCATGTCTGCATCTCC |
| | | AGGGGAGAAGGTCACCATGACCTGCAGTGCCAGCTCAA |
| | | GTGTAAGTTACATGTACTGGTACCAGCAGAAGCCAGGAT |
| | | CCTCCCCCAGACTCCTGATTTATGACACATCCAACCTGG |
| | | CTTCTGGAGTCCCTGTTCGCTTCAGTGGCAGTGGGTCT |
| | | GGGACCTCTTACTCTCTCACAATCAGCCGAATGGAGGC |
| | | TGAAGATGCTGCCACTTATTACTGCCAGCAGTGGAGTAG |
| | | TTACCCGCTCACGTTCGGTGCTGGGACCAAGCTGGAGC |
| | | TGAAACGTGCGGCCGCA |
| 62 | CRT1 CAR1 | M G V L L T Q R T L L S L V L A L L F P S M A |
| | protein | S M A E V Q L Q Q S G A E L M K P G A S V K |
| | | I S C K A T G Y T F S S Y W I E W V K Q R P |
| | | G H G L E W I G E I L P G S G S T N Y N E K |
| | | F K G K A T F T A D T S S N T A Y M Q L S S |
| | | L T S E D S A V Y Y C A R G G D Y D E E Y Y |
| | | V M D Y W G Q G T S V T V S S G G G G S G |
| | | G G G S G G G G S Q I V L T Q S P A I M S A |
| | | S P G E K V T M T C S A S S S V S Y M Y W Y |
| | | Q Q K P G S S P R L L I Y D T S N L A S G V |
| | | P V R F S G S G S G T S Y S L T I S R M E A |
| | | E D A A T Y Y C Q Q W S S Y P L T F G A G T |
| | | K L E L K R A A A I E V M Y P P P Y L D N E |
| | | K S N G T I I H V K G K H L C P S P L F P G P |
| | | S K P F W V L V V V G G V L A C Y S L L V T |
| | | V A F I I F W V R S K R S R L L H S D Y M N |
| | | M T P R R P G P T R K H Y Q P Y A P P R D F |
| | | A A Y R S R V K F S R S A D A P A Y Q Q G Q |
| | | N Q L Y N E L N L G R R E E Y D V L D K R R |
| | | G R D P E M G G K P Q R R K N P Q E G L Y |
| | | N E L Q K D K M A E A Y S E I G M K G E R R |
| | | R G K G H D G L Y Q G L S T A T K D T Y D A |
| | | L H M Q A L P P R |
| 63 | CRT1 CAR1 | ATGGGCGTGCTGCTGACCCAGAGGACCCTGCTGAGCCT |
| | nucleotide | GGTGCTGGCCCTGCTGTTTCCATCTATGGCATCGATGG |
| | | CCGAGGTTCAGCTTCAGCAGTCTGGAGCTGAGCTGATG |
| | | AAGCCTGGGGCCTCAGTGAAGATATCCTGCAAGGCTAC |
| | | TGGCTACACATTCAGTAGCTACTGGATAGAGTGGGTAAA |
| | | GCAGAGGCCTGGACATGGCCTTGAGTGGATTGGAGAGA |
| | | TTTTACCTGGAAGTGGTAGTACTAATTACAATGAGAAGTT |
| | | CAAGGGCAAGGCCACATTCACTGCAGATACATCCTCCA |
| | | ACACAGCCTACATGCAACTCAGCAGCCTGACATCTGAG |
| | | GACTCTGCCGTCTATTACTGTGCAAGAGGGGGGGATTA |
| | | CGACGAAGAATACTATGTCATGGACTACTGGGGTCAAG |
| | | GAACCTCAGTCACTGTCTCCTCAGGTGGAGGCGGTTCA |
| | | GGCGGAGGTGGCTCTGGCGGTGGCGGATCGCAAATTG |
| | | TTCTCACCCAGTCTCCAGCAATCATGTCTGCATCTCCAG |
| | | GGGAGAAGGTCACCATGACCTGCAGTGCCAGCTCAAGT |
| | | GTAAGTTACATGTACTGGTACCAGCAGAAGCCAGGATC |
| | | CTCCCCCAGACTCCTGATTTATGACACATCCAACCTGGC |
| | | TTCTGGAGTCCCTGTTCGCTTCAGTGGCAGTGGGTCTG |
| | | GGACCTCTTACTCTCTCACAATCAGCCGAATGGAGGCT |
| | | GAAGATGCTGCCACTTATTACTGCCAGCAGTGGAGTAGT |
| | | TACCCGCTCACGTTCGGTGCTGGGACCAAGCTGGAGCT |
| | | GAAACGTGCGGCCGCAATTGAAGTTATGTATCCTCCTCC |
| | | TTACCTAGACAATGAGAAGAGCAATGGAACCATTATCCA |
| | | TGTGAAAGGGAAACACCTTTGTCCAAGTCCCCTATTTCC |
| | | CGGACCTTCTAAGCCCTTTTGGGTGCTGGTGGTGGTTG |
| | | GTGGAGTCCTGGCTTGCTATAGCTTGCTAGTAACAGTG |
| | | GCCTTTATTATTTTCTGGGTGAGGAGTAAGAGGAGCAGG |
| | | CTCCTGCACAGTGACTACATGAACATGACTCCCCGCCG |
| | | CCCCGGGCCCACCCGCAAGCATTACCAGCCCTATGCCC |
| | | CACCACGCGACTTCGCAGCCTATCGCTCCAGAGTGAAG |
| | | TTCAGCAGGAGCGCAGACGCCCCCGCGTACCAGCAGG |
| | | GCCAGAACCAGCTCTATAACGAGCTCAATCTAGGACGA |
| | | AGAGAGGAGTACGATGTTTTGGACAAGAGACGTGGCCG |
| | | GGACCCTGAGATGGGGGGAAAGCCGCAGAGAAGGAAG |
| | | AACCCTCAGGAAGGCCTGTACAATGAACTGCAGAAAGA |
| | | TAAGATGGCGGAGGCCTACAGTGAGATTGGGATGAAAG |
| | | GCGAGCGCCGGAGGGGCAAGGGGCACGATGGCCTTTA |
| | | CCAGGGTCTCAGTACAGCCACCAAGGACACCTACGACG |
| | | CCCTTCACATGCAGGCCCTGCCCCCTCGCTAATAAAAG |
| | | CTTAACACGAGCCA |

CRT3 Version 1 (V1) - complementarity determining regions

| | | |
|---|---|---|
| 64 | CRT3 V1 HC | TSYWMH |
| | CDR1 | |
| 65 | CRT3 V1 HC | WIGAIYPGNSDTS |
| | CDR2 | |
| 66 | CRT3 V1 HC | TH---YYGSDYAMD |
| | CDR3 | |
| 67 | CRT3 V1 LC | SSSYLHWY |
| | CDR1 | |
| 68 | CRT3 V1 LC | LWIYSTSNLA |
| | CDR2 | |
| 69 | CRT3 V1 LC | HQYHRSPR |
| | CDR3 | |
| 70 | CRT3 V1 HC | ACCAGCTACTGGATGCAC |
| | CDR1 | |
| | (nucleotide) | |
| 71 | CRT3 V1 HC | TGGATTGGCGCTATTTATCCTGGAAATAGTGATACTAGC |
| | CDR2 | |
| | (nucleotide) | |
| 72 | CRT3 V1 HC | ACACATTACTACGGTAGTGACTATGCTATGGAC |
| | CDR3 | |
| | (nucleotide) | |
| 73 | CRT3 V1 LC | AGTTCCAGTTACTTGCACTGGTAC |
| | CDR1 | |
| | (nucleotide) | |
| 74 | CRT3 V1 LC | CTCTGGATTTATAGCACATCCAACCTGGCT |
| | CDR2 | |
| | (nucleotide) | |
| 75 | CRT3 V1 LC | CCACCAGTATCATCGTTCCCCACGG |
| | CDR3 | |
| | (nucleotide) | |

CRT3 Version 2 - complementarity determining regions

| | | |
|---|---|---|
| 76 | CRT3 V2 HC | GYTFTSYW |
| | CDR1 | |
| 77 | CRT3 V2 HC | IYPGNSDT |
| | CDR2 | |
| 78 | CRT3 V2 HC | THYYGSDYAMDY |
| | CDR3 | |
| 79 | CRT3 V2 LC | SSVSSSY |
| | CDR1 | |
| | CRT3 V2 LC | STS |
| | CDR2 | |
| 81 | CRT3 V2 LC | HQYHRSPRT |
| | CDR3 | |
| 82 | CRT3 V2 HC | GGCTACACCTTTACCAGCTACTGG |
| | CDR1 | |
| | (nucleotide) | |
| 83 | CRT3 V2 HC | ATTTATCCTGGAAATAGTGATACT |
| | CDR2 | |
| | (nucleotide) | |
| 84 | CRT3 V2 HC | ACACATTACTACGGTAGTGACTATGCTATGGACTAC |
| | CDR3 | |
| | (nucleotide) | |
| 85 | CRT3 V2 LC | TCAAGTGTAAGTTCCAGTTAC |
| | CDR1 | |
| | (nucleotide) | |
| | CRT3 V2 LC | AGCACATCC |
| | CDR2 | |
| | (nucleotide) | |
| 87 | CRT3 V2 LC | CACCAGTATCATCGTTCCCCACGGACG |
| | CDR3 | |
| | (nucleotide) | |

CRT3 heavy and light chain variable regions, scFv and CARs

| | | |
|---|---|---|
| 88 | CRT3 HC V1 | M A E V Q L Q Q S G T V L A R P G A S V K M |
| | | S C K A S G Y T F T S Y W M H W V K Q R P |
| | | G Q G L E W I G A I Y P G N S D T S Y N Q K |
| | | F K G K A K L T A V T S T S T A Y M E L S S |
| | | L T N E D S A V F Y C T H Y Y G S D Y A M D |
| | | Y W G Q G T S V T ISSG |
| 89 | CRT3 LC V1 | Q I V L T Q S P A I M S A S L G E R V T M T |
| | | C T A S S S V S S S Y L H W Y Q Q K P G S S |
| | | P K L W I Y S T S N L A S G V P A R F S G S |
| | | G S G T S Y S L T I S S M E A E D A A T Y Y |
| | | C H Q Y H R S P R T F G G G T K L E I K R A |
| | | A |
| 90 | CRT3 HC V2 | M A E V Q L Q Q S G T V L A R P G A S V K M |
| | | S C K A S G Y T F T S Y W M H W V K Q R P |
| | | G Q G L E W I G A I Y P G N S D T S Y N Q K |
| | | F K G K A K L T A V T S T S T A Y M E L S S |
| | | L T N E D S A V F Y C T H Y Y G S D Y A M D |
| | | Y W G Q G T S V T V |
| 91 | CRT3 LC V2 | Q I V L T Q S P A I M S A S L G E R V T M T |
| | | C T A S S S V S S S Y L H W Y Q Q K P G S S |
| | | P K L W I Y S T S N L A S G V P A R F S G S |
| | | G S G T S Y S L T I S S M E A E D A A T Y Y |
| | | C H Q Y H R S P R T F G G G T K L E I K R A |
| | | A A |
| 92 | CRT3 HC V1 | ATGGCCGAGGTCCAGCTGCAGCAGTCTGGGACTGTGCT |
| | nucleotide | GGCAAGGCCTGGGGCTTCAGTGAAGATGTCCTGC |
| | | AAGGCTTCTGGCTACACCTTTACCAGCTACTGGATGCAC |
| | | TGGGTAAAACAGAGGCCTGGACAGGGTCTGGAATGGAT |
| | | TGGCGCTATTTATCCTGGAAATAGTGATACTAGCTACAA |
| | | CCAGAAGTTCAAGGGCAAGGCCAAACTG |
| | | ACTGCAGTCACATCCACCAGCACTGCCTACATGGAGCT |
| | | CAGCAGCCTGACAAATGAGGACTCTGCGGTCTTT |
| | | TACTGTACACATTACTACGGTAGTGACTATGCTATGGAC |
| | | TACTGGGGTCAAGGAACCTCAGTCACTGTCTCC |
| | | TCA |
| 93 | CRT3 LC V1 | CAAATTGTTCTCACCCAGTCTCCAGCAATCATGTCTGCA |
| | nucleotide | TCTCTAGGGGAACGGGTCACCATGACCTGCACTGCCAG |
| | | CTCAAGTGTAAGTTCCAGTTACTTGCACTGGTACCAGCA |
| | | GAAGCCAGGATCCTCCCCCAAACTCTGGATTTATAGCAC |
| | | ATCCAACCTGGCTTCTGGAGTCCCAGCTCGCTTCAGTG |
| | | GCAGTGGGTCTGGGACCTCTTACTCTCTCACAATCAGCA |
| | | GCATGGAGGCTGAAGATGCTGCCACTTATTACTGCCAC |
| | | CAGTATCATCGTTCCCCACGGACGTTCGGTGGAGGCAC |
| | | CAAGCTGGAAATCAAACGTGCGGCCGC |
| 94 | CRT3 HC V2 | ATGGCCGAGGTCCAGCTGCAGCAGTCTGGGACTGTGCT |
| | nucleotide | GGCAAGGCCTGGGGCTTCAGTGAAGATGTCCTGCAAGG |
| | | CTTCTGGCTACACCTTTACCAGCTACTGGATGCACTGGG |
| | | TAAAACAGAGGCCTGGACAGGGTCTGGAATGGATTGGC |
| | | GCTATTTATCCTGGAAATAGTGATACTAGCTACAACCAG |
| | | AAGTTCAAGGGCAAGGCCAAACTGACTGCAGTCACATC |
| | | CACCAGCACTGCCTACATGGAGCTCAGCAGCCTGACAA |
| | | ATGAGGACTCTGCGGTCTTTTACTGTACACATTACTACG |
| | | GTAGTGACTATGCTATGGACTACTGGGGTCAAGGAACC |
| | | TCAGTCACTGTC |
| 95 | CRT3 LC V2 | CAAATTGTTCTCACCCAGTCTCCAGCAATCATGTCTGCA |
| | nucleotide | TCTCTAGGGGAACGGGTCACCATGACCTGCACTGCCAG |
| | | CTCAAGTGTAAGTTCCAGTTACTTGCACTGGTACCAGCA |
| | | GAAGCCAGGATCCTCCCCCAAACTCTGGATTTATAGCAC |
| | | ATCCAACCTGGCTTCTGGAGTCCCAGCTCGCTTCAGTG |
| | | GCAGTGGGTCTGGGACCTCTTACTCTCTCACAATCAGCA |
| | | GCATGGAGGCTGAAGATGCTGCCACTTATTACTGCCAC |
| | | CAGTATCATCGTTCCCCACGGACGTTCGGTGGAGGCAC |
| | | CAAGCTGGAAATCAAACGT |
| 96 | CRT3 scFv | M A E V Q L Q Q S G T V L A R P G A S V K M |
| | | S C K A S G Y T F T S Y W M H W V K Q R P |
| | | G Q G L E W I G A I Y P G N S D T S Y N Q K |
| | | F K G K A K L T A V T S T S T A Y M E L S S |
| | | L T N E D S A V F Y C T H Y Y G S D Y A M D |
| | | Y W G Q G T S V T V S S G G G G S G G G G |
| | | S G G G G S Q I V L T Q S P A I M S A S L G |
| | | E R V T M T C T A S S S V S S S Y L H W Y Q |
| | | Q K P G S S P K L W I Y S T S N L A S G V P |
| | | A R F S G S G S G T S Y S L T I S S M E A E |
| | | D A A T Y Y C H Q Y H R S P R T F G G G T K |
| | | L E I K R A A A |
| 97 | CRT3 scFv | ATGGCCGAGGTCCAGCTGCAGCAGTCTGGGACTGTGCT |
| | nucleotide | GGCAAGGCCTGGGGCTTCAGTGAAGATGTCCTGCAAGG |
| | | CTTCTGGCTACACCTTTACCAGCTACTGGATGCACTGGG |
| | | TAAAACAGAGGCCTGGACAGGGTCTGGAATGGATTGGC |
| | | GCTATTTATCCTGGAAATAGTGATACTAGCTACAACCAG |
| | | AAGTTCAAGGGCAAGGCCAAACTGACTGCAGTCACATC |
| | | CACCAGCACTGCCTACATGGAGCTCAGCAGCCTGACAA |
| | | ATGAGGACTCTGCGGTCTTTTACTGTACACATTACTACG |
| | | GTAGTGACTATGCTATGGACTACTGGGGTCAAGGAACC |
| | | TCAGTCACTGTCTCCTCAGGTGGAGGCGGTTCAGGCGG |
| | | AGGTGGCTCTGGCGGTGGCGGATCGCAAATTGTTCTCA |
| | | CCCAGTCTCCAGCAATCATGTCTGCATCTCTAGGGGAAC |
| | | GGGTCACCATGACCTGCACTGCCAGCTCAAGTGTAAGT |
| | | TCCAGTTACTTGCACTGGTACCAGCAGAAGCCAGGATC |
| | | CTCCCCCAAACTCTGGATTTATAGCACATCCAACCTGGC |
| | | TTCTGGAGTCCCAGCTCGCTTCAGTGGCAGTGGGTCTG |
| | | GGACCTCTTACTCTCTCACAATCAGCAGCATGGAGGCT |
| | | GAAGATGCTGCCACTTATTACTGCCACCAGTATCATCGT |
| | | TCCCCACGGACGTTCGGTGGAGGCACCAAGCTGGAAAT |
| | | CAAACGTGCGGCCGCA |
| 98 | CRT3 CAR3 | M G V L L T Q R T L L S L V L A L L F P S M A |
| | | S M A E V Q L Q Q S G T V L A R P G A S V K |
| | | M S C K A S G Y T F T S Y W M H W V K Q R |
| | | P G Q G L E W I G A I Y P G N S D T S Y N Q |
| | | K F K G K A K L T A V T S T S T A Y M E L S |
| | | S L T N E D S A V F Y C T H Y Y G S D Y A M |
| | | D Y W G Q G T S V T V S S G G G G S G G G |
| | | G S G G G G S Q I V L T Q S P A M S A S L |
| | | G E R V T M T C T A S S S V S S S Y L H W Y |
| | | Q Q K P G S S P K L W I Y S T S N L A S G V |
| | | P A R F S G S G S G T S Y S L T I S S M E A |
| | | E D A A T Y Y C H Q Y H R S P R T F G G G T |
| | | K L E I K R A A A I E V M Y P P P Y L D N E K |
| | | S N G T I I H V K G K H L C P S P L F P G P S |
| | | K P F W V L V V V G G V L A C Y S L L V T V |
| | | A F I I F W V R S K R S R L L H S D Y M N M |
| | | T P R R P G P T R K H Y Q P Y A P P R D F A |
| | | A Y R S R V K F S R S A D A P A Y Q Q G Q N |
| | | Q L Y N E L N L G R R E E Y D V L D K R R G |
| | | R D P E M G G K P Q R R K N P Q E G L Y N |
| | | E L Q K D K M A E A Y S E I G M K G E R R R |
| | | G K G H D G L Y Q G L S T A T K D T Y D A L |
| | | H M Q A L P P R |
| 99 | CRT3 CAR3 | atgggcgtgctgctgacccagaggaccctgctgagcctggtgctggccctgctgttt |
| | nucleotide | ccatctatggcatcgatggccgaggtccagctgcagcagtctgggactgtgctggc |
| | | aaggcctggggcttcagtgaagatgtcctgcaaggcttctggctacacctttaccag |
| | | ctactggatgcactgggtaaaacagaggcctggacagggtctggaatggattggc |
| | | gctatttatcctggaaatagtgatactagctacaaccagaagttcaagggcaaggc |
| | | caaactgactgcagtcacatccaccagcactgcctacatggagctcagcagcctg |
| | | acaaatgaggactctgcggtcttttactgtacacattactacggtagtgactatgctat |
| | | ggactactggggtcaaggaacctcagtcactgtctcctcaggtggaggcggttcag |
| | | gcggaggtggctctggcggtggcggatcgcaaattgttctcacccagtctccagca |
| | | atcatgtctgcatctctaggggaacgggtcaccatgacctgcactgccagctcaagt |
| | | gtaagttccagttacttgcactggtaccagcagaagccaggatcctcccccaaact |
| | | ctggatttatagcacatccaacctggcttctggagtcccagctcgcttcagtggcagt |
| | | gggtctgggacctcttactctctcacaatcagcagcatggaggctgaagatgctgc |
| | | cacttattactgccaccagtatcatcgttccccacggacgttcggtggaggcaccaa |
| | | gctggaaatcaaacgtgcggccgcaattgaagttatgtatcctcctccttacctaga |
| | | caatgagaagagcaatggaaccattatccatgtgaaagggaaacacctttgtcca |
| | | agtcccctatttcccggaccttctaagcccttttgggtgctggtggtggttggtggagtc |
| | | ctggcttgctatagcttgctagtaacagtggcctttattattttctgggtgaggagtaag |
| | | aggagcaggctcctgcacagtgactacatgaacatgactccccgccgccccggg |
| | | cccacccgcaagcattaccagccctatgccccaccacgcgacttcgcagcctatc |
| | | gctccagagtgaagttcagcaggagcgcagacgcccccgcgtaccagcagggc |
| | | cagaaccagctctataacgagctcaatctaggacgaagagaggagtacgatgttt |
| | | tggacaagagacgtggccgggaccctgagatggggggaaagccgcagagaa |
| | | ggaagaaccctcaggaaggcctgtacaatgaactgcagaaagataagatggcg |
| | | gaggcctacagtgagattgggatgaaaggcgagcgccggaggggcaaggggc |
| | | acgatggcctttaccagggtctcagtacagccaccaaggacacctacgacgccct |
| | | tcacatgcaggccctgccccctcgctaataaaagcttaacacgagcca |

CRT4 Version 1 (V1) - complementarity determining regions

| | | |
|---|---|---|
| 32 | CRT4 V1 HC | SSYWIE |
| | CDR1 | |
| 33 | CRT4 V1 HC | WIGEILPGSGSTN |
| | CDR2 | |
| 100 | CRT4 V1 HC | ARGGDYDEEYYLMD |
| | CDR3 | |
| 35 | CRT4 V1 LC | SYMYWY |
| | CDR1 | |
| 36 | CRT4 V1 LC | LLIYDTSNLA |
| | CDR2 | |
| 37 | CRT4 V1 LC | QQWSSYPL |
| | CDR3 | |
| 38 | CRT4 V1 HC | AGTAGCTACTGGATAGAG |
| | CDR1 | |
| | (nucleotide) | |
| 39 | CRT4 V1 HC | TGGATTGGAGAGATTTTACCTGGAAGTGGTAGTACTAAT |
| | CDR2 | |
| | (nucleotide) | |
| 101 | CRT4 V1 HC | GCGAGAGGGGGGGATTACGACGAAGAATACTATCTCAT |
| | CDR3 | GGAC |
| | (nucleotide) | |
| 41 | CRT4 V1 LC | AGTTACATGTACTGGTAC |
| | CDR1 | |
| | (nucleotide) | |
| 42 | CRT4 V1 LC | CTCCTGATTTATGACACATCCAACCTGGCT |
| | CDR2 | |
| | (nucleotide) | |
| 43 | CRT4 V1 LC | CAGCAGTGGAGTAGTTACCCGCTC |
| | CDR3 | |
| | (nucleotide) | |

CRT4 Version 2 (V2) - Complementarity determining regions

| | | |
|---|---|---|
| 44 | CRT4 V2 HC | GYTFSSYW |
| | CDR1 | |
| 45 | CRT4 V2 HC | ILPGSGST |
| | CDR2 | |
| 102 | CRT4 V2 HC | ARGGDYDEEYYLMDY |
| | CDR3 | |
| 47 | CRT4 V2 LC | SSVSY |
| | CDR1 | |
| | CRT4 V2 LC | DTS |
| | CDR2 | |
| 49 | CRT4 V2 LC | QQWSSYPLT |
| | CDR3 | |
| 50 | CRT4 V2 HC | GGCTACACATTCAGTAGCTACTGG |
| | CDR1 | |
| | (nucleotide) | |
| 51 | CRT4 V2 HC | ATTTTACCTGGAAGTGGTAGTACT |
| | CDR2 | |
| | (nucleotide) | |
| 103 | CRT4 V2 HC | GCGAGAGGGGGGGATTACGACGAAGAATACTATCTCAT |
| | CDR3 | TAC |
| | (nucleotide) | |
| 53 | CRT4 V2 LC | TCAAGTGTAAGTTAC |
| | CDR1 | |
| | (nucleotide) | |
| | CRT4 V2 LC | GACACATCC |
| | CDR2 | |
| | (nucleotide) | |
| 55 | CRT4 V2 LC | CAGCAGTGGAGTAGTTACCCGCTCACG |
| | CDR3 | |
| | (nucleotide) | |

CRT4 heavy and light chain variable regions, scFv and CARs

| | | |
|---|---|---|
| 104 | CRT4 V1 HC | MAQVQLQQSGAELMKPGASVKISCKATGYTFSSYWIEWV |
| | | NRRPGHGLEWIGEILPGSGSTNYNEKFKGKATFTADTSSN |
| | | TAYMQLSSLTSEDSAVYYCARGGDYDEEYYLMDYWGQGT |
| | | TLTVSS |
| 105 | CRT4 V1 LC | QIVLTQSPAIMSASPGEKVTMTCSASSSVSYMYWYQQKPG |
| | | SSPRLLIYDTSNLASGVPVRFSGSGSGTSYSLTISRMEAED |
| | | AATYYCQQWSSYPLTFGAGTKLEIKRAA |
| 106 | CRT4 V2 HC | MAQVQLQQSGAELMKPGASVKISCKATGYTFSSYWIEWV |
| | | NRRPGHGLEWIGEILPGSGSTNYNEKFKGKATFTADTSSN |
| | | TAYMQLSSLTSEDSAVYYCARGGDYDEEYYLMDYWGQGT |
| | | TLTV |
| 107 | CRT4 V2 LC | QIVLTQSPAIMSASPGEKVTMTCSASSSVSYMYWYQQKPG |
| | | SSPRLLIYDTSNLASGVPVRFSGSGSGTSYSLTISRMEAED |
| | | AATYYCQQWSSYPLTFGAGTKLEIKRAAA |
| 108 | CRT4 V1 HC | ATGGCCCAGGTTCAGCTGCAGCAGTCTGGAGCTGAGCT |
| | (nucleotide) | GATGAAGCCTGGGGCCTCAGTGAAGATATCCTGCAAGG |
| | | CTACTGGCTACACATTCAGTAGCTACTGGATAGAGTGGG |
| | | TAAACCGGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAATACAGCCTACATGCAACTCAGCAGCCTCACATCT |
| | | GAGGACTCTGCCGTCTATTACTGTGCGAGAGGGGGGGA |
| | | TTACGACGAAGAATACTATCTCATGGACTACTGGGGTCA |
| | | AGGCACCACTCTCACAGTCTCCTCA |
| 109 | CRT4 V1 LC | CAAATTGTTCTCACCCAGTCTCCAGCAATCATGTCTGCA |
| | (nucleotide) | TCTCCAGGGGAGAAGGTCACCATGACCTGCAGTGCCAG |
| | | CTCAAGTGTAAGTTACATGTACTGGTACCAGCAGAAGCC |
| | | AGGATCCTCCCCCAGACTCCTGATTTATGACACATCCAA |
| | | CCTGGCTTCTGGAGTCCCTGTTCGCTTCAGTGGCAGTG |
| | | GGTCTGGGACCTCTTACTCTCTCACAATCAGCCGAATGG |
| | | AGGCTGAAGATGCTGCCACTTATTACTGCCAGCAGTGG |
| | | AGTAGTTACCCGCTCACGTTCGGTGCTGGGACCAAGCT |
| | | GGAAATCAAACGTGCGGCCGC |
| 110 | CRT4 V2 HC | ATGGCCCAGGTTCAGCTGCAGCAGTCTGGAGCTGAGCT |
| | (nucleotide) | GATGAAGCCTGGGGCCTCAGTGAAGATATCCTGCAAGG |
| | | CTACTGGCTACACATTCAGTAGCTACTGGATAGAGTGGG |
| | | TAAACCGGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAATACAGCCTACATGCAACTCAGCAGCCTCACATCT |
| | | GAGGACTCTGTCGTCTATTACTGTGCGAGAGGGGGGGA |
| | | TTACGACGAAGAATACTATCTCATGGACTACTGGGGTCA |
| | | AGGCACCACTCTCACAGTC |
| 111 | CRT4 V2 LC | CAAATTGTTCTCACCCAGTCTCCAGCAATCATGTCTGCA |
| | (nucleotide) | TCTCCAGGGGAGAAGGTCACCATGACCTGCAGTGCCAG |
| | | CTCAAGTGTAAGTTACATGTACTGGTACCAGCAGAAGCC |
| | | AGGATCCTCCCCCAGACTCCTGATTTATGACACATCCAA |
| | | CCTGGCTTCTGGAGTCCCTGTTCGCTTCAGTGGCAGTG |
| | | GGTCTGGGACCTCTTACTCTCTCACAATCAGCCGAATGG |
| | | AGGCTGAAGATGCTGCCACTTATTACTGCCAGCAGTGG |
| | | AGTAGTTACCCGCTCACGTTCGGTGCTGGGACCAAGCT |
| | | GGAAATCAAACGT |
| 112 | CRT4 scFv | MAQVQLQQSGAELMKPGASVKISCKATGYTFSSYWIEWV |
| | | NRRPGHGLEWIGEILPGSGSTNYNEKFKGKATFTADTSSN |
| | | TAYMQLSSLTSEDSVVYYCARGGDYDEEYYLMDYWGQGT |
| | | TLTVSSGGGGSGGGGSGGGGSQIVLTQSPAIMSASPGEK |
| | | VTMTCSASSSVSYMYWYQQKPGSSPRLLIYDTSNLASGVP |
| | | VRFSGSGSGTSYSLTISRMEAEDAATYYCQQWSSYPLTFG |
| | | AGTKLEIKRAAA |
| 113 | CRT4 scFv | ATGGCCCAGGTTCAGCTGCAGCAGTCTGGAGCTGAGCT |
| | (nucleotide) | GATGAAGCCTGGGGCCTCAGTGAAGATATCCTGCAAGG |
| | | CTACTGGCTACACATTCAGTAGCTACTGGATAGAGTGGG |
| | | TAAACCGGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAATACAGCCTACATGCAACTCAGCAGCCTCACATCT |
| | | GAGGACTCTGTCGTCTATTACTGTGCGAGAGGGGGGGA |
| | | TTACGACGAAGAATACTATCTCATGGACTACTGGGGTCA |
| | | AGGCACCACTCTCACAGTCTCCTCAGGTGGAGGCGGTT |
| | | CAGGCGGAGGTGGCTCTGGCGGTGGCGGATCGCAAAT |
| | | TGTTCTCACCCAGTCTCCAGCAATCATGTCTGCATCTCC |
| | | AGGGGAGAAGGTCACCATGACCTGCAGTGCCAGCTCAA |
| | | GTGTAAGTTACATGTACTGGTACCAGCAGAAGCCAGGAT |
| | | CCTCCCCCAGACTCCTGATTTATGACACATCCAACCTGG |
| | | CTTCTGGAGTCCCTGTTCGCTTCAGTGGCAGTGGGTCT |
| | | GGGACCTCTTACTCTCTCACAATCAGCCGAATGGAGGC |
| | | TGAAGATGCTGCCACTTATTACTGCCAGCAGTGGAGTAG |
| | | TTACCCGCTCACGTTCGGTGCTGGGACCAAGCTGGAAA |
| | | TCAAACGTGCGGCCGCA |
| 114 | CAR4 CRT4 | M G V L L T Q R T L L S L V L A L L F P S M A |
| | | S M A Q V Q L Q Q S G A E L M K P G A S V K |
| | | I S C K A T G Y T F S S Y W I E W V N R R P |
| | | G H G L E W I G E I L P G S G S T N Y N E K |
| | | F K G K A T F T A D T S S N T A Y M Q L S S |
| | | L T S E D S V V Y Y C A R G G D Y D E E Y Y |
| | | L M D Y W G Q G T T L T V S S G G G G S G |
| | | G G G S G G G G S Q I V L T Q S P A I M S A |
| | | S P G E K V T M T C S A S S S V S Y M Y W Y |
| | | Q Q K P G S S P R L L I Y D T S N L A S G V |
| | | P V R F S G S G S G T S Y S L T I S R M E A |
| | | E D A A T Y Y C Q Q W S S Y P L T F G A G T |
| | | K L E I K R A A A I E V M Y P P P Y L D N E K |
| | | S N G T I I H V K G K H L C P S P L F P G P S |
| | | K P F W V L V V V G G V L A C Y S L L V T V |
| | | A F I I F W V R S K R S R L L H S D Y M N M |
| | | T P R R P G P T R K H Y Q P Y A P P R D F A |
| | | A Y R S R V K F S R S A D A P A Y Q Q G Q N |
| | | Q L Y N E L N L G R R E E Y D V L D K R R G |
| | | R D P E M G G K P Q R R K N P Q E G L Y N |
| | | E L Q K D K M A E A Y S E I G M K G E R R R |
| | | G K G H D G L Y Q G L S T A T K D T Y D A L |
| | | H M Q A L P P R |
| 115 | CAR4 CRT4 | atggcccaggttcagctgcagcagtctggagctgagctgatgaagcctggggcct |
| | (nucleotide) | cagtgaagatatcctgcaaggctactggctacacattcagtagctactggatagagt |
| | | gggtaaaccggaggcctggacatggccttgagtggattggagagattttacctgga |
| | | agtggtagtactaattacaatgagaagttcaagggcaaggccacattcactgcag |
| | | atacatcctccaatacagcctacatgcaactcagcagcctcacatctgaggactct |
| | | gtcgtctattactgtgcgagagggggggattacgacgaagaatactatctcatgga |
| | | ctactggggtcaaggcaccactctcacagtctcctcaggtggaggcggttcaggcg |
| | | gaggtggctctggcggtggcggatcgcaaattgttctcacccagtctccagcaatc |
| | | atgtctgcatctccaggggagaaggtcaccatgacctgcagtgccagctcaagtgt |
| | | aagttacatgtactggtaccagcagaagccaggatcctcccccagactcctgattta |
| | | tgacacatccaacctggcttctggagtccctgttcgcttcagtggcagtgggtctggg |
| | | acctcttactctctcacaatcagccgaatggaggctgaagatgctgccacttattact |
| | | gccagcagtggagtagttacccgctcacgttcggtgctgggaccaagctggaaat |
| | | caaacgtgcggccgcaattgaagttatgtatcctcctccttacctagacaatgagaa |
| | | gagcaatggaaccattatccatgtgaaagggaaacacctttgtccaagtcccctatt |
| | | tcccggaccttctaagcccttttgggtgctggtggtggttggtggagtcctggcttgcta |
| | | tagcttgctagtaacagtggcctttattattttctgggtgaggagtaagaggagcagg |
| | | ctcctgcacagtgactacatgaacatgactccccgccgccccgggcccacccgc |
| | | aagcattaccagccctatgccccaccacgcgacttcgcagcctatcgctccagagt |
| | | gaagttcagcaggagcgcagacgcccccgcgtaccagcagggccagaaccag |
| | | ctctataacgagctcaatctaggacgaagagaggagtacgatgttttggacaaga |
| | | gacgtggccgggaccctgagatggggggaaagccgcagagaaggaagaacc |
| | | ctcaggaaggcctgtacaatgaactgcagaaagataagatggcggaggcctac |
| | | agtgagattgggatgaaaggcgagcgccggaggggcaaggggcacgatggcc |
| | | tttaccagggtctcagtacagccaccaaggacacctacgacgcccttcacatgca |
| | | ggccctgccccctcgctaataa |

CRT5 Version 1 (V1) - complementarity determining regions

| | | |
|---|---|---|
| 32 | CRT5 V1 HC | SSYWIE |
| | CDR1 | |
| 33 | CRT5 V1 HC | WIGEILPGSGSTN |
| | CDR2 | |
| 116 | CRT5 V1 HC | ARGGDYDEEYYAMD |
| | CDR3 | |
| 35 | CRT5 V1 LC | SYMYWY |
| | CDR1 | |
| 36 | CRT5 V1 LC | LLIYDTSNLA |
| | CDR2 | |
| 37 | CRT5 V1 LC | QQWSSYPL |
| | CDR3 | |
| 38 | CRT5 V1 HC | AGTAGCTACTGGATAGAG |
| | CDR1 | |
| | (nucleotide) | |
| 39 | CRT5 V1 HC | TGGATTGGAGAGATTTTACCTGGAAGTGGTAGTACTAAT |
| | CDR2 | |
| | (nucleotide) | |
| 117 | CRT5 V1 HC | GCAAGAGGGGGGGATTACGACGAAGAATACTATGCTAT |
| | CDR3 | GGAC |
| | (nucleotide) | |
| 41 | CRT5 V1 LC | AGTTACATGTACTGGTAC |
| | CDR1 | |
| | (nucleotide) | |
| 42 | CRT5 V1 LC | CTCCTGATTTATGACACATCCAACCTGGCT |
| | CDR2 | |
| | (nucleotide) | |
| 43 | CRT5 V1 LC | CAGCAGTGGAGTAGTTACCCGCTC |
| | CDR3 | |
| | (nucleotide) | |

CRT5 Version 2 (V2) - complementarity determining regions

| | | |
|---|---|---|
| 44 | CRT5 V2 HC | GYTFSSYW |
| | CDR1 | |
| 45 | CRT5 V2 HC | ILPGSGST |
| | CDR2 | |
| 118 | CRT5 V2 HC | ARGGDYDEEYYAMDY |
| | CDR3 | |
| 47 | CRT5 V2 LC | SSVSY |
| | CDR1 | |
| | CRT5 V2 LC | DTS |
| | CDR2 | |
| 49 | CRT5 V2 LC | QQWSSYPLT |
| | CDR3 | |
| 50 | CRT5 V2 HC | GGCTACACATTCAGTAGCTACTGG |
| | CDR1 | |
| | (nucleotide) | |
| 51 | CRT5 V2 HC | ATTTTACCTGGAAGTGGTAGTACT |
| | CDR2 | |
| | (nucleotide) | |
| 120 | CRT5 V2 HC | GCAAGAGGGGGGGATTACGACGAAGAATACTATGCTAT |
| | CDR3 | GGACTAC |
| | (nucleotide) | |
| 53 | CRT5 V2 LC | TCAAGTGTAAGTTAC |
| | CDR1 | |
| | (nucleotide) | |
| | CRT5 V2 LC | GACACATCC |
| | CDR2 | |
| | (nucleotide) | |
| 55 | CRT5 V2 LC | CAGCAGTGGAGTAGTTACCCGCTCACG |
| | CDR3 | |
| | (nucleotide) | |

CRT5 heavy and light chain variable regions, scFv and CARs

| | | |
|---|---|---|
| 121 | CRT5 HC | MAEVQLQQSGAELMKPGASVKISCKATGYTFSSYWIEWV |
| | | NQRPGHGLEWIGEILPGSGST |
| | | NYNEKFKGKATFTADTSSNTAYMQLSSLTSEDSAVYYCAR |
| | | GGDYDEEYYAMDYWGQGTSV TL |
| 122 | CRT5 LC | QIVLTQSPAIMSASPGEKVTMTCSASSSV-- |
| | | SYMYWYQQKPGSSPRLLIYDTSNLASGVP |
| | | VRFSGSGSGTSYSLTISRMEAEDGATYYCQQWSSYPLTF |
| | | GAGTKLELKR |
| 123 | CRT5 HC | ATGGCCGAGGTTCAGCTTCAGCAGTCTGGAGCTGAGCT |
| | (nucleotide) | GATGAAGCCTGGGGCCTCAGTGAAGATATCCTGCAAGG |
| | | CTACTGGCTACACATTCAGTAGCTACTGGATAGAGTGGG |
| | | TAAATCAGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAACACAGCCTACATGCAACTCAGCAGCCTGACATCT |
| | | GAGGACTCTGCCGTCTATTACTGTGCAAGAGGGGGGGA |
| | | TTACGACGAAGAATACTATGCTATGGACTACTGGGGTCA |
| | | AGGAACCTCAGTCACCCTC |
| 124 | CRT5 LC | CAAATTGTTCTCACCCAGTCTCCAGCAATCATGTCTGCA |
| | (nucleotide) | TCTCCAGGGGAGAAGGTCACCATGACCTGCAGTGCCAG |
| | | CTCAAGTGTAAGTTACATGTACTGGTACCAGCAGAAGCC |
| | | AGGATCCTCCCCCAGACTCCTGATTTATGACACATCCAA |
| | | CCTGGCTTCTGGAGTCCCTGTTCGCTTCAGTGGCAGTG |
| | | GGTCTGGGACCTCTTACTCTCTCACAATCAGCCGAATGG |
| | | AGGCTGAAGATGCTGCCACTTATTACTGCCAGCAGTGG |
| | | AGTAGTTACCCGCTCACGTTCGGTGCTGGGACCAAGCT |
| | | GGAGCTGAAACGT |
| 125 | CRT5 scFv | MAEVQLQQSGAELMKPGASVKISCKATGYTFSSYWIEWV |
| | | NQRPGHGLEWIGEILPGSGSTNYNEKFKGKATFTADTSSN |
| | | TAYMQLSSLTSEDSAVYYCARGGDYDEEYYAMDYWGQG |
| | | TSVTLSSGGGGSGGGGSGGGGSQIVLTQSPAIMSASPGE |
| | | KVTMTCSASSSVSYMYWYQQKPGSSPRLLIYDTSNLASGV |
| | | PVRFSGSGSGTSYSLTISRMEAEDAATYYCQQWSSYPLTF |
| | | GAGTKLELKRAAA |
| 126 | CRT5 scFv | ATGGCCGAGGTTCAGCTTCAGCAGTCTGGAGCTGAGCT |
| | (nucleotide) | GATGAAGCCTGGGGCCTCAGTGAAGATATCCTGCAAGG |
| | | CTACTGGCTACACATTCAGTAGCTACTGGATAGAGTGGG |
| | | TAAATCAGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAACACAGCCTACATGCAACTCAGCAGCCTGACATCT |
| | | GAGGACTCTGCCGTCTATTACTGTGCAAGAGGGGGGGA |
| | | TTACGACGAAGAATACTATGCTATGGACTACTGGGGTCA |
| | | AGGAACCTCAGTCACCCTCTCCTCAGGTGGAGGCGGTT |
| | | CAGGCGGAGGTGGCTCTGGCGGTGGCGGATCGCAAAT |
| | | TGTTCTCACCCAGTCTCCAGCAATCATGTCTGCATCTCC |
| | | AGGGGAGAAGGTCACCATGACCTGCAGTGCCAGCTCAA |
| | | GTGTAAGTTACATGTACTGGTACCAGCAGAAGCCAGGAT |
| | | CCTCCCCCAGACTCCTGATTTATGACACATCCAACCTGG |
| | | CTTCTGGAGTCCCTGTTCGCTTCAGTGGCAGTGGGTCT |
| | | GGGACCTCTTACTCTCTCACAATCAGCCGAATGGAGGC |
| | | TGAAGATGCTGCCACTTATTACTGCCAGCAGTGGAGTAG |
| | | TTACCCGCTCACGTTCGGTGCTGGGACCAAGCTGGAGC |
| | | TGAAACGTGCGGCCGCA |
| 127 | CAR5 CRT5- | M G V L L T Q R T L L S L V L A L L F P S M |
| | oncostatin leader | A S M A E V Q L Q Q S G A E L M K P G A S V |
| | (bold), scFv | K I S C K A T G Y T F S S Y W I E W V N Q R |
| | CRT5 (italics), | P G H G L E W I G E I L P G S G S T N Y N E |
| | CD28-CD3 zeta | K F K G K A T F T A D T S S N T A Y M Q L S |
| | rest of sequence | S L T S E D S A V Y Y C A R G G D Y D E E Y |
| | | Y A M D Y W G Q G T S V T L S S G G G G S |
| | | G G G G S G G G G S Q I V L T Q S P A I M S |
| | | A S P G E K V T M T C S A S S S V S Y M Y W |
| | | Y Q Q K P G S S P R L L I Y D T S N L A S G |
| | | V P V R F S G S G S G T S Y S L T I S R M E |
| | | A E D A A T Y Y C Q Q W S S Y P L T F G A G |
| | | T K L E L K R A A A I E V M Y P P P Y L D N E |
| | | K S N G T I I H V K G K H L C P S P L F P G P |
| | | S K P F W V L V V V G G V L A C Y S L L V T |
| | | V A F I I F W V R S K R S R L L H S D Y M N |
| | | M T P R R P G P T R K H Y Q P Y A P P R D F |
| | | A A Y R S R V K F S R S A D A P A Y Q Q G Q |
| | | N Q L Y N E L N L G R R E E Y D V L D K R R |
| | | G R D P E M G G K P Q R R K N P Q E G L Y |
| | | N E L Q K D K M A E A Y S E I G M K G E R R |
| | | R G K G H D G L Y Q G L S T A T K D T Y D A |
| | | L H M Q A L P P R |
| 128 | CAR5 CRT5 | atgggcgtgctgctgacccagaggaccctgctgagcctggtgctggccctgctgttt |
| | (nucleotide) | ccatctatggcatcgatggccgaggttcagcttcagcagtctggagctgagctgatg |
| | | aagcctggggcctcagtgaagatatcctgcaaggctactggctacacattcagtag |
| | | ctactggatagagtgggtaaatcagaggcctggacatggccttgagtggattggag |
| | | agattttacctggaagtggtagtactaattacaatgagaagttcaagggcaaggcc |
| | | acattcactgcagatacatcctccaacacagcctacatgcaactcagcagcctga |
| | | catctgaggactctgccgtctattactgtgcaagagggggggattacgacgaaga |
| | | atactatgctatggactactggggtcaaggaacctcagtcaccctctcctcaggtgg |
| | | aggcggttcaggcggaggtggctctggcggtggcggatcgcaaattgttctcaccc |
| | | agtctccagcaatcatgtctgcatctccaggggagaaggtcaccatgacctgcagt |
| | | gccagctcaagtgtaagttacatgtactggtaccagcagaagccaggatcctcccc |
| | | cagactcctgatttatgacacatccaacctggcttctggagtccctgttcgcttcagtg |
| | | gcagtgggtctgggacctcttactctctcacaatcagccgaatggaggctgaagat |
| | | gctgccacttattactgccagcagtggagtagttacccgctcacgttcggtgctggg |
| | | accaagctggagctgaaacgtgcggccgcaattgaagttatgtatcctcctccttac |
| | | ctagacaatgagaagagcaatggaaccattatccatgtgaaagggaaacaccttt |
| | | gtccaagtcccctatttcccggaccttctaagcccttttgggtgctggtggtggttggtg |
| | | gagtcctggcttgctatagcttgctagtaacagtggcctttattattttctgggtgagga |
| | | gtaagaggagcaggctcctgcacagtgactacatgaacatgactccccgccgcc |
| | | ccgggcccacccgcaagcattaccagccctatgccccaccacgcgacttcgcag |
| | | cctatcgctccagagtgaagttcagcaggagcgcagacgcccccgcgtaccagc |
| | | agggccagaaccagctctataacgagctcaatctaggacgaagagaggagtac |
| | | gatgttttggacaagagacgtggccgggaccctgagatggggggaaagccgca |
| | | gagaaggaagaaccctcaggaaggcctgtacaatgaactgcagaaagataag |
| | | atggcggaggcctacagtgagattgggatgaaaggcgagcgccggaggggca |
| | | aggggcacgatggcctttaccagggtctcagtacagccaccaaggacacctacg |
| | | acgcccttcacatgcaggccctgccccctcgctaataaaagcttaacacgagcca |

CRT2 complementarity determining regions

| | | |
|---|---|---|
| 129 | CRT2 LC CDR1 | SYMHWF |
| 68 | CRT2 LC CDR2 | LWIYSTSNLA |
| 130 | CRT2 LC CDR3 | QQRSSYPL |
| 131 | CRT2 LC CDR1 | AGTTACATGCACTGGTTC |
| | (nucleotide) | |
| 74 | CRT2 LC CDR2 | CTCTGGATTTATAGCACATCCAACCTGGCT |
| | (nucleotide) | |
| 132 | CRT2 LC CDR3 | CAGCAAAGGAGTAGTTACCCCCTC |
| | (nucleotide) | |
| 133 | CRT2 LC | EIVLTQSPAIMSASPGEKVTITCSASSSVSYMHWFQQKPGT |
| | | SPKLWIYSTSNLASGVPARFSGSGSGTSYSLTISRMEAEDA |
| | | ATYYCQQRSSYPLTFGAPGKLELKRAA |
| 134 | CRT2 LC | GAAATTGTTCTCACCCAGTCTCCAGCAATCATGTCTGCA |
| | (nucleotide) | TCTCCAGGGGAGAAGGTCACC |
| | | ATAACCTGCAGTGCCAGCTCAAGTGTAAGTTACATGCAC |
| | | TGGTTCCAGCAGAAG |
| | | CCAGGCACTTCTCCCAAACTCTGGATTTATAGCACATCC |
| | | AACCTGGCTTCTGGAGTCCCT |
| | | GCTCGCTTCAGTGGCAGTGGATCTGGGACCTCTTACTC |
| | | TCTCACAATCAGCCGAATGGAG |
| | | GCTGAAGATGCTGCCACTTATTACTGCCAGCAAAGGAGT |
| | | AGTTACCCCCTCACGTTCGGT |
| | | GCTGGGACCAAGCTGGAGCTGAAACGTGCGGCCGC |

Others

| | | |
|---|---|---|
| 135 | Oncostatin M | MGVLLTQRTLLSLVLALLFPSMAS |
| | leader sequence | |
| 136 | CAR5-CRT5: | M P R G W T A L C L L S L L P S G F M S L D |
| | truncated CD34 | N N G T A T P E L P T Q G T F S N V S T N V |
| | plus peptide 2A | S Y Q E T T T P S T L G S T S L H P V S Q H |
| | linker (bold), scFv | G N E A T T N I T E T T V K F T S T S V I T S |
| | CRT5 (italics), | V Y G N T N S S V Q S Q T S V I S T V F T T |
| | CD28-CD3 zeta, | P A N V S T P E T T L K P S L S P G N V S D |
| | rest of sequence | L S T T S T S L A T S P T K P Y T S S S P I L |
| | | S D I K A E I K C S G I R E V K L T Q G I C L |
| | | E Q N K T S S C A E F K K D R G E G L A R V |
| | | L C G E E Q A D A D A G A Q V C S L L L A |
| | | Q S E V R P Q C L L L V L A N R T E I S S K |
| | | L Q L M K K H Q S D L K K L G I L D F T E Q |
| | | D V A S H Q S Y S Q K T L I A L V T S G A L |
| | | L A V L G I T G Y F L M N R R S W S P T G E |
| | | R L E L E P V D R V K Q T L N F D L L K L A |
| | | G D V E S N P G P G N M G V L L T Q R T L L |
| | | S L V L A L L F P S M A S M A E V Q L Q Q S |
| | | G A E L M K P G A S V K I S C K A T G Y T F |
| | | S S Y W I E W V N Q R P G H G L E W I G E I |
| | | L P G S G S T N Y N E K F K G K A T F T A D |
| | | T S S N T A Y M Q L S S L T S E D S A V Y Y |
| | | C A R G G D Y D E E Y Y A M D Y W G Q G T |
| | | S V T L S S G G G G S G G G G S G G G G S |
| | | Q I V L T Q S P A I M S A S P G E K V T M T |
| | | C S A S S S V S Y M Y W Y Q Q K P G S S P |
| | | R L L I Y D T S N L A S G V P V R F S G S G |
| | | S G T S Y S L T I S R M E A E D A A T Y Y C |
| | | Q Q W S S Y P L T F G A G T K L E L K R A A |
| | | A I E V M Y P P P Y L D N E K S N G T I I H V |
| | | K G K H L C P S P L F P G P S K P F W V L V |
| | | V V G G V L A C Y S L L V T V A F I I F W V R |
| | | S K R S R L L H S D Y M N M T P R R P G P T |
| | | R K H Y Q P Y A P P R D F A A Y R S R V K F |
| | | S R S A D A P A Y Q Q G Q N Q L Y N E L N L |
| | | G R R E E Y D V L D K R R G R D P E M G G |
| | | K P Q R R K N P Q E G L Y N E L Q K D K M A |
| | | E A Y S E I G M K G E R R R G K G H D G L Y |
| | | Q G L S T A T K D T Y D A L H M Q A L P P R |
| 137 | CAR5-CRT5: | atgcctcgcggctggacagccctgtgcctgctgtctctgctgccatccggct |
| | truncated CD34 | tcatgagcctggataataacggcacagccaccccagagctgcctacacag |
| | plus peptide 2A | ggcaccttcagcaatgtgtccacaaacgtgagctatcaggagaccacaac |
| | linker (bold), scFv | cccttctaccctgggatccacaagcctgcaccccgtgtctcagcacggcaa |
| | CRT5 (italics), | cgaagccaccaccaacatcaccgagaccacagtgaagtttacctccacct |
| | CD28-CD3 zeta | ctgtgattacctctgtgtacggaaatacaaactccagcgtgcagtctcagac |
| | (nucleotide) rest | atctgtgatctccacagtgtttacaacacctgccaatgtgtccaccccagaga |
| | of sequence | caaccctgaagcccagcctgtctcctggaaatgtgtccgatctgtctaccac |
| | | ctccaccagcctggccacctctcccaccaagccctatacctcctcttctccc |
| | | atcctgagcgatatcaaagccgagatcaaatgcagcgggattcgggaagt |
| | | gaaactgacacagggcatctgcctggaacagaataagacatccagctgcg |
| | | ccgagtttaagaaagatagaggagagggactggccagggtgctgtgtggc |
| | | gaagagcaggccgacgccgatgccggcgcccaggtgtgttccctgctgct |
| | | ggcccagtctgaggtgcgcccccagtgcctgctgctggtgctggccaatcg |
| | | gacagaaattagcagcaagctgcagctgatgaaaaaacaccagagcgatc |
| | | tgaaaaagctgggcatcctggactttaccgagcaggacgtggcctctcacc |
| | | agagctacagccagaaaacactgatcgccctggtgaccagcggagccct |
| | | gctggccgtgctgggcatcaccggatatttcctgatgaataggcgcagctg |
| | | gagccccaccggcgagcggctggagctggagcctgtcgaccgagtgaa |
| | | gcagaccctgaactttgatctgctgaagctggccggcgacgtggagtccaa |
| | | ccccgggccagggaatatgggcgtgctgctgacccagaggaccctgctg |
| | | agcctggtgctggccctgctgtttccatctatggcatcgatggccgaggttcag |
| | | cttcagcagtctggagctgagctgatgaagcctggggcctcagtgaagatatcctg |
| | | caaggctactggctacacattcagtagctactggatagagtgggtaaatcagagg |
| | | cctggacatggccttgagtggattggagagattttacctggaagtggtagtactaatt |
| | | acaatgagaagttcaagggcaaggccacattcactgcagatacatcctccaaca |
| | | cagcctacatgcaactcagcagcctgacatctgaggactctgccgtctattactgtg |
| | | caagagggggggattacgacgaagaatactatgctatggactactggggtcaag |
| | | gaacctcagtcaccctctcctcaggtggaggcggttcaggcggaggtggctctggc |
| | | ggtggcggatcgcaaattgttctcacccagtctccagcaatcatgtctgcatctccag |
| | | gggagaaggtcaccatgacctgcagtgccagctcaagtgtaagttacatgtactgg |
| | | taccagcagaagccaggatcctcccccagactcctgatttatgacacatccaacct |
| | | ggcttctggagtccctgttcgcttcagtggcagtgggtctgggacctcttactctctcac |
| | | aatcagccgaatggaggctgaagatgctgccacttattactgccagcagtggagta |
| | | gttacccgctcacgttcggtgctgggaccaagctggagctgaaacgtgcggccgc |
| | | aattgaagttatgtatcctcctccttacctagacaatgagaagagcaatggaaccatt |
| | | atccatgtgaaagggaaacacctttgtccaagtcccctatttcccggaccttctaagc |
| | | ccttttgggtgctggtggtggttggtggagtcctggcttgctatagcttgctagtaacag |
| | | tggcctttattattttctgggtgaggagtaagaggagcaggctcctgcacagtgacta |
| | | catgaacatgactccccgccgccccgggcccacccgcaagcattaccagcccta |
| | | tgccccaccacgcgacttcgcagcctatcgctccagagtgaagttcagcaggagc |
| | | gcagacgcccccgcgtaccagcagggccagaaccagctctataacgagctcaa |
| | | tctaggacgaagagaggagtacgatgttttggacaagagacgtggccgggaccc |
| | | tgagatggggggaaagccgcagagaaggaagaaccctcaggaaggcctgtac |
| | | aatgaactgcagaaagataagatggcggaggcctacagtgagattgggatgaa |
| | | aggcgagcgccggaggggcaaggggcacgatggcctttaccagggtctcagta |
| | | cagccaccaaggacacctacgacgcccttcacatgcaggccctgccccctcgct |
| | | aataa |
| 138 | CAR1 CRT1: | M P R G W T A L C L L S L L P S G F M S L D |
| | truncated CD34 | N N G T A T P E L P T Q G T F S N V S T N V |
| | plus peptide 2A | S Y Q E T T T P S T L G S T S L H P V S Q H |
| | linker (bold), scFv | G N E A T T N I T E T T V K F T S T S V I T S |
| | CRT1 (italics), | V Y G N T N S S V Q S Q T S V I S T V F T T |
| | CD28-CD3 zeta, | P A N V S T P E T T L K P S L S P G N V S D |
| | rest of sequence | L S T T S T S L A T S P T K P Y T S S S P I L |
| | | S D I K A E I K C S G I R E V K L T Q G I C L |
| | | E Q N K T S S C A E F K K D R G E G L A R V |
| | | L C G E E Q A D A D A G A Q V C S L L L A |
| | | Q S E V R P Q C L L L V L A N R T E I S S K |
| | | L Q L M K K H Q S D L K K L G I L D F T E Q |
| | | D V A S H Q S Y S Q K T L I A L V T S G A L |
| | | L A V L G I T G Y F L M N R R S W S P T G E |
| | | R L E L E P V D R V K Q T L N F D L L K L A |
| | | G D V E S N P G P G N M G V L L T Q R T L L |
| | | S L V L A L L F P S M A S |
| | | MAEVQLQQSGAELMKPGASVKISCKATGYTFSSYWIEWVK |
| | | QRPGHGLEWIGEILPGSGSTNYNEKFKGKATFTADTSSNT |
| | | AYMQLSSLTSEDSAVYYCARGGDYDEEYYVMDYWGQGT |
| | | SVTVSSGGGGSGGGGSGGGGSQIVLTQSPAIMSASPGEK |
| | | VTMTCSASSSVSYMYWYQQKPGSSPRLLIYDTSNLASGVP |
| | | VRFSGSGSGTSYSLTISRMEAEDAATYYCQQWSSYPLTFG |
| | | AGTKLELKRAAA I E V M Y P P P Y L D N E K S N |
| | | G T I I H V K G K H L C P S P L F P G P S K P |
| | | F W V L V V V G G V L A C Y S L L V T V A F I |
| | | I F W V R S K R S R L L H S D Y M N M T P R |
| | | R P G P T R K H Y Q P Y A P P R D F A A Y R |
| | | S R V K F S R S A D A P A Y Q Q G Q N Q L Y |
| | | N E L N L G R R E E Y D V L D K R R G R D P |
| | | E M G G K P Q R R K N P Q E G L Y N E L Q |
| | | K D K M A E A Y S E I G M K G E R R R G K G |
| | | H D G L Y Q G L S T A T K D T Y D A L H M Q |
| | | A L P P R |
| 139 | CAR1 CRT1: | atgcctcgcggctggacagccctgtgcctgctgtctctgctgccatccggct |
| | truncated CD34 | tcatgagcctggataataacggcacagccaccccagagctgcctacacag |
| | plus peptide 2A | ggcaccttcagcaatgtgtccacaaacgtgagctatcaggagaccacaac |
| | linker (bold), scFv | cccttctaccctgggatccacaagcctgcaccccgtgtctcagcacggcaa |
| | CRT1 (italics), | cgaagccaccaccaacatcaccgagaccacagtgaagtttacctccacct |
| | CD28-CD3 zeta, | ctgtgattacctctgtgtacggaaatacaaactccagcgtgcagtctcagac |
| | rest of sequence | atctgtgatctccacagtgtttacaacacctgccaatgtgtccaccccagaga |
| | (nucleotide) | caaccctgaagcccagcctgtctcctggaaatgtgtccgatctgtctaccac |
| | | ctccaccagcctggccacctctcccaccaagccctatacctcctcttctccc |
| | | atcctgagcgatatcaaagccgagatcaaatgcagcgggattcgggaagt |
| | | gaaactgacacagggcatctgcctggaacagaataagacatccagctgcg |
| | | ccgagtttaagaaagatagaggagagggactggccagggtgctgtgtggc |
| | | gaagagcaggccgacgccgatgccggcgcccaggtgtgttccctgctgct |
| | | ggcccagtctgaggtgcgcccccagtgcctgctgctggtgctggccaatcg |
| | | gacagaaattagcagcaagctgcagctgatgaaaaaacaccagagcgatc |
| | | tgaaaaagctgggcatcctggactttaccgagcaggacgtggcctctcacc |
| | | agagctacagccagaaaacactgatcgccctggtgaccagcggagccct |
| | | gctggccgtgctgggcatcaccggatatttcctgatgaataggcgcagctg |
| | | gagccccaccggcgagcggctggagctggagcctgtcgaccgagtgaa |
| | | gcagaccctgaactttgatctgctgaagctggccggcgacgtggagtccaa |
| | | ccccgggccagggaatatgggcgtgctgctgacccagaggaccctgctg |
| | | agcctggtgctggccctgctgtttccatctatggcatcg |
| | | ATGGCCGAGGTTCAGCTTCAGCAGTCTGGAGCTGAGCT |
| | | GATGAAGCCTGGGGCCTCAGTGAAGATATCCTGCAAGG |
| | | CTACTGGCTACACATTCAGTAGCTACTGGATAGAGTGGG |
| | | TAAAGCAGAGGCCTGGACATGGCCTTGAGTGGATTGGA |
| | | GAGATTTTACCTGGAAGTGGTAGTACTAATTACAATGAG |
| | | AAGTTCAAGGGCAAGGCCACATTCACTGCAGATACATCC |
| | | TCCAACACAGCCTACATGCAACTCAGCAGCCTGACATCT |
| | | GAGGACTCTGCCGTCTATTACTGTGCAAGAGGGGGGGA |
| | | TTACGACGAAGAATACTATGTCATGGACTACTGGGGTCA |
| | | AGGAACCTCAGTCACTGTCTCCTCAGGTGGAGGCGGTT |
| | | CAGGCGGAGGTGGCTCTGGCGGTGGCGGATCGCAAAT |
| | | TGTTCTCACCCAGTCTCCAGCAATCATGTCTGCATCTCC |
| | | AGGGGAGAAGGTCACCATGACCTGCAGTGCCAGCTCAA |
| | | GTGTAAGTTACATGTACTGGTACCAGCAGAAGCCAGGAT |
| | | CCTCCCCCAGACTCCTGATTTATGACACATCCAACCTGG |
| | | CTTCTGGAGTCCCTGTTCGCTTCAGTGGCAGTGGGTCT |
| | | GGGACCTCTTACTCTCTCACAATCAGCCGAATGGAGGC |
| | | TGAAGATGCTGCCACTTATTACTGCCAGCAGTGGAGTAG |
| | | TTACCCGCTCACGTTCGGTGCTGGGACCAAGCTGGAGC |
| | | TGAAACGTGCGGCCGCAaattgaagttatgtatcctcctccttacctaga |
| | | caatgagaagagcaatggaaccattatccatgtgaaagggaaacacctttgtcca |
| | | agtcccctatttcccggaccttctaagcccttttgggtgctggtggtggttggtggagtc |
| | | ctggcttgctatagcttgctagtaacagtggcctttattattttctgggtgaggagtaag |
| | | aggagcaggctcctgcacagtgactacatgaacatgactccccgccgccccggg |
| | | cccacccgcaagcattaccagccctatgccccaccacgcgacttcgcagcctatc |
| | | gctccagagtgaagttcagcaggagcgcagacgcccccgcgtaccagcagggc |
| | | cagaaccagctctataacgagctcaatctaggacgaagagaggagtacgatgttt |
| | | tggacaagagacgtggccgggaccctgagatggggggaaagccgcagagaa |
| | | ggaagaaccctcaggaaggcctgtacaatgaactgcagaaagataagatggcg |
| | | gaggcctacagtgagattgggatgaaaggcgagcgccggaggggcaaggggc |
| | | acgatggcctttaccagggtctcagtacagccaccaaggacacctacgacgccct |
| | | tcacatgcaggccctgccccctcgctaataa |
| 140 | CAR3 CRT3: | M P R G W T A L C L L S L L P S G F M S L D |
| | truncated CD34 | N N G T A T P E L P T Q G T F S N V S T N V |
| | plus peptide 2A | S Y Q E T T T P S T L G S T S L H P V S Q H |
| | linker (bold), scFv | G N E A T T N I T E T T V K F T S T S V I T S |
| | CRT3 (italics), | V Y G N T N S S V Q S Q T S V I S T V F T T |
| | CD28-CD3 zeta, | P A N V S T P E T T L K P S L S P G N V S D |
| | rest of sequence | L S T T S T S L A T S P T K P Y T S S S P I L |
| | | S D I K A E I K C S G I R E V K L T Q G I C L |
| | | E Q N K T S S C A E F K K D R G E G L A R V |
| | | L C G E E Q A D A D A G A Q V C S L L L A |
| | | Q S E V R P Q C L L L V L A N R T E I S S K |
| | | L Q L M K K H Q S D L K K L G I L D F T E Q |
| | | D V A S H Q S Y S Q K T L I A L V T S G A L |
| | | L A V L G I T G Y F L M N R R S W S P T G E |
| | | R L E L E P V D R V K Q T L N F D L L K L A |
| | | G D V E S N P G P G N M G V L L T Q R T L L |
| | | S L V L A L L F P S M A S M A E V Q L Q Q S |
| | | G T V L A R P G A S V K M S C K A S G Y T F |
| | | T S Y W M H W V K Q R P G Q G L E W I G A I |
| | | Y P G N S D T S Y N Q K F K G K A K L T A V |
| | | T S T S T A Y M E L S S L T N E D S A V F Y |
| | | C T H Y Y G S D Y A M D Y W G Q G T S V T |
| | | V S S G G G G S G G G G S G G G G S Q I V |
| | | L T Q S P A I M S A S L G E R V T M T C T A |
| | | S S S V S S S Y L H W Y Q Q K P G S S P K L |
| | | W I Y S T S N L A S G V P A R F S G S G S G |
| | | T S Y S L T I S S M E A E D A A T Y Y C H Q |
| | | Y H R S P R T F G G G T K L E I K R A A A I E |
| | | V M Y P P P Y L D N E K S N G T I I H V K G |
| | | K H L C P S P L F P G P S K P F W V L V V V |
| | | G G V L A C Y S L L V T V A F I I F W V R S K |
| | | R S R L L H S D Y M N M T P R R P G P T R K |
| | | H Y Q P Y A P P R D F A A Y R S R V K F S R |
| | | S A D A P A Y Q Q G Q N Q L Y N E L N L G R |
| | | R E E Y D V L D K R R G R D P E M G G K P |
| | | Q R R K N P Q E G L Y N E L Q K D K M A E A |
| | | Y S E I G M K G E R R R G K G H D G L Y Q G |
| | | L S T A T K D T Y D A L H M Q A L P P R |
| 141 | CAR3 CRT3: | ATGCCTCGCGGCTGGACAGCCCTGTGCCTGCTGTCTCT |
| | truncated CD34 | GCTGCCATCCGGCTTCATGAGCCTGGATAATAACGGCA |
| | plus peptide 2A | CAGCCACCCCAGAGCTGCCTACACAGGGCACCTTCAG |
| | linker (bold), scFv | CAATGTGTCCACAAACGTGAGCTATCAGGAGACCACA |
| | CRT3 (italics), | ACCCCTTCTACCCTGGGATCCACAAGCCTGCACCCCGT |
| | D28-CD3 zeta, | GTCTCAGCACGGCAACGAAGCCACCACCAACATCACC |
| | rest of sequence | GAGACCACAGTGAAGTTTACCTCCACCTCTGTGATTAC |
| | (nucleotide) | CTCTGTGTACGGAAATACAAACTCCAGCGTGCAGTCTC |
| | | AGACATCTGTGATCTCCACAGTGTTTACAACACCTGCC |
| | | AATGTGTCCACCCCAGAGACAACCCTGAAGCCCAGCC |
| | | TGTCTCCTGGAAATGTGTCCGATCTGTCTACCACCTCC |
| | | ACCAGCCTGGCCACCTCTCCCACCAAGCCCTATACCTC |
| | | CTCTTCTCCCATCCTGAGCGATATCAAAGCCGAGATCA |
| | | AATGCAGCGGGATTCGGGAAGTGAAACTGACACAGGG |
| | | CATCTGCCTGGAACAGAATAAGACATCCAGCTGCGCC |
| | | GAGTTTAAGAAAGATAGAGGAGAGGGACTGGCCAGGG |
| | | TGCTGTGTGGCGAAGAGCAGGCCGACGCCGATGCCGG |
| | | CGCCCAGGTGTGTTCCCTGCTGCTGGCCCAGTCTGAG |
| | | GTGCGCCCCCAGTGCCTGCTGCTGGTGCTGGCCAATC |
| | | GGACAGAAATTAGCAGCAAGCTGCAGCTGATGAAAAA |
| | | ACACCAGAGCGATCTGAAAAAGCTGGGCATCCTGGAC |
| | | TTTACCGAGCAGGACGTGGCCTCTCACCAGAGCTACA |
| | | GCCAGAAAACACTGATCGCCCTGGTGACCAGCGGAGC |
| | | CCTGCTGGCCGTGCTGGGCATCACCGGATATTTCCTGA |
| | | TGAATAGGCGCAGCTGGAGCCCCACCGGCGAGCGGCT |
| | | GGAGCTGGAGCCTGTCGACCGAGTGAAGCAGACCCTG |
| | | AACTTTGATCTGCTGAAGCTGGCCGGCGACGTGGAGT |
| | | CCAACCCCGGGCCAGGGAATATGGGCGTGCTGCTGAC |
| | | CCAGAGGACCCTGCTGAGCCTGGTGCTGGCCCTGCTG |
| | | TTTCCATCTATGGCATCGATGGCCGAGGTCCAGCTGCA |
| | | GCAGTCTGGGACTGTGCTGGCAAGGCCTGGGGCTTCA |
| | | GTGAAGATGTCCTGCAAGGCTTCTGGCTACACCTTTACC |
| | | AGCTACTGGATGCACTGGGTAAAACAGAGGCCTGGACA |
| | | GGGTCTGGAATGGATTGGCGCTATTTATCCTGGAAATAG |
| | | TGATACTAGCTACAACCAGAAGTTCAAGGGCAAGGCCA |
| | | AACTGACTGCAGTCACATCCACCAGCACTGCCTACATG |
| | | GAGCTCAGCAGCCTGACAAATGAGGACTCTGCGGTCTT |
| | | TTACTGTACACATTACTACGGTAGTGACTATGCTATGGA |
| | | CTACTGGGGTCAAGGAACCTCAGTCACTGTCTCCTCAG |
| | | GTGGAGGCGGTTCAGGCGGAGGTGGCTCTGGCGGTGG |
| | | CGGATCGCAAATTGTTCTCACCCAGTCTCCAGCAATCAT |
| | | GTCTGCATCTCTAGGGGAACGGGTCACCATGACCTGCA |
| | | CTGCCAGCTCAAGTGTAAGTTCCAGTTACTTGCACTGGT |
| | | ACCAGCAGAAGCCAGGATCCTCCCCCAAACTCTGGATT |
| | | TATAGCACATCCAACCTGGCTTCTGGAGTCCCAGCTCG |
| | | CTTCAGTGGCAGTGGGTCTGGGACCTCTTACTCTCTCAC |
| | | AATCAGCAGCATGGAGGCTGAAGATGCTGCCACTTATTA |
| | | CTGCCACCAGTATCATCGTTCCCCACGGACGTTCGGTG |
| | | GAGGCACCAAGCTGGAAATCAAACGTGCGGCCGCAAAT |
| | | TGAAGTTATGTATCCTCCTCCTTACCTAGACAATGAGAA |
| | | GAGCAATGGAACCATTATCCATGTGAAAGGGAAACACCT |
| | | TTGTCCAAGTCCCCTATTTCCCGGACCTTCTAAGCCCTT |
| | | TTGGGTGCTGGTGGTGGTTGGTGGAGTCCTGGCTTGCT |
| | | ATAGCTTGCTAGTAACAGTGGCCTTTATTATTTTCTGGGT |
| | | GAGGAGTAAGAGGAGCAGGCTCCTGCACAGTGACTACA |
| | | TGAACATGACTCCCCGCCGCCCCGGGCCCACCCGCAA |
| | | GCATTACCAGCCCTATGCCCCACCACGCGACTTCGCAG |
| | | CCTATCGCTCCAGAGTGAAGTTCAGCAGGAGCGCAGAC |
| | | GCCCCCGCGTACCAGCAGGGCCAGAACCAGCTCTATAA |
| | | CGAGCTCAATCTAGGACGAAGAGAGGAGTACGATGTTT |
| | | TGGACAAGAGACGTGGCCGGGACCCTGAGATGGGGGG |
| | | AAAGCCGCAGAGAAGGAAGAACCCTCAGGAAGGCCTGT |
| | | ACAATGAACTGCAGAAAGATAAGATGGCGGAGGCCTAC |
| | | AGTGAGATTGGGATGAAAGGCGAGCGCCGGAGGGGCA |
| | | AGGGGCACGATGGCCTTTACCAGGGTCTCAGTACAGCC |
| | | ACCAAGGACACCTACGACGCCCTTCACATGCAGGCCCT |
| | | GCCCCCTCGCTAATAA |
| 142 | CAR4 CRT4: | M P R G W T A L C L L S L L P S G F M S L D |
| | truncated CD34 | N N G T A T P E L P T Q G T F S N V S T N V |
| | plus peptide 2A | S Y Q E T T T P S T L G S T S L H P V S Q H |
| | linker (bold), scFv | G N E A T T N I T E T T V K F T S T S V I T S |
| | CRT4 (italics), | V Y G N T N S S V Q S Q T S V I S T V F T T |
| | CD28-CD3 zeta, | P A N V S T P E T T L K P S L S P G N V S D |
| | rest of sequence | L S T T S T S L A T S P T K P Y T S S S P I L |
| | | S D I K A E I K C S G I R E V K L T Q G I C L |
| | | E Q N K T S S C A E F K K D R G E G L A R V |
| | | L C G E E Q A D A D A G A Q V C S L L L A |
| | | Q S E V R P Q C L L L V L A N R T E I S S K |
| | | L Q L M K K H Q S D L K K L G I L D F T E Q |
| | | D V A S H Q S Y S Q K T L I A L V T S G A L |
| | | L A V L G I T G Y F L M N R R S W S P T G E |
| | | R L E L E P V D R V K Q T L N F D L L K L A |
| | | G D V E S N P G P G N M G V L L T Q R T L L |
| | | S L V L A L L F P S M A S |
| | | MAQVQLQQSGAELMKPGASVKISCKATGYTFSSYWIEWV |
| | | NRRPGHGLEWIGEILPGSGSTNYNEKFKGKATFTADTSSN |
| | | TAYMQLSSLTSEDSVVYYCARGGDYDEEYYLMDYWGQGT |
| | | TLTVSSGGGGSGGGGSGGGGSQIVLTQSPAIMSASPGEK |
| | | VTMTCSASSSVSYMYWYQQKPGSSPRLLIYDTSNLASGVP |
| | | VRFSGSGSGTSYSLTISRMEAEDAATYYCQQWSSYPLTFG |
| | | AGTKLEIKRAAA I E V M Y P P P Y L D N E K S N |
| | | G T I I H V K G K H L C P S P L F P G P S K P |
| | | F W V L V V V G G V L A C Y S L L V T V A F I |
| | | I F W V R S K R S R L L H S D Y M N M T P R |
| | | R P G P T R K H Y Q P Y A P P R D F A A Y R |
| | | S R V K F S R S A D A P A Y Q Q G Q N Q L Y |
| | | N E L N L G R R E E Y D V L D K R R G R D P |
| | | E M G G K P Q R R K N P Q E G L Y N E L Q |
| | | K D K M A E A Y S E I G M K G E R R R G K G |
| | | H D G L Y Q G L S T A T K D T Y D A L H M Q |
| | | A L P P R |
| 143 | CAR4 CRT4: | ATGCCTCGCGGCTGGACAGCCCTGTGCCTGCTGTCTCT |
| | truncated CD34 | GCTGCCATCCGGCTTCATGAGCCTGGATAATAACGGCA |
| | plus peptide 2A | CAGCCACCCCAGAGCTGCCTACACAGGGCACCTTCAG |
| | linker (bold), scFv | CAATGTGTCCACAAACGTGAGCTATCAGGAGACCACA |
| | CRT4 (italics), | ACCCCTTCTACCCTGGGATCCACAAGCCTGCACCCCGT |
| | CD28-CD3 zeta, | GTCTCAGCACGGCAACGAAGCCACCACCAACATCACC |
| | rest of sequence | GAGACCACAGTGAAGTTTACCTCCACCTCTGTGATTAC |
| | (nucleotide) | CTCTGTGTACGGAAATACAAACTCCAGCGTGCAGTCTC |
| | | AGACATCTGTGATCTCCACAGTGTTTACAACACCTGCC |
| | | AATGTGTCCACCCCAGAGACAACCCTGAAGCCCAGCC |
| | | TGTCTCCTGGAAATGTGTCCGATCTGTCTACCACCTCC |
| | | ACCAGCCTGGCCACCTCTCCCACCAAGCCCTATACCTC |
| | | CTCTTCTCCCATCCTGAGCGATATCAAAGCCGAGATCA |
| | | AATGCAGCGGGATTCGGGAAGTGAAACTGACACAGGG |
| | | CATCTGCCTGGAACAGAATAAGACATCCAGCTGCGCC |
| | | GAGTTTAAGAAAGATAGAGGAGAGGGACTGGCCAGGG |
| | | TGCTGTGTGGCGAAGAGCAGGCCGACGCCGATGCCGG |
| | | CGCCCAGGTGTGTTCCCTGCTGCTGGCCCAGTCTGAG |
| | | GTGCGCCCCCAGTGCCTGCTGCTGGTGCTGGCCAATC |
| | | GGACAGAAATTAGCAGCAAGCTGCAGCTGATGAAAAA |
| | | ACACCAGAGCGATCTGAAAAAGCTGGGCATCCTGGAC |
| | | TTTACCGAGCAGGACGTGGCCTCTCACCAGAGCTACA |
| | | GCCAGAAAACACTGATCGCCCTGGTGACCAGCGGAGC |
| | | CCTGCTGGCCGTGCTGGGCATCACCGGATATTTCCTGA |
| | | TGAATAGGCGCAGCTGGAGCCCCACCGGCGAGCGGCT |
| | | GGAGCTGGAGCCTGTCGACCGAGTGAAGCAGACCCTG |
| | | AACTTTGATCTGCTGAAGCTGGCCGGCGACGTGGAGT |
| | | CCAACCCCGGGCCAGGGAATATGGGCGTGCTGCTGAC |
| | | CCAGAGGACCCTGCTGAGCCTGGTGCTGGCCCTGCTG |
| | | TTTCCATCTATGGCATCGATGGCCCAGGTTCAGCTGCA |
| | | GCAGTCTGGAGCTGAGCTGATGAAGCCTGGGGCCTCAG |
| | | TGAAGATATCCTGCAAGGCTACTGGCTACACATTCAGTA |
| | | GCTACTGGATAGAGTGGGTAAACCGGAGGCCTGGACAT |
| | | GGCCTTGAGTGGATTGGAGAGATTTTACCTGGAAGTGG |
| | | TAGTACTAATTACAATGAGAAGTTCAAGGGCAAGGCCAC |
| | | ATTCACTGCAGATACATCCTCCAATACAGCCTACATGCA |
| | | ACTCAGCAGCCTCACATCTGAGGACTCTGTCGTCTATTA |
| | | CTGTGCGAGAGGGGGGGATTACGACGAAGAATACTATC |
| | | TCATGGACTACTGGGGTCAAGGCACCACTCTCACAGTC |
| | | TCCTCAGGTGGAGGCGGTTCAGGCGGAGGTGGCTCTG |
| | | GCGGTGGCGGATCGCAAATTGTTCTCACCCAGTCTCCA |
| | | GCAATCATGTCTGCATCTCCAGGGGAGAAGGTCACCAT |
| | | GACCTGCAGTGCCAGCTCAAGTGTAAGTTACATGTACTG |
| | | GTACCAGCAGAAGCCAGGATCCTCCCCCAGACTCCTGA |
| | | TTTATGACACATCCAACCTGGCTTCTGGAGTCCCTGTTC |
| | | GCTTCAGTGGCAGTGGGTCTGGGACCTCTTACTCTCTC |
| | | ACAATCAGCCGAATGGAGGCTGAAGATGCTGCCACTTA |
| | | TTACTGCCAGCAGTGGAGTAGTTACCCGCTCACGTTCG |
| | | GTGCTGGGACCAAGCTGGAAATCAAACGTGCGGCCGCA |
| | | AATTGAAGTTATGTATCCTCCTCCTTACCTAGACAATGAG |
| | | AAGAGCAATGGAACCATTATCCATGTGAAAGGGAAACAC |
| | | CTTTGTCCAAGTCCCCTATTTCCCGGACCTTCTAAGCCC |
| | | TTTTGGGTGCTGGTGGTGGTTGGTGGAGTCCTGGCTTG |
| | | CTATAGCTTGCTAGTAACAGTGGCCTTTATTATTTTCTGG |
| | | GTGAGGAGTAAGAGGAGCAGGCTCCTGCACAGTGACTA |
| | | CATGAACATGACTCCCCGCCGCCCCGGGCCCACCCGC |
| | | AAGCATTACCAGCCCTATGCCCCACCACGCGACTTCGC |
| | | AGCCTATCGCTCCAGAGTGAAGTTCAGCAGGAGCGCAG |
| | | ACGCCCCCGCGTACCAGCAGGGCCAGAACCAGCTCTAT |
| | | AACGAGCTCAATCTAGGACGAAGAGAGGAGTACGATGT |
| | | TTTGGACAAGAGACGTGGCCGGGACCCTGAGATGGGG |
| | | GGAAAGCCGCAGAGAAGGAAGAACCCTCAGGAAGGCC |
| | | TGTACAATGAACTGCAGAAAGATAAGATGGCGGAGGCC |
| | | TACAGTGAGATTGGGATGAAAGGCGAGCGCCGGAGGG |
| | | GCAAGGGGCACGATGGCCTTTACCAGGGTCTCAGTACA |
| | | GCCACCAAGGACACCTACGACGCCCTTCACATGCAGGC |
| | | CCTGCCCCCTCGCTAATAA |
| 144 | Truncated CD34 | M P R G W T A L C L L S L L P S G F M S L D |
| | plus peptide 2A | N N G T A T P E L P T Q G T F S N V S T N V |
| | linker | S Y Q E T T T P S T L G S T S L H P V S Q H |
| | | G N E A T T N I T E T T V K F T S T S V I T S |
| | | V Y G N T N S S V Q S Q T S V I S T V F T T |
| | | P A N V S T P E T T L K P S L S P G N V S D |
| | | L S T T S T S L A T S P T K P Y T S S S P I L |
| | | S D I K A E I K C S G I R E V K L T Q G I C L |
| | | E Q N K T S S C A E F K K D R G E G L A R V |
| | | L C G E E Q A D A D A G A Q V C S L L L A Q |
| | | S E V R P Q C L L L V L A N R T E I S S K L Q |
| | | L M K K H Q S D L K K L G I L D F T E Q D V |
| | | A S H Q S Y S Q K T L I A L V T S G A L L A V |
| | | L G I T G Y F L M N R R S W S P T G E R L E |
| | | L E P V D R V K Q T L N F D L L K L A G D V |
| | | E S N P G P G N M G V L L T Q R T L L S L V |
| | | L A L L F P S M A S |
| 145 | Truncated CD34 | ATGCCTCGCGGCTGGACAGCCCTGTGCCTGCTGTCTCT |
| | plus peptide 2A | GCTGCCATCCGGCTTCATGAGCCTGGATAATAACGGCA |
| | linker (nucleotide | CAGCCACCCCAGAGCTGCCTACACAGGGCACCTTCAGC |
| | | AATGTGTCCACAAACGTGAGCTATCAGGAGACCACAAC |
| | | CCCTTCTACCCTGGGATCCACAAGCCTGCACCCCGTGT |
| | | CTCAGCACGGCAACGAAGCCACCACCAACATCACCGAG |
| | | ACCACAGTGAAGTTTACCTCCACCTCTGTGATTACCTCT |
| | | GTGTACGGAAATACAAACTCCAGCGTGCAGTCTCAGAC |
| | | ATCTGTGATCTCCACAGTGTTTACAACACCTGCCAATGT |
| | | GTCCACCCCAGAGACAACCCTGAAGCCCAGCCTGTCTC |
| | | CTGGAAATGTGTCCGATCTGTCTACCACCTCCACCAGCC |
| | | TGGCCACCTCTCCCACCAAGCCCTATACCTCCTCTTCTC |
| | | CCATCCTGAGCGATATCAAAGCCGAGATCAAATGCAGC |
| | | GGGATTCGGGAAGTGAAACTGACACAGGGCATCTGCCT |
| | | GGAACAGAATAAGACATCCAGCTGCGCCGAGTTTAAGA |
| | | AAGATAGAGGAGAGGGACTGGCCAGGGTGCTGTGTGG |
| | | CGAAGAGCAGGCCGACGCCGATGCCGGCGCCCAGGTG |
| | | TGTTCCCTGCTGCTGGCCCAGTCTGAGGTGCGCCCCCA |
| | | GTGCCTGCTGCTGGTGCTGGCCAATCGGACAGAAATTA |
| | | GCAGCAAGCTGCAGCTGATGAAAAAACACCAGAGCGAT |
| | | CTGAAAAAGCTGGGCATCCTGGACTTTACCGAGCAGGA |
| | | CGTGGCCTCTCACCAGAGCTACAGCCAGAAAACACTGA |
| | | TCGCCCTGGTGACCAGCGGAGCCCTGCTGGCCGTGCT |
| | | GGGCATCACCGGATATTTCCTGATGAATAGGCGCAGCT |
| | | GGAGCCCCACCGGCGAGCGGCTGGAGCTGGAGCCTGT |
| | | CGACCGAGTGAAGCAGACCCTGAACTTTGATCTGCTGA |
| | | AGCTGGCCGGCGACGTGGAGTCCAACCCCGGGCCAGG |
| | | GAATATGGGCGTGCTGCTGACCCAGAGGACCCTGCTGA |
| | | GCCTGGTGCTGGCCCTGCTGTTTCCATCTATGGCATCG |
| 146 | CD28 | I E V M Y P P P Y L D N E K S N G T I I H V K |
| | | G K H L C P S P L F P G P S K P F W V L V V |
| | | V G G V L A C Y S L L V T V A F I I F W V R S |
| | | K R S R L L H S D Y M N M T P R R P G P T R |
| | | K H Y Q P Y A P P R D F A A Y R S |
| 147 | CD28 nucleotide | AATTGAAGTTATGTATCCTCCTCCTTACCTAGACAATGAG |
| | | AAGAGCAATGGAACCATTATCCATGTGAAAGGGAAACAC |
| | | CTTTGTCCAAGTCCCCTATTTCCCGGACCTTCTAAGCCC |
| | | TTTTGGGTGCTGGTGGTGGTTGGTGGAGTCCTGGCTTG |
| | | CTATAGCTTGCTAGTAACAGTGGCCTTTATTATTTTCTGG |
| | | GTGAGGAGTAAGAGGAGCAGGCTCCTGCACAGTGACTA |
| | | CATGAACATGACTCCCCGCCGCCCCGGGCCCACCCGC |
| | | AAGCATTACCAGCCCTATGCCCCACCACGCGACTTCGC |
| | | AGCCTATCGCTCC |
| 148 | CD3 zeta | R V K F S R S A D A P A Y Q Q G Q N Q L Y N |
| | | E L N L G R R E E Y D V L D K R R G R D P E |
| | | M G G K P Q R R K N P Q E G L Y N E L Q K |
| | | D K M A E A Y S E I G M K G E R R R G K G H |
| | | D G L Y Q G L S T A T K D T Y D A L H M Q A |
| | | L P P R |
| 149 | CD3 zeta | AGAGTGAAGTTCAGCAGGAGCGCAGACGCCCCCGCGT |
| | (nucleotide) | ACCAGCAGGGCCAGAACCAGCTCTATAACGAGCTCAAT |
| | | CTAGGACGAAGAGAGGAGTACGATGTTTTGGACAAGAG |
| | | ACGTGGCCGGGACCCTGAGATGGGGGGAAAGCCGCAG |
| | | AGAAGGAAGAACCCTCAGGAAGGCCTGTACAATGAACT |
| | | GCAGAAAGATAAGATGGCGGAGGCCTACAGTGAGATTG |
| | | GGATGAAAGGCGAGCGCCGGAGGGGCAAGGGGCACGA |
| | | TGGCCTTTACCAGGGTCTCAGTACAGCCACCAAGGACA |
| | | CCTACGACGCCCTTCACATGCAGGCCCTGCCCCCTCGC |
| | | TAATAA |
| 150 | Consensus | S/T SYW I/M E/H |
| | sequence for | |
| | CRT1, 3, 4 and 5 | |
| | V1 heavy chain | |
| | CDR1 | |
| 151 | Consensus | GYTF S/T SYW |
| | sequence for | |
| | CRT1, 3, 4 and 5 | |
| | V2 heavy chain | |
| | CDR1 | |
| 152 | Consensus | WIG E/A I L/Y PG S/N G/S S/D T N/S |
| | sequence for | |
| | CRT1, 3, 4 and 5 | |
| | V1 heavy chain | |
| | CDR2 | |
| 153 | Consensus | I L/Y PG S/N G/S S/D T |
| | sequence for | |
| | CRT1, 3, 4 and 5 | |
| | V2 heavy chain | |
| | CDR2 | |
| 154 | Consensus | A/T R/H G/X G/X D/X Y D/Y E/G E/S Y/D Y V/A/L MD |
| | sequence for | |
| | CRT1, 3, 4 and 5 | |
| | V1 heavy chain | |
| | CDR3 | |
| 155 | Consensus | A/T R/H G/X G/X D/X Y D/Y E/G E/S Y/D Y V/A/L MDY |
| | sequence for | |
| | CRT1, 3, 4 and 5 | |
| | V2 heavy chain | |
| | CDR3 | |
| 156 | Consensus | S/X S/X S Y M/L Y/H W Y |
| | sequence for | |
| | CRT1, 3, 4 and 5 | |
| | V1 light chain | |
| | CDR1 | |
| 157 | Consensus | SSVS Y/S S/X Y/X |
| | sequence for | |
| | CRT1, 3, 4, and 5 | |
| | V2 light chain | |
| | CDR1 | |
| 158 | Consensus | L L/W IY D/S TSNLA |
| | sequence for | |
| | CRT1, 3, 4 and 5 | |
| | V1 light chain | |
| | CDR2 | |
| | Consensus | D/S TS |
| | sequence for | |
| | CRT1, 3, 4 and 5 | |
| | V2 light chain | |
| | CDR2 | |
| 160 | Consensus | Q/H Q W/Y S/H S/R Y/S P L/R |
| | sequence for | |
| | CRT1, 3, 4 and 5 | |
| | V1 light chain | |
| | CDR3 | |
| 161 | Consensus | Q/H Q W/Y S/H S/R Y/S P L/R T F/X |
| | sequence for | |
| | CRT1, 3, 4 and 5 | |
| | V2 light chain | |
| | CDR3 | |
| 119 | Nucleotide | ATCTACATCTGGGCGCCCTTGGCCGGGACTTGTGGGGT |
| | sequence for | CCTTCTCCTGTCACTGGTTATCACCCTTTACTGC |
| | CD8α | |
| | transmembrane | |
| | domain | |
| 162 | Nucleotide | ATGGCCTTACCAGTGACCGCCTTGCTCCTGCCGCTGGC |
| | sequence of | CTTGCTGCTCCACGCCGCCAGGCCG |
| | CD8α leader | |
| 163 | CH2CH3 hinge | DPAEPKSPDK THTCPPCPAP ELLGGPSVFL |
| | | FPPKPKDTLM ISRTPEVTCV |
| | | VVDVSHEDPEVKFNWYVDGV EVHNAKTKPR |
| | | EEQYNSTYRV VSVLTVLHQD WLNGKEYKCK |
| | | VSNKALPAPI EKTISKAKGQ PREPQVYTLP |
| | | PSRDELTKNQ VSLTCLVKGF YPSDIAVEWE |
| | | SNGQPENNYKTTPPVLDSDG SFFLYSKLTV |
| | | DKSRWQQGNV FSCSVMHEAL HNHYTQKSLS |
| | | LSPGKKDPK |
| 164 | Nucleotide for | AAAATCTTGTGACAAAACTCACACATGCCCACCGTGCCC |
| | CH2CH3 hinge | AGCACCTGAACTCCTGGGGGGACCGTCAGTCTTCCTCT |
| | | TCCCCCCAAAACCCAAGGACACCCTCATGATCTCCCGG |
| | | ACCCCTGAGGTCACATGCGTGGTGGTGGACGTGAGCCA |
| | | CGAAGACCCTGAGGTCAAGTTCAACTGGTACGTGGACG |
| | | GCGTGGAGGTGCATAATGCCAAGACAAAGCCGCGGGA |
| | | GGAGCAGTACAACAGCACGTACCGTGTGGTCAGCGTCC |
| | | TCACCGTCCTGCACCAGGACTGGCTGAATGGCAAGGAG |
| | | TACAAGTGCAAGGTCTCCAACAAAGCCCTCCCAGCCCC |
| | | CATCGAGAAAACCATCTCCAAAGCCAAAGGGCAGCCCC |
| | | GAGAACCACAGGTGTACACCCTGCCCCCATCCCGGGAT |
| | | GAGCTGACCAAGAACCAGGTCAGCCTGACCTGCCTGGT |
| | | CAAAGGCTTCTATCCCAGCGACATCGCCGTGGAGTGGG |
| | | AGAGCAATGGGCAGCCGGAGAACAACTACAAGACCACG |
| | | CCTCCCGTGCTGGACTCCGACGGCTCCTTCTTCCTCTA |
| | | CAGCAAGCTCACCGTGGACAAGAGCAGGTGGCAGCAG |
| | | GGGAACGTCTTCTCATGCTCCGTGATGCATGAGGGTCT |
| | | GCACAACCACTACACGCAGAAGAGCCTCTCCCTGTCTC |
| | | CGGGTAAAGGGCCGGCCGCT |
| 165 | CD8α hinge | FVPVFLPAKP TTTPAPRPPT PAPTIASQPL SLRPEACRPA |
| | | AGGAVHTRGL DFACD |
| 166 | Shortened IgG | AEPKSPDKTHTCP |
| | hinge | |
| 159 | Linker | KDPK |
| 86 | OX40 (CD134) | RDQRLPPDAH KPPGGGSFRT PIQEEQADAH STLAKI |
| | co-stimulatory | |
| | domain | |
| 80 | Nucleotide for 4- | AAACGGGGCAGAAAGAAACTCCTGTATATATTCAAACAA |
| | 1BB | CCATTTATGAGACCAGTACAAACTACTCAAGAGGAAGAT |
| | costimulatory | GGCTGTAGCTGCCGATTTCCAGAAGAAGAAGAAGGAGG |
| | domain | ATGTGAACTG |
| 54 | Nucleotide for | TTTTGGGTGCTGGTGGTGGTTGGTGGAGTCCTGGCTTG |
| | CD28 | CTATAGCTTGCTAGTAACAGTGGCCTTTATTATTTTCTGG |
| | costimulatory | GTGAGGAGTAAGAGGAGCAGGCTCCTGCACAGTGACTA |
| | domain | CATGAACATGACTCCCCGCCGCCCCGGGCCCACCCGC |
| | | AAGCATTACCAGCCCTATGCCCCACCACGCGACTTCGC |
| | | AGCCTATCGCTCC |
| 48 | Nucleotide for | AGGGACCAGAGGCTGCCCCCCGATGCCCACAAGCCCC |
| | OX40 | CTGGGGGAGGCAGTTTCCGGACCCCCATCCAAGAGGA |
| | costimulatory | GCAGGCCGACGCCCACTCCACCCTGGCCAAGATC |
| | domain | |

CRT2 heavy chain CDRs and sequences

| | | |
|---|---|---|
| 167 | Heavy chain | GFTFNTYA |
| | CDR1 | |
| 168 | Heavy chain | IRSKSNNYAT |
| | CDR2 | |
| 169 | Heavy chain | VREGVYYYGSSGYYAMDY |
| | CDR3 | |
| 170 | Nt heavy chain | GGTTTCACCTTCAATACCTATGCC |
| | CDR1 | |
| 171 | Nt heavy chain | ATAAGAAGTAAAAGTAATAATTATGCAACA |
| | CDR2 | |
| 172 | Nt heavy chain | GTGAGAGAAGGGGTTTATTACTACGGTAGTAGTGGGTACTATGCTATGGACTAC |
| | CDR3 | |
| 173 | Heavy chain | M A E V Q G V E S G G G L V Q P K G S L K L S C A A S |
| | | G F T F N T Y A M H W V C Q A P G K G L E W V A R I |
| | | R S K S N N Y A T Y Y A D S V K D R F T I S R D D S Q S |
| | | M L Y L Q M N N L K T E D T A M Y Y C V R E G V Y Y |
| | | Y G S S G Y Y A M D Y W G Q G T S V T V S S G |
| 174 | Nt heavy chain | GACGCTTATCGATGGCCGAGGTGCAGGGGGTGGAGTCTGGTGGAGGATTGGTGCA |
| | | GCCTAAAGGATCATTGAAACTCTCATGTGCCGCCTCTGGTTTCACCTTCAATACC |
| | | TATGCCA |
| | | TGCACTGGGTCTGCCAGGCTCCAGGAAAGGGTTTGGAATGGGTTGCTCGCATAAG |
| | | AAGTAAAAGTAATAATTATGCAACATATTATGCCGATTCAGTGAAAGACAGATTC |
| | | ACCATCTCCAGAGATGATTCACAAAGCATGCTCTATCTGCAAATGAACAACCTGA |
| | | AAACTGAGGACACAGCCATGTATTACTGTGTGAGAGAAGGGGTTTATTACTACGG |
| | | TAGTAGTGGGTACTATGCTATGGACTACTGGGGTCAAGGAACCTCAGTCACCGTC |
| | | TCCTCAGGT |
| 175 | CRT2 scFv | M A E V Q G V E S G G G L V Q P K G S L K L S C A A S |
| | | G F T F N T Y A M H W V C Q A P G K G L E W V A R I |
| | | R S K S N N Y A T Y Y A D S V K D R F T I S R D D S Q S |
| | | M L Y L Q M N N L K T E D T A M Y Y C V R E G V Y Y |
| | | Y G S S G Y Y A M D Y W G Q G T S V T V S S GGGGGS |
| | | GGGGSGGGGSEIVLTQSPAIMSASPGEKVTITCSASSSVS |
| | | YMHWFQQKPGTSPKLWIYSTSNLASGVPARFSGSGSGTS |
| | | YSLTISRMEAEDAATYYCQQRSSYPLTFGAPGKLELKRAA |
| 176 | CRT2 scFv | GACGCTTATCGATGGCCGAGGTGCAGGGGGTGGAGTCTGGTGGAGGATTGGTGCA |
| | Nucleotide | GCCTAAAGGATCATTGAAACTCTCATGTGCCGCCTCTGGTTTCACCTTCAATACC |
| | | TATGCCA |
| | | TGCACTGGGTCTGCCAGGCTCCAGGAAAGGGTTTGGAATGGGTTGCTCGCATAAG |
| | | AAGTAAAAGTAATAATTATGCAACATATTATGCCGATTCAGTGAAAGACAGATTC |
| | | ACCATCTCCAGAGATGATTCACAAAGCATGCTCTATCTGCAAATGAACAACCTGA |
| | | AAACTGAGGACACAGCCATGTATTACTGTGTGAGAGAAGGGGTTTATTACTACGG |
| | | TAGTAGTGGGTACTATGCTATGGACTACTGGGGTCAAGGAACCTCAGTCACCGTC |
| | | TCCTCAGGT |
| | | TCCTCAGGTGGAGGCGGTTCAGGCGGAGGTGGCTCTG |
| | | GCGGTGGCGGATCG |
| | | GAAATTGTTCTCACCCAGTCTCCAGCAATCATGTCTGCA |
| | | TCTCCAGGGGAGAAGGTCACC |
| | | ATAACCTGCAGTGCCAGCTCAAGTGTAAGTTACATGCAC |
| | | TGGTTCCAGCAGAAG |
| | | CCAGGCACTTCTCCCAAACTCTGGATTTATAGCACATCC |
| | | AACCTGGCTTCTGGAGTCCCT |
| | | GCTCGCTTCAGTGGCAGTGGATCTGGGACCTCTTACTC |
| | | TCTCACAATCAGCCGAATGGAG |
| | | GCTGAAGATGCTGCCACTTATTACTGCCAGCAAAGGAGT |
| | | AGTTACCCCCTCACGTTCGGT |
| | | GCTGGGACCAAGCTGGAGCTGAAACGTGCGGCCGC |

Codon optimised scFv nucleotide sequences (hu - codon optimised for expression in
human cells, mu - codon optimised for expression in murine cells)

| | | |
|---|---|---|
| 177 | CRT1 scFv hu | ATGGCAGAGGTGCAGCTGCAGCAGAGCGGAGCAGAGCTGATGAAGC |
| | | CAGGAGCCTCTGTGAAGATCAGCTGTAAGGCCACCGGCTATACATTCA |
| | | GCTCCTACTGGATCGAGTGGGTGAAGCAGCGGCCTGGCCACGGCCTG |
| | | GAGTGGATCGGAGAGATCCTGCCAGGCAGCGGCTCCACCAACTATAA |
| | | TGAGAAGTTCAAGGGCAAGGCCACCTTTACAGCCGACACCTCTAGCAA |
| | | CACAGCCTACATGCAGCTGTCCTCTCTGACAAGCGAGGATTCCGCCGT |
| | | GTACTATTGCGCCAGGGGCGGCGACTATGATGAGGAGTACTATGTGA |
| | | TGGACTACTGGGGCCAGGGCACCTCCGTGACCGTGAGCAGCGGcGGA |
| | | GGcGGCAGCGGAGGAGGAGGCTCCGGCGGCGGCGGCTCTCAGATCG |
| | | TGCTGACCCAGAGCCCAGCAATCATGTCTGCCAGCCCAGGAGAGAAG |
| | | GTGACCATGACATGTTCCGCCTCTAGCTCCGTGAGCTACATGTATTGGT |
| | | ATCAGCAGAAGCCCGGCTCTAGCCCTCGGCTGCTGATCTATAGAACCT |
| | | CCAATCTGGCATCTGGCGTGCCCGCAAGGTTCTCCGGCTCTGGCAGCG |
| | | GCACCTCCTACTCTCTGACCATCGGCACAATGGAGGCCGAGGATGCCG |
| | | CCACATACTATTGCCAGCAGTGGTCCTCTTACCCTCTGACCTTTGGCGC |
| | | CGGCACAAAGCTGGAGATCAAGCGCGCGGCCGCA |
| 178 | CRT1 scFv mu | ATGGCTGAGGTGCAGCTGCAGCAGTCCGGAGCTGAGCTGATGAAGCC |
| | | AGGCGCCTCTGTGAAGATCAGCTGTAAGGCCACCGGCTACACATTCAG |
| | | CTCCTACTGGATCGAGTGGGTGAAGCAGAGGCCTGGCCACGGACTGG |
| | | AGTGGATCGGAGAGATCCTGCCAGGCAGCGGCAGCACCAACTACAAC |
| | | GAGAAGTTCAAGGGCAAGGCTACCTTTACAGCCGACACCTCTAGCAAC |
| | | ACAGCTTACATGCAGCTGTCCTCTCTGACAAGCGAGGATAGCGCCGTG |
| | | TACTACTGCGCCAGGGGCGGAGACTACGATGAGGAGTACTACGTGAT |
| | | GGACTACTGGGGCCAGGGAACCTCTGTGACCGTGAGCAGCGGAGGA |
| | | GGAGGAAGCGGCGGAGGAGGCAGCGGAGGAGGAGGATCTCAGATC |
| | | GTGCTGACCCAGAGCCCAGCTATCATGTCTGCCAGCCCCGGCGAGAAG |
| | | GTGACCATGACATGTAGCGCCTCTAGCTCCGTGTCCTACATGTACTGGT |
| | | ATCAGCAGAAGCCCGGATCTAGCCCTAGGCTGCTGATCTACAGAACAT |
| | | CCAACCTGGCTTCTGGCGTGCCCGCTCGGTTCTCCGGCTCTGGAAGCG |
| | | GCACCTCCTACTCTCTGACCATCGGCACAATGGAGGCTGAGGATGCCG |
| | | CTACATACTACTGCCAGCAGTGGTCCTCTTACCCTCTGACCTTTGGAGC |
| | | CGGCACAAAGCTGGAGATCAAGCGCGCGGCCGCA |
| 179 | CRT2 scFv hu | ATGGCAGAGGTGCAGGGAGTGGAGAGCGGAGGCGGCCTGGTGCAGC |
| | | CTAAGGGCTCCCTGAAGCTGTCTTGCGCCGCCAGCGGCTTCACCTTTAA |
| | | CACATATGCAATGCACTGGGTGTGCCAGGCACCAGGCAAGGGCCTGG |
| | | AGTGGGTGGCACGGATCAGAAGCAAGTCCAACAATTATGCCACCTACT |
| | | ATGCCGACAGCGTGAAGGATAGGTTCACAATCTCCCGCGACGATTCTC |
| | | AGAGCATGCTGTACCTGCAGATGAACAATCTGAAGACCGAGGACACA |
| | | GCCATGTACTATTGCGTGCGGGAGGGCGTGTACTATTACGGCAGCTCC |
| | | GGCTATTACGCTATGGACTACTGGGGCCAGGGCACCAGCGTGACAGT |
| | | GTCTAGCGGAGGAGGAGGCTCCGGAGGAGGAGGCTCTGGCGGCGGC |
| | | GGCAGCGAGATCGTGCTGACCCAGTCCCCAGCAATCATGTCCGCCTCT |
| | | CCAGGAGAGAAGGTGACCATCACATGCTCCGCCTCCTCTAGCGTGTCT |
| | | TATATGCACTGGTTCCAGCAGAAGCCCGGCACCTCTCCTAAGCTGTGG |
| | | ATCTACAGCACATCCAATCTGGCATCCGGCGTGCCCGCAAGGTTTTCTG |
| | | GCAGCGGCTCCGGCACCTCTTATAGCCTGACAATCAGCCGGATGGAG |
| | | GCAGAGGACGCAGCAACCTATTACTGTCAGCAGAGATCCTCTTACCCT |
| | | CTGACCTTTGGCGCCGGCACAAAGCTGGAGCTGAAGCGCGCGGCCGC |
| | | A |
| 180 | CRT2 scFv mu | ATGGCTGAGGTGCAGGGAGTGGAGAGCGGAGGAGGCCTGGTGCAGC |
| | | CTAAGGGCTCCCTGAAGCTGTCTTGCGCCGCTAGCGGATTCACCTTTAA |
| | | CACATACGCTATGCACTGGGTGTGCCAGGCTCCAGGAAAGGGCCTGG |
| | | AGTGGGTGGCCAGGATCAGAAGCAAGTCCAACAACTACGCTACCTACT |
| | | ACGCCGACAGCGTGAAGGATCGGTTCACAATCTCCCGCGACGATTCTC |
| | | AGAGCATGCTGTACCTGCAGATGAACAACCTGAAGACCGAGGACACA |
| | | GCTATGTACTACTGCGTGCGGGAGGGCGTGTACTACTACGGCAGCTCC |
| | | GGATACTACGCTATGGACTACTGGGGACAGGGCACCTCCGTGACAGT |
| | | GTCTAGCGGAGGAGGAGGCTCCGGAGGAGGAGGCTCTGGAGGCGGA |
| | | GGCAGCGAGATCGTGCTGACCCAGTCTCCAGCTATCATGTCCGCCTCT |
| | | CCCGGCGAGAAGGTGACCATCACATGCTCCGCCTCCTCTAGCGTGTCT |
| | | TACATGCACTGGTTCCAGCAGAAGCCCGGCACCTCTCCTAAGCTGTGG |
| | | ATCTACAGCACATCCAACCTGGCTAGCGGAGTGCCCGCTCGGTTTTCT |
| | | GGAAGCGGCTCCGGAACCTCTTACAGCCTGACAATCTCCAGGATGGA |
| | | GGCTGAGGACGCCGCTACATACTACTGTCAGCAGAGATCCTCTTACCC |
| | | TCTGACCTTTGGCGCCGGAACAAAGCTGGAGCTGAAGCGCGCGGCCG |
| | | CA |
| 181 | CRT3 scFv hu | ATGGCCGAGGTGCAGCTGCAGCAGTCTGGCACCGTGCTGGCCAGGCC |
| | | CGGAGCAAGCGTGAAGATGTCCTGCAAGGCCTCTGGCTACACCTTCAC |
| | | AAGCTATTGGATGCACTGGGTGAAGCAGCGCCCAGGACAGGGCCTGG |
| | | AGTGGATCGGAGCAATCTACCCCGGCAACTCCGACACCTCTTATAATC |
| | | AGAAGTTCAAGGGCAAGGCCAAGCTGACAGCCGTGACCTCTACAAGC |
| | | ACCGCCTACATGGAGCTGAGCAGCCTGACCAACGAGGATAGCGCCGT |
| | | GTTTTATTGCACACACTACTATGGCTCCGACTACGCTATGGACTATTGG |
| | | GGCCAGGGCACCTCCGTGACAGTGTCTAGCGGAGGAGGAGGCAGCG |
| | | GAGGAGGAGGCTCCGGCGGCGGCGGCTCTCAGATCGTGCTGACCCAG |
| | | AGCCCTGCCATCATGTCCGCCTCTCTGGGCGAGCGGGTGACAATGACC |
| | | TGTACAGCCTCCTCTAGCGTGTCCTCTAGCTACCTGCACTGGTATCAGC |
| | | AGAAGCCCGGCTCCTCTCCTAAGCTGTGGATCTACAGCACCTCCAATCT |
| | | GGCATCCGGCGTGCCTGCAAGGTTCTCTGGCAGCGGCTCCGGCACCTC |
| | | TTACAGCCTGACAATCAGCAGCATGGAGGCAGAGGACGCAGCAACAT |
| | | ACTATTGTCACCAGTATCACCGGAGCCCAAGAACCTTTGGCGGCGGCA |
| | | CAAAGCTGGAGATCAAGCGGGCGGCCGCA |
| 182 | CRT3 scFv mu | ATGGCCGAGGTGCAGCTGCAGCAGTCTGGCACCGTGCTGGCTCGGCC |
| | | CGGAGCTAGCGTGAAGATGTCCTGCAAGGCTTCTGGCTACACCTTCAC |
| | | AAGCTACTGGATGCACTGGGTGAAGCAGCGCCCAGGACAGGGCCTGG |
| | | AGTGGATCGGCGCCATCTACCCCGGAAACTCCGACACCTCTTACAACC |
| | | AGAAGTTCAAGGGCAAGGCTAAGCTGACAGCCGTGACCTCTACAAGC |
| | | ACCGCTTACATGGAGCTGAGCAGCCTGACCAACGAGGATAGCGCCGT |
| | | GTTTTACTGCACACACTACTACGGCTCCGACTACGCTATGGATTACTGG |
| | | GGACAGGGCACCTCCGTGACAGTGTCTAGCGGAGGAGGAGGAAGCG |
| | | GCGGAGGcGGCAGCGGAGGAGGAGGATCTCAGATCGTGCTGACCCA |
| | | GTCTCCTGCTATCATGTCCGCCTCTCTGGGCGAGAGGGTGACAATGAC |
| | | CTGTACAGCCTCCTCTAGCGTGTCCTCTAGCTACCTGCACTGGTATCAG |
| | | CAGAAGCCCGGCTCCTCTCCTAAGCTGTGGATCTACAGCACCTCCAACC |
| | | TGGCTTCCGGAGTGCCTGCTCGGTTCTCTGGAAGCGGCTCCGGAACCT |
| | | CTTACAGCCTGACAATCAGCAGCATGGAGGCTGAGGACGCCGCTACAT |
| | | ACTACTGTCACCAGTACCACAGGAGCCCAAGAACCTTTGGCGGAGGCA |
| | | CAAAGCTGGAGATCAAGAGGGCGGCCGCA |
| 183 | CRT4 scFv hu | ATGGCACAGGTGCAGCTGCAGCAGAGCGGAGCAGAGCTGATGAAGC |
| | | CAGGAGCCTCTGTGAAGATCAGCTGTAAGGCCACCGGCTATACATTCA |
| | | GCTCCTACTGGATCGAGTGGGTGAACAGACGGCCCGGCCACGGCCTG |
| | | GAGTGGATCGGAGAGATCCTGCCAGGCAGCGGCTCCACCAACTATAA |
| | | TGAGAAGTTCAAGGGCAAGGCCACCTTTACAGCCGACACCTCTAGCAA |
| | | TACAGCCTACATGCAGCTGTCCTCTCTGACAAGCGAGGATTCCGTGGT |
| | | GTACTATTGCGCCAGGGGCGGCGACTATGATGAGGAGTACTATCTGAT |
| | | GGACTACTGGGGCCAGGGCACCACACTGACCGTGAGCAGCGGAGGA |
| | | GGAGGCAGCGGAGGAGGAGGCTCCGGCGGCGGCGGCTCTCAGATCG |
| | | TGCTGACACAGTCCCCAGCAATCATGTCTGCCAGCCCAGGAGAGAAGG |
| | | TGACCATGACATGTTCCGCCTCTAGCTCCGTGAGCTACATGTATTGGTA |
| | | TCAGCAGAAGCCCGGCTCTAGCCCTAGGCTGCTGATCTATGACACCTC |
| | | CAACCTGGCATCTGGCGTGCCCGTGCGCTTCTCCGGCTCTGGCAGCGG |
| | | CACCTCCTACTCTCTGACAATCAGCCGGATGGAGGCAGAGGATGCAGC |
| | | AACCTACTATTGCCAGCAGTGGTCCTCTTACCCTCTGACCTTTGGCGCC |
| | | GGCACAAAGCTGGAGATCAAGCGGGCGGCCGCA |
| 184 | CRT4 scFv mu | ATGGCTCAGGTGCAGCTGCAGCAGTCCGGAGCTGAGCTGATGAAGCC |
| | | AGGCGCCTCTGTGAAGATCAGCTGTAAGGCCACCGGCTACACATTCAG |
| | | CTCCTACTGGATCGAGTGGGTGAACAGGAGGCCCGGCCACGGACTGG |
| | | AGTGGATCGGAGAGATCCTGCCAGGCAGCGGCAGCACCAACTACAAC |
| | | GAGAAGTTCAAGGGCAAGGCTACCTTTACAGCCGACACCTCTAGCAAC |
| | | ACAGCTTACATGCAGCTGTCCTCTCTGACAAGCGAGGATTCCGTGGTG |
| | | TACTACTGCGCCAGGGGCGGAGACTACGATGAGGAGTACTACCTGAT |
| | | GGACTACTGGGGCCAGGGAACCACACTGACCGTGAGCAGCGGAGGA |
| | | GGAGGAAGCGGCGGAGGAGGCAGCGGAGGAGGAGGATCTCAGATC |
| | | GTGCTGACACAGTCTCCAGCTATCATGTCTGCCAGCCCCGGCGAGAAG |
| | | GTGACCATGACATGTAGCGCCAGCAGCAGCGTGAGCTACATGTACTG |
| | | GTATCAGCAGAAGCCCGGATCTAGCCCTCGGCTGCTGATCTACGACAC |
| | | CTCCAACCTGGCTTCTGGCGTGCCCGTGCGCTTCTCCGGCTCTGGAAGC |
| | | GGCACCTCCTACTCTCTGACAATCAGCAGGATGGAGGCTGAGGATGCC |
| | | GCTACATACTACTGCCAGCAGTGGTCCTCTTACCCTCTGACCTTTGGAG |
| | | CCGGCACAAAGCTGGAGATCAAGAGGGCGGCCGCA |
| 185 | CRT5 scFv hu | ATGGCAGAGGTGCAGCTGCAGCAGTCCGGAGCAGAGCTGATGAAGCC |
| | | AGGAGCCTCTGTGAAGATCAGCTGTAAGGCCACCGGCTATACATTCAG |
| | | CTCCTACTGGATCGAGTGGGTGAACCAGCGCCCTGGCCACGGCCTGG |
| | | AGTGGATCGGAGAGATCCTGCCAGGCAGCGGCTCCACCAACTATAAT |
| | | GAGAAGTTCAAGGGCAAGGCCACCTTTACAGCCGACACCTCTAGCAAT |
| | | ACAGCCTACATGCAGCTGTCCTCTCTGACAAGCGAGGATTCCGCCGTG |
| | | TACTATTGCGCCAGAGGCGGCGACTATGATGAGGAGTACTATGCTATG |
| | | GACTACTGGGGCCAGGGCACCTCTGTGACCCTGAGCAGCGGAGGAGG |
| | | AGGCAGCGGcGGAGGAGGCTCCGGCGGCGGCGGCTCTCAGATCGTG |
| | | CTGACCCAGAGCCCAGCAATCATGTCTGCCAGCCCAGGAGAGAAGGT |
| | | GACCATGACATGTAGCGCCTCTAGCTCCGTGTCCTACATGTATTGGTAT |
| | | CAGCAGAAGCCCGGCTCTAGCCCTCGGCTGCTGATCTATGACACCTCC |
| | | AACCTGGCCTCTGGCGTGCCCGTGAGATTCTCCGGCTCTGGCAGCGGC |
| | | ACCTCCTACTCTCTGACAATCAGCAGGATGGAGGCCGAGGATGCCGCC |
| | | ACATACTATTGCCAGCAGTGGTCCTCTTACCCTCTGACCTTTGGCGCCG |
| | | GCACAAAGCTGGAGCTGAAGAGGGCGGCCGCA |
| 186 | CRT5 scFv mu | ATGGCTGAGGTGCAGCTGCAGCAGTCCGGAGCTGAGCTGATGAAGCC |
| | | AGGCGCCTCTGTGAAGATCAGCTGTAAGGCCACCGGCTACACATTCAG |
| | | CTCCTACTGGATCGAGTGGGTGAACCAGCGCCCTGGCCACGGACTGG |
| | | AGTGGATCGGAGAGATCCTGCCAGGCAGCGGCAGCACCAACTACAAC |
| | | GAGAAGTTCAAGGGCAAGGCTACCTTTACAGCCGACACCTCTAGCAAC |
| | | ACAGCTTACATGCAGCTGTCCTCTCTGACAAGCGAGGATAGCGCCGTG |
| | | TACTACTGCGCCAGGGGCGGAGACTACGATGAGGAGTACTACGCTAT |
| | | GGACTACTGGGGCCAGGGAACCTCTGTGACCCTGAGCAGCGGAGGAG |
| | | GAGGAAGCGGCGGAGGAGGCAGCGGAGGAGGAGGATCTCAGATCGT |
| | | GCTGACCCAGAGCCCAGCTATCATGTCTGCCAGCCCCGGCGAGAAGGT |
| | | GACCATGACATGTAGCGCCAGCAGCAGCGTGAGCTACATGTACTGGT |
| | | ATCAGCAGAAGCCCGGATCTAGCCCTAGGCTGCTGATCTACGACACCT |
| | | CCAACCTGGCCTCTGGCGTGCCCGTGAGATTCTCCGGCTCTGGAAGCG |
| | | GCACCTCCTACTCTCTGACAATCAGCCGGATGGAGGCTGAGGATGCCG |
| | | CTACATACTACTGCCAGCAGTGGTCCTCTTACCCTCTGACCTTTGGAGC |
| | | CGGCACAAAGCTGGAGCTGAAGCGGGCGGCCGCA |

TCR molecule sequences

| | | |
|---|---|---|
| 187 | Human TCR | YIQNPDPAVYQLRDSKSSDKSVCLFTDFDSQTNVSQSKDSDVYITDKTVLD |
| | alpha chain | MRSMDFKSNSAVAWSNKSDFACANAFNNSIIPEDTFFPSPESSCDVKLVE |
| | constant region | KSFETDTNLNFQNLSVIGFRILLLKVAGFNLLMTLRLWSS |
| 188 | Human TCR beta | EDLKNVFPPEVAVFEPSEAEISHTQKATLVCLATGFYPDHVELSWWVNGK |
| | chain constant | EVHSGVSTDPQPLKEQPALNDSRYCLSSRLRVSATFWQNPRNHFRCQVQ |
| | region | FYGLSENDEWTQDRAKPVTQIVSAEAWGRADCGFTSESYQQGVLSATILY |
| | | EILLGKATLYAVLVSALVLMAMVKRKDSRG |
| 189 | VVT1 epitope | RMFPNAPYL |
| 190 | CDR1 alpha | SSYSPS |
| 191 | CDR2 alpha | YTSAATL |
| 192 | CDR3 alpha | VVSPFSGGGADGLT |
| 193 | CDR3 alpha | SPFSGGGADGLT |
| 194 | CDR1 beta | DFQATT |
| 195 | CDR2 beta | SNEGSKA |
| 196 | CDR3 beta | SARDGGEG |
| 197 | CDR3 beta | RDGGEGSETQY |
| 198 | Human TCR | MLLLLVPVLEVIFTLGGTRAQSVTQLDSHVSVSEGTPVLLRCNYSSSYSPSLF |
| | alpha chain | WYVQHPNKGLQLLLKYTSAATLVKGINGFEAEFKKSETSFHLTKPSAHMS |
| | (WT1) | DAAEYFCVVSPFSGGGADGLTFGKGTHLIIQPYIQNPDPAVYQLRDSKSSD |
| | | KSVCLFTDFDSQTNVSQSKDSDVYITDKTVLDMRSMDFKSNSAVAWSNK |
| | | SDFACANAFNNSIIPEDTFFPSPESSCDVKLVEKSFETDTNLNFQNLSVIGF |
| | | RILLLKVAGFNLLMTLRLWSS |
| 199 | Human TCR beta | MLLLLLLLGPGSGLGAVVSQHPSWVICKSGTSVKIECRSLDFQATTMFWY |
| | chain (WT1) | RQFPKQSLMLMATSNEGSKATYEQGVEKDKFLINHASLTLSTLTVTSAHPE |
| | | DSSFYICSARDGGEGSETQYFGPGTRLLVLEDLKNVFPPEVAVFEPSEAEIS |
| | | HTQKATLVCLATGFYPDHVELSWWVNGKEVHSGVSTDPQPLKEQPALN |
| | | DSRYCLSSRLRVSATFWQNPRNHFRCQVQFYGLSENDEWTQDRAKPVT |
| | | QIVSAEAWGRADCGFTSESYQQGVLSATILYEILLGKATLYAVLVSALVLM |
| | | AMVKRKDSRG |
| 200 | Human TCR | MLLLLVPVLEVIFTLGGTRAQSVTQLDSHVSVSEGTPVLLRCNYSSSYSPSLF |
| | alpha chain | WYVQHPNKGLQLLLKYTSAATLVKGINGFEAEFKKSETSFHLTKPSAHMS |
| | (WT1) with Thr 48 | DAAEYFCVVSPFSGGGADGLTFGKGTHLIIQPYIQNPDPAVYQLRDSKSSD |
| | to Cys | KSVCLFTDFDSQTNVSQSKDSDVYITDKCVLDMRSMDFKSNSAVAWSNK |
| | substitution | SDFACANAFNNSIIPEDTFFPSPESSCDVKLVEKSFETDTNLNFQNLSVIGF |
| | | RILLLKVAGFNLLMTLRLWSS |
| 201 | Human TCR beta | MLLLLLLLGPGSGLGAVVSQHPSWVICKSGTSVKIECRSLDFQATTMFWY |
| | chain (WT1) with | RQFPKQSLMLMATSNEGSKATYEQGVEKDKFLINHASLTLSTLTVTSAHPE |
| | Ser 57 to Cys | DSSFYICSARDGGEGSETQYFGPGTRLLVLEDLKNVFPPEVAVFEPSEAEIS |
| | substitution | HTQKATLVCLATGFYPDHVELSWWVNGKEVHSGVCTDPQPLKEQPALN |
| | | DSRYCLSSRLRVSATFWQNPRNHFRCQVQFYGLSENDEWTQDRAKPVT |
| | | QIVSAEAWGRADCGFTSESYQQGVLSATILYEILLGKATLYAVLVSALVLM |
| | | AMVKRKDSRG |
| 202 | WT1 235-243 | CMTWNQMNL |
| | epitope | |
| 203 | Framework | FGKGTHLIIQP |
| | sequence | |
| 204 | Framework | FGPGTRLLVL |
| | sequence | |
| 205 | Consensus | ARGGDYDEEYY(A/V/L)MD |
| | sequence of | |
| | CRT5, 1 and 4 | |
| | Variant 1, heavy | |
| | chain CDR3 | |
| | (SEQ ID Nos 116, | |
| | 34 and 100) | |
| 206 | Consensus | ARGGDYDEEYY(A/V/L)MDY |
| | sequence of | |
| | CRT5, 1 and 4 | |
| | Variant 2, heavy | |
| | chain CDR3 | |
| | (SEQ ID Nos 118, | |
| | 46 and 102) | |
| 207 | Consensus of | ARGGDYDEEYY(A/V/L)MD(Y/X) |
| | CRT5, 1 and 4 | |
| | Variants 1 and 2, | |
| | heavy chain | |
| | CDR3 (SEQ ID | |
| | Nos 116, 34, 100, | |
| | 118, 46, 102) | |
| 208 | Consensus of | QQWSSYPL(T/X) |
| | CRT5, 1 and 4 | |
| | Variants 1 and 2, | |
| | light chain CDR3 | |
| | (SEQ ID Nos 37 | |
| | and 49) | |
| 209 | Consensus of | ((GYTF)/X) SSYW ((IE)/X) |
| | CRT5, 1 and 4 | |
| | Variants 1 and 2, | |
| | heavy chain | |
| | CDR1 (SEQ ID | |
| | Nos 32 and 34) | |
| 210 | Consensus of | ((WIGE)/X) ILPGSGST (N/X) |
| | CRT 5, 1 and 4 | |
| | Variants 1 and 2, | |
| | heavy chain | |
| | CDR2 (SEQ ID | |
| | NOs 33 and 45) | |
| 211 | Consensus of | ((GYTF)/X) (S/T)SYW (((I/M)(E/H))/X) |
| | CRT5, 1, 4 and 3 | |
| | Variants 1 and 2 | |
| | heavy chains | |
| | CDR1 (SEQ ID | |
| | NOs 150 and | |
| | 151) | |
| 212 | Consensus of | (((WIG)(E/A))/X) I(L/Y)PG(S/N)(G/S)(S/D)T ((N/S)/X)) |
| | CRT5, 1, 4 and 3 | |
| | Variants 1 and 2 | |
| | heavy chain | |
| | CDR2 (SEQ ID | |
| | NOs 152 and | |
| | 153) | |
| 213 | Consensus of | (A/T)(R/H)(G/X)(G/X)(D/X)Y(D/Y)(E/G)(E/S)(Y/D)Y(V/A/L)MD |
| | CRT 5, 1, 4 and 3 | (Y/X) |
| | Variants 1 and 2 | |
| | heavy chains | |
| | CDR3 (SEQ ID | |
| | NOs 154 and | |
| | 155) | |
| 214 | Consensus of | ((L(L/W)IY)/X) (D/S)TS ((NLA)/X) |
| | CRT5, 1, 4 and 3 | |
| | Variants 1 and 2 | |
| | light chain CDR2 | |
| 215 | Consensus of | (Q/H)Q(W/Y)(S/H)(S/R)(Y/S)P(L/R)((TF)/X) |
| | CRT5, 1, 4 and 3 | |
| | Variants 1 and 2 | |
| | light chain CDR3 | |
| 216 | Consensus of | ((GYTF)/X) TSYW ((MH)/X) |
| | CRT3 Variants 1 | |
| | and 2 heavy | |
| | chain CDR1 | |
| | (SEQ ID NOs 64 | |
| | and 76) | |
| 217 | Consensus of | ((WIGA)/X)IYPGNSDT(S/X) |
| | CRT3 Variants 1 | |
| | and 2 heavy | |
| | chain CDR2 | |
| | (SEQ ID NOs 65 | |
| | and 77) | |
| 218 | Consensus of | THYYGSDYAMD(Y/X) |
| | CRT3 Variants 1 | |
| | and 2 heavy | |
| | chain CDR3 | |
| | (SEQ ID NOs 66 | |
| | and 78) | |
| 219 | Consensus of | ((SSV)/X) SSSY ((LHWY)/X) |
| | CRT3 Variants 1 | |
| | and 2 light chain | |
| | CDR1 (SEQ ID | |
| | NOs 67 and 79) | |
| 220 | Consensus of | ((LWIY)/X) STS ((NLA)/X) |
| | CRT3 Variants 1 | |
| | and 2 light chain | |
| | CDR2 (SEQ ID | |
| | NOs 68 and STS) | |
| 221 | Consensus of | HQYHRSPR(T/X) |
| | CRT3 Variants 1 | |
| | and 2 light chain | |
| | CDR3 (SEQ ID | |
| | NOs 69 and 81) | |
| 222 | Nucleotide | actactaccaagccagtgctgcgaactccctcacctgtgcaccctaccgggacatc |
| | sequence | tcagccccagagaccagaagattgtcggccccgtggctcagtgaaggggaccg |
| | encoding Hinge | gattggacttcgcctgtgatatttacatctgggcacccttggccggaatctgcgtggc |
| | and | ccttctgctgtccttgatcatcactctcatctgctaccacaggagccga |
| | transmembrane | |
| | regions of mouse | |
| | CD8α | |
| 223 | Nucleotide | aatagtagaaggaacagactccttcaaagtgactacatgaacatgactccccgga |
| | sequence | ggcctgggctcactcgaaagccttaccagccctacgcccctgccagagactttgca |
| | encoding mouse | gcgtaccgcccc |
| | intracellular | |
| | signalling | |
| | sequences from | |
| | mouse CD28 | |
| 224 | Nucleotide | aaatggatcaggaaaaaattcccccacatattcaagcaaccatttaagaaga |
| | sequence | ccactggagcagctcaagaggaagatgcttgtagctgccgatgtccacag |
| | encoding mouse | gaagaagaaggaggaggaggaggctatgagctg |
| | 4-1BB domain | |
| 225 | Nucleotide | agagcaaaattcagcaggagtgcagagactgctgccaacctgcaggaccccaa |
| | sequence | ccagctctacaatgagctcaatctagggcgaagagaggaatatgacgtcttggag |
| | encoding mouse | aagaagcgggctcgggatccagagatgggaggcaaacagcagaggaggagg |
| | CD3 zeta chain | aacccccaggaaggcgtatacaatgcactgcagaaagacaagatggcagaag |
| | | cctacagtgagatcggcacaaaaggcgagaggcggagaggcaaggggcacg |
| | | atggcctttaccagggtctcagcactgccaccaaggacacctatgatgccctgcat |
| | | atgcagaccctggccc |
| 226 | Nucleotide | cggaaggcttggagattgcctaacactcccaaaccttgttggggaaacagcttcag |
| | sequence | gaccccgatccaggaggaacacacagacgcacactttactctggccaagatc |
| | encoding mouse | |
| | OX40 domain | |
| 227 | Nucleotide | ggaggcaccaagctggaaatcaaacgtgcggccgcaactactaccaagccagtgctgcgaa |
| | sequence | ctccctcacctgtgcaccctaccgggacatctcagccccagagaccagaagattgtcggcccc |
| | encoding murine | gtggctcagtgaaggggaccggattggacttcgcctgtgatatttacatctgggcacccttgg |
| | CD8α hinge and | ccggaatctgcgtggcccttctgctgtccttgatcatcactctcatctgctaccacaggagccg |
| | transmembrane | aaatagtagaaggaacagactccttcaaagtgactacatgaacatgactccccggaggcct |
| | regions, CD28 | gggctcactcgaaagccttaccagccctacgcccctgccagagactttgcagcgtaccgcccc |
| | intracellular | agagcaaaattcagcaggagtgcagagactgctgccaacctgcaggaccccaaccagctct |
| | signalling domain | acaatgagctcaatctagggcgaagagaggaatatgacgtcttggagaagaagcgggctcg |
| | and CD3ζ | ggatccagagatgggaggcaaacagcagaggaggaggaacccccaggaaggcgtataca |
| | intracellular | atgcactgcagaaagacaagatggcagaagcctacagtgagatcggcacaaaaggcgaga |
| | signalling domain | ggcggagaggcaaggggcacgatggcctttaccagggtctcagcactgccaccaaggacac |
| | | ctatgatgccctgcatatgcagaccctggcccctcgctaataaaagcttaacacgagccatag |
| | | atagaataaaag |
| 228 | Nucleotide | ggaggcaccaagctggaaatcaaacgtgcggcgcaactactaccaagccagtgctgcgaa |
| | sequence | ctccctcacctgtgcaccctaccgggacatctcagccccagagaccagaagattgtcggcccc |
| | encoding murine | gtggctcagtgaaggggaccggattggacttcgcctgtgatatttacatctgggcacccttgg |
| | CD8α hinge and | ccggaatctgcgtggcccttctgctgtccttgatcatcactctcatctgctaccacaggagccg |
| | transmembrane | aaaatggatcaggaaaaaattcccccacatattcaagcaaccatttaagaagaccactgga |
| | domains, 4-1BB | gcagctcaagaggaagatgcttgtagctgccgatgtccacaggaagaagaaggaggagga |
| | intracellular | ggaggctatgagctgagagcaaaattcagcaggagtgcagagactgctgccaacctgcagg |
| | signalling domain | accccaaccagctctacaatgagctcaatctagggcgaagagaggaatatgacgtcttgga |
| | and CD3ζ | gaagaagcgggctcgggatccagagatgggaggcaaacagcagaggaggaggaaccccc |
| | intracellular | aggaaggcgtatacaatgcactgcagaaagacaagatggcagaagcctacagtgagatcg |
| | signalling domain | gcacaaaaggcgagaggcggagaggcaaggggcacgatggcctttaccagggtctcagca |
| | | ctgccaccaaggacacctatgatgccctgcatatgcagaccctggcccctcgctaataaaag |
| | | cttaacacgagccatagatagaataaaag |
| 229 | Nucleotide | ggaggcaccaagctggaaatcaaacgtgcggccgcaactactaccaagccagtgctgcgaa |
| | sequence | ctccctcacctgtgcaccctaccgggacatctcagccccagagaccagaagattgtcggcccc |
| | encoding murine | gtggctcagtgaaggggaccggattggacttcgcctgtgatatttacatctgggcacccttgg |
| | CD8α hinge and | ccggaatctgcgtggcccttctgctgtccttgatcatcactctcatctgctaccacaggagccg |
| | transmembrane | acggaaggcttggagattgcctaacactcccaaaccttgttggggaaacagcttcaggaccc |
| | domains, OX40 | cgatccaggaggaacacacagacgcacactttactctggccaagatcagagcaaaattcag |
| | intracellular | caggagtgcagagactgctgccaacctgcaggaccccaaccagctctacaatgagctcaatc |
| | signalling domain | tagggcgaagagaggaatatgacgtcttggagaagaagcgggctcgggatccagagatgg |
| | and CD3ζ | gaggcaaacagcagaggaggaggaacccccaggaaggcgtatacaatgcactgcagaaa |
| | intracellular | gacaagatggcagaagcctacagtgagatcggcacaaaaggcgagaggcggagaggcaa |
| | signalling domain | ggggcacgatggcctttaccagggtctcagcactgccaccaaggacacctatgatgccctgc |
| | | atatgcagaccctggcccctcgctaataaaagcttaacacgagccatagatagaataaaag |
| 230 | Nucleotide | ggaggcaccaagctggaaatcaaacgtgcggccgcaactactaccaagccagtgctgcgaa |
| | sequence | ctccctcacctgtgcaccctaccgggacatctcagccccagagaccagaagattgtcggcccc |
| | encoding murine | gtggctcagtgaaggggaccggattggacttcgcctgtgatatttacatctgggcacccttgg |
| | CD8α hinge and | ccggaatctgcgtggcccttctgctgtccttgatcatcactctcatctgctaccacaggagccg |
| | transmembrane | aaatagtagaaggaacagactccttcaaagtgactacatgaacatgactccccggaggcct |
| | domains, CD28 | gggctcactcgaaagccttaccagccctacgcccctgccagagactttgcagcgtaccgcccc |
| | and 4-1BB | aaatggatcaggaaaaaattcccccacatattcaagcaaccatttaagaagaccactggag |
| | intracellular | cagctcaagaggaagatgcttgtagctgccgatgtccacaggaagaagaaggaggaggag |
| | signalling | gaggctatgagctgagagcaaaattcagcaggagtgcagagactgctgccaacctgcagga |
| | domains and | ccccaaccagctctacaatgagctcaatctagggcgaagagaggaatatgacgtcttggag |
| | CD3ζ intracellular | aagaagcgggctcgggatccagagatgggaggcaaacagcagaggaggaggaaccccca |
| | signalling domain | ggaaggcgtatacaatgcactgcagaaagacaagatggcagaagcctacagtgagatcgg |
| | | cacaaaaggcgagaggcggagaggcaaggggcacgatggcctttaccagggtctcagcac |
| | | tgccaccaaggacacctatgatgccctgcatatgcagaccctggcccctcgctaataaaagct |
| | | taacacgagccatagatagaataaaag |
| 231 | Nucleotide | ggaggcaccaagctggaaatcaaacgtgcggccgcaactactaccaagccagtgctgcgaa |
| | sequence | ctccctcacctgtgcaccctaccgggacatctcagccccagagaccagaagattgtcggcccc |
| | encoding murine | gtggctcagtgaaggggaccggattggacttcgcctgtgatatttacatctgggcacccttgg |
| | CD8α hinge and | ccggaatctgcgtggcccttctgctgtccttgatcatcactctcatctgctaccacaggagccg |
| | transmembrane | aaatagtagaaggaacagactccttcaaagtgactacatgaacatgactccccggaggcct |
| | domains, CD28 | gggctcactcgaaagccttaccagccctacgcccctgccagagactttgcagcgtaccgcccc |
| | and OX40 | cggaaggcttggagattgcctaacactcccaaaccttgttggggaaacagcttcaggacccc |
| | intracellular | gatccaggaggaacacacagacgcacactttactctggccaagatcagagcaaaattcagc |
| | signalling | aggagtgcagagactgctgccaacctgcaggaccccaaccagctctacaatgagctcaatct |
| | domains and | agggcgaagagaggaatatgacgtcttggagaagaagcgggctcgggatccagagatggg |
| | CD3ζ intracellular | aggcaaacagcagaggaggaggaacccccaggaaggcgtatacaatgcactgcagaaag |
| | signalling domain | acaagatggcagaagcctacagtgagatcggcacaaaaggcgagaggcggagaggcaag |
| | | gggcacgatggcctttaccagggtctcagcactgccaccaaggacacctatgatgccctgca |
| | | tatgcagaccctggcccctcgctaataaaagcttaacacgagccatagatagaataaaag |
| 232 | Nucleotide | Ggaggcaccaagctggaaatcaaacgtgcggccgcaactactaccaagccagtgctgcga |
| | sequence | actccctcacctgtgcaccctaccgggacatctcagccccagagaccagaagattgtcggcc |
| | encoding murine | ccgtggctcagtgaaggggaccggattggacttcgcctgtgatatttacatctgggcaccctt |
| | CD8α hinge and | ggccggaatctgcgtggcccttctgctgtccttgatcatcactctcatctgctaccacaggagc |
| | transmembrane | cgaaaatggatcaggaaaaaattcccccacatattcaagcaaccatttaagaagaccactg |
| | domains, 4-1BB | gagcagctcaagaggaagatgcttgtagctgccgatgtccacaggaagaagaaggaggag |
| | and OX40 | gaggaggctatgagctgcggaaggcttggagattgcctaacactcccaaaccttgttgggga |
| | intracellular | aacagcttcaggaccccgatccaggaggaacacacagacgcacactttactctggccaaga |
| | signalling | tcagagcaaaattcagcaggagtgcagagactgctgccaacctgcaggaccccaaccagct |
| | domains and | ctacaatgagctcaatctagggcgaagagaggaatatgacgtcttggagaagaagcgggct |
| | CD3ζ intracellular | cgggatccagagatgggaggcaaacagcagaggaggaggaacccccaggaaggcgtata |
| | signalling domain | caatgcactgcagaaagacaagatggcagaagcctacagtgagatcggcacaaaaggcga |
| | | gaggcggagaggcaaggggcacgatggcctttaccagggtctcagcactgccaccaaggac |
| | | acctatgatgccctgcatatgcagaccctggcccctcgctaataaaagcttaacacgagccat |
| | | agatagaataaaag |

In the above table the designation of an amino acid as “X” indicates that no amino acid may be present at that position.

### EXAMPLES

### Example 1: Experimental Studies on CLEC14A

### Materials and Methods

### HUVEC Preparation and Culture

Human umbilical vein endothelial cells (HUVECs) were isolated from umbilical cords donated by the UK National Health Service after informed consent of the donors. Cords were dissected from placentas and the vein was washed in sterile PBS to remove blood. 1 mg/ml of collagenase diluted in M199 medium (Sigma) was injected into the vein and then incubated at 37° C. for 20 minutes to detach the endothelial cells. HUVECs were collected by washing in M199 complete medium containing 10% FCS, 10% large vessel endothelial cell growth supplement (TCS Cell Works), and 4 mM L-glutamine, and plated on 0.1% Type 1 gelatin from porcine skin (Sigma) coated dishes.

### Primary Cells Source

Human aortic smooth muscle cells (HASMC) and human bronchial epithelial cells (HBE) were purchased from TCS Cell Works. Human lung fibroblasts (MRCS) were obtained from Cancer Research UK Central Services. Human peripheral blood mononuclear cells (PBMCs) were obtained from the Institute of Cancer Studies at the University of Birmingham. Hepatocytes were a gift from Professor David Adams, School of Immunity and Infection, University of Birmingham.

### HUVEC Immunofluorescence

HUVECs were grown in glass micro-well chambers (Nunc) fixed in ice-cold methanol, washed with PBST blocked in 10% FCS 3% BSA in PBST. Cells were then stained with CLEC14A antibody following the same protocol used for paraffin embedded sections or co-stained with 5 μg/ml mouse monoclonal IgG antibody against human VE-cadherin, kindly donated by Professor Maria Grazia Lampugnani, Firc Institute for Molecular Oncology, Milan. Sections staining were analyzed with a 510 laser scanning confocal microscope (Carl Zeiss).

Scratch Wound Healing Assay with CLEC14A Monoclonal Antibodies

A scratch with a 10 μl pipette tip was made in confluent HUVECs. New medium containing 1 μg/ml or 10 μg/ml of a monoclonal CLEC14A antibody raised in mice against the extracellular domain of CLEC14A was applied. Chemokinetic migration of HUVECs was assessed by acquiring images of wound closure at time zero, 4, 6, 12 hours with a Leica DM 1000 light microscope and USB 2.0 2M Xli camera. The open area of the wound was quantitated using Image J software.

### Immunofluorescence on Paraffin Embedded Tissues

Immunofluorescence was performed on paraffin embedded normal and cancer human tissue collection obtained from Cancer Research UK histology service and on cancer and normal tissue arrays (Superbiochips) (Data not shown). Human common cancers 1 (MA2) including 10 cores of each of the following carcinoma: stomach, oesophagus, lung, colon/rectum, thyroid and kidney, and common cancers 2 (MB3) including 10 cores of each of the following carcinomas: breast, liver, bladder, ovarian, pancreas and prostate were used. Two additional control arrays of matching adjacent normal tissues were also analysed. After removal of paraffin, tissues were rehydrated and microwaved for 3 minutes on medium power in citrate buffer pH6 for antigen retrieval. Sections were blocked in PBST containing 10% FCS and 3% BSA. Sections were probed with 10 μg/ml of sheep IgG primary polyclonal antibody against the extracellular domain of human CLEC14A (R&D system) and 15 μg/ml of FITC conjugated rabbit IgG secondary anti-sheep polyclonal antibody (Zymax). Vessel endothelial cells were stained with 20 μg/ml of Ulex europeaus agglutinin I (UEAI) conjugated with rhodamine (Vector labs). Slides were permanently mounted with prolong gold anti-fade reagent with DAPI (Invitrogen) to counterstain cell nuclei. Section staining was analysed using a 510 laser scanning confocal microscope (Carl Zeiss).

### Preparation of Monoclonal Antibodies

The antigens used for the preparation of monoclonal antibodies were murine CLEC14A-Fc (CM) and human CLEC14A-Fc (CH), optionally conjugated with adjuvant protein (AP). These four antigens (CM, CH, CM-AP, CH-AP) were used for mice immunisation using the following protocol:

| Day | Operation |
|---|---|
| 0 | Pre-immune sample taken |
| | Immunisation of 100 μg of antigen in |
| | complete Freunds adjuvant (foot pads) |
| 14 | Immunisation of 100 μg of antigen in |
| | incomplete Freunds adjuvant (foot pads) |
| 17 | Test bleed |
| 18 | Popliteal lymph node harvest for fusion |

Sera were tested by ELISA against three antigens: CM, CH and Fc. A non-immune serum was taken as a negative control.
The fusion protocol was as follows:
(1) Popliteal lymph nodes were harvested from the immune mice and homogenised.
(2) Cells were washed with warm DMEM.
(3) Cells were mixed with sp2/0 myeloma cells.
(4) The mixture was centrifuged (1000 g)
(5) The pellet was suspended in 50% PEG 1500 and incubated for 1 min.
(6) The suspension was slowly diluted with warm DMEM.
(7) Suspension was centrifuged (1000 g).
(8) Cells were seeded into plates with peritoneal macrophages.
(9) Cells were cultivated at 37° C. and 5% CO₂

More than 500 HAT-resistant hybridoma clones from each mouse were obtained. All of the clone supernatants were tested twice with 4 days interval by ELISA against three absorbed antigens (CM, CH and Fc). Testing resulted in 5 clones (all subclass IgG1) that reacted with both CM and CH and did not react with Fc. All positives were cloned 2-4 times by the limiting dilution method, propagated in culture flasks and injected into mice for ascites. Three clones were derived as a result of immunisation with CLEC14a human (CH), one clone (CRT-3) was the result of immunisation with CLEC14a human-AP (CH-AP), and one clone (CRT-2) was the result of immunisation with CLEC14a mouse-AP (CM-AP).

### Tubule Formation Assays

HUVECs were treated with 20 μg/ml of CRT2, CRT3 or CRT4 or IgG isotype control. Images of the tubules were taken at 16 hours and were analysed for total tubule length, number of junctions, number of branches, branch length, number of meshes and total mesh area. The experiments were repeated three times, with five data points analysed per experiment.

### Results

FIG. 2 is a graph showing the relative expression of CLEC14A in HUVECs and other primary cells. CLEC14A was expressed specifically in endothelial cells. This confirms our previous finding that CLEC14A was endothelial-specific (Herbert et al, 2008).

The ability of CLEC14A monoclonal antibodies to inhibit angiogenesis was examined. Scratch wound healing assays using monoclonal antibodies were carried out. As shown in FIG. 3, when HUVECs were treated with 10 μg/ml of monoclonal antibody CRT-3, 25% of the wound area remained open at 12 h compared to 13% in the control. These results show that CLEC14A antibodies, have an inhibitory effect on endothelial cell migration. Endothelial cell migration is an essential feature of angiogenesis. Accordingly, this assay provides evidence that a CLEC14A antibody, inhibits angiogenesis.

Further, the tubule formation assays showed that the number and total length of branches was significantly increased by treatment with CRT4 and that CRT4 also significantly reduced the number of meshes per filed. These results suggest that CRT4 does not affect tube formation but that it affects the connection of tubes. This is evidenced by the increased number and length of branches, indication that the tubules are less well interconnected. CRT2 and CRT3 treatment produced a significant reduction in tubule length and the number of junctions and CRT2 also significantly reduced the mesh area per field. Thus these assays provide further evidence that distinct CLEC14A antibodies inhibit angiogenesis (albeit by having differing effects on tube formation.

The expression of CLEC14A in sections of solid tumours and normal tissue was examined using CLEC14A-specific probes (data not shown). CLEC14A expression was seen in the blood vessels in all tumour tissues analysed. Ovarian, bladder, liver, breast, kidney and prostate tumours were strongly positive for CLEC14A expression, whereas stomach, oesophagus, lung, colon, rectal, pancreatic and thyroid tumour tissues showed a lower level of specific CLEC14A expression. CLEC14A expression was not detected in any of the corresponding normal control (non-tumour) tissues. Accordingly, it has been demonstrated that CLEC14A is specifically expressed in tumour vasculature.

### Example 2: Blocking CLEC14A-MMRN2 Binding Inhibits Sprouting Angiogenesis and Tumour Growth

### Materials and Methods

### Reagents

For Western blotting and immunoprecipitation; primary antibodies: sheep polyclonal anti-human CLEC14A (R&D systems), mouse monoclonal anti-human Tubulin (Sigma), mouse polyclonal anti-human MMRN2 (Abnova); secondary antibodies: goat polyclonal anti-mouse IgG conjugated to horseradish peroxidase (HRP) (Dako), donkey polyclonal anti-sheep IgG conjugated to HRP (R&D systems). For immunofluorescence; primary antibodies: rabbit polyclonal anti-murine PECAM (Santa Cruz); secondary antibodies: donkey polyclonal anti-rabbit conjugated to Alexa Fluor488 (Invitrogen). For flow cytometry; primary antibodies: mouse monoclonal anti-HA tag (CRUK), mouse monoclonal anti-CLEC14A (C2, C4 described below); secondary antibodies: goat polyclonal anti-mouse IgG conjugated to Alexa Fluor488 (Invitrogen).

### Plasmids

For protein production; lentiviral plasmids psPAX2 (lentiviral packaging; Addgene), pMD2G (Envelope plasmid; Addgene) and pWPI hCLEC14A-ECD-Fc (lentiviral mammalian expression plasmid containing IRES-EGFP; Addgene) were used. pWPI hCLEC14A-Fc and mCLEC14A-Fc was generated by initial PCR subcloning from clec14a IMAGE clone (Origene) into pcDNA3-Fc plasmid. The primers used were as follows: human CLEC14A fwd 5′TAGTAGGAATTCGAGAGAATGAGGCCGGCGTTCGCCCTG3′ (SEQ ID NO: 4); human CLEC14A rev—5′AGAACCGCGGCCGCTGGAGGAGTCGAAAGCCTGAGGAGT3′ (SEQ ID NO: 5); murine CLEC14A fwd—5′TAGTAGGAATTCGAGAGAATGAGGCCAGCGCTTGCCCTG3′ (SEQ ID NO: 6; murine CLEC14A rev—5′CTACTAGCGGCCGCTCGTGGAAGAGGTGTCGAAAGT3′ (SEQ ID NO: 7). EcoR1 and Not1 restriction sites were used to insert CLEC14A. A further round of PCR subcloning was performed to transfer the CLEC14A-Fc fusion into pWPI. The primers used were as follows: human CLEC14A fwd—5′TAGTAGTTAATTAAGAGAGAATGAGGCCGGCGTTC3′ (SEQ ID NO: 8); murine CLEC14A fwd—5′TAGTAGTTAATTAAGAGAGAATGAGGCCAGCGCTT3′ (SEQ ID NO: 9); human Fc rev—5′CTACTAGTTTAAACTCATTTACCCGGAGACAGGGA3′ (SEQ ID NO: 10). For this step, Pac1 and Pme1 restriction sites were used.

MMRN2 mammalian expression plasmid was constructed by PCR cloning from mmrn2 IMAGE clone (Thermo) into pHL-Avitag3, using the following primers: fwd—CCGGACCGGTCAGGCTTCCAGTACTAGCC (SEQ ID NO: 11); rev—CGGGGTACCGGTCTTAAACATCAGGAAGC (SEQ ID NO: 12). Age1 and Kpn1 restriction enzymes were used.

### Cell Culture

Human Umbilical Vein Endothelial Cells were isolated as described previously. Umbilical cords were obtained from Birmingham Women's Health Care NHS Trust with informed consent. HUVECs were used between passages 1-6 and were cultured in M199 complete medium (cM199) containing 10% fetal calf serum (PAA), 1% bovine brain extract, 90 μg/ml heparin, and 4 mM L-glutamine, 100 U/ml penicillin and 100 μg/ml streptomycin (Invitrogen) and were seeded on plates coated in 0.1% type 1 gelatin from porcine skin. HEK293T cells were cultured in DMEM (Sigma) complete medium (cDMEM) containing 10% fetal calf serum (PAA), 4 mM L-glutamine, 100 U/ml penicillin and 100 μg/ml streptomycin (Invitrogen).

SiRNA transfections in HUVEC were performed as previously described. Lentivirus was produced in HEK293T cells by transient transfection with the lentiviral packaging, envelope and expression plasmids above. Plasmids were incubated in OptiMEM (Invitrogen) with polyethylenimine (36 μg/ml) at a 1:4 ratio for 10 minutes at room temperature prior to adding to HEK293T cells in cDMEM. Media supernatant was used to transduce fresh HEK293T cells. GFP positive HEK293T cells were sorted and used for protein production. Expression of MMRN2 in HEK293T cells was achieved by polyethylenimine transient transfection as above using pHL-Avitag3 hMMRN2.

### Quantitative PCR

cDNA was prepared using the High-Capacity cDNA Archive kit (Applied Biosystems), from 1 μg of extracted total RNA. qPCR reactions were performed with Express qPCR supermix (Invitrogen) on a RG-3000 (Corbett/Qiagen, Manchester, UK) thermocycler. Primers for human clec14a and flotillin-2 were as previously described. Primers for murine clec14a 5′ UTR, CDS and 3′ UTR and murine beta-actin, are as follows: 5′UTR fwd—TTCCTTTTCCAGGGTTTGTG (SEQ ID NO: 13); 5′ UTR rev—GCCTACAAGGTGGCTTGAAT (SEQ ID NO: 14); CDS fwd—AAGCTGTGCTCCTGCTCTTG (SEQ ID NO: 15; CDS rev—TCCTGAGTGCACTGTGAGATG (SEQ ID NO: 16); 3′ UTR fwd—CTGTAGAGGGCGGTGACTTT (SEQ ID NO 17); 3′ UTR rev—AGCTGCTCCCAAGTCCTCT (SEQ ID NO: 18); mACTB fwd—CTAAGGCCAACCGTGAAAAG (SEQ ID NO: 19); mACTB rev—ACCAGAGGCATACAGGGACA (SEQ ID NO: 20). Relative expression ratios were calculated according to the efficiency adjusted mathematical model.

### Western Blotting and Immunoprecipitation

Whole cell protein lysates were made and co-immunoprecipitation experiments were performed as previously described, except protein was extracted from 2×10⁷ HUVECs. For initial isolation of CLEC14A interacting proteins 5 μg CLEC14A-Fc or an equimolar amount of hFc was used. For endogenous immunoprecipitation experiments 0.4 μg anti-CLEC14A antibody or sheep IgG was used. For blocking experiments 5 μg CLEC14A-Fc or hFc were bound to protein G beads overnight in PBS. Beads were blocked for 5-6 hours in PBS containing 20% FCS (PAA). Bound CLEC14A-Fc or hFc protein was blocked with increasing concentrations of mIgG, C2 or C4 in binding buffer overnight. Lysates from MMRN2 transfected HEK293T cells were then incubated overnight with the bead complexes before washing and analysing by Western blot. Standard protocols were used for Western blotting and SDS-PAGE. Primary antibodies were used as indicated in the text with corresponding HRP conjugated secondary antibodies.

### Flow Cytometry

Cells were detached with cell dissociation buffer (Invitrogen), rinsed in PBS before incubation in blocking buffer (PBS, 3% BSA, 1% NaN₃) for 15 minutes. Subsequent staining using 10 μg/ml anti-HA tag (CRUK), 10 μg/ml anti-CLEC14A (C2, C4 described below), as primary antibodies, in blocking buffer for 30 minutes. Cells were rinsed in PBS and stained with goat polyclonal anti-mouse IgG conjugated to Alexa Fluor488 (Invitrogen) in blocking buffer. Data (15,000 events/sample) were collected using a FACSCalibur apparatus (Becton Dickinson, Oxford, UK), and results were analysed with Becton Dickinson Cell Quest software.

### HUVEC Spheroid Sprouting Assay and In Vitro Matrigel Tube Forming Assay

Generation of HUVEC spheroids and induction of endothelial sprouting in a collagen gel was performed as previously described, using 1000 HUVECs per spheroid. Quantification was performed 16 hours after embedding. To quantify sprout growth the number of sprouts were counted, the cumulative sprout length and the maximal sprout length was assessed. For two colour sprouting experiments, HUVECs were pre-labelled with orange and green CellTracker dyes (Invitrogen). After 24 hours spheroids were fixed in 4% formaldehyde and mounted with Vectorshield (Vector labs). Slides were imaged with an Axioskop2 microscope and AxioVision SE64 Re14.8 software (Zeiss, Cambridge, UK).

For the Matrigel tube forming assays 1.4×10⁵ HUVECs were seeded onto 70 μl basement membrane extract (Matrigel, BD Bioscience, Oxford, UK) in a 12 well plate. After 16 hours, images were taken of 5 fields of view per well using a Leica DM IL microscope (Leica, Milton Keynes, UK) with a USB 2.0 2M Xli digital camera (XL Imaging LLC, Carrollton, Tex., USA) at 10× magnification. Images were analysed with the Angiogenesis analyser plugin for Image J (Carpentier G. et al., Angiogenesis Analyzer for ImageJ. 4th ImageJ User and Developer Conference proceedings) and available at the NIH website (http://imagej.nih.gov/ij/macros/toolsets/Angiogenesis%20Analyzer.txt).

### Protein Production

Culture media (CM) from CLEC14A-Fc expressing HEK293T cells was collected. CM was flowed over a HiTrap protein A HP column (GE healthcare, Amersham, UK) and protein eluted using a 0-100% gradient of 100 mM sodium citrate (pH 3) before neutralising with 1 M Tris base. Fractions were run on a SDS-PAG and assessed for protein purity and specificity by Coomassie staining and Western blotting. Fractions containing similar concentrations of protein were combined and dialysed in PBS prior to functional assays.

### Monoclonal Antibody Generation

Mouse monoclonal antibodies were commercially prepared by Serotec Ltd (Oxford, UK) using the following protocol to break tolerance supplied by us. Purified mouse CLEC14A-Fc fusion protein was given at 50 μg in Freunds complete adjuvant subcutaneously. Two weeks later mice were given another 50 μg subcutaneously but this time in Freunds adjuvant. Mice were culled and spleens harvested for fusion two weeks later.

Generation of clec14a −/− Mice

Mice were housed at the Birmingham Biomedical Services Unit (Birmingham, UK). C57BL/6N VGB6 feeder-dependent embryonic stem cells containing the CLEC14A deletion cassette (Clec14atm1(KOMP)Vlcg; project ID VG10554) were procured from the Knockout Mouse Project (University of California, Davis, USA). The Transgenic Mouse Facility at the University of Birmingham generated chimeric mice by injection of embryonic stem cells into albino C57BL/6 mice and were bred to C57BL/6 females to generate mice heterozygous for the cassette. Animal maintenance had appropriate

### Home Office Approval and Licensing

### Aortic Ring and Murine Subcutaneous Sponge Angiogenesis Assay

Aortas were isolated and processed for aortic ring assays in collagen. Tube/sprout outgrowth, maximal endothelial migration and total endothelial outgrowth was quantitated. Themurine subcutaneous sponge angiogenesis assay was performed as previously described, with slight modification. Male C57 black mice were implanted with a subcutaneous sterile polyether sponge disc (10×5×5 mm) under the dorsal skin of each flank at day 0. 100 μl bFGF (40 ng/ml; R&D systems) was injected through the skin directly into the sponges every other day for 14 days. Sponges were excised on day 14, fixed in 10% formalin, and paraffin embedded. Sections were stained with haematoxylin and eosin, sponge cross-sections were taken using a Leica MZ 16 microscope (Leica, Milton Keynes, UK) with a USB 2.0 2M Xli digital camera (XL Imaging LLC, Carrollton, Tex., USA) at ×1 magnification for cellular invasion analysis. Images captured by Leica DM E microscope (Leica, Milton Keynes, UK) at 40× magnification were analysed for vessel density. Vessel counts were assessed in five fields per section per sponge. All animal experimentation was carried out in accordance with Home Office License number PPL 40/3339 held by RB.

### Tumour Implantation Assays

10⁶ Lewis lung carcinoma cells were injected subcutaneously into the flank of male mice at 8-10 weeks of age. Tumour growth was monitored by daily calliper measurements and after two-four weeks growth, tumour mass was determined by weight, fixed in 4% PFA, paraffin embedded and serial sections cut at 6 μm.

### Immunofluorescence and X-Gal Staining

Immunofluorescence staining and X-Gal staining were performed using methods known in the art.

### Results

### CLEC14A Regulates Sprouting Angiogenesis In Vitro

CLEC14A has previously been shown to be involved in endothelial migration and tube formation in vitro. To investigate the role of CLEC14A in sprouting angiogenesis in vitro, HUVEC spheroids were generated from HUVECs treated with siRNA targeting clec14a or a non-complementary siRNA duplex. Knockdown of clec14a expression was confirmed at the mRNA level by qPCR with an average reduction of 74% across three experiments (FIG. 5A) and at the protein level by Western blot analysis of protein extracts probed with an anti-CLEC14A polyclonal antisera (FIG. 5B). VEGF induced sprouting from CLEC14A knockdown spheroids was impaired, knockdown spheroids produced on average 6.9 sprouts per spheroid, compared to 13.2 for control cells (FIGS. 5C and 5D). To determine the role of CLEC14A in tip/stalk cell formation, control HUVECs and knockdown HUVECs were stained either red or green and mixed, prior to spheroid formation and induced sprouting (FIG. 5E). Knockdown of CLEC14A reduced the percentage of cells at the tip position (33%) compared to control cells (67%), however, there was no effect on the percentage of stalk cells that were derived from CLEC14A knockdown HUVECs (FIG. 5F). These data suggest CLEC14A has a role in sprout initiation and migration.

### CLEC14A Regulates Sprouting Angiogenesis In Vivo

Previously published data for CLEC14A has demonstrated its role in endothelial biology in vitro, however its in vivo role has not been reported. To investigate the role of CLEC14A in vivo and ex vivo, mice were generated to replace the clec14a coding sequence with a lacZ reporter (FIG. 6A). Breeding of heterozygotes (clec14a −/+) produced equal proportions of male and female mice (49.5%/50.5% respectively) and a Mendelian ratio of wildtype:heterozygote:homozygote mice (26.4%:47.2%:26.4% respectively). As clec14a is an endothelial-restricted gene, aortas were isolated from clec14a +1+ and clec14a −/−mice. Extracted cDNA was analysed by qPCR and confirmed loss of the clec14a coding region but expression of the 5′ and 3′ untranslated regions were retained (FIG. 6B). Loss of CLEC14A at the protein level was also confirmed by Western blot analysis of lung tissue lysates (FIG. 6C).

To confirm the role of CLEC14A in sprouting angiogenesis in multicellular three dimensional co-culture, aortas were isolated, cut into rings and embedded in collagen. Cellular outgrowth was stimulated by VEGF and monitored over 7 days before end-point quantitation of endothelial sprouting. Again, loss of CLEC14A impaired endothelial sprout outgrowth and migration (FIG. 6D). Aortic rings from wildtype mice produced over double the number of tubes compared to that observed for CLEC14A knockout mice (30.6 tubes compared to 13.4 tubes respectively) (FIG. 6E). In addition, the maximum migration, which is defined by the furthest distance migrated away from each aortic ring, was also reduced in knockout cultures (FIG. 6F). To assess whether CLEC14A has a similar function in vivo, sponge barrels were implanted subcutaneously into CLEC14A knockout mice. Cellular infiltration and neo-angiogenesis were stimulated using bFGF injections into the sponge every two days for two weeks. Macroscopic analysis of sponge sections stained with haematoxylin and eosin revealed impaired infiltration of cells into the sponge in clec14a −/− animals (FIGS. 6G and 6H). In addition, vascularity was significantly reduced (p<0.01) for clec14a −/− animals (FIG. 6I). To confirm the endothelial cells lining the neoangiogenic vessels express clec14a in this model, sponges and livers from CLEC14A KO mice were stained with x-gal. Strong x-gal staining was observed on blood vessels within the sponge compared to matched liver sections (FIG. 6J). From these data we can conclude that mouse CLEC14A expression regulates endothelial migration and angiogenic sprouting in vivo, as well as in vitro, and CLEC14A is upregulated on sprouting endothelium.

### CLEC14A Promotes Tumour Growth

CLEC14A expression is found highly up-regulated on human tumour vessels compared to vessels from healthy tissue, suggesting that cancer therapies could be targeted against CLEC14A. Therefore, to investigate whether loss of CLEC14A effects tumour growth we used the syngeneic Lewis lung carcinoma (LLC) model. For this 1×10⁶ LLC cells were injected subcutaneously into the right flank of either clec14a +/+ or clec14a −/− mice. Tumour growth was impaired in the clec14a −/− mice compared to clec14a +1+ littermates (FIG. 7A). This was confirmed by three independent experiments. Excised tumours taken from clec14a −/− mice were smaller in size (FIG. 7B) and smaller in weight (FIG. 7C) than clec14a +1+ littermates. To determine whether the vascular density within these tumours was also effected, tissue sections were stained with an anti-CD31 antibody. Analysis shows a reduced density of discrete vessels (FIGS. 7D and 7E) and reduced percentage endothelial coverage (FIG. 7F). Furthermore, x-gal staining of tumour and liver sections taken from clec14a −/− mice reveals high expression of clec14a on both mature vessels, with erythrocyte filled lumens (FIG. 3G, black arrows), and immature microvessels within the tumour (FIG. 7G, red arrows), confirming clec14a is upregulated on tumour vessels.

### Identification and Confirmation of CLEC14A-MMRN2 Interaction

To identify potential binding partners for the extracellular domain for CLEC14A, we first purified CLEC14A extracellular domain protein tagged with human Fc. This protein or Fc alone was incubated with HUVEC whole cell lysates and precipitated using protein A agarose beads. The precipitated proteins were then washed and separated on a SDS-PAG. Seven gel regions were excised, digested and analysed by mass spectrometry. The most abundant protein identified was MMRN2 with 12 peptides (11 unique), and no peptides in the corresponding control pulldown fraction. Western blot analysis of the precipitates confirmed the presence of MMRN2 in the CLEC14A-ECD-Fc pull-down and was not detected in the Fc alone pull-down (FIG. 8A). To further confirm this interaction, endogenous CLEC14A was immunoprecipitated from HUVEC whole cell lysates. Western blot analysis confirmed MMRN2 co-precipitation in the CLEC14A precipitate but was not detected in the IgG control (FIG. 8B).

### Development and Validation of CLEC14A Monoclonal Antibodies

To further our understanding of CLEC14A, we next produced cross-species reactive antibodies. To enable this, murine CLEC14A protein with a human Fc tag was expressed in HEK293T cells and purified on a protein A column. Mice were then immunised with 50 μg mCLEC14A with complete Freund's adjuvant to break tolerance. Clones were screened for activity against human CLEC14A or human Fc. To confirm the clones could recognise cell bound CLEC14A, HEK293T cells overexpressing HA-CLEC14A were stained with clone C2 or C4 or a monoclonal HA tag antibody. FACs analysis shows increased fluorescence for each of the antibodies in the HA-CLEC14A overexpressing cells compared to control transfected cells (data not shown). To confirm that antibodies recognise the endogenous form of CLEC14A, these clones were used to stain HUVEC treated with control or clec14a targeted siRNAs. Control HUVEC were stained strongly by clone C2 and C4 and this staining was reduced to isotype control levels by knockdown of CLEC14A (data not shown). These results confirmed the specificity of the CLEC14A monoclonal antibodies.

To determine whether the C2 and C4 clones bind to the same region of CLEC14A, HUVECs were pre-treated with BSA, C2 or C4 antibody prior to C2-FITC staining. C2 incubation blocked C2-FITC staining effectively, but C4 had little effect. The same pre-treatment was repeated prior to C4-FITC staining. C2 antibody did not affect C4-FITC staining however, HUVECs pre-treated with C4 showed reduced binding of C4-FITC. From these results we can conclude that C2 and C4 bind to discrete regions of CLEC14A.

### A CLEC14A Monoclonal Antibody Blocks CLEC14A-MMRN2 Binding

To determine whether either of these CLEC14A monoclonal antibodies could inhibit the binding of MMRN2 to CLEC14A, CLEC14A-ECD-Fc was pre-incubated with increasing concentrations of mIgG1, or C2, or C4, prior to incubation with lysates from HEK293T cells overexpressing MMRN2. Precipitates were then separated and probed for MMRN2 or CLEC14A-ECD-Fc. MMRN2 binding was observed for CLEC14A-ECD-Fc precipitates blocked with mIgG1 or C2 but no MMRN2 binding was observed in the C4 blocked precipitates. This confirms that the C4 but not the C2 monoclonal antibody blocks MMRN2 binding to CLEC14A. (Data not shown)

### CLEC14A-MMRN2 Blocking Antibody Inhibits Tumour Growth

Mice with LLC tumours were injected intraperitoneally twice per week with 10 μg C4 or mIgG1 (control) for the duration of the experiment. Tumour growth was slowed for mice treated with C4 antibody compared to the control, mIgG1, treatment group (FIG. 9A). Tumours from the C4 treated mice were smaller in size (FIG. 9B) and weight (FIG. 9C) than control animals. Again we examined the vascular density within these tumours. Tissue sections were stained with an anti-CD31 antibody and fluorescent analysis revealed a reduced density of discrete vessels (FIGS. 9D and 9E) and the percentage endothelial coverage (FIG. 9F), suggesting that CLEC14A binding to MMRN2 is an important functional component of tumour induced angiogenesis.

### Discussion

CLEC14A is one of a small group of endothelial genes that contribute to tumour angiogenesis in multiple tumour types. Here we demonstrate that through loss of CLEC14A, tumour growth is inhibited in vivo (FIG. 7). A similar phenotype has also been observed for other tumour endothelial markers, such as TEM8, Endoglin, Galectin, ELTD1, and Endosialin and this demonstrates the importance of these tumour endothelial expressed genes in vascularisation and tumour growth.

Upregulation of CLEC14A has been observed in human tumours and murine models of pancreatic and cervical cancer which supports the findings that clec14a expression is upregulated on tumour vessels in the LLC model (FIG. 7). CLEC14A has been shown to regulate multiple aspects of endothelial biology including adhesion, migration, tube formation, and the results show that it is also important for sprouting angiogenesis in vitro and in vivo (FIGS. 5 and 6). We can infer that this role of CLEC14A is through endothelial-endothelial interactions or endothelial-extracellular matrix interactions, because in vitro HUVEC sprouting is perturbed by CLEC14A knockdown, suggesting the presence of other cell types is dispensable. We also observed for the first time upregulation of clec14a expression on neoangiogenic vessels in the subcutaneous sponge assay (FIG. 6). This is expected as newly formed endothelial sprouts have been modelled to experience extremely low shear stress (0.2 Pa) from the 4.2 μm of the bifurcation point to the tip of the sprout, and clec14a expression is known to be upregulated by low shear stress.

The interaction of CLEC14A with MMRN2 has been shown through pulldown of proteins from HUVEC lysates using the extracellular domain of CLEC14A, as well as co-immunoprecipitation of the endogenous proteins (FIG. 8). Through the generation and validation of CLEC14A monoclonal antibodies, we identified two antibodies that bind to discrete regions of CLEC14A (C2 and C4) and have shown that the C4 but not the C2 clone blocks the interaction of CLEC14A with MMRN2. To probe the function of the CLEC14A-MMRN2 interaction, we used the C4 antibody in Matrigel tube forming assays and found an increase in branching and decrease in evolved meshes. It was further found that the CLEC14A-MMRN2 interaction is important for tumour growth (FIG. 9), C4 treatment recapitulated tumour growth and reduced tumour vascularity as seen in clec14a −/− mice (FIG. 7). Although in this example no ligand or mode of activity was identified, this is the first time that CLEC14A and a specific extracellular interaction has been shown to be important for tumour growth, and suggests a hitherto avenue into new anti-angiogenic therapies.

### Example 3 CLEC14A Monoclonal Antibodies C1, C4 and C5 Block CLEC14A-MMRN2 Interaction

To determine which CLEC14A monoclonal antibodies could inhibit the binding of MMRN2 to CLEC14A, CLEC14A-ECD-Fc was pre-incubated with increasing concentrations of mIgG1, or CR1-5, prior to incubation with lysates from HEK293T cells overexpressing MMRN2. Precipitates were then separated and probed for MMRN2 or CLEC14A-ECD-Fc. MMRN2 binding was observed for CLEC14A-ECD-Fc precipitates blocked with mIgG1 or C2 and C3 but no MMRN2 binding was observed in the C1, 4 and 5 blocked precipitates. This confirms that antibodies C1, 4 and 5 bind CLEC14a on an epitope that is distinct from the one that C2 and 3 monoclonal antibodies bind and thus specifically block the MMRN2 interaction with CLEC14A.

### Example 4 Mapping of MMRN2 Binding Domain and CRT Antibodies

### 1) MMRN2 Binds to Either the CTLD or SUSHI Domain or CLEC14a

The binding of MMRN2 to CLEC14A was narrowed down to the CTLD or SUSHI domain of CLEC14A. It is likely that without the CTLD or SUSHI domain present in the domain deletions, CLEC14A is not properly folded resulting in it no longer binding to MMRN2 (Or the CRT antibodies). This was found out using deletion constructs of CLEC14A far Western blotted with MMRN2 shown in FIG. 11.

### 2) CRT Antibodies Bind to CTLD Domain of CLEC and not SUSHI

To further determine whether the CTLD or SUSHI was the binding domain and to ensure correct folding Chimeric constructs of CLEC14A were made with CTLD or SUSHI domains swapped with those of thrombomodulin (also known as CD141)—a type 14 CTLD family member which does not bind to MMRN2.

The sequences of Chimera 5 (CLEC14A with CTLD of CD141) and Chimera 6 (CLEC14A with SUSHI of CD141) are shown in FIG. 12.

Binding of CRT antibodies was analysed using flow cytometry. All constructs have a C-terminus GFP tag so green cells were gated and stained red. All CRT antibodies bind WT CLEC14A and—as expected—none binds to WT CD141 (FIG. 13). In addition, none of the antibodies bound to Chimera 5 (except slight binding by CRT2) and all of the antibodies bind to Chimera 6 (except CRT2) (FIG. 13). This confirms that the binding site of the antibodies CRT1, 3, 4 and 5 and MMRN2 are within the C type lectin domain. It is possible that CRT2 binds on a region between the CTLD and sushi domain.

3) CRT Antibodies that Block MMRN Interaction do not Bind to the Regions Specified in WO 2013/187724 but to a Region that Includes aa 97-108 of CLEC14a CTLD

To further determine the binding region of the antibodies and MMRN2, chimeric loop constructs were made. This was based on the structural predictions of CLEC14A CTLD and also the regions that the WO2013/187724 antibodies bind to.

CLEC14A with regions 1-42 of CD141
CD141 sequence -
(SEQ ID NO. 21)
MLGVLVLGALALAGLGFPAPAEPQPGGSQCVEHDCFALYPGP
CLEC14A with regions 97-108 of CD141
CD141 sequence -
(SEQ ID NO. 22)
QLPPGCGDPKRL
CLEC14A with regions 122-142 of CD141
CD141 sequence -
(SEQ ID NO. 23)
TSYSRWARLDLNGAPLCGPL

The alignment is shown in FIG. 12. Unfortunately 1-42 and 122-142 chimeras did not fold correctly. This is thought due to the fact they are present on the cell surface (stain positive for CLEC14A polyclonal antibodies, but they do not stain for any of the C antibodies not even C2.

However the 97-108 chimera does bind C2 and C3 showing that this mutant is correctly folded. This mutant does not bind MMRN2 or C1, 4 or 5 (which are the antibodies thought to block the CLEC14A-MMRN2 interaction) (FIG. 15). Therefore we conclude that the binding domain is dependent upon the loop containing the following residues: ERRRSCHTLENE (SEQ ID NO: 24).

Residues 97-108 were swapped with corresponding regions from thrombomodulin. This resulted in correct folding as C2 and C3 can still bind (FIG. 16). However C1, C4 and C5 cannot recognise this mutant suggesting this to be the binding region.

This experiment has been repeated three times with the same result.

### Example 5—Antibody Drug Conjugate Tumour Data

Wild type male C57BL6 mice aged between 6-8 weeks were subcutaneously injected with 1×10̂6 Lewis lung carcinoma (LLC) cells in the right flank. Once tumours reached a palpable size, mice were randomly assigned to each treatment group, B12-ADC, or C4-ADC/CRT3-ADC. Mice received two intravenous injections into the tail vein one week apart of 1 mg/kg. One week after final injection mice were culled, tumours were excised and wet weights were measured. The data is shown in FIGS. 17A and B.

HUVECs were treated with CRT-3 ADC and fluorescent imaging was carried out to determine the localisation of CRT-3 after 0 and 90 minutes. The results are shown in FIG. 18A. Further, the cytotoxicity was measured using a Cell Titre Glo luminescent cell viability assay and the results are shown in FIG. 18B. As described above, 1 million Lewis lung carcinoma cells were injected subcutaneously into the right flank of 2 mice and allowed to grow to a visible size. 1 mg/kg of CRT-3-ADC or B12-ADC (control) was administered through tail vein injections. The mice were observed for an hour and culled 24 hours later.

### Results

The results shown in FIG. 18A demonstrates that the CRT3-ADC are internalised. Further treatment with CRT3-ADC for 24 hours had no effect on the overall health of the mouse. Extensive haemorrhage at the site of the tumour was observed only in the CRT3-ADC treated mouse and not the control, demonstrating tumour-specific disruption of angiogenesis.

### Example 6—CAR Construction and Experiments

### Materials and Methods

### Generation of CAR Constructs

Hybridomas expressing CLEC14A-specific monoclonal antibodies that cross react with human and mouse forms of the protein were obtained as described in Noy et al (Blocking CLEC14A-MMRN2 binding inhibits sprouting angiogenesis and tumour growth. Oncogene. 2015). Gene constructs encoding an scFv were then isolated from each of the mouse hybridomas by RT-PCR using degenerate primer sets designed to amplify all mouse V-gene families as previously described in Hawkins et al (Idiotypic vaccination against human B-cell lymphoma. Rescue of variable region gene sequences from biopsy material for assembly as single-chain Fv personal vaccines. Blood. 1994; 83(11):3279-88.

The scFv genes were then subcloned into two previously described CAR vectors pMP71.tCD34.2A.CD19ζ and pMP71.tCD34.2A.CD19.IEVζ (Cheadle et al, J. Immunol., 2014, 192(8), 3654-65) as a ClaI, NotI fragment, replacing the CD19-specific scFv region. These vectors were originally constructed using the MP71 retroviral expression plasmid (a kind gift from C. Baum, Hannover) and coexpressed a truncated CD34 marker gene (Fehse et al, Mol Ther., 2000; 105 Pt 1: 448-56).

### Transduction of Human and Mouse T-Cells

To generate recombinant retrovirus for transducing human T cells, Phoenix amphotropic packaging cells were transfected with an MP71 retroviral vector and pCL ampho (Imgenex) using FuGENE HD (Roche) according to the manufacturer's instructions. Recombinant retrovirus for transducing mouse T cells was generated in the same way but using Phoenix ecotropic packaging cells and pCL eco. Human peripheral blood mononuclear cells (PBMCs) were isolated from heparinized blood by density gradient centrifugation on lymphoprep (Axis Shield, Oslo, Norway). PBMCs were pre-activated for 48 hours using anti-CD3 antibody (OKT3, eBioscience; 30 ng/ml), anti-CD28 antibody (R&D Systems; 30 ng/ml) and interleukin-2 (IL2; 300U/ml; Chiron, Emeryville, Calif.) using standard medium (RPMI1640 (Sigma) containing 10% foetal bovine serum (FBS; PAA, Pasching Austria), 2 mM L-glutamine, 100 IU/ml penicillin, and 100 μg/ml streptomycin) plus 1% human AB serum (TCS Biosciences, Buckingham, UK). Transduction of mouse T cells was conducted using mouse splenocytes pre-activated for 48 hours with concanavalin A (2 ug/ml; Sigma) and mouse interleukin 7 (1 ng/ml; eBioscience) in standard medium. Preactivated human and mouse T cells were subsequently transduced (or mock-transduced with conditioned supernatant from non-transfected phoenix cells) by spinfection in retronectin (Takara)-coated plates according to the manufacturer's instructions. Human T cells were then cultured in standard medium plus 1% human AB serum with IL2 (100 U/ml). After spinfection, mouse T cells were cultured for 24 hrs in standard medium with IL2 (100 U/ml), then purified using lymphoprep (Axis Shield). Where indicated, transduced cells were enriched by immunomagnetic selection using anti-CD34 microbeads (Miltenyi Biotec, Germany) according to the manufacturer's instructions. Studies with human donors were approved by the National Research Ethics Service Committee West Midlands (Solihull) and all donors gave written informed consent

### Cell Lines and Recombinant Proteins

Phoenix A or E, CHO and Lewis lung carcinoma cells were maintained in Dulbecco's modified Eagle's medium (DMEM) containing 10% foetal bovine serum (FBS; PAA, Pasching Austria), 2 mM L-glutamine, 100 IU/ml penicillin, and 100 pg/ml streptomycin. CHO cells had been transduced with the pWPI vector (Addgene) expressing full length human CLEC14A (or vector alone). Human umbilical vein endothelial cells (HUVECs) were isolated as described previously using umbilical cords obtained from Birmingham Women's Health Care NHS Trust with informed consent and with ethical approval of the south Birmingham research ethics committee. HUVECs were maintained in M199 complete medium containing 10% FBS, 4 mM L-glutamine, 10% large vessel endothelial cell growth supplement (TCS Cellworks) and cultured in plates coated with 0.1% type 1 gelatin from porcine skin (Sigma). Human and murine CLEC14A proteins with a human Fc tag were expressed in HEK293T cells and purified on a protein A column as described in Noy et al (supra)

### SiRNA Knockdown of CLEC14A

Transfection with siRNA was performed as previously described (Armstrong et al, Arteriosclerosis, thrombosis and vascular biology, 2008, 28(9): 1640-6) using the following siRNA duplexes: D1-GAACAAGACAATTCAGTAA (SEQ ID NO. 30) and D2-CAATCAGGGTCGACGAGAA (SEQ ID NO. 31) (EuroGentec, Liege, Belgium).

### Flow Cytometry

HUVECs were trypsinised and stained for 1 hr on ice with CLEC14A-specific mouse monoclonal antibodies described above (10 ug/ml) or IgG1 isotype control (Dako) in 5% normal goat serum/PBS. Cells were washed and bound antibody detected by incubating with R-PE-conjugated goat-anti mouse antibody (Serotec). Dead cells were identified by staining with propidium iodide. Human T-cells were washed with PBS and stained with Live/Dead Fixable Violet Dead Cell Stain Kit (Life Technologies) for 20 mins in the dark. Cells were then washed with flow buffer (0.5% w/v BSA+2 mM EDTA in PBS; pH7.2) and stained with anti human CD4 (PE-conjugated), anti human CD8 (FITC-conjugated) (all from BD Pharmingen) and anti-human CD34 (Pe-Cy5) (BioLegend) for 30 mins on ice in the dark. Alternatively rather than staining for CD34, CAR expression was detected directly by firstly blocking cells with human Fc fragment (10 ug/ml), then incubating them with 10 ug/ml recombinant human CLEC14A-Fc fusion protein (or Fc control) followed by sheep anti CLEC14A polyclonal antibody (R&D systems, 10 ug/ml). Finally cells were stained with FITC-conjugated rabbit anti-sheep antibody (Invitrogen, diluted 1:10). All incubations were conducted for 1 hour on ice.

When staining mouse T cells from heparinized tail bleeds they were first subject to red blood cell lysis using BD Pharm lyse (Becton Dickinson) before staining as described above but using anti mouse CD4-FITC, CD8-PE and CD45.1 (PE-Cy7 conjugated) (all BD Biosciences). Cells were analyzed using a BD LSR II flow cytometer and FlowJo software (TreeStar Inc, Ashland, Oreg.).

### CFSE Labelling

T-cells were washed twice with PBS and incubated with 2.5 μM Carboxyfluorescein succinimidyl ester (CFSE) for 10 minutes at 37° C. The labelling reaction was quenched by addition of RPMI-1640 containing 10% FBS. Cells were washed, resuspended in standard medium plus 1% human AB serum and IL2 (10 IU/ml) at 1.5×10⁶ cells/ml and added to wells containing HUVECs to give a T-cell:HUVEC ratio of 10:1. After 5 days incubation at 37° C./5% CO₂, cells were analysed by flow cytometry as described above using anti-human CD34 (Pe-Cy5).

### IFNγ Release Assay

Stimulator cells (2.5×10⁴/well) were co-cultured in triplicate with CD34+ CAR-T-cells at responder:stimulator ratios indicated. Alternatively 2×10⁴ CD34+ CAR-T cells were incubated in wells precoated with recombinant protein (1 ug/ml). Cells were incubated at 37° C./5% CO₂ in 100 μl/well of standard medium supplemented with IL2 (25U/ml). After 18 hours, culture supernatant was tested for secreted IFNγ using an ELISA (Pierce Endogen, Rockford, Ill.) according to the manufacturer's instructions.

### Cytotoxicity Assays

Chromium release assays have been described in detail previously. They were set up at known effector:target ratios (1250 targets/well) and harvested after 7.5 hours.

### In Vivo Experiments

### Toxicity Testing

Six to eight week old C57BL6 mice (Charles River Laboratories) received 4 Gy total body irradiation (TBI). Eighteen hours later, each mouse was injected into the tail vein with 2×10⁷ CAR- or Mock-transduced T cell preparations from CD45.1+ congenic BoyJ mice. Mice were monitored for signs of toxicity and immune monitoring was conducted by weekly tail bleeds. Mice were eventually culled 45 days later and major organs removed for histological analysis.

### RipTag2 Transgenic Mouse Tumour Model

Generation of RIP-Tag2 mice as a model of pancreatic islet cell carcinogenesis has been previously reported (Hanahan et al, Nature, 1985, 315 (6015), 115-122). RIP-Tag2 mice were maintained on a C57BL/6J background (The Jackson Laboratory). Cryopreserved CAR-transduced and mock transduced T cells were thawed, washed and 15 million T cells/mouse injected intravenously into the tail vein on a single occasion into 12-week old mice that had been conditioned with 4 Gy TBI the day before. From 12 weeks of age, all RIP-Tag2 mice received 50% sugar food (Harlan Teklad) to relieve hypoglycaemia induced by the insulin-secreting tumours. Total tumour burden in culled CAR-T cell-treated mice was quantified at 16 weeks of age using calipers to measure individually excised macroscopic tumours (>1 mm³) using the formula: volume=a×b²×0.52, where a and b represent the longer and shorter diameter of the tumour, respectively. The volumes of all tumours from each mouse were added to give the total tumour burden per animal. There are no age-matched control comparisons for the 16-week CAR-treated mice, since untreated RIP-Tag2 mice do not survive to 16 weeks, and thus the comparison was made to 14-week old Mock-treated mice.

### Lewis Lung Carcinoma (LLC) Mouse Model

6-8 week old female C57BL6 mice were inoculated subcutaneously on the flank with 10⁶ LLC cells. Three days later mice received 4 Gy TBI and 18 hrs after this each mouse was injected into the tail vein with 2×10⁷ CAR or Mock T cell preparations from CD45.1+ congenic BoyJ mice. Tumour growth was measured with calipers (using the formula: volume=length×width²×0.5) and bioluminescence imaging (IVIS Spectrum, Caliper Life Sciences). Immune monitoring was conducted by weekly tail bleeds.

All procedures with RipTag2 mice were approved by the Ethics Committee of the University of Turin, and by the Italian Ministry of Health, in compliance with international laws and policies. All other mouse studies were performed with appropriate UK Home Office approval.

### Tissue Preparation and Immunofluorescence Analysis

Tissues from mouse experiments were embedded in OCT (Bio Optica), frozen in dry ice and stored at −80° C. Tissue preparation and histology analysis were carried out as described (24) with the following primary antibodies: purified rat monoclonal anti-panendothelial cell antigen (550563, clone Meca32, BD Pharmingen, USA), diluted 1:100; rabbit monoclonal anti-cleaved caspase 3 (asp175, clone 5A1, Cell Signaling, USA), diluted 1:100; rabbit polyclonal anti-Fibrinogen (A0080, Dako), diluted 1:100; and rabbit monoclonal anti-CD34 (ab174720, Abcam) diluted 1:50; sheep polyclonal anti-CLEC14A (AF4968, R&D) diluted 1:50. After incubation and washing, samples were incubated with secondary antibodies anti Rabbit Alexa Fluor-488 and Alexa Fluor-555; anti Rat Alexa Fluor-488 and Alexa Fluor-555; and anti Sheep Alexa Fluor-488 (Molecular Probes) and counterstained with DAPI Nucleic Acid Stain (Invitrogen). To detect CAR-transduced T cells tissues were stained with rabbit monoclonal anti-CD34 (ab174720, Abcam) diluted 1:50 in PBS. After incubation and washing, samples were stained with anti Rabbit Alexa Fluor-555 (Molecular Probes) and counterstained with DAPI.

Human tumour tissue arrays (SuperBiochips Inc., Seoul, Korea) were stained using sheep polyclonal anti-CLEC14A (AF4968, R&D systems) diluted 1:20 and Ulex europaeus agglutinin I conjugated to rhodamine (Vectorlabs, UK) for 1 hour, followed by anti sheep FITC antibody (10 μg/ml, Invitrogen, UK).

For analysis of RipTag2 tumour tissue, the surface area occupied by vessels was quantified through the ImageJ software as the area occupied by Meca32-positive structures, compared with the total tissue area visualised by DAPI. For each animal, the total vessel area of at least four field/images was quantified. To determine the amount of fibrinogen extravasation (red channel) in each image, we drew a region of interest (ROI) close to each blood vessel (Meca32, green channel), and then quantified the mean fluorescence intensity (MFI) of red and green channels using the Leica Confocal Software Histogram Quantification Tool. In order to normalize the vessel number values obtained, we calculated the ratio between red and green channel MFI; values are expressed as percentage of red-green co-staining. To determine the expression levels of caspase 3 (green channel) in each analysed image, we considered 5 random ROIs of the same size. Then we measured the MFI of the green channel, and we normalized the values by comparing caspase 3-stained area with the total cells present in the tissue area. At least 10 images of five mice per treatment group were analyzed for each sample. Tissue from RipTag2 mice were analyzed using a Leica TCS SP2 AOBS confocal laser-scanning microscope (Leica Microsystems). All other tissues were analysed using an Axiovert 100M laser scanning confocal microscope (Carl Zeiss, Welwyn Garden City, UK).

### Statistical Analysis

Statistical analyses of data were conducted using the tests indicated and GraphPad Prism software. A p value <0.05 was considered significant.

### Results

CAR constructs have been successfully made using CRT1, 3, 4, and 5 and the expression of these CAR constructs on cells has been demonstrated (see FIGS. 19B and C, FIG. 22 and FIG. 31). Thus, retroviral CAR vectors (based on pMP71) that co-express a truncated CD34 marker gene and an scFv fragment/CD3 zeta chain chimeric receptor were generated. Expression is driven from the LTR promoter and the 2A peptide linker ensures equimolar expression of both the CD34 and the CAR. Second generation CAR constructs included the CD28 costimulatory domain. As shown in FIG. 19B, CD34 staining shows successful transduction of T cells with the vectors comprising CRT3 and 5 (both first and second generation). Similar results can be sheen for cells stained directly for expression of CAR using CLEC14A-Fc (% values show specific binding of CLEC14A-Fc having subtracted background staining with Fc alone).

Further, the data in FIG. 20 show that T-cells transduced to express a first or second generation CAR based on antibodies CRT3 or 5 can respond to CLEC14A in vitro. Results are shown for plate bound protein, protein expressed on engineered CHO cells or expressed in HUVECs, where IFNγ production can be seen by T cells transduced with the CARs in all three experiments, in response to the CLEC14A present.

CARs based on antibodies CRT3 and 5 were tested for their ability to induce cytotoxicity in CHO CLEC14A expressing cells (FIG. 21A), where an increase in the % specific lysis can be seen in cells exposed to T cells with CAR CRT3/5. Further, the proliferation of CD34+ T-cells when cultured with HUVECs shows that both CRT3 and 5 first and second generation CAR T cells proliferate under such culture conditions (i.e. due to exposure to CLEC14A expressing HUVECs). Thus, CRT3/5 CAR T cells are able to respond to CLEC14A expressing cells, resulting in the proliferation of the T-cells and specific lysis of the cells.

A CAR based on CRT1 antibody also shows activity against CLEC14A expressing targets. Particularly, second generation CRT1 CAR T cells were shown to respond to CLEC14A expressed on CHO cells engineered to express CLEC14A and HUVECs (measured by IFNγ release). Further, first and second generation CRT1 CAR T cells were shown to induce specific lysis in CHO CLEC14A expressing cells (see FIG. 22). FIGS. 32 to 35 further show the T-cell response to CLEC14A when transduced with CRT1-CAR and the % lysis induced by such transduced T-cells.

First or second generation CRT3 and 5 CAR T cells were injected into C57/BL6 mice to determine the toxicity of the CAR T cells to healthy mice. Mice were monitored for 45 days and showed no visible signs of toxicity. FIG. 23 shows that the body weights of the mice increased during this time, and weekly tail bleeds indicate that the CAR T cells persisted for at least 5 weeks after injection and that they comprised at least 30% of the total circulating T cell population during this time. Splenocytes harvested at the end of the experiment were still capable of responding to CLEC14A (both mouse and human). Further, as shown in FIG. 24, histological analysis of several tissues (brain, heart, lung, liver, colon and kidney) showed no pathology for mice infused with second generation CRT3/5 CAR T cells.

The anti-tumour effect of second generation CARs based on CRT3 or CRT5 antibodies was tested in C57BL6 mice which had previously been injected with 1 million Lewis Lung Carcinoma cells. T cells transduced with the CAR constructs were injected into the tail veins of the mice (20 million T cells) and tumour growth was monitored. As can be seen from FIG. 25, mice injected with either CRT3 or 5 second generation CAR T cells had tumours of smaller volumes than control mice, demonstrating the anti-tumour effect of the CAR T cells. Further, FIG. 26 shows a reduced mean tumour weight, reduced vascular density and an increased level of vascular leakage in mice injected with either CRT3 or 5 second generation CART cells.

Second generation CRT5 CAR T cells were injected into RIP-Tag2 mice, where the rat insulin promoter directs expression of the SV40 Large T antigen transgene to beta cells of the pancreatic islets, resulting in tumours at around 10 weeks of age and death by week 14. As can be seen in FIG. 27, tumour size was significantly reduced in the CAR T cell injected mice compared to untreated or mock T-cell control mice. Further, FIG. 28 shows that CAR transduced T cells accumulate in RIP-Tag2 tumours 4 weeks after intravenous injection.

Histological analysis of RipTag2 tumours from mice treated with CAR engineered T cells showed that vascular density is reduced, apoptotic vessels are increased and fibrinogen staining is decreased compared to mice treated with Mock T cells (FIG. 29).

### Example 7—Functionally Active TCR Specific for the WT1 Derived Peptide pWT126 (RMFPNAPYL)

A TCR has been cloned that is specific for a peptide RMFPNAPYL (WT126) of the Wilms Tumour antigen-1 (WT1) which is presented by HLA-A2 class I molecules. The WT1 transcription factor is expressed in various human malignancies, including leukaemia, breast cancer, colon cancer, lung cancer, ovarian cancer and other. The CTL (from which the TCR was cloned) show killing activity against human cancer cells that express WT1, but not against normal human cells that express physiological levels of WT1.

The therapeutic goal was to equip patient T cells with this potent and specific killing activity by transfer of the genes encoding the TCR. For this, TCR genes have been inserted into retroviral vectors and it has been demonstrated that gene transduced human T cells show killing activity against WT1 expressing human cancer and leukemia cell lines. The specificity profile of this CTL line has been described in several research papers and can be summarized as: (1) Killing of HLA-A2-positive targets coated with the WT1-derived peptide pWT126 (Gao et al (2000) Blood 95, 2198-2203); (2) Killing of fresh HLA-A2-positive leukaemia cells expressing WT1 (Gao et al (2000) Blood 95, 2198-2203); (3) Killing of HLA-A2-positive leukemia CFU progenitor cells (Gao et al (2000) Blood 95, 2198-2203; Bellantuono et al (2002) 100, 3835-3837); (4) Killing of HLA-A2-positive leukaemia LTC-IC stem cells (Bellantuono et al (2002) Blood 100, 3835-3837); (5) Killing of HLA-A2-positive NOD/SCID leukaemia initiating cells (Gao et al (2003) Transplantation 75, 1429-1436); and (6) No killing of normal HLA-A2-positive NOD/SCID engrafting hematopoietic stem cells (Gao et al (2003) Transplantation 75, 1429-1436). It has now been shown that human T cells transduced with the WT1-specific TCR display similar specificity as the CTL line from which the TCR was cloned.

The data described in detail in the legends to FIGS. 36 to 43 indicate that TCR gene transfer into human T cells is feasible and that it leads to the surface expression of the introduced TCR chains. The recipient T cells show killing activity against HLA-A2-positive targets coated with the pWT126 peptide. The TCR-transduced T cells also kill human tumour cells expressing WT1 endogenously. In addition, the transduced T cells produce IFN-γ in an HLA-A2-restricted, peptide-specific fashion. Finally, the transferred TCR can function in CD4-positive helper T cells. These CD4-positive T cells show HLA-A2-restricted, antigen-specific killing activity and antigen-specific cytokine production (not shown). This indicates that TCR gene transfer can be used to confer HLA class 1-restricted antigen-specific effector function to both CD8-positive and CD4-positive human T cells.

### Methodology

### Cell Lines

T2 is a transporter associated with antigen processing (TAP)-deficient human HLA-A2+ cell line that can be efficiently loaded with exogenous peptides. The BV173 cell line was established from the peripheral blood of a male patient with CML. All cells were cultured in RPMI plus 10% FCS at 37° C.

### Synthetic Peptides and HLA-A2/Peptide Complex Tetramer

pWT126 (RMFPNAPYL) and pWT235 (CMTWNQMNL) are HLA-A2 binding peptides derived from human WT1. pWT126 was dissolved in PBS and pWT235 was dissolved in DMSO before diluting in PBS to give a concentration of 2 mM.

### Retroviral TCR Constructs and Transduction of TCR Genes

The WT1-specific TCR alpha and beta genes were isolated from the allo-restricted pWT126-specific human CTL line 77. To clone the TCR genes, total RNA was extracted from CTL line 77, and reverse transcribed into cDNAs. cDNAs were amplified using a consensus primer that binds to both variable alpha and beta genes in combination with a set of constant primers. The isolated TCR Valpha or V beta gene was then cloned into pMP71 retroviral vector using the NotI and EcoRI restriction sites.

2×10⁶ amphotropic packaging cells were seeded into a T25 flask and 24 hours later were transiently transfected with retroviral TCR constructs using calcium phosphate precipitation. In preparation for transduction, PBMCs were activated using anti-CD3 antibody and IL-2 for 2 days. Activated T cells (3×10⁶) were then resuspended in 3 ml of normal growth medium plus 3 ml of virus supernatant harvested from transfected packaging cells and plated in 6-well plates costed with fibronectin. Plates were incubated at 37° C. at 5% CO2 and 24 to 48 hours after transduction, expression of TCR transgenes was carried out.

### IFNγ-Secretion Assays

TCR-transduced T cells (5×10⁴) were stimulated with 5×104 leukaemia cells or peptide-coated T2 cells (1:1 ratio) in triplicate in a 96-well plate. After 24 hours incubation, the supernatant was harvested and tested in an interferon γ enzyme linked immunosorbent assay (ELISA) using a human IFNγ determination kit (AMS Biotechnology).

### Example 8: Selection and Treatment of a Patient

Peripheral blood monocyte cells (PBMCs) are taken from an HLA-A2-positive patient who has a WT1-expressing malignancy. The PBMCs are activated with anti-CD3/CD28 antibodies added to the culture or on beads for 3 days and then transduced with TCR encoding retroviral particles as described in Example 1. At day 5 we can demonstrate that transduced CD4 and CD8 T cells express the introduced TCR. At day 6 we can demonstrate antigen-specific activity of the transduced T cells. At day 6 the transduced T cells are reinfused into the patient.

### Example 9: Wound Healing in Tumour Bearing Mice

At day 0, mice were injected subcutaneously with 1×10⁶ Lewis Lung carcinoma cells and on day 3 were irradiated with 4 Gy. On day 3 mice were injected intravenously with 13×10⁶ Mock (n=7) or CAR T cells (n=7) (CRT5 CAR). Cells were 31% CD4+(93% CD34+) and 43% CD8+(62% CD34+). Wound healing was observed for 7 days.

The results in FIG. 44 show that mouse T cells expressing the CRT5 CAR did not impede healing of a skin wound in tumour bearing mice compared to similar mice treated with mock-transduced T cells.

### Example 10: CRT5 CAR in a PDAC Mouse Model

K-RasG12D; Ink4a/Arf−/−; p53R172H cells are injected into the pancreas of syngeneic immunocompetent mice to generate the PDAC mouse model which is a mouse model of pancreatic adenocarcinoma. The staining of tumours from this mouse model has shown CLEC14A expression on the majority of tumour vessels. Treatment of PDAC mice with CRT5 CAR T cells (where the CAR comprises a costimulatory domain from CD28) results in significant tumour control (FIG. 45) 3 weeks after treatment. Results from an additional experiment can be seen in FIG. 50.

### Example 11: Titration of CRT1, 3 and 5 Against CLEC14A

CLEC14A was expressed as an Fc fusion protein for incubation with CRT1, 3 and 5 CAR (CD28 costimulatory domain) T cells. All CAR-T cell lines were diluted with Mock T cells to equalise for transduction efficiencies. The results can be seen in FIG. 46 where it is shown that all of the tested CAR T cells respond well to CLEC14A (data shown are means of triplicate cultures+SD).

### Example 12: CRT1, 3 and 5 CAR T Cell Cytotoxicity and Proliferation Assay

A cytotoxicity study was carried out using CRT1, 3 and 5 CAR (with CD28 costimulatory domain) T cells. The T cells were diluted with Mock T cells to equalise for transduction efficiencies and were incubated with mouse endothelial cells expressing human CLEC14A. The results are shown in FIG. 48A which demonstrate that all three tested CARs can mediate cytotoxicity. The data shown are means of triplicate cultures+SD.

Further, a proliferation assay was carried out (CFSE labelling) with CRT1, 3 and 5 CAR (CD28 costimulatory domain) T cells stimulated with plate-bound recombinant CLEC14A-Fc fusion proteins. All the CAR T cell lines were diluted with Mock T cells to equalise for transduction efficiencies and the results can be seen in FIG. 48B, where all three tested CARs were capable of proliferating after stimulation. The data shown are for CD8+ T cells only but similar data were obtained for CD4+ T cells.

### Example 13: CARs with Different Costimulatory and Transmembrane Regions

The following CARs have been cloned and engineered into T cells from a single donor using a retroviral vector:

1) CRT3-CD28 TM-CD28 costim signal-CD3 (CRT3.28z)

2) CRT3-CD8 TM-4-1BB costim signal-CD3 (CRT3.BBz)

3) CRT3-CD28 TM-CD28 and 4-1BB costim signals-CD3 (CRT3.28BBz)

4) CRT3-CD28 TM-CD28 and OX40 costim signals-CD3 (CRT3.28Oxz)

5) CRT3-CD8 TM-4-1BB and OX40 costim signals-CD3 (CRT3.BBOxz)

All constructs generated transduced well into T cells. The function of the different constructs was assessed in vitro, analysing cytokine production, cytotoxicity and proliferative response (see FIG. 49). Cytokine release indicated strong antigen specific responses especially by T cells expressing CRT3.28z and CRT3.28Oxz. Cytokine production was analysed by measuring IFNgamma production in response to titrated numbers of CHO cells expressing human CLEC14A (or vector only control). All CAR T cell lines were diluted with Mock T cells to equalise for transduction efficiencies. Data shown are means of triplicate cultures+SD. Cytotoxic activity was measured against mouse endothelial cells (SEND) engineered to express CLEC14A and the proliferative response was measured following stimulation with plate-bound recombinant CLEC14A-Fc fusion proteins (data not shown).

### Example 14: Determination of Cytokine Release from CAR T Cells Following Stimulation with Chimeric CLEC14A

Chimeric forms of CLEC14A that contain the human sequence but with the transmembrane and/or intracellular domains of mouse origin were expressed in 293 and SEND cells. These cells were sorted using GFP co-expressed from a lentiviral vector to equalise for CLEC expression and then tested using CAR T cells (CRT1, 3 and 5 with CD28 costimulatory domain). The release of IFN gamma was measured after incubation of the CAR T cells with both the 293 and SEND cells. The results can be seen in FIG. 51. Additionally, the cytotoxicity of the T cells when incubated with the CLEC14A chimera expressing SEND cells was determined. All CAR T cell lines were diluted with Mock T cells to equalise for transduction efficiencies. Data shown are means of triplicate cultures.

As can be seen from FIG. 51, all of the tested CRT1, 3 and 5 CAR T cells result in the release of IFN gamma from 293 and SEND cells expressing either human CLEC14A (huCLEC), human CLEC14A with mouse intracellular domain (A1) and human CLEC14A with mouse transmembrane and intracellular domain (B1).

1. A nucleic acid molecule comprising a polynucleotide sequence encoding a chimeric antigen receptor comprising
(i) an anti-CLEC14A binding domain,
(ii) a transmembrane domain and
(iii) an intracellular signalling domain;
wherein said anti-CLEC14A binding domain is capable of binding to the C-type lectin domain of CLEC14A.

2. The nucleic acid molecule of claim 1 wherein said anti-CLEC14A binding domain inhibits the interaction between CLEC14A and MMRN2.

3. The nucleic acid molecule of claim 1 or 2 wherein said anti-CLEC14A binding domain binds to residues 97-108 of CLEC14A, wherein CLEC14A has the amino acid sequence as set out in SEQ ID NO. 1.

4. The nucleic acid molecule of claim 1, wherein said anti-CLEC14A binding domain has an amino acid sequence comprising a heavy chain CDR3 having an amino acid sequence of SEQ ID NO. 213 or a variant thereof having one, two or three amino acid substitutions.

5. The nucleic acid molecule of claim 1 or 4, wherein said anti-CLEC14A binding domain has an amino acid sequence comprising a light chain CDR3 having an amino acid sequence of SEQ ID NO. 215 or a variant thereof having one, two or three amino acid substitutions.

6. The nucleic acid molecule of any one of claim 1, 4 or 5, wherein said anti-CLEC14A binding domain has an amino acid sequence comprising at least one of:
(a) a heavy chain CDR1 having an amino acid sequence of (S/T) SYW (UM) (E/H) (SEQ ID NO. 150), GYTF (S/T) SYW (SEQ ID NO. 151) or a variant thereof having one, two or three amino acid substitutions,
(b) a heavy chain CDR2 having an amino acid sequence of WIG (E/A) I (L/Y) PG (S/N) (G/S) (S/D) T (N/S) (SEQ ID NO. 152), I (L/Y) PG (S/N) (G/S) (S/D) T (SEQ ID NO. 153) or a variant thereof having one, two or three amino acid substitutions, and/or
(c) a heavy chain CDR3 having an amino acid sequence of (A/T) (R/H) (G/X) (G/X) (D/X) Y (D/Y) (E/G) (E/S) (Y/D) Y (V/A/L) MD (SEQ ID NO. 154), (A/T) (R/H)(G/X) (G/X)(D/X) Y (D/Y) (E/G) (E/S) (Y/D) Y (V/A/L) MDY (SEQ ID NO. 155) or a variant thereof having one, two or three amino acid substitutions,
wherein X is no amino acid residue.

7. The nucleic acid molecule of any one of claims 1 or 4 to 6, wherein said anti-CLEC14A binding domain has an amino acid sequence comprising at least one of
(a) a light chain CDR1 having an amino acid sequence of S/X S/X S Y M/L Y/H WY (SEQ ID NO. 156), SSVS Y/S S/X Y/X (SEQ ID NO. 157) or a variant thereof having one, two or three amino acid substitutions,
(b) a light chain CDR2 having an amino acid sequence of L L/W IY D/S TSNLA (SEQ ID NO. 158), D/S TS or a variant thereof having one, two or three amino acid substitutions and/or
(c) a light chain CDR3 having an amino acid sequence of Q/H Q W/Y S/H S/R Y/S P L/R (SEQ ID NO. 160), Q/H Q W/Y S/H S/R Y/S P L/R T F/X (SEQ ID NO. 161) or a variant thereof having one, two or three amino acid substitutions,
wherein X is no amino acid.

8. The nucleic acid molecule of any one of claims 1 to 7 wherein said anti-CLEC14A binding domain has an amino acid sequence comprising a heavy chain CDR3 having an amino acid sequence of SEQ ID NO. 207 or a variant thereof having one, two or three amino acid substitutions.

9. The nucleic acid molecule of any one of claims 1 to 8, wherein said anti-CLEC14A binding domain has an amino acid sequence comprising at least one of
(a) a heavy chain CDR1 having an amino acid sequence of SEQ ID NO. 32, SEQ ID NO. 44, or a variant thereof having one, two or three amino acid substitutions,
(b) a heavy chain CDR2 having an amino acid sequence of SEQ ID NO. 33, SEQ ID NO. 45, or a variant thereof having one, two or three amino acid substitutions and/or
(c) a heavy chain CDR3 having an amino acid sequence of SEQ ID NO. 116, SEQ ID NO. 118, SEQ ID NO. 34, SEQ ID NO. 46, SEQ ID NO. 100, SEQ ID NO. 102, or a variant thereof having one, two or three amino acid substitutions.

10. The nucleic acid molecule of any one of claims 1 to 9 wherein said anti-CLEC14A binding domain has an amino acid sequence comprising a light chain CDR3 having an amino acid sequence of SEQ ID NO. 208 or a variant thereof having one, two or three amino acid substitutions.

11. The nucleic acid molecule of any one of claims 1 to 10, wherein said anti-CLEC14A binding domain has an amino acid sequence comprising at least one of
(a) a light chain CDR1 having an amino acid sequence of SEQ ID NO. 35, SEQ ID NO. 47, or a variant thereof having one, two or three amino acid substitutions,
(b) a light chain CDR2 having an amino acid sequence of SEQ ID NO. 36, DTS, or a variant thereof having one, two or three amino acid substitutions, and/or
(c) a light chain CDR3 having an amino acid sequence of SEQ ID NO. 37, SEQ ID NO. 49, or a variant thereof having one, two or three amino acid substitutions.

12. The nucleic acid molecule of any one of claims 1 to 11, wherein said anti-CLEC14A binding domain has an amino acid sequence comprising
(a) SEQ ID NO. 32, SEQ ID NO. 33 and SEQ ID NO. 116, or a variant thereof having one, two or three amino acid substitutions in any one or more of SEQ ID NO. 32, 33 and/or 116,
(b) SEQ ID NO. 44, SEQ ID NO. 45 and SEQ ID NO. 118, or a variant thereof having one, two or three amino acid substitutions in any one or more of SEQ ID NO. 44, 45 and/or 118,
(c) SEQ ID NO. 32, SEQ ID NO. 33 and SEQ ID NO. 100, or a variant thereof having one, two or three amino acid substitutions in any one or more of SEQ ID NO. 32, 33 and/or 100,
(d) SEQ ID NO. 44, SEQ ID NO. 45 and SEQ ID NO. 102, or a variant thereof having one, two or three amino acid substitutions in any one or more of SEQ ID NO. 44, 45 and/or 102,
(e) SEQ ID NO. 32, SEQ ID NO. 33 and SEQ ID NO. 34, or a variant thereof having one, two or three amino acid substitutions in any one or more of SEQ ID NO. 32, 33 and/or 34; or
(f) SEQ ID NO. 44, SEQ ID NO. 45 and SEQ ID NO. 46, or a variant thereof having one, two or three amino acid substitutions in any one or more of SEQ ID NO. 44, 45 and/or 46.

13. The nucleic acid molecule of any one of claims 1 to 12, wherein said anti-CLEC14A binding domain has an amino acid sequence comprising
(a) SEQ ID NO. 35, SEQ ID NO. 36, and SEQ ID NO. 37, or a variant thereof having one, two or three amino acid substitutions in any one or more of SEQ ID NO. 35, 36 and/or 37, or
(b) SEQ ID NO. 47, DTS and SEQ ID NO 0.49, or a variant thereof having one, two or three amino acid substitutions in any one or more of SEQ ID NO. 47, 49 and/or DTS.

14. The nucleic acid molecule of any one of claims 1 to 13 wherein said anti-CLEC14A binding domain has an amino acid sequence comprising
(a) SEQ ID NO. 32, SEQ ID NO. 33, SEQ ID NO. 116, SEQ ID NO. 35, SEQ ID NO. 36, and SEQ ID NO. 37, or a variant thereof having one, two or three amino acid substitutions in any one or more of SEQ ID Nos 32, 33, 116, 35, 36 and/or 37;
(b) SEQ ID NO. 44, SEQ ID NO. 45, SEQ ID NO. 118, SEQ ID NO. 47, DTS and SEQ ID NO. 49, or a variant thereof having one, two or three amino acid substitutions in any one or more of SEQ ID Nos 44, 45, 118, 47, 49 and/or DTS
(c) SEQ ID NO. 32, SEQ ID NO. 33, SEQ ID NO. 100, SEQ ID NO. 35, SEQ ID NO. 36, and SEQ ID NO. 37, or a variant thereof having one, two or three amino acid substitutions in any one or more of SEQ ID Nos 32, 33, 100, 35, 36 and/or 37;
(d) SEQ ID NO. 44, SEQ ID NO. 45, SEQ ID NO. 102, SEQ ID NO. 47, DTS, and SEQ ID NO. 49, or a variant thereof having one, two or three amino acid substitutions in any one or more of SEQ ID Nos 44, 45, 102, 47, 49 and/or DTS;
(e) SEQ ID NO. 32, SEQ ID NO. 33, SEQ ID NO. 34, SEQ ID NO. 35, SEQ ID NO. 36 and SEQ ID NO. 37, or a variant thereof having one, two or three amino acid substitutions in any one or more of SEQ ID Nos 32, 33, 34, 35, 36 and/or 37; or
(f) SEQ ID NO. 44, SEQ ID NO. 45, SEQ ID NO. 46, SEQ ID NO. 47, DTS, and SEQ ID NO. 49, or a variant thereof having one, two or three amino acid substitutions in any one or more of SEQ ID Nos 44, 45, 46, 47, 49 and/or DTS.

15. The nucleic acid molecule of any one of claims 1 or 4 to 7, wherein said anti-CLEC14A binding domain has an amino acid sequence comprising at least one of
(a) a heavy chain CDR1 having an amino acid sequence of SEQ ID NO. 216 or a variant thereof having one, two or three amino acid substitutions,
(b) a heavy chain CDR2 having an amino acid sequence of SEQ ID NO. 217 or a variant thereof having one, two or three amino acid substitutions, and/or
(c) a heavy chain CDR3 having an amino acid sequence of SEQ ID NO. 218 or a variant thereof having one, two or three amino acid substitutions.

16. The nucleic acid molecule of any one of claim 1, 4 to 7 or 15 wherein said anti-CLEC14A binding domain has an amino acid sequence comprising at least one of
(a) a heavy chain CDR1 having an amino acid sequence of SEQ ID NO. 64, SEQ ID NO.76, or a variant thereof having one, two or three amino acid substitutions,
(b) a heavy chain CDR2 having an amino acid sequence of SEQ ID NO. 65, SEQ ID NO. 77, or a variant thereof having one, two or three amino acid substitutions, and/or
(c) a heavy chain CDR3 having an amino acid sequence of SEQ ID NO. 66, SEQ ID NO. 78, or a variant thereof having one, two or three amino acid substitutions.

17. The nucleic acid molecule of any one of claim 1, 4 to 7, 15 or 16 wherein said anti-CLEC14A binding domain has an amino acid sequence comprising at least one of
(a) a light chain CDR1 having an amino acid sequence of SEQ ID NO. 219 or a variant thereof having one, two or three amino acid substitutions,
(b) a light chain CDR2 having an amino acid sequence of SEQ ID NO. 220 or a variant thereof having one, two or three amino acid substitutions, and/or
(c) a light chain CDR3 having an amino acid sequence of SEQ ID NO. 221 or a variant thereof having one, two or three amino acid substitutions.

18. The nucleic acid molecule of any one of claims 1, 4 to 7 or 15 to 17 wherein said anti-CLEC14A binding domain has an amino acid sequence comprising at least one of
(a) a light chain CDR1 having an amino acid sequence of SEQ ID NO. 67, SEQ ID NO. 79 or a variant thereof having one, two or three amino acid substitutions,
(b) a light chain CDR2 having an amino acid sequence of SEQ ID NO. 68, STS, or a variant thereof having one, two or three amino acid substitutions, and/or
(c) a light chain CDR3 having an amino acid sequence of SEQ ID NO. 69, SEQ ID NO. 81, or a variant thereof having one, two or three amino acid substitutions.

19. The nucleic acid molecule of any one of claims 1, 4 to 7 or 15 to 18 wherein said anti-CLEC14A binding domain has an amino acid sequence comprising
(a) SEQ ID NO. 64, SEQ ID NO. 65 and SEQ ID NO. 66 or a variant thereof having one, two or three amino acid substitutions in any one or more of SEQ ID Nos 64, 65 and/or 66; or
(b) SEQ ID NO. 76, SEQ ID NO. 77 and SEQ ID NO. 78 or a variant thereof having one, two and/or three amino acid substitutions in any one or more of SEQ ID Nos 76, 77 and/or 78.

20. The nucleic acid molecule of any one of claims 1, 4 to 7 or 15 to 19 wherein said anti-CLEC14A binding domain has an amino acid sequence comprising
(a) SEQ ID NO. 67, SEQ ID NO. 68 and SEQ ID NO. 69 or a variant thereof having one, two or three amino acid substitutions in any one or more of SEQ ID Nos 67, 68 and/or 69; or
(b) SEQ ID NO. 79, STS and SEQ ID NO. 81 or a variant thereof having one, two or three amino acid substitutions in any one or more of SEQ ID Nos 79, 81 and/or STS.

21. The nucleic acid molecule of any one of claims 1, 4 to 7 or 15 to 20 wherein said anti-CLEC14A binding domain has an amino acid sequence comprising
(a) SEQ ID NO 64, SEQ ID NO. 65, SEQ ID No. 66, SEQ ID NO. 67, SEQ ID NO 0.68 and SEQ ID NO. 69 or a variant thereof having one, two or three amino acid substitutions in any one or more of SEQ ID Nos 64, 65, 66, 67, 68 and/or 69; or
(b) SEQ ID NO. 76, SEQ ID NO. 77, SEQ ID NO. 78, SEQ ID NO. 79, STS and SEQ ID NO. 81 or a variant thereof having one, two or three amino acid substitutions in any one or more of SEQ ID Nos 76, 77, 78, 79, 81 and/or STS.

22. A nucleic acid molecule comprising a polynucleotide sequence encoding a chimeric antigen receptor comprising
(i) an anti-CLEC14A binding domain
(ii) a transmembrane domain and
(iii) an intracellular signalling domain,
wherein said anti-CLEC14A binding domain comprises at least one of
(a) a heavy chain CDR having the amino acid sequence of SEQ ID NO. 167 or a variant thereof having one, two or three amino acid substitutions,
(b) a heavy chain CDR having the amino acid sequence of SEQ ID NO. 168 or a variant thereof having one, two or three amino acid substitutions,
(c) a heavy chain CDR having the amino acid sequence of SEQ ID NO. 169 or a variant thereof having one, two or three amino acid substitutions,
(d) a light chain CDR having the amino acid sequence of SEQ ID NO. 129 or a variant thereof having one, two or three amino acid substitutions,
(e) a light chain CDR having the amino acid sequence of SEQ ID NO. 68 or a variant thereof having one, two or three amino acid substitutions, and/or
(f) a light chain CDR having the amino acid sequence of SEQ ID NO. 130 or a variant thereof having one, two or three amino acid substitutions.

23. The nucleic acid molecule of claim 1 or 22, wherein said anti-CLEC14A binding domain has an amino acid sequence comprising SEQ ID NO. 129, SEQ ID NO. 68 and SEQ ID NO. 130, or a variant thereof having one, two or three amino acid substitutions in any one or more of SEQ ID Nos 129, 68 and/or 130 and/or comprising SEQ ID NO. 167, SEQ ID NO. 168 and SEQ ID NO. 169, or a variant thereof having one, two or three amino acid substitutions in any one or more of SEQ ID Nos 167, 168 and/or 169.

24. The nucleic acid molecule of any one of claims 1 to 14 wherein said anti-CLEC14A binding domain comprises
(a) a heavy chain variable region comprising an amino acid sequence of SEQ ID NO. 56, SEQ ID NO. 104, SEQ ID NO. 106 or SEQ ID NO. 121, or a variant thereof having at least 80% identity thereto, and/or
(b) a light chain variable region comprising an amino acid sequence of SEQ ID NO. 57, SEQ ID NO. 105, SEQ ID NO. 107 or SEQ ID NO. 122, or a variant thereof having at least 80% identity thereto.

25. The nucleic acid molecule of any one of claims 1, 4 to 7 or 15 to 21 wherein said anti-CLEC14A binding domain comprises
(a) a heavy chain variable region comprising an amino acid sequence of SEQ ID NO. 88 or SEQ ID NO. 90, or a variant thereof having at least 80% identity thereto, and/or
(b) a light chain variable region comprising an amino acid sequence of SEQ ID NO. 89 or SEQ ID NO. 91, or a variant thereof having at least 80% identity thereto.

26. The nucleic acid molecule of claim 22 or 23, wherein said anti-CLEC14A binding domain comprises a heavy chain variable region comprising an amino acid sequence of SEQ ID NO. 173 or a variant thereof having at least 80% identity thereto and/or a light chain variable region comprising an amino acid sequence of SEQ ID NO. 133 or a variant thereof having at least 80% identity thereto.

27. The nucleic acid molecule of any one of claims 1 to 26 wherein said anti-CLEC14A binding domain comprises at least one heavy chain variable region that comprises three CDRs and at least one light chain variable region that comprises three CDRs.

28. The nucleic acid molecule of any one of claim 1 to 14, 24 or 27, wherein said anti-CLEC14A binding domain comprises a scFv comprising an amino acid sequence of SEQ ID NO. 58, SEQ ID NO. 112 or SEQ ID NO. 125, or an amino acid sequence having at least 80% identity thereto.

29. The nucleic acid molecule of any one of claim 1, 4 to 7, 15 to 21 or 27 wherein said CLEC14A binding domain comprises a scFv comprising an amino acid sequence of SEQ ID NO. 96, or an amino acid sequence having at least 80% identity thereto.

30. The nucleic acid molecule of any one of claim 1 to 14, 24, 27 or 28 wherein said polynucleotide comprises at least one of the following nucleotide sequences:
(a) SEQ ID NO. 38, SEQ ID NO. 50 or a variant thereof having one, two or three nucleotide substitutions,
(b) SEQ ID NO. 39, SEQ ID NO. 51, or a variant thereof having one, two or three nucleotide substitutions, and/or
(c) SEQ ID NO. 40, SEQ ID NO. 52, SEQ ID NO. 101, SEQ ID NO. 103, SEQ ID NO. 117, SEQ ID NO. 120 or a variant thereof having one, two or three nucleotide substitutions.

31. The nucleic acid molecule of any one of claim 1 to 14, 24, 27, 28 or 30 wherein said polynucleotide comprises at least one of the following nucleotide sequences:
(a) SEQ ID NO. 41, SEQ ID NO. 53 or a variant thereof having one, two or three nucleotide substitutions,
(b) SEQ ID NO. 42, GACACATCC or a variant thereof having one, two or three nucleotide substitutions and/or
(c) SEQ ID NO. 43, SEQ ID NO. 55 or a variant thereof having one, two or three nucleotide substitutions.

32. The nucleic acid molecule of any one of claim 1, 4 to 7, 15 to 21, 27 or 29 wherein said polynucleotide comprises at least one of the following nucleotide sequences:
(a) SEQ ID NO. 82 or a variant thereof having one, two or three nucleotide substitutions,
(b) SEQ ID NO. 83 or a variant thereof having one, two or three nucleotide substitutions, and/or
(c) SEQ ID NO. 84 or a variant thereof having one, two or three nucleotide substitutions.

33. The nucleic acid molecule of any one of claim 1, 4 to 7, 15 to 21, 27, 29 or 32 wherein said polynucleotide comprises at least one of the following nucleotide sequences
(a) SEQ ID NO. 85 or a variant thereof having one, two or three nucleotide substitutions,
(b) AGCACATCC or a variant thereof having one, two or three nucleotide substitutions, and/or
(c) SEQ ID NO. 87 or a variant thereof having one, two or three nucleotide substitutions.

34. The nucleic acid molecule of any one of claim 1 to 14, 24, 27, 28, 30 or 31 wherein said polynucleotide comprises
(a) SEQ ID NO. 59, SEQ ID NO. 108, SEQ ID NO. 110 or SEQ ID NO. 123, or a variant thereof having from one to ten nucleotide substitutions, and/or
(b) SEQ ID NO. 60, SEQ ID NO. 109, SEQ ID NO. 111 or SEQ ID NO. 124, or a variant thereof having from one to ten nucleotide substitutions.

35. The nucleic acid molecule of any one of claim 1 to 14, 24, 27, 28, 30, 31 or 34, wherein said polynucleotide comprises SEQ ID No. 61, SEQ ID NO. 113 or SEQ ID NO. 126, or a sequence having at least 80% identity thereof.

36. The nucleic acid molecule of any one of claim 1, 4 to 7, 15 to 21, 27, 29, 32 or 33 wherein said polynucleotide comprises
(a) SEQ ID NO. 92, SEQ ID NO. 94 or a variant thereof having from one to ten nucleotide substitutions, and/or
(b) SEQ ID NO. 93, SEQ ID NO. 95 or a variant thereof having from one to ten nucleotide substitutions.

37. The nucleic acid molecule of any one of claim 1, 4 to 7, 15 to 21, 27, 29, 32, 33 or 36 wherein said polynucleotide comprises SEQ ID NO. 97 or a sequence having at least 80% identity thereto.

38. The nucleic acid molecule of any one of claims 1 to 37 wherein said transmembrane domain is derived from CD8 alpha or from CD28, and preferably comprises the amino acid sequence of SEQ ID NO. 146 or a sequence having at least 95% identity thereto.

39. The nucleic acid molecule of any one of claims 1 to 38 wherein said intracellular signalling domain is derived from CD3 zeta, and preferably comprises the amino acid sequence of SEQ ID NO. 148 or a sequence having at least 95% identity thereto.

40. The nucleic acid molecule of any one of claims 1 to 39 wherein said CAR comprises at least one costimulatory domain, preferably selected from any one of CD28, 4-1BB, OX40, ICOS, DAP10, CD27, CD30, CD40, ICOS, lymphocyte function-associated antigen-1, CD2, CD7, LIGHT, NKG2C, and/or B7-H3.

41. The nucleic acid molecule of claim 40, wherein said transmembrane domain and said costimulatory domain are derived from CD28.

42. The nucleic acid molecule of any one of claims 1 to 41 wherein said CAR comprises a leader sequence, preferably an oncostatin M leader sequence of SEQ ID NO. 135, a CD8a leader sequence encoded by SEQ ID NO. 162 or a sequence having at least 95% identity thereto, and/or wherein said CAR comprises a hinge or spacer, preferably derived from CD8alpha.

43. The nucleic acid molecule of claim 1 wherein said CAR
(a) comprises an amino acid sequence of SEQ ID NO. 62, SEQ ID NO. 98, SEQ ID NO. 114 or SEQ ID NO. 127 or a sequence having at least 80% identity thereto, and/or
(b) is encoded by a polynucleotide sequence comprising a nucleotide sequence of SEQ ID NO. 63, SEQ ID NO. 99, SEQ ID NO. 115 or SEQ ID NO. 128 or a sequence having at least 80% identity thereto.

44. The nucleic acid molecule of any one of claims 1 to 29 wherein said nucleic acid molecule is RNA.

45. A chimeric antigen receptor (CAR) encoded by a nucleic acid molecule of any one of claims 1 to 44.

46. A vector comprising the nucleic acid molecule of any one of claims 1 to 43, and optionally comprising a nucleic acid molecule comprising a polynucleotide sequence encoding a T cell receptor molecule.

47. The vector of claim 46 wherein said vector is a viral vector, preferably a gamma retroviral vector.

48. The vector of claim 46 or 47, wherein said vector further comprises a nucleotide sequence encoding a suicide gene, a cytokine, a dominant-negative TGFβ receptor and/or a CD34 molecule, preferably a truncated CD34 molecule

49. A cell comprising a nucleic acid of any one of claims 1 to 44, a CAR of claim 45 or a vector of any one of claims 46 to 48, and optionally comprising a nucleic acid molecule or vector comprising a polynucleotide sequence encoding a T cell receptor molecule.

50. The cell of claim 49, wherein said cell is an immune cell, preferably a T-cell or a natural killer cell.

51. The cell of claim 49 or 50 wherein said cell comprises a further polynucleotide or vector encoding a suicide gene, a dominant-negative TGFβ receptor or a cytokine.

52. A nucleic acid molecule of any one of claims 1 to 44, a vector of any one of claims 46 to 48 or a cell of any one of claims 49 to 51 for use in therapy.

53. A method of combating or treating disease comprising the step of administering a nucleic acid molecule of any one of claims 1 to 44, a vector of any one of claims 46 to 48 or a cell of any one of claims 49 to 51 to a subject in need thereof.

54. The nucleic acid molecule of any one of claims 1 to 44, the vector of any one of claims 46 to 48 or the cell of any one of claims 49 to 51 for use in treating a condition associated with expression of CLEC14A.

55. A method of treating a condition associated with expression of CLEC14A comprising the step of administering a nucleic acid molecule of any one of claims 1 to 44, a vector of any one of claims 46 to 48 or a cell of any one of claims 49 to 51 to a subject in need thereof.

56. Use of a nucleic acid molecule of any one of claims 1 to 44, a vector of any one of claims 46 to 48 or a cell of any one of claims 49 to 51 for use in the manufacture of a medicament for treating a condition associated with CLEC14A expression.

57. The nucleic acid molecule, vector or cell for use according to claim 54, the method of claim 55 or the use of claim 56, wherein said condition associated with expression of CLEC14A is a CLEC14A expressing solid tumour, or tumour angiogenesis.

58. The nucleic acid molecule, vector or cell for use according to claim 54, the method of claim 55 or the use of claim 56, wherein said nucleic acid molecule, vector or cell is for administration separately, simultaneously or sequentially in combination with one or more therapeutic agents, preferably (i) an anti-cancer agent, more preferably tirapazamine; (ii) a nucleic acid molecule comprising a polynucleotide sequence encoding a TCR molecule; (iii) a vector comprising a nucleic acid molecule of (ii); or (iv) a cell comprising a nucleic acid molecule of (ii) or a vector of (iii).

59. The nucleic acid molecule, vector or cell for use, the method or the use of claim 58, wherein the TCR molecule binds to WT1, preferably to HLA A2/RMFPNAPYL.

60. The nucleic acid molecule, vector or cell for use, the method or the use of claim 59, wherein the TCR molecule comprises an alpha chain and a beta chain, wherein the alpha chain comprises CDR1alpha of SEQ ID NO. 190, CDR2 alpha of SEQ ID NO. 191 and CDR3 alpha of SEQ ID NO. 192 or 193, and wherein the beta chain comprises CDR1 beta of SEQ ID NO. 194, CDR2 beta of SEQ ID NO. 195 and CDR3 beta of SEQ ID NO. 196 or 197, or a variant thereof wherein one or more of the CDRs comprise one, two or three amino acid substitutions, wherein said TCR molecule is capable of binding to an HLA A2/RMFPNAPYL complex.

61. A pharmaceutical composition comprising a nucleic acid molecule according to any one of claims 1 to 44, a vector according to any one of claims 46 to 48 or a cell according to any one of claims 49 to 51 and optionally comprising a nucleic acid molecule comprising a polynucleotide sequence encoding a TCR, a vector comprising a nucleic acid molecule comprising a polynucleotide sequence encoding a TCR or a cell comprising a nucleic acid molecule comprising a polynucleotide encoding a TCR or a vector comprising a nucleic acid molecule comprising a polynucleotide encoding a TCR.


SEQUENCE LISTING (232 sequences)

SEQ ID NO: 1 - protein, 490 aa, Homo sapiens
MRPAFALCLLWQALWPGPGGGEHPTADRAGCSASGACYSLHHATMKRQAAEEACILRGGALSTVRAGAELRAVLALLRAGPGPGGGSKDLLFWVALERRRSHCTLENEPLRGFSWLSSDPGGLESDTLQWVEEPQRSCTARRCAVLQATGGVEPAGWKEMRCHLRANGYLCKYQFEVLCPAPRPGAASNLSYRAPFQLHSAALDFSPPGTEVSALCRGQLPISVTCIADEIGARWDKLSGDVLCPCPGRYLRAGKCAELPNCLDDLGGFACECATGFELGKDGRSCVTSGEGQPTLGGTGVPTRRPPATATSPVPQRTWPIRVDEKLGETPLVPEQDNSVTSIPEIPRWGSQSTMSTLQMSLQAESKATITPSGSVISKFNSTTSSATPQAFDSSSAVVFIFVSTAVVVLVILTMTVLGLVKLCFHESPSSQPRKESMGPPGLESDPEPAALGSSSAHCTNNGVKVGDCDLRDRAEGALLAESPLGSSDA

SEQ ID NO: 2 - DNA, 2256 bp, Homo sapiens
ctcctcttgctctaagcagggtgtttgaccttctagtcgactgcgtcccctgtacccggcgccagctgtgttcctgaccccagaataactcagggctgcaccgggcctggcagcgctccgcacacatttcctgtcgcggcctaagggaaactgttggccgctgggcccgcggggggattcttggcagttggggggtccgtcgggagcgagggcggaggggaagggagggggaaccgggttggggaagccagctgtagagggcggtgaccgcgctccagacacagctctgcgtcctcgagcgggacagatccaagttgggagcagctctgcgtgcggggcctcagagaatgaggccggcgttcgccctgtgcctcctctggcaggcgctctggcccgggccgggcggcggcgaacaccccactgccgaccgtgctggctgctcggcctcgggggcctgctacagcctgcaccacgctaccatgaagcggcaggcggccgaggaggcctgcatcctgcgaggtggggcgctcagcaccgtgcgtgcgggcgccgagctgcgcgctgtgctcgcgctcctgcgggcaggcccagggcccggagggggctccaaagacctgctgttctgggtcgcactggagcgcaggcgttcccactgcaccctggagaacgagcctttgcggggtttctcctggctgtcctccgaccccggcggtctcgaaagcgacacgctgcagtgggtggaggagccccaacgctcctgcaccgcgcggagatgcgcggtactccaggccaccggtggggtcgagcccgcaggctggaaggagatgcgatgccacctgcgcgccaacggctacctgtgcaagtaccagtttgaggtcttgtgtcctgcgccgcgccccggggccgcctctaacttgagctatcgcgcgcccttccagctgcacagcgccgctctggacttcagtccacctgggaccgaggtgagtgcgctctgccggggacagctcccgatctcagttacttgcatcgcggacgaaatcggcgctcgctgggacaaactctcgggcgatgtgttgtgtccctgccccgggaggtacctccgtgctggcaaatgcgcagagctccctaactgcctagacgacttgggaggctttgcctgcgaatgtgctacgggcttcgagctggggaaggacggccgctcttgtgtgaccagtggggaaggacagccgacccttggggggaccggggtgcccaccaggcgcccgccggccactgcaaccagccccgtgccgcagagaacatggccaatcagggtcgacgagaagctgggagagacaccacttgtccctgaacaagacaattcagtaacatctattcctgagattcctcgatggggatcacagagcacgatgtctacccttcaaatgtcccttcaagccgagtcaaaggccactatcaccccatcagggagcgtgatttccaagtttaattctacgacttcctctgccactcctcaggctttcgactcctcctctgccgtggtcttcatatttgtgagcacagcagtagtagtgttggtgatcttgaccatgacagtactggggcttgtcaagctctgctttcacgaaagcccctcttcccagccaaggaaggagtctatgggcccgccgggcctggagagtgatcctgagcccgctgctttgggctccagttctgcacattgcacaaacaatggggtgaaagtcggggactgtgatctgcgggacagagcagagggtgccttgctggcggagtcccctcttggctctagtgatgcatagggaaacaggggacatgggcactcctgtgaacagtttttcacttttgatgaaacggggaaccaagaggaacttacttgtgtaactgacaatttctgcagaaatcccccttcctctaaattccctttactccactgaggagctaaatcagaactgcacactccttccctgatgatagaggaagtggaagtgcctttaggatggtgatactgggggaccgggtagtgctggggagagatattttcttatgtttattcggagaatttggagaagtgattgaacttttcaagacattggaaacaaatagaacacaatataatttacattaaaaaataatttctaccaaaatggaaaggaaatgttctatgttgttcaggctaggagtatattggttcgaaatcccagggaaaaaaataaaaataaaaaattaaaggattgt

SEQ ID NO: 3 - DNA, 1473 bp, Artificial Sequence
  CLEC14A cds
atgaggccggcgttcgccctgtgcctcctctggcaggcgctctggcccgggccgggcggcggcgaacaccccactgccgaccgtgctggctgctcggcctcgggggcctgctacagcctgcaccacgctaccatgaagcggcaggcggccgaggaggcctgcatcctgcgaggtggggcgctcagcaccgtgcgtgcgggcgccgagctgcgcgctgtgctcgcgctcctgcgggcaggcccagggcccggagggggctccaaagacctgctgttctgggtcgcactggagcgcaggcgttcccactgcaccctggagaacgagcctttgcggggtttctcctggctgtcctccgaccccggcggtctcgaaagcgacacgctgcagtgggtggaggagccccaacgctcctgcaccgcgcggagatgcgcggtactccaggccaccggtggggtcgagcccgcaggctggaaggagatgcgatgccacctgcgcgccaacggctacctgtgcaagtaccagtttgaggtcttgtgtcctgcgccgcgccccggggccgcctctaacttgagctatcgcgcgcccttccagctgcacagcgccgctctggacttcagtccacctgggaccgaggtgagtgcgctctgccggggacagctcccgatctcagttacttgcatcgcggacgaaatcggcgctcgctgggacaaactctcgggcgatgtgttgtgtccctgccccgggaggtacctccgtgctggcaaatgcgcagagctccctaactgcctagacgacttgggaggctttgcctgcgaatgtgctacgggcttcgagctggggaaggacggccgctcttgtgtgaccagtggggaaggacagccgacccttggggggaccggggtgcccaccaggcgcccgccggccactgcaaccagccccgtgccgcagagaacatggccaatcagggtcgacgagaagctgggagagacaccacttgtccctgaacaagacaattcagtaacatctattcctgagattcctcgatggggatcacagagcacgatgtctacccttcaaatgtcccttcaagccgagtcaaaggccactatcaccccatcagggagcgtgatttccaagtttaattctacgacttcctctgccactcctcaggctttcgactcctcctctgccgtggtcttcatatttgtgagcacagcagtagtagtgttggtgatcttgaccatgacagtactggggcttgtcaagctctgctttcacgaaagcccctcttcccagccaaggaaggagtctatgggcccgccgggcctggagagtgatcctgagcccgctgctttgggctccagttctgcacattgcacaaacaatggggtgaaagtcggggactgtgatctgcgggacagagcagagggtgccttgctggcggagtcccctcttggctctagtgatgcatag

SEQ ID NO: 4 - DNA, 39 bp, Artificial Sequence
  Human CLEC14A forward primer
tagtaggaattcgagagaatgaggccggcgttcgccctg

SEQ ID NO: 5 - DNA, 39 bp, Artificial Sequence
  human CLEC14A reverse primer
agaaccgcggccgctggaggagtcgaaagcctgaggagt

SEQ ID NO: 6 - DNA, 39 bp, Artificial Sequence
  Murine CLEC14A forward primer
tagtaggaattcgagagaatgaggccagcgcttgccctg

SEQ ID NO: 7 - DNA, 36 bp, Artificial Sequence
  Murine CLEC14A reverse primer
ctactagcggccgctcgtggaagaggtgtcgaaagt

SEQ ID NO: 8 - DNA, 35 bp, Artificial Sequence
  human CLEC14A forward primer
tagtagttaattaagagagaatgaggccggcgttc

SEQ ID NO: 9 - DNA, 35 bp, Artificial Sequence
  Murine CLEC14A forward primer
tagtagttaattaagagagaatgaggccagcgctt

SEQ ID NO: 10 - DNA, 35 bp, Artificial Sequence
  Human Fc reverse primer
ctactagtttaaactcatttacccggagacaggga

SEQ ID NO: 11 - DNA, 29 bp, Artificial Sequence
  MMRN2 forward primer
ccggaccggtcaggcttccagtactagcc

SEQ ID NO: 12 - DNA, 29 bp, Artificial Sequence
  MMRN2 reverse primer
cggggtaccggtcttaaacatcaggaagc

SEQ ID NO: 13 - DNA, 20 bp, Artificial Sequence
  5′UTR forward primer
ttccttttccagggtttgtg

SEQ ID NO: 14 - DNA, 20 bp, Artificial Sequence
  5′UTR reverse primer
gcctacaaggtggcttgaat

SEQ ID NO: 15 - DNA, 20 bp, Artificial Sequence
  CDS forward primer
aagctgtgctcctgctcttg

SEQ ID NO: 16 - DNA, 21 bp, Artificial Sequence
  CDS reverse primer
tcctgagtgcactgtgagatg

SEQ ID NO: 17 - DNA, 20 bp, Artificial Sequence
  3′UTR forward primer
ctgtagagggcggtgacttt

SEQ ID NO: 18 - DNA, 19 bp, Artificial Sequence
  3′ UTR reverse primer
agctgctcccaagtcctct

SEQ ID NO: 19 - DNA, 20 bp, Artificial Sequence
  mACTB forward primer
ctaaggccaaccgtgaaaag

SEQ ID NO: 20 - DNA, 20 bp, Artificial Sequence
  mACTB reverse primer
accagaggcatacagggaca

SEQ ID NO: 21 - protein, 39 aa, Unknown
  N-terminal portion of CD141
MLGVLVLGALALAGLGFPAPAEPQPGGSQCVEHDCFALY

SEQ ID NO: 22 - protein, 12 aa, Unknown
  Residues 97-108 of CD141
QLPPGCGDPKRL

SEQ ID NO: 23 - protein, 20 aa, Unknown
  Residues 122-142 of CD141
TSYSRWARLDLNGAPLCGPL

SEQ ID NO: 24 - protein, 12 aa, Unknown
  Residues 97-108 human CLEC14A
ERRRSCHTLENE

SEQ ID NO: 25 - protein, 169 aa, Unknown
  CTLD of CD141
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEF

SEQ ID NO: 26 - protein, 740 aa, Artificial Sequence
  Chimera of CLEC14A, CD141 and GFP
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFQFEVLCPAPRPGAASNLSYRAPFQLHSAALDFSPPGTEVSALCRGQLPISVTCIADEIGARWDKLSGDVLCPCPGRYLRAGKCAELPNCLDDLGGFACECATGFELGKDGRSCVTSGEGQPTLGGTGVPTRRPPATATSPVPQRTWPIRVDEKLGETPLVPEQDNSVTSIPEIPRWGSQSTMSTLQMSLQAESKATITPSGSVISKFNSTTSSATPQAFDSSSAVVFIFVSTAVVVLVILTMTVLGLVKLCFHESPSSQPRKESMGPPGLESDPEPAALGSSSAHCTNNGVKVGDCDLRDRAEGALLAESPLGSSDALQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 27 - protein, 745 aa, Artificial Sequence
  Chimera of CLEC14A, CD141 and GFP
MRPAFALCLLWQALWPGPGGGEHPTADRAGCSASGACYSLHHATMKRQAAEEACILRGGALSTVRAGAELRAVLALLRAGPGPGGGSKDLLFWVALERRRSHCTLENEPLRGFSWLSSDPGGLESDTLQWVEEPQRSCTARRCAVLQATGGVEPAGWKEMRCHLRANGYLCKYHFPATCRPLAVEPGAAAAAVSITYGTPFAARGADFQALPVGSSAAVAPLGLQLMCTAPPGAVQGHWAREAPGACPGRYLRAGKCAELPNCLDDLGGFACECATGFELGKDGRSCVTSGEGQPTLGGTGVPTRRPPATATSPVPQRTWPIRVDEKLGETPLVPEQDNSVTSIPEIPRWGSQSTMSTLQMSLQAESKATITPSGSVISKFNSTTSSATPQAFDSSSAVVFIFVSTAVVVLVILTMTVLGLVKLCFHESPSSQPRKESMGPPGLESDPEPAALGSSSAHCTNNGVKVGDCDLRDRAEGALLAESPLGSSDALQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 28 - protein, 949 aa, Unknown [sequence omitted]

SEQ ID NO: 29 - DNA, 2850 bp, Unknown
  MMRN2 nucleotide
atgatcctgagcttgctgttcagccttgggggccccctgggctgggggctgctgggggcatgggcccaggcttccagtactagcctctctgatctgcagagctccaggacacctggggtctggaaggcagaggctgaggacaccggcaaggaccccgttggacgtaactggtgcccctacccaatgtccaagctggtcaccttactagctctttgcaaaacagagaaattcctcatccactcgcagcagccgtgtccgcagggagctccagactgccagaaagtcaaagtcatgtaccgcatggcccacaagccagtgtaccaggtcaagcagaaggtgctgacctctttggcctggaggtgctgccctggctacacgggccccaactgcgagcaccacgattccatggcaatccctgagcctgcagatcctggtgacagccaccaggaacctcaggatggaccagtcagcttcaaacctggccaccttgctgcagtgatcaatgaggttgaggtgcaacaggaacagcaggaacatctgctgggagatctccagaatgatgtgcaccgggtggcagacagcctgccaggcctgtggaaagccctgcctggtaacctcacagctgcagtgatggaagcaaatcaaacagggcacgagttccctgatagatccttggagcaggtgctgctaccccacgtggacaccttcctacaagtgcatttcagccccatctggaggagctttaaccaaagcctgcacagccttacccaggccataagaaacctgtctcttgacgtggaggccaaccgccaggccatctccagagtccaggacagtgccgtggccagggctgacttccaggagcttggtgccaaatttgaggccaaggtccaggagaacactcagagagtgggtcagctgcgacaggacgtggaggaccgcctgcacgcccagcactttaccctgcaccgctcgatctcagagctccaagccgatgtggacaccaaattgaagaggctgcacaaggctcaggaggccccagggaccaatggcagtctggtgttggcaacgcctggggctggggcaaggcctgagccggacagcctgcaggccaggctgggccagctgcagaggaacctctcagagctgcacatgaccacggcccgcagggaggaggagttgcagtacaccctggaggacatgagggccaccctgacccggcacgtggatgagatcaaggaactgtactccgaatcggacgagactttcgatcagattagcaaggtggagcggcaggtggaggagctgcaggtgaaccacacggcgctccgtgagctgcgcgtgatcctgatggagaagtctctgatcatggaggagaacaaggaggaggtggagcggcagctcctggagctcaacctcacgctgcagcacctgcagggtggccatgccgacctcatcaagtacgtgaaggactgcaattgccagaagctctatttagacctggacgtcatccgggagggccagagggacgccacgcgtgccctggaggagacccaggtgagcctggacgagcggcggcagctggacggctcctccctgcaggccctgcagaacgccgtggacgccgtgtcgctggccgtggacgcgcacaaagcggagggcgagcgggcgcgggcggccacgtcgcggctccggagccaagtgcaggcgctggatgacgaggtgggcgcgctgaaggcggccgcggccgaggcccgccacgaggtgcgccagctgcacagcgccttcgccgccctgctggaggacgcgctgcggcacgaggcggtgctggccgcgctcttcggggaggaggtgctggaggagatgtctgagcagacgccgggaccgctgcccctgagctacgagcagatccgcgtggccctgcaggacgccgctagcgggctgcaggagcaggcgctcggctgggacgagctggccgcccgagtgacggccctggagcaggcctcggagcccccgcggccggcagagcacctggagcccagccacgacgcgggccgcgaggaggccgccaccaccgccctggccgggctggcgcgggagctccagagcctgagcaacgacgtcaagaatgtcgggcggtgctgcgaggctgaggccggggccggggccgcctccctcaacgcctcccttgacggcctccacaacgcactcttcgccactcagcgcagcttggagcagcaccagcggctcttccacagcctctttgggaacttccaagggctcatggaagccaacgtcagcctggacctggggaagctgcagaccatgctgagcaggaaagggaagaagcagcagaaagacctggaagctccccggaagagggacaagaaggaagcggagcctttggtggacatacgggtcacagggcctgtgccaggtgccttgggcgcggcgctctgggaggcaggatcccctgtggccttctatgccagcttttcagaagggacggctgccctgcagacagtgaagttcaacaccacatacatcaacattggcagcagctacttccctgaacatggctacttccgagcccctgagcgtggtgtctacctgtttgcagtgagcgttgaatttggcccagggccaggcaccgggcagctggtgtttggaggtcaccatcggactccagtctgtaccactgggcaggggagtggaagcacagcaacggtctttgccatggctgagctgcagaagggtgagcgagtatggtttgagttaacccagggatcaataacaaagagaagcctgtcgggcactgcatttgggggcttcctgatgtttaagacctga

SEQ ID NO: 30 - DNA, 19 bp, Artificial Sequence
  siRNA duplex D1
gaacaagacaattcagtaa

SEQ ID NO: 31 - DNA, 19 bp, Artificial Sequence
  siRNA duplex D2
caatcagggtcgacgagaa

SEQ ID NO: 32 - protein, 6 aa, Artificial Sequence
  CDR1 heavy chain variant 1 of CRT1
SSYWIE

SEQ ID NO: 33 - protein, 13 aa, Artificial Sequence
  CDR2 of heavy chain variant 1 of CRT1
WIGEILPGSGSTN

SEQ ID NO: 34 - protein, 14 aa, Artificial Sequence
  CDR3 of heavy chain variant 1 of CRT1
ARGGDYDEEYYVMD

SEQ ID NO: 35 - protein, 6 aa, Artificial Sequence
  CDR 1 of light chain variant 1 of CRT1
SYMYWY

SEQ ID NO: 36 - protein, 10 aa, Artificial Sequence
  CDR2 of light chain variant 1 of CRT1
LLIYDTSNLA

SEQ ID NO: 37 - protein, 8 aa, Artificial Sequence
  CDR3 of light chain variant 1 of CRT1
QQWSSYPL

SEQ ID NO: 38 - DNA, 18 bp, Artificial Sequence
  CDR1 of heavy chain variant 1 of CRT1
agtagctactggatagag

SEQ ID NO: 39 - DNA, 39 bp, Artificial Sequence
  CDR2 of heavy chain variant 1 of CRT1
tggattggagagattttacctggaagtggtagtactaat

SEQ ID NO: 40 - DNA, 42 bp, Artificial Sequence
  CDR3 heavy chain variant 1 of CRT1
gcaagagggggggattacgacgaagaatactatgtcatggac

SEQ ID NO: 41 - DNA, 18 bp, Artificial Sequence
  CDR1 of light chain variant 1 of CRT1
agttacatgtactggtac

SEQ ID NO: 42 - DNA, 30 bp, Artificial Sequence
  CDR2 of light chain variant 1 of CRT1
ctcctgatttatgacacatccaacctggct

SEQ ID NO: 43 - DNA, 24 bp, Artificial Sequence
  CDR3 of light chain variant 1 of CRT1
cagcagtggagtagttacccgctc

SEQ ID NO: 44 - protein, 8 aa, Artificial Sequence
  CDR1 of heavy chain variant 2 of CRT1
GYTFSSYW

SEQ ID NO: 45 - protein, 8 aa, Artificial Sequence
  CDR2 of heavy chain variant 2 of CRT1
ILPGSGST

SEQ ID NO: 46 - protein, 15 aa, Artificial Sequence
  CDR3 of heavy chain variant 2 of CRT1
ARGGDYDEEYYVMDY

SEQ ID NO: 47 - protein, 5 aa, Artificial Sequence
  CDR1 of light chain variant 2 of CRT1
SSVSY

SEQ ID NO: 48 - DNA, 108 bp, Artificial Sequence
  Nucleotide sequence for OX40 costimulatory domain
agggaccagaggctgccccccgatgcccacaagccccctgggggaggcagtttccggacccccatccaagaggagcaggccgacgcccactccaccctggccaagatc

SEQ ID NO: 49 - protein, 9 aa, Artificial Sequence
  CDR3 light chain variant 2 of CRT1
QQWSSYPLT

SEQ ID NO: 50 - DNA, 24 bp, Artificial Sequence
  CDR1 heavy chain variant 2 of CRT1
ggctacacattcagtagctactgg

SEQ ID NO: 51 - DNA, 24 bp, Artificial Sequence
  CDR2 heavy chain variant 2 of CRT1
attttacctggaagtggtagtact

SEQ ID NO: 52 - DNA, 45 bp, Artificial Sequence
  CDR3 of heavy chain variant 2 of CRT1
gcaagagggggggattacgacgaagaatactatgtcatggactac

SEQ ID NO: 53 - DNA, 15 bp, Artificial Sequence
  CDR1 of light chain variant 2 of CRT1
tcaagtgtaagttac

SEQ ID NO: 54 - DNA, 204 bp, Artificial Sequence
  Nucleotide sequence for CD28 costimulatory domain
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 55 - DNA, 27 bp, Artificial Sequence
  CDR3 of light chain variant 2 for CRT1
cagcagtggagtagttacccgctcacg

SEQ ID NO: 56 - protein, 122 aa, Artificial Sequence
  CRT1 heavy chain protein
MAEVQLQQSGAELMKPGASVKISCKATGYTFSSYWIEWVKQRPGHGLEWIGEILPGSGSTNYNEKFKGKATFTADTSSNTAYMQLSSLTSEDSAVYYCARGGDYDEEYYVMDYWGQGTSVTV

SEQ ID NO: 57 - protein, 107 aa, Artificial Sequence
  CRT1 light chain protein
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKR

SEQ ID NO: 58 - protein, 249 aa, Artificial Sequence
  CRT1 scFv protein
MAEVQLQQSGAELMKPGASVKISCKATGYTFSSYWIEWVKQRPGHGLEWIGEILPGSGSTNYNEKFKGKATFTADTSSNTAYMQLSSLTSEDSAVYYCARGGDYDEEYYVMDYWGQGTSVTVSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRAAA

SEQ ID NO: 59 - DNA, 366 bp, Artificial Sequence
  CRT1 heavy chain nucleotide
atggccgaggttcagcttcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgtcatggactactggggtcaaggaacctcagtcactgtc

SEQ ID NO: 60 - DNA, 321 bp, Artificial Sequence
  CRT1 light chain nucleotide
caaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgt

SEQ ID NO: 61 - DNA, 747 bp, Artificial Sequence
  CRT1 scFv nucleotide
atggccgaggttcagcttcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgtcatggactactggggtcaaggaacctcagtcactgtctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgtgcggccgca

SEQ ID NO: 62 - protein, 493 aa, Artificial Sequence
  CRT1 CAR1 protein
MGVLLTQRTLLSLVLALLFPSMASMAEVQLQQSGAELMKPGASVKISCKATGYTFSSYWIEWVKQRPGHGLEWIGEILPGSGSTNYNEKFKGKATFTADTSSNTAYMQLSSLTSEDSAVYYCARGGDYDEEYYVMDYWGQGTSVTVSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 63 - DNA, 1502 bp, Artificial Sequence
  CRT1 CAR1 nucleotide
atgggcgtgctgctgacccagaggaccctgctgagcctggtgctggccctgctgtttccatctatggcatcgatggccgaggttcagcttcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgtcatggactactggggtcaaggaacctcagtcactgtctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgtgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataaaagcttaacacgagcca

SEQ ID NO: 64 - protein, 6 aa, Artificial Sequence
  CDR1 of heavy chain variant 1 of CRT3
TSYWMH

SEQ ID NO: 65 - protein, 13 aa, Artificial Sequence
  CDR2 of heavy chain variant 1 of CRT3
WIGAIYPGNSDTS

SEQ ID NO: 66 - protein, 11 aa, Artificial Sequence
  CDR3 of heavy chain variant 1 of CRT3
THYYGSDYAMD

SEQ ID NO: 67 - protein, 8 aa, Artificial Sequence
  CDR1 of light chain variant 1 of CRT3
SSSYLHWY

SEQ ID NO: 68 - protein, 10 aa, Artificial Sequence
  CDR2 of light chain variant 1 of CRT3
LWIYSTSNLA

SEQ ID NO: 69 - protein, 8 aa, Artificial Sequence
  CDR3 of light chain variant 1 of CRT3
HQYHRSPR

SEQ ID NO: 70 - DNA, 18 bp, Artificial Sequence
  CDR1 of heavy chain variant 1 of CRT3
accagctactggatgcac

SEQ ID NO: 71 - DNA, 39 bp, Artificial Sequence
  CDR2 of heavy chain variant 1 of CRT3
tggattggcgctatttatcctggaaatagtgatactagc

SEQ ID NO: 72 - DNA, 33 bp, Artificial Sequence
  CDR3 of heavy chain variant 1 of CRT3
acacattactacggtagtgactatgctatggac

SEQ ID NO: 73 - DNA, 24 bp, Artificial Sequence
  CDR1 of light chain variant 1 of CRT3
agttccagttacttgcactggtac

SEQ ID NO: 74 - DNA, 30 bp, Artificial Sequence
  CDR2 of light chain variant 1 of CRT3
ctctggatttatagcacatccaacctggct

SEQ ID NO: 75 - DNA, 25 bp, Artificial Sequence
  CDR3 of light chain variant 1 of CRT3
ccaccagtatcatcgttccccacgg

SEQ ID NO: 76 - protein, 8 aa, Artificial Sequence
  CDR1 of heavy chain variant 2 of CRT3
GYTFTSYW

SEQ ID NO: 77 - protein, 8 aa, Artificial Sequence
  CDR2 of heavy chain variant 2 of CRT3
IYPGNSDT

SEQ ID NO: 78 - protein, 12 aa, Artificial Sequence
  CDR3 of heavy chain variant 2 of CRT3
THYYGSDYAMDY

SEQ ID NO: 79 - protein, 7 aa, Artificial Sequence
  CDR1 of light chain variant 2 of CRT3
SSVSSSY

SEQ ID NO: 80 - DNA, 126 bp, Artificial Sequence
  Nucleotide sequence for 41BB costimulatory domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 81 - protein, 9 aa, Artificial Sequence
  CDR3 of light chain variant 2 of CRT3
HQYHRSPRT

SEQ ID NO: 82 - DNA, 24 bp, Artificial Sequence
  CDR1 of heavy chain variant 2 of CRT3
ggctacacctttaccagctactgg

SEQ ID NO: 83 - DNA, 24 bp, Artificial Sequence
  CDR2 of heavy chain variant 2 of CRT3
atttatcctggaaatagtgatact

SEQ ID NO: 84 - DNA, 36 bp, Artificial Sequence
  CDR3 of heavy chain variant 2 of CRT3
acacattactacggtagtgactatgctatggactac

SEQ ID NO: 85 - DNA, 21 bp, Artificial Sequence
  CDR1 of light chain variant 2 of CRT3
tcaagtgtaagttccagttac

SEQ ID NO: 86 - protein, 36 aa, Artificial Sequence
  OX40 costimulatory domain
RDQRLPPDAHKPPGGGSFRTPIQEEQADAHSTLAKI

SEQ ID NO: 87 - DNA, 27 bp, Artificial Sequence
  CDR3 of light chain variant 2 of CRT3
caccagtatcatcgttccccacggacg

SEQ ID NO: 88 - protein, 122 aa, Artificial Sequence
  CRT3 heavy chain variant 1
MAEVQLQQSGTVLARPGASVKMSCKASGYTFTSYWMHWVKQRPGQGLEWIGAIYPGNSDTSYNQKFKGKAKLTAVTSTSTAYMELSSLTNEDSAVFYCTHYYGSDYAMDYWGQGTSVTISSG

SEQ ID NO: 89 - protein, 111 aa, Artificial Sequence
  CRT3 light chain variant 1
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIKRAA

SEQ ID NO: 90 - protein, 119 aa, Artificial Sequence
  CRT3 heavy chain variant 2
MAEVQLQQSGTVLARPGASVKMSCKASGYTFTSYWMHWVKQRPGQGLEWIGAIYPGNSDTSYNQKFKGKAKLTAVTSTSTAYMELSSLTNEDSAVFYCTHYYGSDYAMDYWGQGTSVTV

SEQ ID NO: 91 - protein, 112 aa, Artificial Sequence
  CRT3 light chain variant 2
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIKRAAA

SEQ ID NO: 92 - DNA, 363 bp, Artificial Sequence
  CRT3 heavy chain variant 1 nucleotide sequence
atggccgaggtccagctgcagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtcttttactgtacacattactacggtagtgactatgctatggactactggggtcaaggaacctcagtcactgtctcctca

SEQ ID NO: 93 - DNA, 335 bp, Artificial Sequence
  CRT3 light chain variant 1 nucleotide sequence
caaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacggacgttcggtggaggcaccaagctggaaatcaaacgtgcggccgc

SEQ ID NO: 94 - DNA, 357 bp, Artificial Sequence
  CRT3 heavy chain variant 2 nucleotide sequence
atggccgaggtccagctgcagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtcttttactgtacacattactacggtagtgactatgctatggactactggggtcaaggaacctcagtcactgtc

SEQ ID NO: 95 - DNA, 327 bp, Artificial Sequence
  CRT3 light chain variant 2 nucleotide sequence
caaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacggacgttcggtggaggcaccaagctggaaatcaaacgt

SEQ ID NO: 96 - protein, 248 aa, Artificial Sequence
  CRT3 scFV
MAEVQLQQSGTVLARPGASVKMSCKASGYTFTSYWMHWVKQRPGQGLEWIGAIYPGNSDTSYNQKFKGKAKLTAVTSTSTAYMELSSLTNEDSAVFYCTHYYGSDYAMDYWGQGTSVTVSSGGGGSGGGGSGGGGSQIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIKRAAA

SEQ ID NO: 97 - DNA, 744 bp, Artificial Sequence
  CRT3 scFV nucleotide sequence
atggccgaggtccagctgcagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtcttttactgtacacattactacggtagtgactatgctatggactactggggtcaaggaacctcagtcactgtctcctcaggtggaggcggttcaggcggaggtggctctggcggtggcggatcgcaaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacggacgttcggtggaggcaccaagctggaaatcaaacgtgcggccgca

SEQ ID NO: 98 - protein, 492 aa, Artificial Sequence
  CRT3 CAR3
MGVLLTQRTLLSLVLALLFPSMASMAEVQLQQSGTVLARPGASVKMSCKASGYTFTSYWMHWVKQRPGQGLEWIGAIYPGNSDTSYNQKFKGKAKLTAVTSTSTAYMELSSLTNEDSAVFYCTHYYGSDYAMDYWGQGTSVTVSSGGGGSGGGGSGGGGSQIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 99 - DNA, 1499 bp, Artificial Sequence
  CRT3 CAR3 nucleotide sequence
atgggcgtgctgctgacccagaggaccctgctgagcctggtgctggccctgctgtttccatctatggcatcgatggccgaggtccagctgcagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtcttttactgtacacattactacggtagtgactatgctatggactactggggtcaaggaacctcagtcactgtctcctcaggtggaggcggttcaggcggaggtggctctggcggtggcggatcgcaaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacggacgttcggtggaggcaccaagctggaaatcaaacgtgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataaaagcttaacacgagcca

SEQ ID NO: 100 - protein, 14 aa, Artificial Sequence
  CDR3 heavy chain variant 1 of CRT4
ARGGDYDEEYYLMD

SEQ ID NO: 101 - DNA, 42 bp, Artificial Sequence
  CDR3 heavy chain variant 1 of CRT4
gcgagagggggggattacgacgaagaatactatctcatggac

SEQ ID NO: 102 - protein, 15 aa, Artificial Sequence
  CDR3 of heavy chain variant 2 of CRT4
ARGGDYDEEYYLMDY

SEQ ID NO: 103 - DNA, 41 bp, Artificial Sequence
  CDR3 of heavy chain variant 2 of CRT4
gcgagagggggggattacgacgaagaatactatctcattac

SEQ ID NO: 104 - protein, 124 aa, Artificial Sequence
  CRT4 variant 1 heavy chain
MAQVQLQQSGAELMKPGASVKISCKATGYTFSSYWIEWVNRRPGHGLEWIGEILPGSGSTNYNEKFKGKATFTADTSSNTAYMQLSSLTSEDSAVYYCARGGDYDEEYYLMDYWGQGTTLTVSS

SEQ ID NO: 105 - protein, 109 aa, Artificial Sequence
  CRT4 variant 1 light chain
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLEIKRAA

SEQ ID NO: 106 - protein, 122 aa, Artificial Sequence
  CRT4 variant 2 heavy chain
MAQVQLQQSGAELMKPGASVKISCKATGYTFSSYWIEWVNRRPGHGLEWIGEILPGSGSTNYNEKFKGKATFTADTSSNTAYMQLSSLTSEDSAVYYCARGGDYDEEYYLMDYWGQGTTLTV

SEQ ID NO: 107 - protein, 110 aa, Artificial Sequence
  CRT4 variant 2 light chain
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLEIKRAAA

SEQ ID NO: 108 - DNA, 372 bp, Artificial Sequence
  CRT4 variant 1 heavy chain
atggcccaggttcagctgcagcagtctggagctgagctgatgaagcctggggcctcagtgaagatatcctgcaaggctactggctacacattcagtagctactggatagagtgggtaaaccggaggcctggacatggccttgagtggattggagagattttacctggaagtggtagtactaattacaatgagaagttcaagggcaaggccacattcactgcagatacatcctccaatacagcctacatgcaactcagcagcctcacatctgaggactctgccgtctattactgtgcgagagggggggattacgacgaagaatactatctcatggactactggggtcaaggcaccactctcacagtctcctca

SEQ ID NO: 109 - DNA, 329 bp, Artificial Sequence
  CRT4 variant 1 light chain nucleotide sequence
caaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggaaatcaaacgtgcggccgc

SEQ ID NO: 110 - DNA, 366 bp, Artificial Sequence
  CRT4 variant 2 heavy chain nucleotide sequence
atggcccaggttcagctgcagcagtctggagctgagctgatgaagcctggggcctcagtgaagatatcctgcaaggctactggctacacattcagtagctactggatagagtgggtaaaccggaggcctggacatggccttgagtggattggagagattttacctggaagtggtagtactaattacaatgagaagttcaagggcaaggccacattcactgcagatacatcctccaatacagcctacatgcaactcagcagcctcacatctgaggactctgtcgtctattactgtgcgagagggggggattacgacgaagaatactatctcatggactactggggtcaaggcaccactctcacagtc

SEQ ID NO: 111 - DNA, 321 bp, Artificial Sequence
  CRT4 variant 2 light chain nucleotide sequence
caaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggaaatcaaacgt

SEQ ID NO: 112 - protein, 249 aa, Artificial Sequence
  CRT4 scFv
MAQVQLQQSGAELMKPGASVKISCKATGYTFSSYWIEWVNRRPGHGLEWIGEILPGSGSTNYNEKFKGKATFTADTSSNTAYMQLSSLTSEDSVVYYCARGGDYDEEYYLMDYWGQGTTLTVSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLEIKRAAA

SEQ ID NO: 113 - DNA, 747 bp, Artificial Sequence
  CRT4 scFv nucleotide sequence
atggcccaggttcagctgcagcagtctggagctgagctgatgaagcctggggcctcagtgaagatatcctgcaaggctactggctacacattcagtagctactggatagagtgggtaaaccggaggcctggacatggccttgagtggattggagagattttacctggaagtggtagtactaattacaatgagaagttcaagggcaaggccacattcactgcagatacatcctccaatacagcctacatgcaactcagcagcctcacatctgaggactctgtcgtctattactgtgcgagagggggggattacgacgaagaatactatctcatggactactggggtcaaggcaccactctcacagtctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggaaatcaaacgtgcggccgca

SEQ ID NO: 114 - protein, 493 aa, Artificial Sequence
  CRT4 CAR4
MGVLLTQRTLLSLVLALLFPSMASMAQVQLQQSGAELMKPGASVKISCKATGYTFSSYWIEWVNRRPGHGLEWIGEILPGSGSTNYNEKFKGKATFTADTSSNTAYMQLSSLTSEDSVVYYCARGGDYDEEYYLMDYWGQGTTLTVSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 115 - DNA, 1413 bp, Artificial Sequence
  CRT4 CAR4 nucleotide sequence
atggcccaggttcagctgcagcagtctggagctgagctgatgaagcctggggcctcagtgaagatatcctgcaaggctactggctacacattcagtagctactggatagagtgggtaaaccggaggcctggacatggccttgagtggattggagagattttacctggaagtggtagtactaattacaatgagaagttcaagggcaaggccacattcactgcagatacatcctccaatacagcctacatgcaactcagcagcctcacatctgaggactctgtcgtctattactgtgcgagagggggggattacgacgaagaatactatctcatggactactggggtcaaggcaccactctcacagtctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggaaatcaaacgtgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataa

SEQ ID NO: 116 - protein, 14 aa, Artificial Sequence
  CDR3 of heavy chain variant 1 of CRT5
ARGGDYDEEYYAMD

SEQ ID NO: 117 - DNA, 42 bp, Artificial Sequence
  CDR3 heavy chain variant 1 of CRT5
gcaagagggggggattacgacgaagaatactatgctatggac

SEQ ID NO: 118 - protein, 15 aa, Artificial Sequence
  CDR3 of heavy chain variant 2 of CRT5
ARGGDYDEEYYAMDY

SEQ ID NO: 119 - DNA, 72 bp, Artificial Sequence
  Nucleotide sequence for CD8alpha transmembrane domain
atctacatctgggcgcccttggccgggacttgtggggtccttctcctgtcactggttatcaccctttactgc

SEQ ID NO: 120 - DNA, 45 bp, Artificial Sequence
  CDR3 of heavy chain variant 2 of CRT5
gcaagagggggggattacgacgaagaatactatgctatggactac

SEQ ID NO: 121 - protein, 122 aa, Artificial Sequence
  CRT5 heavy chain
MAEVQLQQSGAELMKPGASVKISCKATGYTFSSYWIEWVNQRPGHGLEWIGEILPGSGSTNYNEKFKGKATFTADTSSNTAYMQLSSLTSEDSAVYYCARGGDYDEEYYAMDYWGQGTSVTL

SEQ ID NO: 122 - protein, 107 aa, Artificial Sequence
  CRT5 light chain
QIVLTQSPAIMSASPGEKVTMTCSASSSVSYMYWYQQKPGSSPRLLIYDTSNLASGVPVRFSGSGSGTSYSLTISRMEAEDGATYYCQQWSSYPLTFGAGTKLELKR

SEQ ID NO: 123 - DNA, 366 bp, Artificial Sequence
  Nucleotide sequence for CRT5 heavy chain
atggccgaggttcagcttcagcagtctggagctgagctgatgaagcctggggcctcagtgaagatatcctgcaaggctactggctacacattcagtagctactggatagagtgggtaaatcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgctatggactactggggtcaaggaacctcagtcaccctc

SEQ ID NO: 124 - DNA, 321 bp, Artificial Sequence; identical to SEQ ID NO: 60
  Nucleotide sequence of CRT5 light chain

SEQ ID NO: 125 - protein, 249 aa, Artificial Sequence
  CRT5 scFv
MAEVQLQQSGAELMKPGASVKISCKATGYTFSSYWIEWVNQRPGHGLEWIGEILPGSGSTNYNEKFKGKATFTADTSSNTAYMQLSSLTSEDSAVYYCARGGDYDEEYYAMDYWGQGTSVTLSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRAAA

SEQ ID NO: 126 - DNA, 747 bp, Artificial Sequence
  Nucleotide sequence of CRT5 scFv
atggccgaggttcagcttcagcagtctggagctgagctgatgaagcctggggcctcagtgaagatatcctgcaaggctactggctacacattcagtagctactggatagagtgggtaaatcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgctatggactactggggtcaaggaacctcagtcaccctctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgtgcggccgca

SEQ ID NO: 127 - protein, 493 aa, Artificial Sequence
  CAR5 CRT5 (oncostatin leader, scFv CRT5, CD28-CD3 portion)
MGVLLTQRTLLSLVLALLFPSMASMAEVQLQQSGAELMKPGASVKISCKATGYTFSSYWIEWVNQRPGHGLEWIGEILPGSGSTNYNEKFKGKATFTADTSSNTAYMQLSSLTSEDSAVYYCARGGDYDEEYYAMDYWGQGTSVTLSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 128 - DNA, 1502 bp, Artificial Sequence
  Nucleotide sequence for CAr5 CRT5
atgggcgtgctgctgacccagaggaccctgctgagcctggtgctggccctgctgtttccatctatggcatcgatggccgaggttcagcttcagcagtctggagctgagctgatgaagcctggggcctcagtgaagatatcctgcaaggctactggctacacattcagtagctactggatagagtgggtaaatcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgctatggactactggggtcaaggaacctcagtcaccctctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgtgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataaaagcttaacacgagcca

SEQ ID NO: 129 - protein, 6 aa, Artificial Sequence
  CDR1 of light chain of CRT2
SYMHWF

SEQ ID NO: 130 - protein, 8 aa, Artificial Sequence
  CDR3 of light chain of CRT2
QQRSSYPL

SEQ ID NO: 131 - DNA, 18 bp, Artificial Sequence
  CDR1 of light chain of CRT2
agttacatgcactggttc

SEQ ID NO: 132 - DNA, 24 bp, Artificial Sequence
  CDR3 of light chain of CRT2
cagcaaaggagtagttaccccctc

SEQ ID NO: 133 - protein, 109 aa, Artificial Sequence
  CRT2 light chain
EIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAPGKLELKRAA

SEQ ID NO: 134 - DNA, 329 bp, Artificial Sequence
  Nucleotide sequence of CRT2 light chain
gaaattgttctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttaccccctcacgttcggtgctgggaccaagctggagctgaaacgtgcggccgc

SEQ ID NO: 135 - protein, 24 aa, Artificial Sequence
  Oncostatin M leader
MGVLLTQRTLLSLVLALLFPSMAS

SEQ ID NO: 136 - protein, 836 aa, Artificial Sequence
  CAR5 CRT5 with truncated CD34
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMGVLLTQRTLLSLVLALLFPSMASMAEVQLQQSGAELMKPGASVKISCKATGYTFSSYWIEWVNQRPGHGLEWIGEILPGSGSTNYNEKFKGKATFTADTSSNTAYMQLSSLTSEDSAVYYCARGGDYDEEYYAMDYWGQGTSVTLSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 137 - DNA, 2514 bp, Artificial Sequence
  Nucleotide sequence of CAR5-CRT5 with truncated CD34
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcctgtcgaccgagtgaagcagaccctgaactttgatctgctgaagctggccggcgacgtggagtccaaccccgggccagggaatatgggcgtgctgctgacccagaggaccctgctgagcctggtgctggccctgctgtttccatctatggcatcgatggccgaggttcagcttcagcagtctggagctgagctgatgaagcctggggcctcagtgaagatatcctgcaaggctactggctacacattcagtagctactggatagagtgggtaaatcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgctatggactactggggtcaaggaacctcagtcaccctctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgtgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataa

SEQ ID NO: 138 - protein, 836 aa, Artificial Sequence
  CAR1 CRT1 with truncated CD34
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMGVLLTQRTLLSLVLALLFPSMASMAEVQLQQSGAELMKPGASVKISCKATGYTFSSYWIEWVKQRPGHGLEWIGEILPGSGSTNYNEKFKGKATFTADTSSNTAYMQLSSLTSEDSAVYYCARGGDYDEEYYVMDYWGQGTSVTVSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 139 - DNA, 2515 bp, Artificial Sequence
  Nucleotide sequence of CAR1 CRT1 with truncated CD34
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcctgtcgaccgagtgaagcagaccctgaactttgatctgctgaagctggccggcgacgtggagtccaaccccgggccagggaatatgggcgtgctgctgacccagaggaccctgctgagcctggtgctggccctgctgtttccatctatggcatcgatggccgaggttcagcttcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaattacaatgagaagttcaagggcaaggccacattcactgcagatacatcctccaacacagcctacatgcaactcagcagcctgacatctgaggactctgccgtctattactgtgcaagagggggggattacgacgaagaatactatgtcatggactactggggtcaaggaacctcagtcactgtctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgtgcggccgcaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataa

SEQ ID NO: 140 - protein, 835 aa, Artificial Sequence
  CAR3 CRT3 with truncated CD34
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMGVLLTQRTLLSLVLALLFPSMASMAEVQLQQSGTVLARPGASVKMSCKASGYTFTSYWMHWVKQRPGQGLEWIGAIYPGNSDTSYNQKFKGKAKLTAVTSTSTAYMELSSLTNEDSAVFYCTHYYGSDYAMDYWGQGTSVTVSSGGGGSGGGGSGGGGSQIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 141 - DNA, 2512 bp, Artificial Sequence
  Nucleotide sequence of CAR3 CRT3 with truncated CD34
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcctgtcgaccgagtgaagcagaccctgaactttgatctgctgaagctggccggcgacgtggagtccaaccccgggccagggaatatgggcgtgctgctgacccagaggaccctgctgagcctggtgctggccctgctgtttccatctatggcatcgatggccgaggtccagctgcagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtcttttactgtacacattactacggtagtgactatgctatggactactggggtcaaggaacctcagtcactgtctcctcaggtggaggcggttcaggcggaggtggctctggcggtggcggatcgcaaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacggacgttcggtggaggcaccaagctggaaatcaaacgtgcggccgcaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataa

SEQ ID NO: 142 - protein, 836 aa, Artificial Sequence
  CAR4 CRT4 with truncated CD34
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMGVLLTQRTLLSLVLALLFPSMASMAQVQLQQSGAELMKPGASVKISCKATGYTFSSYWIEWVNRRPGHGLEWIGEILPGSGSTNYNEKFKGKATFTADTSSNTAYMQLSSLTSEDSVVYYCARGGDYDEEYYLMDYWGQGTTLTVSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 143 - DNA, 2515 bp, Artificial Sequence
  Nucleotide sequence for CAR4 CRT4 with truncated CD34
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcctgtcgaccgagtgaagcagaccctgaactttgatctgctgaagctggccggcgacgtggagtccaaccccgggccagggaatatgggcgtgctgctgacccagaggaccctgctgagcctggtgctggccctgctgtttccatctatggcatcgatggcccaggttcagctgcagcagtctggagctgagctgatgaagcctggggcctcagtgaagatatcctgcaaggctactggctacacattcagtagctactggatagagtgggtaaaccggaggcctggacatggccttgagtggattggagagattttacctggaagtggtagtactaattacaatgagaagttcaagggcaaggccacattcactgcagatacatcctccaatacagcctacatgcaactcagcagcctcacatctgaggactctgtcgtctattactgtgcgagagggggggattacgacgaagaatactatctcatggactactggggtcaaggcaccactctcacagtctcctcaggtggaggcggttcaggcggaggtggctctggcggtggcggatcgcaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggaaatcaaacgtgcggccgcaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataa

SEQ ID NO: 144 - protein, 367 aa, Artificial Sequence
  Truncated CD34 plus peptide 2A linker
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMGVLLTQRTLLSLVLALLFPSMAS

SEQ ID NO: 145 - DNA, 1101 bp, Artificial Sequence
  Nucleotide sequence for truncated CD34 plus peptide 2A
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcctgtcgaccgagtgaagcagaccctgaactttgatctgctgaagctggccggcgacgtggagtccaaccccgggccagggaatatgggcgtgctgctgacccagaggaccctgctgagcctggtgctggccctgctgtttccatctatggcatcg

SEQ ID NO: 146 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 147 - DNA, 322 bp, Homo sapiens
aattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 148 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 149 - DNA, 345 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataa

SEQ ID NO: 150 - protein, 6 aa, Artificial Sequence
  Consensus sequence for CRT1, 3, 4 and 5 variant 1 heavy chain CDR1
  X (1)..(1): X is S or T
  X (5)..(5): X is I or M
  X (6)..(6): X is E or H
XSYWXX

SEQ ID NO: 151 - protein, 8 aa, Artificial Sequence
  Consensus sequence for CRT1, 3, 4 and 5 variant 2 heavy chain CDR1
  X (5)..(5): X is S or T
GYTFXSYW

SEQ ID NO: 152 - protein, 13 aa, Artificial Sequence
  Consensus sequence for CRT1, 3, 4 and 5 variant 1 heavy chain CDR2
  X (4)..(4): X is E or A
  X (6)..(6): X is L or Y
  X (9)..(9): X is S or N
  X (10)..(10): X is G orS
  X (11)..(11): X is S or D
  X (13)..(13): X is N or S
WIGXIXPGXXXTX

SEQ ID NO: 153 - protein, 8 aa, Artificial Sequence
  Consensus sequence for CRT1, 3, 4 and 5 variant 2 heavy chain CDR2
  X (2)..(2): X is L or Y
  X (5)..(5): X is S or N
  X (6)..(6): X is G or S
  X (7)..(7): X is S or D
IXPGXXXT

SEQ ID NO: 154 - protein, 14 aa, Artificial Sequence
  Consensus sequence for CRT1, 3, 4 and 5 variant 1 heavy chain CDR3
  X (1)..(1): X is A or T
  X (2)..(2): X is R or H
  X (3)..(3): X is G or no amino acid
  X (4)..(4): X is G or no amino acid
  X (5)..(5): X is D or no amino acid
  X (7)..(7): X is D or Y
  X (8)..(8): X is E or G
  X (9)..(9): X is E or S
  X (10)..(10): X is Y or D
  X (12)..(12): X is V, A or L
XXXXXYXXXXYXMD

SEQ ID NO: 155 - protein, 15 aa, Artificial Sequence
  Consensus sequence for CRT1, 3, 4 and 5 variant 2 heavy chain CDR3
  X (1)..(1): X is A or T
  X (2)..(2): X is R or H
  X (3)..(3): X is G or no amino acid
  X (4)..(4): X is G or no amino acid
  X (5)..(5): X is D or no amino acid
  X (7)..(7): X is D or Y
  X (8)..(8): X is E or G
  X (9)..(9): X is E or S
  X (10)..(10): X is Y or D
  X (12)..(12): X is V, A or L
XXXXXYXXXXYXMDY

SEQ ID NO: 156 - protein, 8 aa, Artificial Sequence
  Consensus sequence for CRT1, 3, 4 and 5 variant 1 light chain CDR1
  X (1)..(1): X is S or no amino acid
  X (2)..(2): X is S or no amino acid
  X (5)..(5): X is M or L
  X (6)..(6): X is Y or H
XXSYXXWY

SEQ ID NO: 157 - protein, 7 aa, Artificial Sequence
  Consensus sequence for CRT1, 3, 4, and 5 variant 2 light chain CDR1
  X (5)..(5): X is Y or S
  X (6)..(6): X is S or no amino acid
  X (7)..(7): X is Y or no amino acid
SSVSXXX

SEQ ID NO: 158 - protein, 10 aa, Artificial Sequence
  Consensus sequence for CRT1, 3, 4 and 5 variant 1 light chain CDR2
  X (2)..(2): X is L or W
  X (5)..(5): X is D or S
LXIYXTSNLA

SEQ ID NO: 159 - protein, 4 aa, Artificial Sequence
  Linker sequence
KDPK

SEQ ID NO: 160 - protein, 8 aa, Artificial Sequence
  Consensus sequence for CRT1, 3, 4 and 5 variant 1 light chain CDR3
  X (1)..(1): X is Q or H
  X (3)..(3): X is W or Y
  X (4)..(4): X is S or H
  X (5)..(5): X is S or R
  X (6)..(6): X is Y or S
  X (8)..(8): X is L or R
XQXXXXPX

SEQ ID NO: 161 - protein, 10 aa, Artificial Sequence
  Consensus sequence for CRT1, 3, 4 and 5 variant 2 light chain CDR3
  X (1)..(1): X is Q or H
  X (3)..(3): X is W or Y
  X (4)..(4): X is S or H
  X (5)..(5): X is S or R
  X (6)..(6): X is Y or S
  X (8)..(8): X is L or R
  X (10)..(10): X is F or no amino acid
XQXXXXPXTX

SEQ ID NO: 162 - DNA, 63 bp, Artificial Sequence
  Nucleotide sequence of CD8alpha leader
atggccttaccagtgaccgccttgctcctgccgctggccttgctgctccacgccgccaggccg

SEQ ID NO: 163 - protein, 239 aa, Artificial Sequence
  CH2CH3 hinge
DPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 164 - DNA, 703 bp, Artificial Sequence
  Nucleotide sequence for CH2CH3 hinge
aaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaagggccggccgct

SEQ ID NO: 165 - protein, 55 aa, Artificial Sequence
  CD8alpha hinge
FVPVFLPAKPTTTPAPRPPTPAPTIASQPLSLRPEACRPAAGGAVHTRGLDFACD

SEQ ID NO: 166 - protein, 13 aa, Artificial Sequence
  Shortened IgG hinge
AEPKSPDKTHTCP

SEQ ID NO: 167 - protein, 8 aa, Artificial Sequence
  CDR1 of heavy chain variable region of CRT2
GFTFNTYA

SEQ ID NO: 168 - protein, 10 aa, Artificial Sequence
  CDR2 of heavy chain variable region of CRT2
IRSKSNNYAT

SEQ ID NO: 169 - protein, 18 aa, Artificial Sequence
  CDR3 of heavy chain variable region of CRT2
VREGVYYYGSSGYYAMDY

SEQ ID NO: 170 - DNA, 24 bp, Artificial Sequence
  Nucleotide sequence encoding CDR1 of heavy chain of CRT2
ggtttcaccttcaatacctatgcc

SEQ ID NO: 171 - DNA, 30 bp, Artificial Sequence
  Nucleotide sequence encoding CDR2 of CRT2
ataagaagtaaaagtaataattatgcaaca

SEQ ID NO: 172 - DNA, 54 bp, Artificial Sequence
  Nucleotide sequence encoding CDR3 of CRT
gtgagagaaggggtttattactacggtagtagtgggtactatgctatggactac

SEQ ID NO: 173 - protein, 130 aa, Artificial Sequence
  Heavy chain variable region of CRT2
MAEVQGVESGGGLVQPKGSLKLSCAASGFTFNTYAMHWVCQAPGKGLEWVARIRSKSNNYATYYADSVKDRFTISRDDSQSMLYLQMNNLKTEDTAMYYCVREGVYYYGSSGYYAMDYWGQGTSVTVSSG

SEQ ID NO: 174 - DNA, 401 bp, Artificial Sequence
  Nucleotide sequence encoding CRT2 heavy chain variable region
gacgcttatcgatggccgaggtgcagggggtggagtctggtggaggattggtgcagcctaaaggatcattgaaactctcatgtgccgcctctggtttcaccttcaatacctatgccatgcactgggtctgccaggctccaggaaagggtttggaatgggttgctcgcataagaagtaaaagtaataattatgcaacatattatgccgattcagtgaaagacagattcaccatctccagagatgattcacaaagcatgctctatctgcaaatgaacaacctgaaaactgaggacacagccatgtattactgtgtgagagaaggggtttattactacggtagtagtgggtactatgctatggactactggggtcaaggaacctcagtcaccgtctcctcaggt

SEQ ID NO: 175 - protein, 254 aa, Artificial Sequence
  scFv of CRT2
MAEVQGVESGGGLVQPKGSLKLSCAASGFTFNTYAMHWVCQAPGKGLEWVARIRSKSNNYATYYADSVKDRFTISRDDSQSMLYLQMNNLKTEDTAMYYCVREGVYYYGSSGYYAMDYWGQGTSVTVSSGGGGGSGGGGSGGGGSEIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAPGKLELKRAA

SEQ ID NO: 176 - DNA, 781 bp, Artificial Sequence
  CRT2 scFv nucleotide
gacgcttatcgatggccgaggtgcagggggtggagtctggtggaggattggtgcagcctaaaggatcattgaaactctcatgtgccgcctctggtttcaccttcaatacctatgccatgcactgggtctgccaggctccaggaaagggtttggaatgggttgctcgcataagaagtaaaagtaataattatgcaacatattatgccgattcagtgaaagacagattcaccatctccagagatgattcacaaagcatgctctatctgcaaatgaacaacctgaaaactgaggacacagccatgtattactgtgtgagagaaggggtttattactacggtagtagtgggtactatgctatggactactggggtcaaggaacctcagtcaccgtctcctcaggttcctcaggtggaggcggttcaggcggaggtggctctggcggtggcggatcggaaattgttctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttaccccctcacgttcggtgctgggaccaagctggagctgaaacgtgcggccgc

SEQ ID NO: 177 - DNA, 747 bp, Artificial Sequence
  CRT1 scFv human codon optimised sequence
atggcagaggtgcagctgcagcagagcggagcagagctgatgaagccaggagcctctgtgaagatcagctgtaaggccaccggctatacattcagctcctactggatcgagtgggtgaagcagcggcctggccacggcctggagtggatcggagagatcctgccaggcagcggctccaccaactataatgagaagttcaagggcaaggccacctttacagccgacacctctagcaacacagcctacatgcagctgtcctctctgacaagcgaggattccgccgtgtactattgcgccaggggcggcgactatgatgaggagtactatgtgatggactactggggccagggcacctccgtgaccgtgagcagcggcggaggcggcagcggaggaggaggctccggcggcggcggctctcagatcgtgctgacccagagcccagcaatcatgtctgccagcccaggagagaaggtgaccatgacatgttccgcctctagctccgtgagctacatgtattggtatcagcagaagcccggctctagccctcggctgctgatctatagaacctccaatctggcatctggcgtgcccgcaaggttctccggctctggcagcggcacctcctactctctgaccatcggcacaatggaggccgaggatgccgccacatactattgccagcagtggtcctcttaccctctgacctttggcgccggcacaaagctggagatcaagcgcgcggccgca

SEQ ID NO: 178 - DNA, 747 bp, Artificial Sequence
  CRT1 scFv codon optimised murine sequence
atggctgaggtgcagctgcagcagtccggagctgagctgatgaagccaggcgcctctgtgaagatcagctgtaaggccaccggctacacattcagctcctactggatcgagtgggtgaagcagaggcctggccacggactggagtggatcggagagatcctgccaggcagcggcagcaccaactacaacgagaagttcaagggcaaggctacctttacagccgacacctctagcaacacagcttacatgcagctgtcctctctgacaagcgaggatagcgccgtgtactactgcgccaggggcggagactacgatgaggagtactacgtgatggactactggggccagggaacctctgtgaccgtgagcagcggaggaggaggaagcggcggaggaggcagcggaggaggaggatctcagatcgtgctgacccagagcccagctatcatgtctgccagccccggcgagaaggtgaccatgacatgtagcgcctctagctccgtgtcctacatgtactggtatcagcagaagcccggatctagccctaggctgctgatctacagaacatccaacctggcttctggcgtgcccgctcggttctccggctctggaagcggcacctcctactctctgaccatcggcacaatggaggctgaggatgccgctacatactactgccagcagtggtcctcttaccctctgacctttggagccggcacaaagctggagatcaagcgcgcggccgca

SEQ ID NO: 179 - DNA, 762 bp, Artificial Sequence
  CRT2 scFV codon optimised human sequence
atggcagaggtgcagggagtggagagcggaggcggcctggtgcagcctaagggctccctgaagctgtcttgcgccgccagcggcttcacctttaacacatatgcaatgcactgggtgtgccaggcaccaggcaagggcctggagtgggtggcacggatcagaagcaagtccaacaattatgccacctactatgccgacagcgtgaaggataggttcacaatctcccgcgacgattctcagagcatgctgtacctgcagatgaacaatctgaagaccgaggacacagccatgtactattgcgtgcgggagggcgtgtactattacggcagctccggctattacgctatggactactggggccagggcaccagcgtgacagtgtctagcggaggaggaggctccggaggaggaggctctggcggcggcggcagcgagatcgtgctgacccagtccccagcaatcatgtccgcctctccaggagagaaggtgaccatcacatgctccgcctcctctagcgtgtcttatatgcactggttccagcagaagcccggcacctctcctaagctgtggatctacagcacatccaatctggcatccggcgtgcccgcaaggttttctggcagcggctccggcacctcttatagcctgacaatcagccggatggaggcagaggacgcagcaacctattactgtcagcagagatcctcttaccctctgacctttggcgccggcacaaagctggagctgaagcgcgcggccgca

SEQ ID NO: 180 - DNA, 762 bp, Artificial Sequence
  CRT2 scFv codon optimised human sequence
atggctgaggtgcagggagtggagagcggaggaggcctggtgcagcctaagggctccctgaagctgtcttgcgccgctagcggattcacctttaacacatacgctatgcactgggtgtgccaggctccaggaaagggcctggagtgggtggccaggatcagaagcaagtccaacaactacgctacctactacgccgacagcgtgaaggatcggttcacaatctcccgcgacgattctcagagcatgctgtacctgcagatgaacaacctgaagaccgaggacacagctatgtactactgcgtgcgggagggcgtgtactactacggcagctccggatactacgctatggactactggggacagggcacctccgtgacagtgtctagcggaggaggaggctccggaggaggaggctctggaggcggaggcagcgagatcgtgctgacccagtctccagctatcatgtccgcctctcccggcgagaaggtgaccatcacatgctccgcctcctctagcgtgtcttacatgcactggttccagcagaagcccggcacctctcctaagctgtggatctacagcacatccaacctggctagcggagtgcccgctcggttttctggaagcggctccggaacctcttacagcctgacaatctccaggatggaggctgaggacgccgctacatactactgtcagcagagatcctcttaccctctgacctttggcgccggaacaaagctggagctgaagcgcgcggccgca

SEQ ID NO: 181 - DNA, 744 bp, Artificial Sequence
  CRT3 scFv codon optimised human sequence
atggccgaggtgcagctgcagcagtctggcaccgtgctggccaggcccggagcaagcgtgaagatgtcctgcaaggcctctggctacaccttcacaagctattggatgcactgggtgaagcagcgcccaggacagggcctggagtggatcggagcaatctaccccggcaactccgacacctcttataatcagaagttcaagggcaaggccaagctgacagccgtgacctctacaagcaccgcctacatggagctgagcagcctgaccaacgaggatagcgccgtgttttattgcacacactactatggctccgactacgctatggactattggggccagggcacctccgtgacagtgtctagcggaggaggaggcagcggaggaggaggctccggcggcggcggctctcagatcgtgctgacccagagccctgccatcatgtccgcctctctgggcgagcgggtgacaatgacctgtacagcctcctctagcgtgtcctctagctacctgcactggtatcagcagaagcccggctcctctcctaagctgtggatctacagcacctccaatctggcatccggcgtgcctgcaaggttctctggcagcggctccggcacctcttacagcctgacaatcagcagcatggaggcagaggacgcagcaacatactattgtcaccagtatcaccggagcccaagaacctttggcggcggcacaaagctggagatcaagcgggcggccgca

SEQ ID NO: 182 - DNA, 744 bp, Artificial Sequence
  CRT3 scFv codon optimised murine sequence
atggccgaggtgcagctgcagcagtctggcaccgtgctggctcggcccggagctagcgtgaagatgtcctgcaaggcttctggctacaccttcacaagctactggatgcactgggtgaagcagcgcccaggacagggcctggagtggatcggcgccatctaccccggaaactccgacacctcttacaaccagaagttcaagggcaaggctaagctgacagccgtgacctctacaagcaccgcttacatggagctgagcagcctgaccaacgaggatagcgccgtgttttactgcacacactactacggctccgactacgctatggattactggggacagggcacctccgtgacagtgtctagcggaggaggaggaagcggcggaggcggcagcggaggaggaggatctcagatcgtgctgacccagtctcctgctatcatgtccgcctctctgggcgagagggtgacaatgacctgtacagcctcctctagcgtgtcctctagctacctgcactggtatcagcagaagcccggctcctctcctaagctgtggatctacagcacctccaacctggcttccggagtgcctgctcggttctctggaagcggctccggaacctcttacagcctgacaatcagcagcatggaggctgaggacgccgctacatactactgtcaccagtaccacaggagcccaagaacctttggcggaggcacaaagctggagatcaagagggcggccgca

SEQ ID NO: 183 - DNA, 747 bp, Artificial Sequence
  CRT4 scFv codon optimised human sequence
atggcacaggtgcagctgcagcagagcggagcagagctgatgaagccaggagcctctgtgaagatcagctgtaaggccaccggctatacattcagctcctactggatcgagtgggtgaacagacggcccggccacggcctggagtggatcggagagatcctgccaggcagcggctccaccaactataatgagaagttcaagggcaaggccacctttacagccgacacctctagcaatacagcctacatgcagctgtcctctctgacaagcgaggattccgtggtgtactattgcgccaggggcggcgactatgatgaggagtactatctgatggactactggggccagggcaccacactgaccgtgagcagcggaggaggaggcagcggaggaggaggctccggcggcggcggctctcagatcgtgctgacacagtccccagcaatcatgtctgccagcccaggagagaaggtgaccatgacatgttccgcctctagctccgtgagctacatgtattggtatcagcagaagcccggctctagccctaggctgctgatctatgacacctccaacctggcatctggcgtgcccgtgcgcttctccggctctggcagcggcacctcctactctctgacaatcagccggatggaggcagaggatgcagcaacctactattgccagcagtggtcctcttaccctctgacctttggcgccggcacaaagctggagatcaagcgggcggccgca

SEQ ID NO: 184 - DNA, 747 bp, Artificial Sequence
  CRT4 scFv codon optimised murine sequence
atggctcaggtgcagctgcagcagtccggagctgagctgatgaagccaggcgcctctgtgaagatcagctgtaaggccaccggctacacattcagctcctactggatcgagtgggtgaacaggaggcccggccacggactggagtggatcggagagatcctgccaggcagcggcagcaccaactacaacgagaagttcaagggcaaggctacctttacagccgacacctctagcaacacagcttacatgcagctgtcctctctgacaagcgaggattccgtggtgtactactgcgccaggggcggagactacgatgaggagtactacctgatggactactggggccagggaaccacactgaccgtgagcagcggaggaggaggaagcggcggaggaggcagcggaggaggaggatctcagatcgtgctgacacagtctccagctatcatgtctgccagccccggcgagaaggtgaccatgacatgtagcgccagcagcagcgtgagctacatgtactggtatcagcagaagcccggatctagccctcggctgctgatctacgacacctccaacctggcttctggcgtgcccgtgcgcttctccggctctggaagcggcacctcctactctctgacaatcagcaggatggaggctgaggatgccgctacatactactgccagcagtggtcctcttaccctctgacctttggagccggcacaaagctggagatcaagagggcggccgca

SEQ ID NO: 185 - DNA, 747 bp, Artificial Sequence
  CRT5 scFv codon optimised human sequence
atggcagaggtgcagctgcagcagtccggagcagagctgatgaagccaggagcctctgtgaagatcagctgtaaggccaccggctatacattcagctcctactggatcgagtgggtgaaccagcgccctggccacggcctggagtggatcggagagatcctgccaggcagcggctccaccaactataatgagaagttcaagggcaaggccacctttacagccgacacctctagcaatacagcctacatgcagctgtcctctctgacaagcgaggattccgccgtgtactattgcgccagaggcggcgactatgatgaggagtactatgctatggactactggggccagggcacctctgtgaccctgagcagcggaggaggaggcagcggcggaggaggctccggcggcggcggctctcagatcgtgctgacccagagcccagcaatcatgtctgccagcccaggagagaaggtgaccatgacatgtagcgcctctagctccgtgtcctacatgtattggtatcagcagaagcccggctctagccctcggctgctgatctatgacacctccaacctggcctctggcgtgcccgtgagattctccggctctggcagcggcacctcctactctctgacaatcagcaggatggaggccgaggatgccgccacatactattgccagcagtggtcctcttaccctctgacctttggcgccggcacaaagctggagctgaagagggcggccgca

SEQ ID NO: 186 - DNA, 747 bp, Artificial Sequence
  CRT5 scFv codon optimised murine sequence
atggctgaggtgcagctgcagcagtccggagctgagctgatgaagccaggcgcctctgtgaagatcagctgtaaggccaccggctacacattcagctcctactggatcgagtgggtgaaccagcgccctggccacggactggagtggatcggagagatcctgccaggcagcggcagcaccaactacaacgagaagttcaagggcaaggctacctttacagccgacacctctagcaacacagcttacatgcagctgtcctctctgacaagcgaggatagcgccgtgtactactgcgccaggggcggagactacgatgaggagtactacgctatggactactggggccagggaacctctgtgaccctgagcagcggaggaggaggaagcggcggaggaggcagcggaggaggaggatctcagatcgtgctgacccagagcccagctatcatgtctgccagccccggcgagaaggtgaccatgacatgtagcgccagcagcagcgtgagctacatgtactggtatcagcagaagcccggatctagccctaggctgctgatctacgacacctccaacctggcctctggcgtgcccgtgagattctccggctctggaagcggcacctcctactctctgacaatcagccggatggaggctgaggatgccgctacatactactgccagcagtggtcctcttaccctctgacctttggagccggcacaaagctggagctgaagcgggcggccgca

SEQ ID NO: 187 - protein, 141 aa, Artificial Sequence
  Human TCR alpha chain constant region
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 188 - protein, 179 aa, Artificial Sequence
  Human TCR beta chain constant region
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 189 - protein, 9 aa, Artificial Sequence
  WT1 epitope
RMFPNAPYL

SEQ ID NO: 190 - protein, 6 aa, Artificial Sequence
  CDR1 of WT1 TCR alpha chain
SSYSPS

SEQ ID NO: 191 - protein, 7 aa, Artificial Sequence
  CDR2 of WT1 TCR alpha chain
YTSAATL

SEQ ID NO: 192 - protein, 14 aa, Artificial Sequence
  CDR3 of WT1 TCR alpha chain
VVSPFSGGGADGLT

SEQ ID NO: 193 - protein, 12 aa, Artificial Sequence
  CDR3 of WT1 TCR alpha chain
SPFSGGGADGLT

SEQ ID NO: 194 - protein, 6 aa, Artificial Sequence
  CDR1 of WT1 TCR beta chain
DFQATT

SEQ ID NO: 195 - protein, 7 aa, Artificial Sequence
  CDR2 of WT1 TCR beta chain
SNEGSKA

SEQ ID NO: 196 - protein, 8 aa, Artificial Sequence
  CDR3 of WT1 TCR beta chain
SARDGGEG

SEQ ID NO: 197 - protein, 11 aa, Artificial Sequence
  CDR3 of WT1 TCR beta chain
RDGGEGSETQY

SEQ ID NO: 198 - protein, 276 aa, Artificial Sequence
  Human TCR alpha chain WT1
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSPFSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 199 - protein, 310 aa, Artificial Sequence
  Human TCR beta chain WT1
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDGGEGSETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 200 - protein, 276 aa, Artificial Sequence
  Human TCR alpha chain (WT1) with Thr 48 to Cys substitution
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSPFSGGGADGLTFGKGTHLIIQPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 201 - protein, 310 aa, Artificial Sequence
  Human TCR beta chain (WT1) with Ser 57 to Cys substitution
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDGGEGSETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 202 - protein, 9 aa, Artificial Sequence
  WT1 epitope 235-243
CMTWNQMNL

SEQ ID NO: 203 - protein, 11 aa, Artificial Sequence
  Framework region
FGKGTHLIIQP

SEQ ID NO: 204 - protein, 10 aa, Artificial Sequence
  Framework region
FGPGTRLLVL

SEQ ID NO: 205 - protein, 14 aa, Artificial Sequence
  Consensus sequence of CRT5, 1 and 4 variant 1 heavy chain CDR3
  X (12)..(12): X is A or V or L
ARGGDYDEEYYXMD

SEQ ID NO: 206 - protein, 15 aa, Artificial Sequence
  Consensus sequence of CRT5, 1 and 4 variant 2 heavy chain CDR3
  Xaa (12)..(12): Xaa is A or V or L
ARGGDYDEEYYXMDY

SEQ ID NO: 207 - protein, 15 aa, Artificial Sequence
  Consensus sequence of CRT5, 1 and 4 variants 1 and 2 heavy chain CDR3
  Xaa (12)..(12): Xaa is A or V or L
  Xaa (15)..(15): Xaa is Y or no amino acid
ARGGDYDEEYYXMDX

SEQ ID NO: 208 - protein, 9 aa, Artificial Sequence
  Consensus of CRT5, 1 and 4 variants 1 and 2 light chain CDR3
  Xaa (9)..(9): Xaa is T or no amino acid
QQWSSYPLX

SEQ ID NO: 209 - protein, 6 aa, Artificial Sequence
  Consensus of CRT5, 1 and 4 variants 1 and 2 heavy chain CDR1
  Xaa (1)..(1): Xaa is GYTF or no amino acid
  Xaa (6)..(6): Xaa is IE or no amino acid
XSSYWX

SEQ ID NO: 210 - protein, 10 aa, Artificial Sequence
  Consensus of CRT5, 1 and 4 variants 1 and 2 heavy chain CDR2
  Xaa (1)..(1): Xaa is WIGE or no amino acid
  Xaa (10)..(10): Xaa is N or no amino acid
XILPGSGSTX

SEQ ID NO: 211 - protein, 6 aa, Artificial Sequence
  Consensus sequenc of CRT5, 1, 4 and 3 variants 1 and 2 heavy chains CDR1
  Xaa (1)..(1): Xaa is GYTF or no amino acid
  Xaa (2)..(2): Xaa is S or T
  Xaa (6)..(6): Xaa is I or M and E or H, or no amino acid
XXSYWX

SEQ ID NO: 212 - protein, 10 aa, Artificial Sequence
  Consensus of CRT5, 1, 4 and 3 variants 1 and 2 heavy chain CDR2
  Xaa (1)..(1): Xaa is WIG and E or A, or no amino acid
  Xaa (3)..(3): Xaa is L or Y
  Xaa (6)..(6): Xaa is S or N
  Xaa (7)..(7): Xaa is G or S
  Xaa (8)..(8): Xaa is S or D
  Xaa (10)..(10): Xaa is N or S or no amino acid
XIXPGXXXTX

SEQ ID NO: 213 - protein, 15 aa, Artificial Sequence
  Consensus of CRT5, 1, 4 and 3 variants 1 and 2 heavy chain CDR3
  Xaa (1)..(1): Xaa is A or T
  Xaa (2)..(2): Xaa is R or H
  Xaa (3)..(3): Xaa is G or no amino acid
  Xaa (4)..(4): Xaa is G or no amino acid
  Xaa (5)..(5): Xaa is D or no amino acid
  Xaa (7)..(7): Xaa is D or Y
  Xaa (8)..(8): Xaa is E or G
  Xaa (9)..(9): Xaa is E or S
  Xaa (10)..(10): Xaa is Y or D
  Xaa (12)..(12): Xaa is V or A or L
  Xaa (15)..(15): Xaa is Y or no amino acid
XXXXXYXXXXYXMDX

SEQ ID NO: 214 - protein, 5 aa, Artificial Sequence
  Consensus of CRT5, 1, 4 and 3 variants 1 and 2 light chain CDR2
  Xaa (1)..(1): Xaa is (L, L or W ,IY) or no amino acid
  Xaa (2)..(2): D or S
  Xaa (5)..(5): Xaa is NLA or no amino acid
XXTSX

SEQ ID NO: 215 - protein, 9 aa, Artificial Sequence
  Consensus of CRT5, 1, 4, and 3 variants 1 and 2 light chain CDR3
  Xaa (1)..(1): Xaa is Q or H
  Xaa (3)..(3): Xaa is W or Y
  Xaa (4)..(4): Xaa is S or H
  Xaa (5)..(5): Xaa is S or R
  Xaa (6)..(6): Xaa is Y or S
  Xaa (8)..(8): Xaa is L or R
  Xaa (9)..(9): Xaa is TF or no amino acid
XQXXXXPXX

SEQ ID NO: 216 - protein, 6 aa, Artificial Sequence
  Consensus of CRT3 variants 1 and 2 heavy chain CDR1
  Xaa (1)..(1): Xaa is GYTF or no amino acid
  Xaa (6)..(6): Xaa is MH or no amino acid
XTSYWX

SEQ ID NO: 217 - protein, 10 aa, Artificial Sequence
  Consensus of CRT3 variants 1 and 2 heavy chain CDR2
  Xaa (1)..(1): Xaa is WIGA or no amino acid
  Xaa (10)..(10): Xaa is S or no amino acid
XIYPGNSDTX

SEQ ID NO: 218 - protein, 12 aa, Artificial Sequence
  Consensus of CRT3 variants 1 and 2 heavy chain CDR3
  Xaa (12)..(12): Xaa is Y or no amino acid
THYYGSDYAMDX

SEQ ID NO: 219 - protein, 6 aa, Artificial Sequence
  Consensus of CRT3 variants 1 and 2 light chain CDR1
  Xaa (1)..(1): Xaa is SSV or no amino acid
  Xaa (6)..(6): Xaa is LHWY or no amino acid
XSSSYX

SEQ ID NO: 220 - protein, 5 aa, Artificial Sequence
  Consensus of CRT3 variants 1 and 2 light chain CDR2
  Xaa (1)..(1): Xaa is LWIY or no amino acid
  Xaa (5)..(5): Xaa is NLA or no amino acid
XSTSX

SEQ ID NO: 221 - protein, 9 aa, Artificial Sequence
  Consensus of CRT3 variants 1 and 2 light chain CDR3
  Xaa (9)..(9): Xaa is T or no amino acid
HQYHRSPRX

SEQ ID NO: 222 - DNA, 216 bp, Mus musculus
actactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccga

SEQ ID NO: 223 - DNA, 123 bp, Mus musculus
aatagtagaaggaacagactccttcaaagtgactacatgaacatgactccccggaggcctgggctcactcgaaagccttaccagccctacgcccctgccagagactttgcagcgtaccgcccc

SEQ ID NO: 224 - DNA, 135 bp, Mus musculus
aaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 225 - DNA, 334 bp, Mus musculus
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggccc

SEQ ID NO: 226 - DNA, 108 bp, Mus musculus
cggaaggcttggagattgcctaacactcccaaaccttgttggggaaacagcttcaggaccccgatccaggaggaacacacagacgcacactttactctggccaagatc

SEQ ID NO: 227 - DNA, 752 bp, Artificial Sequence
  Murine CD8 alpha hinge-CD8 alpha transmembrane domain- CD28 costimulatory domain-CD3 zeta intracellular signalling domain
ggaggcaccaagctggaaatcaaacgtgcggccgcaactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaataaaagcttaacacgagccatagatagaataaaag

SEQ ID NO: 228 - DNA, 764 bp, Artificial Sequence
  Murine CD8alpha hinge-CD8alpha transmembrane domain-41BB costimulatory domain- CD3 zeta intracellular signalling domain
ggaggcaccaagctggaaatcaaacgtgcggccgcaactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaataaaagcttaacacgagccatagatagaataaaag

SEQ ID NO: 229 - DNA, 737 bp, Artificial Sequence
  Murine CD8alpha hinge-CD8alpha transmembrane domain-OX40 costimulatory domain-CD3 zeta intracellular signalling domain
ggaggcaccaagctggaaatcaaacgtgcggccgcaactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgacggaaggcttggagattgcctaacactcccaaaccttgttggggaaacagcttcaggaccccgatccaggaggaacacacagacgcacactttactctggccaagatcagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaataaaagcttaacacgagccatagatagaataaaag

SEQ ID NO: 230 - DNA, 887 bp, Artificial Sequence
  Murine CD8alpha hinge-CD8alpha transmembrane domain-CD28 costimulatory domain- 41BB costimulatory domain-CD3 zeta intracellular signalling domain
ggaggcaccaagctggaaatcaaacgtgcggccgcaactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaatagtagaaggaacagactccttcaaagtgactacatgaacatgactccccggaggcctgggctcactcgaaagccttaccagccctacgcccctgccagagactttgcagcgtaccgccccaaatggatcaggaaaaaattcccccacatattcaagcaaccatttaagaagaccactggagcagctcaagaggaagatgcttgtagctgccgatgtccacaggaagaagaaggaggaggaggaggctatgagctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaataaaagcttaacacgagccatagatagaataaaag

SEQ ID NO: 231 - DNA, 860 bp, Artificial Sequence
  Murine CD8alpha hinge-CD8alpha transmembrane domain- CD28 and OX40 costimulatory domains- CD3 zeta intracellular signalling domain
ggaggcaccaagctggaaatcaaacgtgcggccgcaactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaatagtagaaggaacagactccttcaaagtgactacatgaacatgactccccggaggcctgggctcactcgaaagccttaccagccctacgcccctgccagagactttgcagcgtaccgcccccggaaggcttggagattgcctaacactcccaaaccttgttggggaaacagcttcaggaccccgatccaggaggaacacacagacgcacactttactctggccaagatcagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaataaaagcttaacacgagccatagatagaataaaag

SEQ ID NO: 232 - DNA, 872 bp, Artificial Sequence
  Murine CD8alpha hinge-CD8alpha transmembrane domain- 41BB and OX40 costimulatory domains-CD3 zeta intracellular signalling domain
ggaggcaccaagctggaaatcaaacgtgcggccgcaactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgcggaaggcttggagattgcctaacactcccaaaccttgttggggaaacagcttcaggaccccgatccaggaggaacacacagacgcacactttactctggccaagatcagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaataaaagcttaacacgagccatagatagaataaaag